(19) United States
(12) Patent Application Publication
(10) Pub. No.: US 2013/0237454 A1
(43) Pub. Date: Sep. 12, 2013

(54) DIAGNOSTIC MARKERS FOR NEUROPSYCHIATRIC DISEASE

(71) Applicant: Steven E. Schutzer, Water Mill, NY (US)

(72) Inventor: Steven E. Schutzer, Water Mill, NY (US)

(21) Appl. No.: 13/697,417

(22) PCT Filed: May 12, 2011
(86) PCT No.: PCT/US11/00843

§ 371 Date: May 23, 2013

Related U.S. Application Data
(60) Provisional application No. 61/395,354, filed on May 12, 2010.

(51) Int. Cl. G01N 33/68

(52) U.S. Cl. CPC G01N 33/6896
USPC 506/12

(57) ABSTRACT

Biomarkers for the diagnosis of neuropsychiatric diseases are presented herein. In particular embodiments, biomarkers are identified that are useful for diagnosing multiple sclerosis, chronic fatigue syndrome, or Neurologic Lyme disease. Also encompassed is a method for diagnosing a patient with a neuropsychiatric disease, such as multiple sclerosis, chronic fatigue syndrome, or Neurologic Lyme disease, by analyzing biological samples isolated from the patient or the patient as a whole to assess levels of the biomarkers described herein.

### FIELD OF THE INVENTION

The present invention relates to identifying biologic markers (biomarkers) that can be used for diagnosis of a neuropsychiatric disease. As described herein, neuropsychiatric disease may be used to refer to diseases having neurologic or psychiatric or combined neurologic and psychiatric features. Such neuropsychiatric diseases include, without limitation, multiple sclerosis, chronic fatigue syndrome, and Neurologic Lyme disease. In particular embodiments, the present invention relates to identifying biomarkers for diagnosis of multiple sclerosis, chronic fatigue syndrome (CFS; also referred to herein as CF), or Neurologic Lyme disease. In a more particular aspect, the invention pertains to the identification of a biomarker signature that is diagnostic for each of multiple sclerosis, chronic fatigue syndrome, Neurologic Lyme disease. Accordingly, the invention further relates to a method for diagnosing a person or other mammal with a neuropsychiatric disease, such as multiple sclerosis, or at risk of developing same. The biomarkers and diagnostic signatures described herein also identify metabolic/biochemical pathways as potential candidates for therapeutic targeting. The invention further relates to guidance pertaining to appropriate treatment of the person or mammal diagnosed with a particular neuropsychiatric disease in accordance the methods described herein. Accurate diagnosis of neuropsychiatric disease, particularly with respect to diseases that are difficult to distinguish clinically, should reduce the duration and/or severity of the disease by ensuring that the patient is treated using an appropriate therapeutic regimen.

### BACKGROUND OF THE INVENTION

Knowledge of the entire protein content, the proteome, of normal human cerebrospinal fluid (CSF) would provide a critical standard to allow meaningful comparisons with and between neurologic and psychiatric disorders. CSF contains both cellular and soluble components providing insights into processes occurring in the central nervous system (CNS). As much as 30 to 40% of CSF is formed by the extracellular fluid of the brain and spinal cord. CSF contains both normal and disease specific components, and provides an accessible liquid window into the brain. In fact, recent data suggest CSF may provide more relevant evidence for initial or propagating pathology than the brain parenchyma itself in certain neuropsychiatric diseases [Ransohoff (2009) Nature 457: 155-156]. Comprehensive characterization of the normal CSF proteome, furthermore, facilitates identification of disease-specific markers [Ekegren et al. (2008) J Mass Spectrom 43: 559-571]. Knowledge of which proteins are present, absent, or of changed concentrations may lead to diagnostic, prognostic, or disease-activity biomarkers as well as provide insights into disease etiology and pathogenesis. An advantage of a full proteome analysis is the ability to identify not just one but a multitude of proteins at a single instance.

Multiple sclerosis, for example, is an excellent candidate disease for such an approach because it is often a progressive debilitating disease that is difficult to diagnose definitively, especially during the early stages of the disease. Multiple sclerosis is a common demyelinating disease of the central nervous system (CNS) that affects up to 0.1% of the Caucasian population of northern European descent. Multiple sclerosis is more common in women than men and generally begins between ages 20 and 40, but can develop at any age. Multiple sclerosis is generally viewed as an autoimmune syndrome directed against unidentified central nervous tissue antigens. The determination of susceptibility to multiple sclerosis development is complex and appears to be governed by both environmental and genetic factors. Some of the symptoms of multiple sclerosis are caused by damage to the myelin sheath, the protective covering that surrounds nerve cells. When this nerve covering is damaged, nerve impulses are slowed down or stopped. Ultimately, damage to the myelin sheath results in nerve damage. Multiple sclerosis is, moreover, commonly a progressive disease that leads to more severe neurologic dysfunction over time. Nerve damage may be caused by inflammation that occurs when the body's immune cells attack the nervous system. Repeated episodes of inflammation can occur along any area of the brain and spinal cord, which is why the disease is often referred to as one characterized by symptoms and signs over time and space.

Multiple sclerosis is difficult to diagnose because the progress, severity and specific symptoms of multiple sclerosis are quite variable and unpredictable. There are no laboratory tests, symptoms or physical findings that can singly determine if a person has multiple sclerosis. The differential diagnosis of multiple sclerosis is quite varied and includes metabolic, genetic, oncologic, immunologic, and infectious disease assessment. Other diseases that may need to be considered in the differential diagnoses, but should be considered depending on the clinical presentation, include: Acute disseminated encephalomyelitis, CNS vasculitis, Behçet disease, Sjögren syndrome, Sarcoid, neoplasms, CADASIL (cerebral autosomal dominant arteriopathy with subcortical infarcts and leukoencephalopathy), Migrainous ischemia, Cerebrovascular disease, Progressive multifocal leukoencephalopathy, Inherited white matter diseases, effects of radiation therapy or drugs, CNS lymphoma, Lyme disease, HTLV-1 infection, CNS lupus, Mitochondrial encephalopathies, Antiphospholipid antibody syndrome, cerebral emboli, Thrombocytopenic purpura, Progressive multifocal leukoencephalopathy, Mycoplasma encephalopathy, Vitamin B12 deficiency, Paraneoplastic syndromes, Psychiatric syndromes (Rolak L A, Fleming J O. The Neurologist 2007; 13: 57-72). Over the last twenty years, tests such as magnetic resonance imaging (MRI), examination of CSF, and evoked response testing have played an increasingly important role in the diagnostic process. In 2005, revised McDonald criteria for multiple sclerosis were published (Polman et al. Diagnostic Criteria for Multiple Sclerosis: 2005 Revisions to the “McDonald” Criteria. Ann Neurol. (2005) 58:840-846 and Polman et al. Ann Neurol (2011) 69:292-302). In addition to the traditional diagnostic tools, the revised criteria provide specific guidelines for using findings of MRI, cerebrospinal fluid analysis and visual evoked potentials to support a diagnosis of multiple sclerosis. However, even with these revised criteria, diagnosis of multiple sclerosis is still challenging and frequently takes several months or even years.

Rendering a conclusive diagnosis of multiple sclerosis on an expedited basis would be of great benefit to patients in light of the potential for recurrence of attacks and progression of the disease. Drugs for the treatment of multiple sclerosis are now available which slow or prevent progression of the disease in many patients, and an early diagnosis would, therefore, allow early intervention and could significantly improve the quality of life for many multiple sclerosis patients.

It is therefore an object of the present invention to provide an efficient diagnosis system for neuropsychiatric disease in general, a particular example of which is multiple sclerosis, so as to provide biomarkers with which a clinician can diagnose or exclude a particular disease in a patient or other mammal. The same reasoning applies to Neurologic Lyme Disease and Chronic Fatigue syndrome.

The citation of references herein shall not be construed as an admission that such is prior art to the present invention. Several publications and patent documents are referenced in this application in order to more fully describe the state of the art to which this invention pertains. The disclosure of each of these publications and documents is incorporated by reference herein.

Other features and advantages of the invention will be apparent from the detailed description, the drawings, and the claims.

### SUMMARY OF THE INVENTION

The present invention is based on the discovery of biomarkers useful for diagnosing and monitoring neuropsychiatric diseases. Neuropsychiatric diseases which may be diagnosed or monitored using the biomarkers of the invention include, without limitation: multiple sclerosis (especially focused on first attack multiple sclerosis), Neurologic Lyme Disease, and Chronic Fatigue Syndrome. Biomarkers identified for such neuropsychiatric diseases are listed herein in Tables 1m, 2m, 3, 4H-m, 4H-1, 4H-c, 5c, 6c, 7L, 8L, 9, and 10. As indicated in the tables presented herein, the presence or absence of at least one biomarker in a biological sample isolated from a patient is indicative of a positive diagnosis of a neuropsychiatric disease. As indicated herein, the identification of a plurality of biomarkers correlated with a particular disease may strengthen the confidence level of the diagnostic prediction. Biomarkers identified herein have also been assessed using software programs (such as, e.g., Ingenuity Pathway Analysis) to identify metabolic pathways implicated in disease etiology, maintenance, and/or progression. Multiple hits in a particular metabolic pathway underscore the potential importance of the pathway for the disease and direct therapeutic intervention toward appropriate modulation of same.

Accordingly, the present invention is directed to a method for diagnosing a neuropsychiatric disease in a patient, wherein said method comprises isolating a biological sample from the patient and analyzing the biological sample to determine if at least one of the biomarkers indicative of the presence of the neuropsychiatric disease is identified in the biological sample, thereby diagnosing the neuropsychiatric disease in the patient. As described herein, the presence or absence of at least one biomarker in a biological sample isolated from a patient is indicative of a positive diagnosis of a neuropsychiatric disease.

In another aspect, a method for diagnosing multiple sclerosis in a patient is presented, the method comprising: a) isolating a biological sample from the patient; and b) analyzing the biological sample for expression of a panel of biomarkers, wherein the panel of biomarkers is set forth in any one of Tables 1m, 3m, 4H-m or 2m, to determine if at least one of the biomarkers listed in any one of Tables 1m, 3m, 4H-m or Table 2m is identified in the biological sample, wherein identification of at least one of the biomarkers in the biological sample indicates a positive diagnosis or is indicative of multiple sclerosis in the patient. In an embodiment thereof, the at least one biomarker identified in the biological sample is present in the biological sample and set forth in Table 1m, 3m, or 4H-m, and the presence of the at least one biomarker indicates a positive diagnosis or is indicative of multiple sclerosis. In another embodiment, the at least one biomarker identified in the biological sample is absent from the biological sample and set forth in Table 2m and the absence of the at least one biomarker indicates a positive diagnosis or is indicative of multiple sclerosis.

In yet another aspect, a method for differentially diagnosing a neuropsychiatric disease in a patient is presented, wherein the patient exhibits clinical symptoms common to both Neurologic Lyme disease and Chronic Fatigue Syndrome, the method comprising: a) isolating a biological sample from the patient; and b) analyzing the biological sample for expression of a first panel of biomarkers, wherein the first panel of biomarkers is set forth in any one of Tables 4H-1, 7L, or 8L, to determine if at least one of the biomarkers listed in any one of Tables 4H-1, 7L, or 8L is identified in the biological sample, wherein identification of at least one of the biomarkers in the biological sample indicates a positive diagnosis or is indicative of Neurologic Lyme disease. In an embodiment thereof, the at least one biomarker identified in the biological sample is present in the biological sample and set forth in Table 4H-1 or 7L and the presence of the at least one biomarker indicates a positive diagnosis or is indicative of Neurologic Lyme disease. In another embodiment, the at least one biomarker identified in the biological sample is absent from the biological sample and set forth in Table 8L and the absence of the at least one biomarker indicates a positive diagnosis or is indicative of Neurologic Lyme disease.

The method may further comprise analyzing the biological sample for expression of a second panel of biomarkers, wherein the second panel of biomarkers is set forth in any one of Tables 4H-c, 5c, or 6c, to determine if at least one of the biomarkers listed in any one of Tables 4H-c, 5c, or 6c is identified in the biological sample, wherein identification of at least one of the biomarkers in the biological sample indicates a positive diagnosis or is indicative of Chronic Fatigue Syndrome. In an embodiment thereof, the at least one biomarker identified in the biological sample is present in the biological sample and set forth in Table 4H-c or 5c and the presence of the at least one biomarker indicates a positive diagnosis or is indicative of Chronic Fatigue Syndrome. In another embodiment, the at least one biomarker identified in the biological sample is absent from the biological sample and set forth in Table 6c and the absence of the at least one biomarker indicates a positive diagnosis or is indicative of Chronic Fatigue Syndrome.

In yet another aspect, a method for diagnosing Neurologic Lyme disease in a patient is presented, the method comprising: a) isolating a biological sample from the patient; and b) analyzing the biological sample for expression of a panel of biomarkers, wherein the panel of biomarkers is set forth in any one of Tables 4H-1, 7L, or to determine if at least one of the biomarkers listed in any one of Tables 4H-1, 7L, or 8L is identified in the biological sample, wherein identification of at least one of the biomarkers in the biological sample indicates a positive diagnosis or is indicative of Neurologic Lyme disease in the patient. In an embodiment thereof, the at least one biomarker identified in the biological sample is present in the biological sample and set forth in Table 4H-1 or 7L, and the presence of the at least one biomarker indicates a positive diagnosis or is indicative of Neurologic Lyme disease. In another embodiment, the at least one biomarker identified in the biological sample is absent from the biological sample and set forth in Table 8L and the absence of the at least one biomarker indicates a positive diagnosis or is indicative of Neurologic Lyme disease.

In yet another aspect, a method for diagnosing Chronic Fatigue Syndrome in a patient is presented, the method comprising: a) isolating a biological sample from the patient; and b) analyzing the biological sample for expression of a panel of biomarkers, wherein the panel of biomarkers is set forth in any one of Tables 4H-c, 5c, or 6c, to determine if at least one of the biomarkers listed in any one of Tables 4H-c, 5c, or 6c is identified in the biological sample, wherein identification of at least one of the biomarkers in the biological sample indicates a positive diagnosis or is indicative of Chronic Fatigue Syndrome in the patient. In an embodiment thereof, the at least one biomarker identified in the biological sample is present in the biological sample and set forth in Table 4H-c or 5c, and the presence of the at least one biomarker indicates a positive diagnosis or is indicative of Chronic Fatigue Syndrome. In another embodiment, the at least one biomarker identified in the biological sample is absent from the biological sample and set forth in Table 6c and the absence of the at least one biomarker indicates a positive diagnosis or is indicative of Chronic Fatigue Syndrome.

In accordance with the methods described herein, the biological sample may be cerebrospinal fluid, blood or a component thereof (e.g., plasma, serum, cells), tissue or tissue-related fluids, urine, or saliva.

Also encompassed herein is a method for diagnosing a neuropsychiatric disease, the method comprising: a) isolating a biological sample from the patient; b) analyzing the biological sample to determine a first level of at least one of the biomarkers indicative of the presence of the neuropsychiatric disease in the biological sample; and c) comparing the first level of the at least one of the biomarkers to a second level of the at least one of the biomarkers determined for a subject, wherein the subject does not have the neuropsychiatric disease, and wherein at least a two-fold difference in the ratio of the first level to the second level indicates a positive diagnosis of the neuropsychiatric disease. In a particular embodiment, the at least two-fold difference is an increase, which is indicative of a positive diagnosis of the neuropsychiatric disease. In an alternative embodiment, the at least two-fold difference is a decrease, which is indicative of a positive diagnosis of the neuropsychiatric disease. As described herein, in certain circumstances the subject is a healthy subject. In accordance with the invention, the subject may have a second neuropsychiatric disease, wherein the second neuropsychiatric disease differs from the neuropsychiatric disease for which the instant method is diagnostic. Under such a circumstance, the two-fold difference in the ratio may be used to confer a differential diagnosis between the neuropsychiatric disease for which the instant method is diagnostic and the second neuropsychiatric disease. This is a particularly useful feature of the present method when the clinical presentation for the neuropsychiatric disease for which the instant method is diagnostic and the second neuropsychiatric disease is similar.

In a particular embodiment thereof, a method is presented for diagnosing a neuropsychiatric disease in a patient, wherein the patient exhibits clinical symptoms common to both Chronic Fatigue Syndrome and Neurologic Lyme disease, the method comprising: a) isolating a biological sample from the patient; b) analyzing the biological sample to evaluate expression levels of the biomarkers listed in Table 9 to determine a first level of at least one of the biomarkers indicative of the presence of the neuropsychiatric disease in the biological sample; and c) comparing the first level of the at least one of the biomarkers to a second level of the at least one of the biomarkers determined for a subject, wherein the subject does not have the neuropsychiatric disease, and wherein at least a two-fold difference in the ratio of the first level to the second level indicates a positive diagnosis of the neuropsychiatric disease. In an embodiment thereof, the diagnosis is directed to determining if the patient has Chronic Fatigue Syndrome or Neurologic Lyme disease. In a particular embodiment, the at least a two-fold difference is at least a two-fold increase or at least a two-fold decrease. In another particular embodiment, the subject is a healthy subject.

Also encompassed herein are kits for diagnosing neuropsychiatric diseases. In a particular aspect, a kit for diagnosing multiple sclerosis is presented, wherein the kit comprises probes (e.g., antibodies or fragments or derivatives thereof) for detecting expression of at least one biomarker listed in Table 1m, 3m, 4H-m, or Table 2m, wherein detectable expression of at least one biomarker listed in Table 1m, 3m, or 4H-m is indicative of a positive diagnosis of multiple sclerosis and wherein an absence of detectable expression of at least one biomarker listed in Table 2m is indicative of a positive diagnosis of multiple sclerosis.

In another aspect, a kit for diagnosing Chronic Fatigue Syndrome is presented, wherein the kit comprises probes (e.g., antibodies or fragments or derivatives thereof) for detecting expression of at least one biomarker listed in Table 4H-c, 5c, or Table 6c, wherein detectable expression of at least one biomarker listed in Table 4H-c or 5c is indicative of a positive diagnosis of Chronic Fatigue Syndrome and wherein an absence of detectable expression of at least one biomarker listed in Table 6c is indicative of a positive diagnosis of Chronic Fatigue Syndrome.

In yet another aspect, a kit for diagnosing Neurologic Lyme Disease is presented, wherein the kit comprises probes (e.g., antibodies or fragments or derivatives thereof) for detecting expression of at least one biomarker listed in Table 7L or Table 8L, wherein detectable expression of at least one biomarker listed in Table 7L is indicative of a positive diagnosis of Neurologic Lyme Disease and wherein an absence of detectable expression of at least one biomarker listed in Table 8L is indicative of a positive diagnosis of Neurologic Lyme Disease.

As described herein, the kits embodied herein may comprise probes for detecting expression of at least 2, 3, 4, 5, 6, 7, 8, 9, 10, 15, 20, 25, or 30 of the biomarkers listed in any one of Tables 1m, 3m, 4H-m, 2m, 4H-c, 5c, 6c, 7L or 8L, wherein the number of biomarkers listed in any one of Tables 1m, 3m, 4H-m, 2m, 4H-c, 5c, 6c, 7L or 8L establishes the upper limit of biomarkers detected by the kit. In other words, each of the Tables encompasses a maximum number of biomarkers as indicated therein and the number of biomarkers listed in the Tables may differ between each Table. In a particular embodiment, a kit may comprise probes for detecting expression of all of the biomarkers listed in any one of Tables 1m, 3m, 4H-m, 2m, 4H-c, 5c, 6c, 7L or 8L. In particular embodiments, the probes may be immobilized on a solid surface.

In another aspect, the invention is directed to a method for monitoring efficacy of a treatment for a neuropsychiatric disease in a patient, wherein said method comprises isolating a first biological sample from the patient prior to initiating the treatment and isolating a second biological sample from the patient after initiating the treatment, analyzing the biological samples to determine the level of at least one of the biomarkers indicative of the presence of the neuropsychiatric disease in the first and second biological samples, and comparing the level of the at least one of the biomarkers in the first and second biological samples, wherein a change in the level of the at least one of the biomarkers reflects the efficacy of the treatment. In one embodiment, wherein the presence of the at least one biomarker is associated with a positive diagnosis or presence of the disease, a detectable reduction in the level of the at least one biomarker following initiating the treatment indicates that the treatment is efficacious. In another embodiment, wherein the absence of the at least one biomarker is associated with a positive diagnosis or presence of the disease, a detectable increase in level in the at least one biomarker following initiating the treatment indicates that the treatment is efficacious.

In another aspect, the invention is directed to a method for monitoring disease progression of a neuropsychiatric disease in a patient, wherein said method comprises isolating a first biological sample from the patient at an initial or first temporal window and isolating a second biological sample from the patient at a later (e.g., second) temporal window, analyzing the biological samples to determine the level of at least one of the biomarkers indicative of the presence of the neuropsychiatric disease in the first and second biological samples, and comparing the level of the at least one of the biomarkers in the first and second biological samples, wherein a change in the level of the at least one of the biomarkers in the first and second biological samples reflects progression of the disease. In one embodiment, wherein the presence of the at least one biomarker is associated with a positive diagnosis or presence of the disease, a detectable increase in the level of the at least one biomarker at the later temporal time window relative to the initial temporal window indicates that the disease has progressed or worsened. In another embodiment, wherein the absence of the at least one biomarker is associated with a positive diagnosis or presence of the disease, a detectable decrease in the level of the at least one biomarker at the later temporal time window relative to the initial temporal window indicates that the disease has improved.

The present invention is, moreover, directed to a method for diagnosing a neuropsychiatric disease in a patient, wherein said method comprises visualizing or detecting at least one of the biomarkers indicative of the presence of the neuropsychiatric disease in the patient, thereby diagnosing the neuropsychiatric disease in the patient. As described herein, the presence or absence of at least one biomarker in a patient is indicative of a positive diagnosis of a neuropsychiatric disease. In accordance with such an embodiment, various detection imaging techniques may be utilized including, but not limited to, magnetic resonance imaging (MRI), positron emission tomography (PET), and CAT scans.

In one embodiment, the present invention relates to the identification and use of biomarkers in methods for diagnosing multiple sclerosis, particularly diagnosis of first attack multiple sclerosis. The invention also pertains to the use of these biomarkers in methods for assessing relapse of multiple sclerosis patients and disease progression in multiple sclerosis patients. Biomarkers identified in accordance with the invention are also useful for identifying treatments for and monitoring efficacy of treatment of patients with multiple sclerosis. As shown herein, the inventors have identified a plurality of biomarkers whose presence is indicative of multiple sclerosis; such biomarkers are listed in Tables 1m, 3m, or 4H-m. The inventors have also identified a plurality of biomarkers whose absence is indicative of multiple sclerosis; such biomarkers are listed in Table 2m.

In another embodiment, the present invention relates to the identification and use of biomarkers in methods for diagnosing chronic fatigue syndrome (CFS) in patients. Biomarkers identified in accordance with the invention are also useful for identifying treatments for and monitoring efficacy of treatment of patients with CFS. As shown herein, the inventors have identified a plurality of biomarkers whose presence is indicative of CFS; such biomarkers are listed in Table 4H-c, and 5c. The inventors have also identified a plurality of biomarkers whose absence is indicative of CFS; such biomarkers are listed in Table 6c.

In yet another embodiment, the present invention relates to the identification and use of biomarkers in methods for diagnosing Neurologic Lyme disease in patients. Biomarkers identified in accordance with the invention are also useful for identifying treatments for and monitoring efficacy of treatment of patients with Neurologic Lyme disease. As shown herein, the inventors have identified a plurality of biomarkers whose presence is indicative of Neurologic Lyme disease; such biomarkers are listed in Table 7L. The inventors have also identified a plurality of biomarkers whose absence is indicative of Neurologic Lyme disease; such biomarkers are listed in Table 8L.

Also encompassed herein are diagnostic signatures for neuropsychiatric diseases. Such diagnostic signatures comprise biomarkers for diagnosing a neuropsychiatric disease, wherein the presence or absence of at least one biomarker indicates a positive diagnosis of the neuropsychiatric disease. In particular aspects, the diagnostic signature is indicative of multiple sclerosis, Chronic Fatigue Syndrome, or Neurologic Lyme Disease. In a particular embodiment, the diagnostic signature relates to the presence of at least one biomarker listed in Table 1m, 3m, or 4H-m and is indicative of a positive diagnosis of multiple sclerosis. In another embodiment, the diagnostic signature relates to the absence of at least one biomarker listed in Table 2m and is indicative of a positive diagnosis of multiple sclerosis. In yet another embodiment, the diagnostic signature relates to the presence of at least one biomarker listed in Table 4H-c or 5c and is indicative of a positive diagnosis of Chronic Fatigue Syndrome. In a further embodiment, the diagnostic signature relates to the absence of at least one biomarker listed in Table 6c and is indicative of a positive diagnosis of Chronic Fatigue Syndrome. In another embodiment, the diagnostic signature relates to the presence of at least one biomarker listed in Table 7L and is indicative of a positive diagnosis of Neurologic Lyme Disease. In yet another embodiment, the diagnostic signature relates to the absence of at least one biomarker listed in Table 8L and is indicative of a positive diagnosis of Neurologic Lyme Disease.

### BRIEF DESCRIPTION OF THE DRAWINGS

FIG. 1 shows a Venn diagram revealing the amount of overlap of the present dataset with a comparable dataset of proteins detected in the CSF of “normal clinical value” or “neurologic surrogate-normal” individuals who required a lumbar puncture for clinical reasons as reported by Zougman et al. [(2008) J Proteome Res 7: 386-399]. The large circle represents the 2630 proteins observed in the present comprehensive dataset of proteins detected in the CSF of normal individuals. The small circle represents the 798 proteins identified in the analysis by Zougman et al. [(2008) J Proteome Res 7: 386-399].

### DETAILED DESCRIPTION OF THE INVENTION

The present inventors had a unique opportunity to generate what may be the most comprehensive database of true normal CSF proteins to date. The inventors were able to do this because they had sufficient numbers and total volume of true normal CSF samples to employ immunoaffinity depletion, followed by extensive fractionation and high-resolution liquid chromatography (LC) separation and mass spectrometry (MS) analysis. The combination of the normal CSF samples, including a set of serial CSF samples, and advanced technology contribute to the uniqueness and value of the present study. Knowledge as to the normal CSF proteome, in turn, makes possible a meaningful comparison with disease proteomes (i.e., proteomes characteristic of a particular disease).

Until recently, technological limitations have prevented full characterization of the CSF proteome. Comprehensive analysis of CSF has been challenged by low protein levels (0.3 to 0.7 mg/ml) compared to plasma, protein concentration variability up to twelve orders of magnitude, potential masking of brain-specific proteins by highly abundant proteins [Rozek et al. (2007) J Proteome Res 6: 4189-4199], and limited access to an adequate number of appropriate biological samples. Despite some of these limitations, a number of earlier studies have provided increasing levels of characterization of the CSF proteome. For the most part, these studies have used pooled samples from patient populations with diseases or from people with normal CSF clinical laboratory values (chemistries, cell counts, and microbiology) who underwent lumbar puncture for investigation of neurological complaints. These CSF samples were used as substitutes or surrogates for true normals (healthy volunteers) possibly due to lack of availability of such normal CSF samples. Sickmann et al., [(2000) Electrophoresis 21: 2721-2728] used two-dimensional polyacrylamide gel electrophoresis (2D-PAGE) followed by mass spectrometry (MS) to identify close to 70 CSF proteins. Yuan et al., [(2005) Proteomics 5:541-550] used matrix-assisted laser desorption ionization (MALDI) MS to identify 22 proteins in desalted CSF. Wenner et al., [(2004) J Proteome Res 3: 97-103] used 2D liquid chromatography (LC) coupled to tandem mass spectrometry (2D-LC-MS/MS) to identify 249 proteins in pooled CSF. Maccarrone et al. [(2004) Electrophoresis 25: 2402-2412]; used immunodepletion techniques and shotgun LC-MS/MS, to identify more than 100 proteins in CSF from a patient with normal pressure hydrocephalus. More recently Zougman et al. [(2008) J Proteome Res 7: 386-399], using LC-MS/MS, reported 798 proteins in a pool of 6 patients with neurologic complaints that warranted a lumbar puncture, but whose subsequent clinical CSF laboratory values were reported as normal; for the purposes of this application these types of patients as referred to as neurologic surrogate-normals. A notable exception to use of surrogates was the work by Zhang et al. [(2005) Neurobiol Aging 26: 207-227], who used 2D-LC-MS/MS to identify 315 proteins in pools of CSF comparing healthy younger versus older individuals. Subsequently, with Xu et al. [(2006) Int Rev Neurobiol 73: 29-98], they analyzed CSF from the younger group with two different LC-MS/MS platforms and identified a combined total of 915 proteins. Pan et al., reported a total of 2594 CSF protein identifications from different combined (cumulative) results of several CSF studies with a focus on neurodegenerative diseases such as Alzheimer's disease [(2007) Proteomics 7: 469-473].

Towards the principal purpose of establishing a comprehensive list or proteome of normal CSF, the present inventors have prepared CSF samples from healthy normal people for analysis by using immunoaffinity depletion of abundant proteins (with masking potential) to enhance coverage and detection of low abundance proteins [Qian et al. (2009) J Proteome Res 8: 290-299]. It is noteworthy that the present inventors also analyzed the abundant sample fraction in parallel. Samples were then analyzed employing high throughput, high sensitivity, and high resolution nanocapillary liquid chromatography-mass spectrometry (LC-MS [Petyuk et al. (2007) Genome Res 17: 328-336] and LC-MS/MS [Qian et al. (2009) J Proteome Res 8: 290-299]). Pre-fractionation (immunoaffinity depletion chromatography) and ultra-high resolution nanocapillary LC separations were used to effectively reduce the sample complexity and concentration dynamic range (thereby reducing or eliminating the “masking” effect [Ramstrom et al. (2005) J Proteome Res 4: 410-416]), high efficiency ion transmission technologies (e.g., electrodynamic ion funnel [Shaffer et al. (1997) Rapid Commun Mass Spectrom 11: 1813-1817]) for highly sensitive global MS analysis, and the accurate mass and time (AMT) tag strategy [Smith et al. (2002) Proteomics 2: 513-523] for high-throughput analysis (e.g. of individual CSF samples) and accurate quantitation. The general approach for AMT tag generation and application as described herein has been successfully implemented for whole microbial [Lipton et al. (2002) Proc Natl Acad Sci USA 99: 11049-11054] and mammalian tissue [Petyuk et al. (2007) Genome Res 17: 328-336] and plasma proteomes [Qian et al. (2009) J Proteome Res 8: 290-299; Qian et al. (2005) Mol Cell Proteomics 4: 700-709], but has not been previously applied to CSF from normal subjects.

As described herein, pooled CSF isolated from 11 normal healthy volunteers (8 women and 3 men, aged 24 to 55, median=28 years) who reported their health as excellent or good and were taking no medications was examined. Standard clinical laboratory testing on their CSF was normal (none had more than 3 white blood cells/mm³ and protein levels ranged from 14 to 40 mg/dl with a median of 25 mg/dl). Also examined were pairs of individual serial CSF samples, obtained at least 4 weeks apart, from 10 additional normal healthy volunteers to assess the potential variability of particular CSF protein levels in an individual from one time point to another.

To illustrate the utility of such a normal database and how one clinical condition might be compared to another the present inventors also analyzed and compared one set of CSF samples to another set processed in the same manner to evaluate, among other issues, if there might be significant differences among different surrogate-normal groups.

In summary and as described in greater detail herein below, the present inventors applied immunoaffinity separation and high sensitivity and resolution liquid chromatography-mass spectrometry to examine CSF from healthy normal individuals. 2630 proteins in CSF from normal subjects were identified, of which 56% were CSF-specific (i.e., as determined using the techniques described herein), not found in the much larger set of 3654 proteins identified in plasma. The above-mentioned 2630 proteins identified in normal subjects were identified with high confidence, meaning that two or more peptides corresponding to a particular protein were required as confirmation that the particular protein was, indeed, present in normal CSF. The present inventors also examined CSF from groups like those of subjects previously examined by others as surrogates for normals, where neurologic symptoms warranted a lumbar puncture but where clinical laboratory were reported as normal. Statistically significant differences between their CSF proteins and those determined for non-neurological normals examined herein were noted. The inventors also examined CSF from 10 volunteer subjects who had lumbar punctures at least 4 weeks apart and found that there was little variability in CSF proteins in an individual as compared from subject to subject.

The present results represent the most comprehensive characterization of true normal CSF to date. This normal CSF proteome establishes a comparative standard and basis for investigations into a variety of diseases with neurological and psychiatric features. With this essential information, the present inventors were able to identify biomarkers diagnostic for a number of neuropsychiatric diseases. See Examples I-IV.

Accordingly, the present invention encompasses identification of biomarkers diagnostic for a number of neuropsychiatric diseases. As indicated in each of Tables 1-10, the present inventors have identified biomarkers, whose presence or absence as indicated herein is a positive indicator of the presence of a neuropsychiatric disease in a patient. Thus, the presence or absence of at least one biomarker for a neuropsychiatric disease serves as a diagnostic tool for clinicians. It is understood that a clinician will evaluate a patient as a whole based on known criteria well established in the field for diagnosis of a neuropsychiatric disease and utilize the diagnostic method of the present invention in conjunction with such known diagnostic criteria to establish a definitive diagnosis.

According to the present invention, the term “neuropsychiatric diseases” refers to, without limitation, multiple sclerosis and in particular first attack or early multiple sclerosis, Neurologic Lyme Disease, Chronic Fatigue Syndrome, and other diseases with similar features that may place them in the differential diagnosis in evaluating a patient.

According to the present invention, the term “multiple sclerosis” is used to describe the art-recognized disease characterized by inflammation, demyelination, oligodendrocyte death, membrane damage, other neurologic damage, and axonal death. Multiple sclerosis can be characterized as one of four main varieties as defined in an international survey of neurologists (Lublin and Reingold, 1996, Neurology 46(4):907-11), which are namely: Relapsing-Remitting multiple sclerosis (RRMS), Primary Progressive multiple sclerosis (PPMS), Secondary-Progressive multiple sclerosis (SPMS), Progressive-Relapsing multiple sclerosis (PRMS).

According to the present invention, the term “Neurologic Lyme Disease” is used to describe a range of neurologic or psychiatric manifestations that may be associated with Lyme disease. As referred to herein, “Neurologic Lyme Disease” encompasses the range from an acute neurologic syndrome in an acute case of active Lyme Disease and late manifestation of Lyme disease (both sometimes referred to as neuroborreliosis), to the more poorly defined post-treatment Lyme disease syndrome and “chronic Lyme Disease”

According to the present invention, the term “Chronic Fatigue Syndrome” is used to describe a constellation of symptoms including fatigue that approach those described in the 1994 proposed case definition [Fukuda et al, Annals of Internal Medicine, Vol. 121, Dec. 15, 1994, pp. 953-959] that are subject to revision. In practice it is often a clinical diagnosis of exclusion.

As used herein, the terms “patient” or “subject” are used interchangeably. Such patients may have a neuropsychiatric disease or disorder, such as multiple sclerosis, CFS, and Neurologic Lyme Disease. With respect to multiple sclerosis, for example, the terms “multiple sclerosis patient”, “a subject who has multiple sclerosis”, “a patient who has multiple sclerosis”, “a multiple sclerosis subject”, and similar phrases, are intended to refer to subjects who have been diagnosed with multiple sclerosis.

The terms “healthy subject” or “normal subject”, and similar phrases, are intended to refer to a subject who has not been diagnosed with a neuropsychiatric disease or disorder. A healthy subject has no other acute systemic disease or, at the least, has no detectable acute systemic disease. With respect to aspects of the invention directed to, for example, multiple sclerosis, the terms “healthy subject”, “non-multiple sclerosis subject”, “a subject who does not have multiple sclerosis”, “a patient who does not have multiple sclerosis”, and similar phrases, are intended to refer to a subject who has not been diagnosed with multiple sclerosis.

As used herein, the term “patient” or “subject” may refer to a mammal, including a human.

As used herein, the term “biological sample” includes a sample of any cell type or from any tissue or body fluid, body fluids including, but not limited to: cerebrospinal fluid (CSF), blood (whole blood, serum, plasma, or cellular components), saliva, urine, prostatic fluid, or fluid from any suitable tissue. In a particular embodiment, the biological sample is CSF. In another embodiment, the biological sample is blood or any component of blood (e.g., serum, plasma, cells, etc.).

The term “protein” is used herein to mean protein, polypeptide, oligopeptide or peptide. The terms “biologic marker”, “biomarker” or “disease-associated protein” are used herein to refer to proteins associated with specific diseases or conditions, including proteins from organs or tissues (“organ-specific” or “tissue-specific” proteins) affected by a disease or condition. In accordance with the present invention, the presence or absence of a biomarker is positively correlated with, indicative of, of diagnostic for the presence of a disease or condition, such as a neuropsychiatric disease, in a patient. Also in accordance with the present invention, a relative increase in levels of a particular biomarker or biomarkers as compared to a control or normal subject can also be positively correlated with, indicative of, or diagnostic for the presence of a disease or condition, such as a neuropsychiatric disease, in a patient.

The present invention, furthermore, encompasses a plurality of biomarkers or a “biomarker signature” that is positively correlated with, indicative of, or diagnostic for the presence of a disease or condition, such as a neuropsychiatric disease, in a patient. Accordingly, the presence or absence of a biomarker signature is positively correlated with, indicative of, or diagnostic for the presence of a disease or condition, such as a neuropsychiatric disease, in a patient. A biomarker signature may also encompass a plurality of biomarkers that exhibit a relative increase or decrease in levels as compared to a healthy or normal subject (e.g., a control subject) and is positively correlated with, indicative of, or diagnostic for the presence of a disease or condition, such as a neuropsychiatric disease, in a patient.

A skilled practitioner would, moreover, appreciate that the presence or absence of a particular protein in a sample may be weakly indicative of disease, but not diagnostic, if noted as a single determinant. If, however, a plurality of such single determinants are noted in a biological sample, the combined detection of several weakly indicative determinants may serve to identify a strong combinatorial diagnostic indicator of disease. Furthermore, the single protein/determinant need not even approach the threshold of weak diagnostic by itself but in combination with the detection or absence of detection of another protein or proteins may serve as a strong combinatorial diagnostic indication of a disease state. Accordingly, also encompassed herein are combinatorial diagnostic indicators that are associated with a particular disease and not observed in healthy subjects or patients with other diseases.

Accordingly, selected sets of one, two, three, and more of the biomarkers of this invention (up to the number equivalent to all of the biomarkers, including any intervening number, in whole number increments, e.g., 1, 2, 3, 4, 5, 6 . . . ) can be indicative of a positive diagnosis or used as diagnostic indicators for methods and/or kits described herein. In one embodiment, larger numbers of the biomarkers identified herein are used in methods or kits of the invention, since the accuracy of the method or kit may improve as the number of biomarkers screened increases. With respect to aspects of the invention pertaining to evaluating therapeutic efficacy, the methods and kits of the present invention include evaluating whether administration of a therapeutic composition causes a change, either a transient change or a long term change, in one or more of the biomarkers; in two or more of the biomarkers; in three or more of the biomarkers; in four or more of the biomarkers; in five or more of the biomarkers, in six or more of the biomarkers, etc.

Also envisioned are sets of at least 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the biomarkers listed in any one of the tables presented herein, including Tables 1m, 2m, 3, 4H-m, 4H-1, 4H-c, 5c, 6c, 7L, 8L, and 9, wherein the envisioned sets can be indicative of a positive diagnosis or used as diagnostic indicators for methods and/or kits described herein. With respect to aspects of the invention pertaining to evaluating therapeutic efficacy, the methods and kits of the present invention include evaluating whether administration of a therapeutic composition causes a change, either a transient change or a long term change, in a percent of the biomarkers listed in any one of the Tables described herein.

Representative permutations of biomarkers and/or biomarker signatures are presented herein below for illustrative purposes. Based on the methods described herein, a biological sample isolated from a subject may be determined to exhibit any one of the following biomarker signatures, which are set forth in exemplary fashion, and are not to be viewed as limiting: a+; a−; a+, b+, c+; or a+, b−, c+; wherein “a”=specific protein “a”, “b”=specific protein “b”, “c”=specific protein “c”, etc. and “+” indicates that the protein is present or quantitatively increased relative to subjects without disease, which could be clinically normal or could have a different disease (e.g., a different disease having overlapping clinical symptoms which is, therefore difficult to distinguish) and “-” indicates that the protein is absent or quantitatively decreased relative to subjects without disease, which could be clinically normal or could have a different disease (e.g., a different disease having overlapping clinical symptoms which is, therefore difficult to distinguish).

For example, the easiest identification of a protein biomarker is the presence of a protein associated with a disease or condition and not with other conditions that might be clinically confused with the disease under consideration. Variations to this scenario include the situation wherein a biomarker is present in an increased quantity or a decreased quantity compared to other conditions or controls. Although not a protein biomarker, an example of this is the presence of glucose in the blood in high quantities in diabetics compared to normal individuals who still have glucose present but not in elevated quantities. Another variation to the first scenario is where the functional biomarker is not just one protein but two or more in combination that can be present or absent or quantitatively different, wherein the ensemble defines its biomarker potential.

In some embodiments, a biomarker of the invention is a member of a biological pathway. As used herein, the term “precursor” or “successor” refers to molecules that precede or follow the biomarker in the biological pathway. Thus, once a biomarker is identified as a member of one or more biological pathways, the present invention can include additional members of the biological pathway that come before (are upstream of or a precursor of) or follow (are downstream of) the biomarker. Such identification of biological pathways and their members is within the skill of one in the art.

Also encompassed herein is the analysis of biomarkers identified and listed in the tables presented herein to identify metabolic pathways implicated in the pathogenesis, maintenance, and/or progression of a disease. Such analyses may utilize a variety of software programs, including but not limited to the commercially available Ingenuity Pathway Analysis. Multiple hits in a particular metabolic pathway underscore the potential importance of the pathway for the disease and direct therapeutic intervention toward appropriate modulation of same. Accordingly, the present methods encompass such analyses and the identification of metabolic pathways of potential significance in a particular disease. Knowing that, for example, activation of a metabolic pathway appears to be linked or associated with a particular disease presents the opportunity to test pharmaceutical modulators of the pathway (i.e., inhibitors) to determine if such modulators could be used as therapeutics for treatment of patients with the disease.

Metabolic pathways may, for example, be associated with several disease conditions based on a quantitative assessment. Examples of pathways that may be markedly involved in one condition and not another or others may also be found. As a specific illustrative example, a comparison of Chronic Fatigue Syndrome (CFS) to Neurologic Post Treatment Lyme disease syndrome (nPTLS), when individual patient samples were analyzed, revealed that four components (CIS, C4B, C1QB, C1QC) associated with activation of the complement cascade were differentially increased in abundance consistently across the nPTLS patients compared to CFS. In another example, a comparison between CFS and nPTLS patiente, looking at the pooled results, revealed that the CDK5 signaling pathway was significantly enriched for proteins identified only in the pooled CFS proteome. This signaling pathway has been linked to Parkinson's (Smith et al. 2003. Proc. Natl. Acad. Sci. 100:13650) and Alzheimer's diseases (Lew et al. 1994. Nature 371:423). Another example is related to pathways that are involved in two conditions, that is, common to both but decreased in abundance in the two disease conditions of CFS and nPTLS, compared to normal, but at different levels. Quantitative distinguishing differences could still be found between the two conditions. A specific example relates to networks relevant to neurological function, such as axonal guidance, where the proteins in CFS were further decreased relative to nPTLS.

Also envisioned are panels of biomarkers comprising at least 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the biomarkers listed in any one of the tables presented herein, including Tables 1m, 2m, 3, 4H-m, 4H-1, 4H-c, 5c, 6c, 7L, 8L, and 9, wherein the envisioned panels can be used to assess a biological sample isolated from a patient to evaluate expression patterns of the biomarkers of the panel, which expression patterns are indicative of a positive diagnosis of a neuropsychiatric disease as described herein. Panels of biomarkers comprising at least one, two, three, and more of the biomarkers listed in any one of Tables 1m, 2m, 3, 4H-m, 4H-1, 4H-c, 5c, 6c, 7L, 8L, and 9 (up to the number equivalent to all of the biomarkers in a particular table, including any intervening number, in whole number increments, e.g., 1, 2, 3, 4, 5, 6 . . . ) are also envisioned herein. Any of the indicated panels may also be used to evaluate efficacy of a therapeutic regimen.

Polypeptide biomarkers may be isolated by any suitable method known in the art. Native polypeptide biomarkers can be purified from natural sources by standard methods known in the art such as chromatography, centrifugation, differential solubility or immunoassay. In one embodiment, polypeptide and metabolite markers may be isolated from a biological sample using standard techniques known in the art, for example, chromatographic methods or affinity purification using substrate-bound antibodies that specifically bind to the marker. As described herein, immunoaffinity depletion of abundant proteins (with masking potential) enhances coverage and detection of low abundance proteins. As indicated herein above, depletion yields separate fractions that are characterized independently.

In a positively-associated example, once a protein or peptide is identified (including one lacking name identification, such as a hypothetical protein), a less expensive platform or assay, as compared to mass spectrometry, can be devised or adapted. The ELISA assay is such an example where an antibody to the biomarker protein/peptide can be used to capture the target biomarker in the biological sample being detected. The sample may be CSF or blood, etc. Variations of this immuno-based technique may also be used such as, but not limited to, Western blots wherein a sample comprising a biomarker is transferred to a membrane and then subsequently allowed to interact with specific antibodies that are coupled to an enzyme (later to be exposed to a substrate that permits colorimetric visualization) or fluorescent material that permit visualization of one or more of the components or subcomponents (such as antigens) of the target biomarker. Alternatively, a non-coupled antibody can be used first and then a second antibody directed to this antibody, which has an enzyme or fluorescent tag attached thereto, can be used in a sandwich-like fashion. Another variation of this protein capturing technique could be multiplexing of several target biomarkers by using beads coated with multiple antibodies such as in the Luminex platform.

In another embodiment, once a disease protein/peptide biomarker is identified subsequent research may demonstrate that the body of disease subjects makes antibodies to the biomarker or biomarkers and the normal or other disease subject are negative for same. Under such circumstances, the presence of antibodies for a disease protein/peptide biomarker is indicative of disease. Experimentally, the presence of such disease specific antibodies is determined by using the disease protein/peptide biomarker as a binding target for the antibodies. This is a common indirect strategy for detection of infectious disease where the presence of an antibody indicates exposure to a foreign agent and in some cases the rise in titers (quantity of antibodies) over a short period of time indicates a very recent exposure.

As used herein, a polypeptide is referred to as “isolated” when it has been removed from its natural milieu (i.e., that has been subject to human manipulation), and can include purified polypeptides, partially purified polypeptides, synthetically produced polypeptides, and recombinantly produced polypeptides, for example. As such, “isolated” does not reflect the extent to which the polypeptide has been purified.

According to the present invention, the phrase “selectively binds to” refers to the ability of an antibody or antigen binding fragment thereof to preferentially bind to specified proteins. More specifically, the phrase “selectively binds” refers to the specific binding of one protein to another (e.g., an antibody or antigen binding fragment thereof to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

As used herein, the term “specifically binding,” refers to the interaction between binding pairs such as an antibody and an antigen with an affinity constant of at most 10⁻⁶ moles/liter, at most 10⁻⁷ moles/liter, or at most 10⁻⁸ moles/liter.

The present invention includes the use of any of the biomarkers as described herein (including genes or their RNA or protein products), as targets for the development or identification of therapeutic compositions and strategies for the treatment of neuropsychiatric diseases, such as for example, multiple sclerosis and/or relapsing multiple sclerosis.

Methods to measure biomarkers of this invention, include, but are not limited to: Western blot, immunoblot, enzyme-linked immunosorbant assay (ELISA), radioimmunoassay (MA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners. More particularly, high throughput, high sensitivity, and high resolution nanocapillary liquid chromatography-mass spectrometry (LC-MS), LC-MS/MS, pre-fractionation (immunoaffinity depletion chromatography) and ultra-high resolution nanocapillary LC separations, high efficiency ion transmission technologies (e.g., electrodynamic ion funnel), and the accurate mass and time (AMT) tag strategy for high-throughput analysis are described herein and known in the art.

As described herein, the term “probe” is used to refer to an agent that specifically binds to a biomarker listed in one of the tables presented herein. Suitable reagents for binding with a polypeptide corresponding to a biomarker of the invention include antibodies, antibody derivatives, labeled antibodies, antibody fragments; and the like. The term “probe” may also be used to refer to an agent that specifically binds to a nucleic acid sequence that encodes a biomarker listed in one of the tables presented herein. Suitable reagents for binding to a nucleic acid (e.g., a genomic DNA, an mRNA, a spliced mRNA, a cDNA, or the like) include complementary nucleic acids.

In accordance with the present disclosure, the tables present information with which an ordinarily skilled practitioner can access the amino acid sequences of the proteins identified herein as biomarkers, as well as nucleic acid sequences encoding same. A stepwise protocol for identification of the amino acid sequences listed in the tables presented herein is as follows: access one of the publicly available databases such as http://www.ebi.ac.uk/IPI/IPIhelp.html or http://www.uniprot.org/ via the Internet and enter the IPI number, or the protein name, or the Gene designation. For example, the first protein listed in Table 1m is designated “14-3-3 protein epsilon” and the corresponding Gene designation is YWHAE, and IPI designation of IPI00000816.1. Entering the IPI number, protein name, or Gene into one of the above publicly available databases will reveal the 255 amino acid sequence corresponding to “14-3-3 protein epsilon”. The above protocol is a matter of routine practice in laboratories skilled in the art and can be performed for any of the proteins listed therein. Such information may be used to design probes for detection of any of the proteins listed therein or to identify commercially available probes for detection of any of the proteins listed therein. Primers for detection nucleic acid sequences encoding any of the proteins listed in the tables presented herein are also envisioned. Such primers may be used to detect expression or lack thereof of a biomarker or biomarker signature of the invention. The design of primers for detecting expression levels of RNA (e.g., mRNA) of a biomarker or biomarkers listed herein is a matter of routine practice with the nucleic acid sequence in hand as provided by publicly available websites such as those mentioned above. Such probes and primers are useful for the kits described herein.

The present invention also includes a method to diagnose a subject as having a neuropsychiatric disease (e.g., multiple sclerosis, chronic fatigue syndrome, or Neurologic Lyme Disease, or one that conventionally may be considered among the differential diagnoses). In one embodiment, the method includes the steps of analyzing a biological sample isolated from the subject to determine if one or more of the biomarkers listed in Tables 1m, 3m, or 4H-m is present or if one or more of the biomarkers listed in Table 2m is absent, and diagnosing multiple sclerosis, wherein the presence (Tables 1m, 3m, or 4H-m) or absence (Table 2m) of one or more of these biomarkers indicates that the subject has multiple sclerosis. The present invention further encompasses circumstances wherein there is a change in the level/amount of a biomarker of the invention and such a change may also reflect the presence of multiple sclerosis or responsiveness to a therapeutic regimen.

In another embodiment, the method includes the steps of analyzing a biological sample isolated from the subject to determine if one or more of the biomarkers listed in Table 4H-c, or 5c is present or if one or more of the biomarkers listed in Table 6c is absent, and diagnosing chronic fatigue syndrome, wherein the presence (Table 4H-c or 5c) or absence (Table 6c) of one or more of these biomarkers indicates that the subject has chronic fatigue syndrome. The present invention further encompasses circumstances wherein there is a change in the level/amount of a biomarker of the invention and such a change may also reflect the presence of chronic fatigue syndrome or responsiveness to a therapeutic regimen.

In a further embodiment, the method includes the steps of analyzing a biological sample isolated from the subject to determine if one or more of the biomarkers listed in Table 7L is present or if one or more of the biomarkers listed in Table 8L is absent (within the limits of detection using methods and assays described herein), and diagnosing Neurologic Lyme Disease, wherein the presence (Table 7L) or absence (Table 8L) of one or more of these biomarkers indicates that the subject has Neurologic Lyme Disease. The present invention further encompasses circumstances wherein there is a change in the level/amount of a biomarker of the invention and such a change may also reflect the presence of Neurologic Lyme Disease or responsiveness to a therapeutic regimen.

The invention includes a kit for assessing the expression and/or expression levels of at least one of the biomarkers listed in the tables presented herein, whereby detecting the expression and/or expression levels of at least one of the biomarkers in a biological sample isolated from a patient is indicative of a positive diagnosis of the relevant disease to which the table in question pertains (e.g., MS, NLD, or CFS). The kit comprises a plurality of reagents, each of which is capable of binding specifically with a polypeptide (e.g., an antibody) or nucleic acid encoding same corresponding to a biomarker of the invention, e.g., one of the proteins listed in any one of the tables presented herein. Suitable reagents for binding with a polypeptide corresponding to a biomarker of the invention include antibodies, antibody derivatives, labeled antibodies, antibody fragments, and the like. Suitable reagents for binding to a nucleic acid (e.g., a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids [e.g., phosphate buffered saline (PBS) or SSC buffer] suitable for binding an antibody to a protein for which it is immunologically specific or for annealing complementary nucleic acids, one or more sample compartments, an instructional material which describes performance of a method of the invention, a positive control or controls, such as panel of proteins corresponding one of the panels set forth in any one of the tables presented herein or a biological sample isolated from a normal subject (a subject who does not manifest clinical symptoms of disease), or a biological sample isolated from a patient known to have the disease in questions, and the like.

The following protocols are provided to facilitate the practice of the present invention.

The following examples are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted) for. Unless indicated otherwise, parts are parts by weight, molecular weight is average molecular weight, temperature is in degrees Centigrade, and pressure is at or near atmospheric.

### Example I

### Materials and Methods

Cerebrospinal Fluid (CSF) Specimens.

All specimens had normal clinical laboratory values with respect to microbiology, chemistry (including protein levels), and cell counts (red blood cell were 0-10/mm³ and white blood cells were 0-5/mm³). Four sets of different types of normal CSF samples were analyzed. The first set, designated as true (healthy) normals was comprised of pooled CSF from 11 healthy normal individual volunteers from the United States (8 women and 3 men; aged 24 to 55 years with a median age of 28 years) was used for the comprehensive analysis using immunoaffinity depletion and 2D-LC-MS/MS. A second set, also true normals included pairs of serial CSF aliquots taken at least 4 weeks apart from 10 healthy volunteers from the United States (age 37-44 years; 5 males and 5 females). A third set, designated as non-neurologic surrogate-normals, was a pool of 200 subjects from Sweden (all without a neurologic or psychiatric disease, most who underwent lumbar puncture for non diagnostic reasons; over 90% were undergoing spinal anesthesia in preparation for orthopedic surgery (e.g. limbs-knees and hips)). Ages ranged from 16 to 65 years with a median of 44 years; 50:50 female:male. They were used in the direct LC-MS analysis using the AMT tag approach. These samples were collected on ice and cells removed by centrifugation [Bergquist et al. (2002) Mass Spectrom Rev 21: 2-15]. A fourth set, designated as neurologic surrogate-normals consisted of a pool of CSF from 10 people from Sweden with headaches (age 18-35 years; 8 female and 2 male) who had a lumbar puncture to investigate possible CNS infection, and who had normal CSF clinical laboratory values (hence designation surrogate-normal), was collected following the same protocol as the third set and analyzed in the same fashion as the second and third sets of normal CSF. CSF from this group was also subjected to centrifugation to remove cells. CSF from this group was collected following the same protocol as the pool of the 200 non-neurological surrogate-normals. The protein concentrations were determined by Coomassie Plus protein assay (Pierce, Rockford, Ill.) using a bovine serum albumin standard.

Immunoaffinity Depletion of 14 High-Abundance CSF Proteins.

A total of 18 mL of the pooled CSF sample (from the 11 healthy volunteers) was subjected to the separations of 14 high-abundance proteins (albumin, IgG, α₁-antitrypsin, IgA, IgM, transferrin, haptoglobin, α₁-acid glycoprotein, α₂-macroglobulin, apolipoprotein A-I, apolipoprotein A-II, fibrinogen, C3 and apolipoprotein B) using a 12.7×79.0 mm Seppro® IgY14 LC10 affinity LC column (Sigma, St Louis, Mo.) on an Agilent 1100 series HPLC system (Agilent, Palo Alto, Calif.), followed the protocols described previously [Liu et al. (2006) Mol Cell Proteomics 5: 2167-2174].

Protein Digestion.

The CSF proteins were incubated in 8 M urea and 10 mM dithiothreitol at 37° C. for 60 min, followed by alkylation with 40 mM iodoacetamide in the dark for 30 min at room temperature. The samples were diluted 10-fold with 50 mM ammonium bicarbonate (pH 8) and 1 mM CaCl₂, and digested for 3 h at 37° C. using sequencing grade, modified porcine trypsin (Promega, Madison, Wis.) at a trypsin/protein ratio of 1:50. Sample cleanup was achieved using a 1-mL SPE C18 column (Supelco, Bellefonte, Pa.) as described previously [Liu et al. (2006) Mol Cell Proteomics 5: 1899-1913]. The final peptide concentration was determined by BCA assay (Pierce). All tryptic digests were snap frozen in liquid nitrogen and stored at −80° C.

Strong Cation Exchange (SCX) Fractionation.

For tryptic digests of the IgY14 bound and flow-through fractions (the first set normal CSF pool from BN), 300 μg of tryptic peptides from CSF samples were resuspended in 300 μL 10 mM ammonium formate, 25% acetonitrile and fractionated by strong cation exchange chromatography as described previously [Liu et al. (2006) Mol Cell Proteomics 5: 1899-1913]. A total of 30 fractions were collected for each sample with each fraction being lyophilized prior to reversed-phase LC-MS/MS analysis.

Reversed-Phase Capillary LC-MS/MS and LC-MS Analysis.

The SCX fractions were analyzed using a custom-built automated four-column high pressure nanocapillary LC system coupled on-line to either a linear ion trap mass spectrometer (LTQ; ThermoFisher) or a linear quadrupole ion trap-orbitrap mass spectrometer (LTQ-Orbitrap, ThermoFisher), both modified in-house with an electrodynamic ion funnel [Shaffer et al. (1997) Rapid Commun Mass Spectrom 11: 1813-1817], via an electrospray ionization interface manufactured in-house. The reversed-phase separation was performed as described previously [Liu et al. (2006) Mol Cell Proteomics 5: 1899-1913]. To analyze the SCX fractions of the IgY14 bound fraction, the LTQ mass spectrometer was operated in a data-dependent MS/MS mode (m/z 400-2000) in which a full MS scan was followed by 10 MS/MS scans. The ten most intensive precursor ions were dynamically selected in the order of highest intensity to lowest intensity and subjected to collision-induced dissociation using a normalized collision energy setting of 35% and a dynamic exclusion duration of 1 min. The heated capillary was maintained at 200° C., while the ESI voltage was kept at 2.2 kV. The SCX fractions of the IgY14 flow-through fraction, which are enriched with lower abundance proteins, were analyzed by the LTQ-Orbitrap instrument operated in a data-dependent MS/MS mode with survey full scan MS spectra (m/z 400-2000) acquired in the orbitrap with resolution of 30,000 at m/z 400 (ion accumulation target: 1,000,000), followed by MS/MS of the 10 most intense ions. In the case of label-free quantitation using the unfractionated CSF samples (the second and third set of normal CSF and the headache CSF), the LTQ-Orbitrap MS was operated in the data dependent mode with survey full scan spectra (m/z 400-2000) acquired in the orbitrap with resolution of 60,000 at m/z 400 (accumulation target: 1,000,000). The six most intense ions were sequentially isolated for fragmentation and detection in the linear ion trap.

Data Analysis.

The LTQ LC-MS/MS raw data were converted into .dta file by Extract_MSn (version 3.0) in Bioworks Cluster 3.2 (Thermo) and the SEQUEST algorithm (version 27 revision 12) was used to independently search all the MS/MS spectra against the human International Protein Index (IPI) database with a total of 69,731 total protein entries (Version 3.40, released at Feb. 7, 2008). The search parameters used were: 3-Da tolerance for precursor ion masses and 1-Da tolerance for fragment ion masses with no enzyme restraint and a maximum of 2 missed tryptic cleavages. Static carboxyamidomethylation of cysteine and dynamic oxidation of methionine were used during the database search. LTQ-Orbitrap MS/MS data were first processed by an in-house software DeconMSn [Mayampurath et al. (2008) Bioinformatics 24: 1021-1023] to accurately determine the monoisotopic mass and charge state of parent ions, followed by SEQUEST search against the IPI database in the same fashion, except that 0.1-Da tolerance for precursor ion masses and 1-Da tolerance for fragment ion masses were used. A set of criteria considering the cross correlation score (Xcorr) and delta correlation (ΔCn) values along with tryptic cleavage and charge states were developed using the decoy database approach and applied for filtering the raw data to limit false positive identifications to <1% at the peptide level [Peng et al. (2003) J Proteome Res 2: 43-50; Elias et al. (2007) Nat Methods 4: 207-214; Qian et al. (2005) J Proteome Res 4: 53-62]. For the LTQ-Orbitrap data, the distribution of mass deviation (from the theoretical masses) was first determined as having a standard deviation (a) of 2.05 part per million (ppm), and peptide identifications with mass error of greater than 3σ were filtered out. In general, slightly lower Xcorr cutoff values were used when combined with ΔCn and the mass error constraint to achieve the same level of false positive rate (<1%). For peptides identified by both LTQ-Orbitrap (IgY14 flow-through fraction) and LTQ (IgY14 bound fraction) analyses, the database matching scores are shown only for the LTQ-Orbitrap analysis, along with their mass errors.

The AMT tag strategy [Smith et al. (2002) Proteomics 2: 513-523] was used for identifying and quantifying LC-MS features measured by LTQ-Orbitrap. The filtered MS/MS peptide identifications obtained from the LTQ and LTQ-Orbitrap analyses of CSF samples were included in an AMT tag database with their theoretical mass and normalized elution time (NET; from 0 to 1) recorded. LC-MS datasets were then analyzed by in-house software VIPER [Monroe et al. (2007) Bioinformatics 23: 2021-2013] that detects features in mass-NET space and assigned them to peptides in the AMT tag database [Zimmer et al. (2006) Mass Spectrom Rev 25: 450-482]. A 11-Da shift strategy analogous to the decoy database approach used for LC-MS/MS identification of peptides was applied for estimating the false discovery rate of the AMT analysis as previously described [Petyuk et al. (2007) Genome Res 17: 328-336]. A false positive rate of <4% was estimated for each of the LC-MS data sets. The resulting lists of peptides from 2D-LC-MS/MS or direct LC-MS analysis was further analyzed by ProteinProphet software [Nesvizhskii et al. (2003) Anal Chem 75: 4646-4658] to remove redundancy in protein identification as described previously [Liu et al. (2006) Mol Cell Proteomics 5: 1899-1913].

Data normalization and quantification of the changes in protein abundance between the normal and headache CSF samples were performed and visualized using in-house software DAnTE [Polpitiya et al. (2008) Bioinformatics 24: 1556-1558]. Briefly, peptide intensities from the LC-MS analyses were log 2 transformed and normalized using a mean central tendency procedure. Peptide abundances were then “rolled up” to the protein level employing the R-rollup method (based on trends observed at peptide level) implemented in DAnTE. ANOVA and clustering analyses were also performed using DAnTE. Gene ontology annotation was performed using a software tool STRAP [Bhatia et al. (2009) Anal Chem 81: 9819-9823]. The final distribution charts were generated using Excel.

### Results

A comprehensive analysis of the CSF proteome from healthy normal individuals is described herein and presents the foundation for future investigation on this biological fluid which may be highly reflective of the status of the brain and central nervous system. The results described herein make available a comprehensive coverage of CSF proteins from normal healthy individuals. From the pool of 11 CSF samples from healthy volunteers, the present inventors identified with high confidence a total of 19,051 tryptic peptides, covering 2630 non-redundant proteins, with 1506 having at least two peptide identifications. The immunoaffinity-based partitioning generated a separate bound fraction consisting of the 14 most abundant proteins and their potential associated proteins, and a flow-through fraction enriched with the less abundant proteins in CSF. Similar to plasma, the bound fraction represents approximately 95% of the total protein mass. Both fractions were subjected to 2D-LC-MS/MS analysis.

The set of 2630 CSF proteins, and a comprehensive set of 3654 proteins from our previous plasma database [Liu et al. (2006) Mol Cell Proteomics 5: 1899-1913], showed very similar distribution of gene ontology terms in biological process and molecular function, but different distributions with respect to cellular component: a total of approximately 35% of the CSF proteins are from plasma membrane, cell surface or extracellular space, while blood plasma has a total of 28% of proteins in those three categories; there are also fewer CSF proteins derived from the nucleus and cytoplasm (10% versus 15% and 11% versus 16%, respectively. Importantly, nearly 56% of the proteins are CSF-specific and are not present in our larger plasma database of 3654 proteins, also analyzed by LC-MS/MS. This is notable because the acquisition and analysis conditions likely favored the set of plasma proteins as opposed to the CSF proteins. This is because more proteins were likely available for detection in the plasma of burn patients due to severe tissue leakage, and there was the additional dimension of sample fractionation via enrichment of cysteinyl and N-linked glycopeptides. It is noteworthy that this cannot be viewed as a head-to-head comparison in light of the differences in sample type and conditions, extensiveness of fractionation and MS instrumentation.

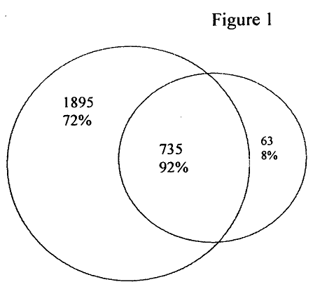

Comparison between proteins detected in this study and those (which we have termed neurologic surrogate-normals) from the CSF study by Zougman et al., reveals a 92% overlap (see FIG. 1.

In order to assess the CSF protein variability from serial sample collections, the present inventors examined individual (non-pooled) samples from another group of 10 healthy volunteers (5 male, 5 female; age range 37-44 years old) who had two CSF samples obtained at least 4 weeks apart using the AMT tag approach. Inter-subject differences were far greater than intra-subject differences. Statistical tests of variance of differences (ANOVA) were performed for these data sets based on different factors (e.g., subject, gender, and time of sampling), followed by unsupervised hierarchical clustering analysis of the statistically significant proteins (p-value<0.01). It is clear that human heterogeneity is the major factor responsible for inter-sample differences; clustering of the “significant” proteins could not distinguish corresponding groups based on the other factors defined in the ANOVA analysis (i.e., gender and time of sampling), except for “subject” (i.e., normal volunteers).

As an example of how CSF proteomic databases may be used to better understand disease states, the present inventors compared the proteomes of two similarly processed (see Methods) pooled samples of patients using the mass spectrometry strategy [Smith et al. (2002) Proteomics 2: 513-523]. The first set, considered as neurologic surrogate-normals was a pool of 10 headache patients. CSF had been obtained to evaluate the possibility of a CNS infection or bleed but all clinical results were normal. The second set, considered as non-neurologic surrogate-normals, was a pool of 200 subjects (without a neurologic disease, who underwent lumbar puncture for non diagnostic reasons; over 90% were undergoing spinal anesthesia in preparation for orthopedic surgery (limbs-knees and hips)). Significantly distinct results were found between each group. Specifically, 191±7 and 211±8 non-redundant proteins were identified from the 3 replicates of each data set. Statistical analysis comparing these CSF data sets showed that the neurologic surrogate-normal CSF pool had distinctive quantitative differences compared to the non-neurologic surrogate normal pool (22 proteins with p-value<0.01 by ANOVA). Unsupervised hierarchical clustering of abundances of all proteins clearly separates these two groups. One interesting difference was the identification and quantification of certain hemoglobin isoforms, which were among significantly changed proteins identified by the statistical analysis, in the neurologic surrogate-normal (headache) samples. Although Zougman et al. [(2008) J Proteome Res 7: 386-399] previously identified these same proteins in a qualitative analysis of their neurologic surrogate-normal samples, the present study reveals that these isoforms were increased by about ten-fold on average. The differences found in the present study suggest that it would be attractive to extend these studies with immunoaffinity depletion applied to different defined categories of headache subjects.

### Summary

The present study provides the most comprehensive CSF protein coverage and list reported to date for healthy normal individuals including serial lumbar punctures. [Schutzer et al. (2010) Establishing the Proteome of Normal Human Cerebrospinal Fluid, PLoS One 5(6): e10980]. The protein set has immediate utility for investigators interested in using CSF to study neurological or psychiatric diseases. As evidenced in Examples II-IV, it serves as a normative base to which disease states may be compared. The present study also suggests that CSF protein variability over a short time may be relatively limited in an individual and thus functionally useful.

The other major previous investigations of CSF from healthy individuals were published by Zhang et al. [(2005) Neurobiol Aging 26: 207-227] and updated by part of that group, Xu et al. [(2006) Int Rev Neurobiol 73: 29-98]. What began as detection of approximately 315 proteins was expanded to 915 using different mass spectrometry methods. Interestingly Xu et al. [(2006) Int Rev Neurobiol 73: 29-98] stated that they believed their coverage of the normal CSF was insufficient because they were unable to detect two well known CSF proteins, α-synuclein [Yuan et al. (2005) Proteomics 5: 541-550] and gelsolin [Yuan et al. (2002) Electrophoresis 23: 1185-1196]. The present methods and approach differed from these references, and included a rigorous separation of abundant from less abundant proteins to mitigate the masking effect of the most abundant proteins, as well as high-resolution LC coupled to MS/MS analysis for highly efficient peptide identification. The present inventors did identified 2630 proteins in total, including α-synuclein and gelsolin.

Due to the challenge in obtaining CSF from healthy people, most previous studies may have used CSF from “surrogate-normals,” that is CSF collected from people with neurological complaints such as headache but with normal clinical CSF laboratory values. As described herein, the present inventors compared proteomes of two different surrogate-normal groups. Significant differences were noted between the two groups. Accordingly, this study supports the potential usefulness of the “true” normal human CSF proteome data library as an invaluable tool in investigating pathophysiological abnormalities in neurological and psychiatric disorders.

Proteomic databases can be used in several ways. The present inventors' perspective with regard to using these proteomic databases for studying diseases involves a stepwise strategy. The first step is a comparison of pooled samples representative of the disease to normal subjects or a comparator disease. The second step involves the selection of specific candidate proteins. The selection of candidate proteins is not likely to be predicted in advance and may require bioinformatic strategies and knowledge related to the disease under study. A third step would involve analysis of the individual samples contributing to the pool to ascertain how many of the samples actually contained one or more of the candidate proteins. This step provides a check in the event that a single individual in the pool disproportionately contributes a protein compared to other subjects. The results would be subjected to statistical analyses in accordance with standard practice. In the case of a search for biomarker proteins, an objective is to select those that meet clinically useful criteria, such as presence, absence or relative abundance in a large percentage of disease subjects and not so in most subjects without the disease under consideration. The fourth step would involve verification of the previous results using independent individual samples with the same disease. A final validation step may involve analyzing a larger number of subjects with the disease and controls using assays targeted to the candidate proteins. In contrast to the discovery phases, it would be advantageous, if feasible, to use assay platforms already having wide clinical use. Immunobased assays such as ELISA and Western blots may serve this purpose being relatively inexpensive. Steps 3 and 4 will likely employ a type of mass spectrometry which targets selected candidate proteins, such as Multiple Reaction Monitoring (MRM) using triple quadrupole instrumentation.

The availability of the data presented here, detailing the normal human CSF proteome, should prove to be a critical base on which to compare proteins, both qualitatively and quantitatively, in studies of patients with a variety of neurological or psychiatric diseases.

### Example II

### Multiple Sclerosis

In the experimental analysis of data on documented first attack Multiple Sclerosis subjects, long lists of proteins found in these subjects (data not shown) and small subset lists as well as a list of those proteins that appear to be absent (below the level of detection) have been found.

### Specifically:

1) Table 1m shows Cerebrospinal Fluid (CSF) Proteins present only in Multiple Sclerosis patients compared to Surrogate Non-Neurologic Normals and to Surrogate Neurologic Normals

(Headaches). This is a Qualitative List of CSF Proteins Present in the Pooled First Attack Multiple Sclerosis Patients compared to the combined lists of Proteins Found in a Similarly Analyzed Group of Non-neurologic Surrogate Normal Subjects and Proteins Found in Neurologic Surrogate-Normals (Headache subjects with normal CSF clinical laboratory values).

2) Table 2m shows a Qualitative List of CSF Proteins Absent only in the Pooled First Attack Multiple Sclerosis Patients compared to the combined lists of a) Proteins Found in a similarly Analyzed Group of Non-neurologic Surrogate Normal Subjects (both groups analyzed without immunoaffinity depletion of most abundant proteins) and b) Proteins Found in an expanded list of True Normals (where immunoaffinity depletion was applied).

3) Table 3m shows a subset of CSF Proteins Present In Multiple Sclerosis not in combined lists of proteins from Normals or Headaches (immunoaffinity depletion was not applied)

4) Table 4H-m shows identified CSF proteins in first attack Multiple Sclerosis that have high potential to be candidate biomarkers because their function is not known and they remain unknown (referred to as a hypothetical or uncharacterized protein).

### Example III

### Chronic Fatigue Syndrome

In the experimental analysis of data on Chronic Fatigue Syndrome subjects, long lists of proteins were found in these subjects and smaller subset lists as well as a list of those proteins that appear to be absent (below the level of detection). Immunoaffinity depletion of abundant proteins was applied in the analyses. In addition, semi-quantitative, or relative protein abundance comparisons were made to proteins found in a disease that has common features with Lyme disease-Chronic Fatigue Syndrome. Also found were proteins associated with this group that are considered hypothetical or uncharacterized proteins.

### Specifically:

1) Table 4H-c shows the list by IPI number of those hypothetical or uncharacterized proteins that were found in Chronic Fatigue Syndrome.

2) Table 5c shows the CSF Proteins Present in Chronic Fatigue and not in Normals or Neurologic Lyme subjects. All had immunoaffinity depletion. They are listed by IPI identification numbers representing 738 CSF Proteins.

3) Table 6c shows the CSF Proteins Absent in Chronic Fatigue but Present in Neurologic Lyme or Normals.

4) Table 9 shows the Quantitative Relative Abundance of CSF Proteins in Chronic Fatigue compared to Neurologic Lyme Disease. A ratio greater than 1 indicates there is a relative quantitative difference in Chronic Fatigue compared to Neurologic Lyme disease. Ratios near 1 suggest equal relative abundances. Ratios less than 1 indicate that the protein is present in a greater relative abundance in Neurologic Lyme disease compared to Chronic Fatigue. There are 168 proteins in Chronic Fatigue that are 2 ford greater in relative abundance than those proteins Neurologic Lyme disease. There are 1081 proteins that are relatively more abundant in Neurologic Lyme disease compared to Chronic Fatigue with a ratio of 0.5 or less. Blank cell is close to 0 ratio.

### Example IV

### Neurologic Lyme Disease

In the experimental analysis of data on documented Neurologic Lyme disease subjects, long lists of proteins were found in these subjects and smaller subset lists as well as a list of those proteins that appear to be absent (below the level of detection). Immunoaffinity depletion of abundant proteins was applied in the analyses. In addition, semi-quantitative, or relative protein abundance comparisons were made to proteins found in a disease that has common features with Lyme disease-Chronic Fatigue Syndrome. Also found were proteins associated with this group that are considered hypothetical or uncharacterized proteins.

### Specifically:

1) Table 4H-L shows the list of by IPI number of those hypothetical or uncharacterized proteins that were found in Neurologic Lyme disease.

2) Table 7 L shows the Qualitative List of CSF Proteins Identified (Present) in Patients with Neurologic Lyme disease and not found in True Normals or Chronic Fatigue. All with immunoaffinity depletion. There are 692 proteins in this list.

3) Table 8 L shows the CSF Proteins Absent in Neurologic Lyme disease but Present in Normals or Chronic Fatigue subjects. There are 1597 Proteins in this list.

4) Table 9 shows the Quantitative Relative Abundance of CSF Proteins in Chronic Fatigue compared to Neurologic Lyme Disease. A ratio greater than 1 indicates there is a relative quantitative difference in Chronic Fatigue compared to Neurologic Lyme disease. Ratios near 1 suggest equal relative abundances. Ratios less than 1 indicate the protein is present in a greater relative abundance in Neurologic Lyme disease compared to Chronic Fatigue. There are 168 proteins in Chronic Fatigue that are 2 fold greater in relative abundance than those proteins Neurologic Lyme disease. There are 1081 proteins that are relatively more abundant in Neurologic Lyme disease compared to Chronic Fatigue with a ratio of 0.5 or less. Blank cell is close to 0 ratio)

While certain of the preferred embodiments of the present invention have been described and specifically exemplified above, it is not intended that the invention be limited to such embodiments. Various modifications may be made thereto without departing from the scope and spirit of the present invention, as set forth in the following claims.

TABLE 1m
Cerebrospinal Fluid (CSF) Proteins present only in Multiple Sclerosis patients
compared to Surrogate Non-Neurologic Normals AND Surrogate Neurologic Normals
(Headaches): Qualitative List of CSF Proteins Present in the Pooled First Attack Multiple
Sclerosis Patients compared to the combined lists of Proteins Found in a Similarly Analyzed
Group of Non-neurologic Surrogate Normal Subjects and Proteins Found in Neurologic
Surrogate-Normals (Headaches subjects with normal CSF clinical laboratory values).

| | CSF Proteins Present In MS not | |
|---|---|---|
| MinOfORF | Normals or Headaches (no IAD) | Gene |
| IPI:IPI00000816.1 | 14-3-3 protein epsilon | YWHAE |
| IPI:IPI00001516.1 | Isoform Long of Protocadherin alpha C2 precursor | PCDHAC2 |
| IPI:IPI00001610.1 | Insulin-like growth factor IA precursor | IGF1 |
| IPI:IPI00001895.1 | Isoform 1 of Protocadherin-8 precursor | PCDH8 |
| IPI:IPI00006444.1 | Isoform 1 of Sodium/potassium/calcium exchanger 2 precursor | SLC24A2 |
| IPI:IPI00006543.2 | Complement factor H-related 5 | CFHR5 |
| IPI:IPI00007778.1 | Di-N-acetylchitobiase precursor | CTBS |
| IPI:IPI00008994.2 | Isoform 1 of Protein NDRG2 | NDRG2 |
| IPI:IPI00012102.1 | N-acetylglucosamine-6-sulfatase precursor | GNS |
| IPI:IPI00012283.2 | Isoform 1 of Semaphorin-3B precursor | SEMA3B |
| IPI:IPI00012792.1 | Cadherin-5 precursor | CDH5 |
| IPI:IPI00015479.5 | UPF0454 protein C12orf49 precursor | C12orf49 |
| IPI:IPI00015911.1 | Dihydrolipoyl dehydrogenase, mitochondrial precursor | DLD |
| IPI:IPI00016666.1 | Metallothionein-3 | MT3 |
| IPI:IPI00017569.5 | Fas apoptotic inhibitory molecule 2 | FAIM2 |
| IPI:IPI00017704.3 | Coactosin-like protein | COTL1 |
| IPI:IPI00021833.1 | Isoform Long of Platelet-derived growth factor A chain | PDGFA |
| | precursor | |
| IPI:IPI00025365.1 | Isoform Long of Endothelin-3 precursor | EDN3 |
| IPI:IPI00026569.3 | HLA class I histocompatibility antigen, A-1 alpha chain | HLA-A |
| | precursor | |
| IPI:IPI00026946.2 | Neuronal pentraxin-2 precursor | NPTX2 |
| IPI:IPI00027765.1 | retbindin isoform 2 | RTBDN |
| IPI:IPI00029131.1 | Neuroendocrine convertase 2 precursor | PCSK2 |
| IPI:IPI00030877.2 | 15 kDa selenoprotein isoform 1 precursor | SEP15 |
| IPI:IPI00060310.4 | Phospholipase D4 | PLD4 |
| IPI:IPI00152540.3 | Isoform 1 of CD109 antigen precursor | CD109 |
| IPI:IPI00157414.3 | Ectonucleotide pyrophosphatase/phosphodiesterase family | ENPP6 |
| | member 6 precursor | |
| IPI:IPI00163187.10 | Fascin | FSCN1 |
| IPI:IPI00171411.4 | Golgi phosphoprotein 2 | GOLM1 |
| IPI:IPI00172636.5 | Isoform Delta 6 of Calcium/calmodulin-dependent protein | CAMK2D |
| | kinase type II delta chain | |
| IPI:IPI00180707.8 | Isoform 1 of FRAS1-related extracellular matrix protein 2 | FREM2 |
| | precursor | |
| IPI:IPI00217542.4 | Putative uncharacterized protein LOC130576 | LOC130576 |
| IPI:IPI00217966.7 | Isoform 1 of L-lactate dehydrogenase A chain | LDHA |
| IPI:IPI00292550.2 | Isoform 1 of Polypeptide N-acetylgalactosaminyltransferase 13 | GALNT13 |
| IPI:IPI00294705.1 | Papilin | PAPLN |
| IPI:IPI00298547.3 | Protein DJ-1 | PARK7 |
| IPI:IPI00298793.4 | Beta-mannosidase precursor | MANBA |
| IPI:IPI00329688.2 | Protein YIPF3 | YIPF3 |
| IPI:IPI00334666.2 | Isoform 1 of Receptor-type tyrosine-protein phosphatase N2 | PTPRN2 |
| | precursor | |
| IPI:IPI00337548.5 | Cell growth regulator with EF hand domain protein 1 | CGREF1 |
| IPI:IPI00376394.4 | Sulfhydryl oxidase 2 precursor | QSOX2 |
| IPI:IPI00382426.1 | Ig lambda chain V-II region TRO | |
| IPI:IPI00382447.1 | Ig lambda chain V-VI region AR | |
| IPI:IPI00382476.1 | Ig heavy chain V-III region WEA | |
| IPI:IPI00382482.1 | Ig heavy chain V-III region CAM | |
| IPI:IPI00382490.1 | Ig heavy chain V-III region BUR | |
| IPI:IPI00384402.1 | Myosin-reactive immunoglobulin kappa chain variable region | |
| | (Fragment) | |
| IPI:IPI00385143.1 | Microfibrillar protein 2 (Fragment) | |
| IPI:IPI00386131.1 | Ig kappa chain V-III region IARC/BL41 precursor | |
| IPI:IPI00387096.1 | Ig kappa chain V-I region Kue | |
| IPI:IPI00387101.1 | Ig kappa chain V-I region Scw | |
| IPI:IPI00387113.1 | Ig kappa chain V-III region B6 | |
| IPI:IPI00419442.3 | IGLV6-57 protein | IGLV6-57 |
| IPI:IPI00455667.5 | hypothetical protein LOC402665 | LOC402665 |
| IPI:IPI00465255.6 | Isoform 1 of Proline-rich acidic protein 1 precursor | PRAP1 |
| IPI:IPI00748265.2 | Rheumatoid factor RF-ET13 | |
| IPI:IPI00816737.1 | Rheumatoid factor D5 heavy chain (Fragment) | |
| IPI:IPI00829810.1 | Uncharacterized protein ENSP00000375027 | |
| IPI:IPI00854624.1 | Uncharacterized protein ENSP00000375043 | |
| IPI:IPI00854745.1 | Uncharacterized protein ENSP00000375019 | |

IAD = immunoaffinity depletion

TABLE 2m
Qualitative List of CSF Proteins Absent only in the Pooled First Attack Multiple Sclerosis
Patients compared to the combined lists of a) Proteins Found in a similarly Analyzed
Group of Non-neurologic Surrogate Normal Subjects (both groups analyzed without
immunoaffinity depletion (IAD) of most abundant proteins) and b) Proteins Found in an
expanded list of True Normals (where immunoaffinity depletion was applied).

| | | CSF Proteins Not Present in MS but Present in Combined |
|---|---|---|
| MinOfORF | Gene | Normals and Headaches (no IAD) |
| IPI:IPI00000024.1 | PCDH1 | Isoform 1 of Protocadherin-1 precursor |
| IPI:IPI00001477.1 | DDR1 | Isoform 1 of Epithelial discoidin domain-containing receptor |
| | | 1 precursor |
| IPI:IPI00003176.1 | HTRA1 | Serine protease HTRA1 precursor |
| IPI:IPI00003648.2 | PVRL1 | Isoform Delta of Poliovirus receptor-related protein 1 |
| | | precursor |
| IPI:IPI00004946.8 | CXCL16 | chemokine (C—X—C motif) ligand 16 |
| IPI:IPI00010471.5 | LCP1 | Plastin-2 |
| IPI:IPI00021263.3 | YWHAZ | 14-3-3 protein zeta/delta |
| IPI:IPI00021856.3 | APOC2 | Apolipoprotein C-II precursor |
| IPI:IPI00022333.1 | BAI1 | Brain-specific angiogenesis inhibitor 1 precursor |
| IPI:IPI00024570.1 | SEMA3G | Semaphorin-3G precursor |
| IPI:IPI00025155.1 | FSTL3 | Follistatin-related protein 3 precursor |
| IPI:IPI00029193.1 | HGFAC | Hepatocyte growth factor activator precursor |
| IPI:IPI00029343.2 | CNTNAP2 | Isoform 1 of Contactin-associated protein-like 2 precursor |
| IPI:IPI00169383.3 | PGK1 | Phosphoglycerate kinase 1 |
| IPI:IPI00216171.3 | ENO2 | Gamma-enolase |
| IPI:IPI00218570.6 | PGAM2 | Phosphoglycerate mutase 2 |
| IPI:IPI00218834.9 | FCGR3A | Low affinity immunoglobulin gamma Fc region receptor III- |
| | | A precursor |
| IPI:IPI00219425.3 | PVR | Isoform Beta of Poliovirus receptor precursor |
| IPI:IPI00297040.2 | SPINK6 | Serine protease inhibitor Kazal-type 6 precursor |
| IPI:IPI00306710.2 | CHRD | Isoform 1 of Chordin precursor |
| IPI:IPI00328703.1 | OAF | Out at first protein homolog precursor |
| IPI:IPI00329775.7 | CPB2 | Isoform 1 of Carboxypeptidase B2 precursor |
| IPI:IPI00385980.8 | ROBO2 | ROBO2 isoform a |
| IPI:IPI00387119.1 | | Ig kappa chain V-III region POM |
| IPI:IPI00411680.8 | PCMT1 | Isoform 1 of Protein-L-isoaspartate(D-aspartate) O- |
| | | methyltransferase |
| IPI:IPI00444605.1 | | CDNA FLJ45296 fis, clone BRHIP3003340, moderately |
| | | similar to Actin, alpha skeletal muscle 2 |
| IPI:IPI00456736.4 | RGMB | Isoform 1 of RGM domain family member B precursor |
| IPI:IPI00479116.1 | CPN2 | Carboxypeptidase N subunit 2 precursor |
| IPI:IPI00554474.3 | LOC284297 | Hypothetical LOC284297 |

MinOfORF = Non redundant IPI designation

TABLE 3m
A subset table of Table 1 illustrating those CSF proteins identified that
have high potential as candidate biomarkers.

| | CSF Proteins Present In | |
|---|---|---|
| | Multiple Sclerosis not in | |
| | combined lists of proteins from | |
| MinOfORF | Normals or Headaches (no IAD) | GENE |
| IPI:IPI00001516.1 | Isoform Long of Protocadherin alpha | PCDHAC2 |
| | C2 precursor | |
| IPI:IPI00026569.3 | HLA class I histocompatibility | HLA-A |
| | antigen, A-1 alpha chain | |
| | precursor | |
| IPI:IPI00382426.1 | Ig lambda chain V-II region TRO | |
| IPI:IPI00382447.1 | Ig lambda chain V-VI region AR | |
| IPI:IPI00382490.1 | Ig heavy chain V-III region BUR | |
| IPI:IPI00829810.1 | Uncharacterized protein | |
| | ENSP00000375027 | |
| IPI:IPI00854624.1 | Uncharacterized protein | |
| | ENSP00000375043 | |
| IPI:IPI00854745.1 | Uncharacterized protein | |
| | ENSP00000375019 | |

MinOfORF = Non redundant IPI designation
IAD = Immunoaffinity depletion

Table 4H. This Table is divided into 3. The tables show identified CSF proteins that have high potential to be candidate biomarkers because their function is not known and they remain unknown (referred to as a hypothetical or uncharacterized protein).

Table 4H-m is a subset of Table 1 and Table 3 containing those hypothetical/uncharacterized CSF proteins identified in Multiple Sclerosis

TABLE 4H

| | CSF Proteins Present In Multiple | |
|---|---|---|
| | Sclerosis but not | |
| | Normals or Headaches | |
| | (no IAD) Hypothetical+++, | |
| MinOfORF | where Ig removed from list | Gene |
| IPI:IPI00001516.1 | Isoform Long of Protocadherin alpha | PCDHAC2 |
| | C2 precursor | |
| IPI:IPI00829810.1 | Uncharacterized protein | |
| | ENSP00000375027 | |
| IPI:IPI00854624.1 | Uncharacterized protein | |
| | ENSP00000375043 | |
| IPI:IPI00854745.1 | Uncharacterized protein | |
| | ENSP00000375019 | |

IAD = Immunoaffinity depletion
MinOfORF = Non redundant IPI designation

TABLE 4H-l
Hypothetical proteins found in neurologic
Post Treatment Lyme Disease IPI

| | |
|---|---|
| IPI00166766 | IPI00852979 |
| IPI00443799 | IPI00374914 |
| IPI00554474 | IPI00003269 |
| IPI00455667 | IPI00749328 |
| IPI00414294 | IPI00644752 |
| IPI00166465 | IPI00749338 |
| IPI00328872 | IPI00794307 |
| IPI00181279 | IPI00847409 |
| IPI00644840 | IPI00411480 |
| IPI00848252 | IPI00739387 |
| IPI00847373 | IPI00855747 |
| IPI00186004 | IPI00384971 |
| IPI00395010 | IPI00061520 |
| IPI00400986 | IPI00006556 |
| IPI00795992 | |

TABLE 4H-c
Hypothetical proteins detected in
Chronic fatigue IPI
IPI00003269
IPI00006556
IPI00061520
IPI00166766
IPI00168255
IPI00176210
IPI00328872
IPI00374223
IPI00374914
IPI00395010
IPI00396025
IPI00398586
IPI00400986
IPI00413868
IPI00414294
IPI00418966
IPI00419675
IPI00443799
IPI00455667
IPI00550862
IPI00554474
IPI00644752
IPI00749328
IPI00789889
IPI00807406
IPI00852979

TABLE 5c
CSF Proteins Present in Chronic Fatigue and not in Normals or
Neurologic Lyme subjects all had immunoaffinity depletion

| IPI | CSF Proteins 738 | Gene |
|---|---|---|
| IPI00103595 | Centrosome-associated protein 350 | CEP350 |
| IPI00006173 | Isoform 1 of Cholesteryl ester transfer protein precursor | CETP |
| IPI00183526 | NCL protein | NCL |
| IPI00020406 | Beta-1,4-mannosyl-glycoprotein 4-beta-N-acetylglucosaminyltransferase | MGAT3 |
| IPI00022479 | guanine nucleotide exchange factor p532 | HERC1 |
| IPI00179452 | Isoform 1 of Protein CBFA2T2 | CBFA2T2 |
| IPI00017480 | Sonic hedgehog protein precursor | SHH |
| IPI00853133 | DEAD (Asp-Glu-Ala-Asp) box polypeptide 60-like | DDX60L |
| IPI00387132 | CDNA FLJ32661 fis, clone TESTI1000055, weakly similar to HOMEOBOX | hCG_2007354 |
| | PROTEIN SIXI | |
| IPI00878369 | 12 kDa protein | — |
| IPI00382492 | Ig heavy chain V-III region LAY | — |
| IPI00334492 | Isoform 3 of Coiled-coil domain-containing protein 144A | LOC731479; CCDC144A; |
| | | LOC652491; CCDC144B |
| IPI00018880 | Tumor necrosis factor receptor superfamily member 1A precursor | TNFRSF1A |
| IPI00784385 | Adenylate cyclase type 1 | ADCY1 |
| IPI00465168 | Isoform 1 of Serine/threonine-protein kinase TAO2 | TAOK2 |
| IPI00028213 | Semaphorin-3D precursor | SEMA3D |
| IPI00296421 | EH domain-binding protein 1-like protein 1 | EHBP1L1 |
| IPI00787587 | similar to Temporarily Assigned Gene name family member | LOC729549 |
| IPI00028980 | Uncharacterized protein KIAA0133 | KIAA0133 |
| IPI00430792 | Isoform 1 of Protein ERGIC-53-like precursor | LMAN1L |
| IPI00018335 | Isoform Flt1 of Vascular endothelial growth factor receptor 1 precursor | FLT1 |
| IPI00795736 | Fer-1-like 4 | — |
| IPI00000656 | Isoform 1 of Uncharacterized protein KIAA0892 precursor | KIAA0892 |
| IPI00169377 | Isoform 1 of GAS2-like protein 2 | GAS2L2 |
| IPI00030648 | Isoform 1 of Zinc finger FYVE domain-containing protein 9 | ZFYVE9 |
| IPI00419675 | hypothetical protein LOC150763 | LOC150763 |
| IPI00003893 | Isoform 1 of Protocadherin gamma B3 precursor | PCDHGB3 |
| IPI00410127 | Transforming, acidic coiled-coil containing protein 2 | TACC2 |
| IPI00005711 | Histone deacetylase 6 | HDAC6 |
| IPI00883997 | Coiled-coil domain-containing protein 19 | CCDC19 |
| IPI00419180 | CDNA FLJ41429 fis, clone BRHIP2005354 | CC2D2B |
| IPI00335711 | Ciliary dynein heavy chain 11 | DNAH11 |
| IPI00411291 | Peroxisome biogenesis factor 1 | PEX1 |
| IPI00397622 | Isoform 1 of Transmembrane and immunoglobulin domain-containing protein 1 | TMIGD1 |
| | precursor | |
| IPI00003564 | Lymphocyte antigen 86 precursor | LY86 |
| IPI00100247 | Thioredoxin domain-containing protein 13 precursor | TXNDC13 |
| IPI00059711 | LRP11 protein (Fragment) | LRP11 |
| IPI00141933 | Mitotic checkpoint serine/threonine-protein kinase BUB1 beta | BUB1B |
| IPI00387098 | Ig kappa chain V-I region OU | — |
| IPI00299554 | Kinesin-like protein KIF14 | KIF14 |
| IPI00515034 | 87 kDa protein | KIFAP3 |
| IPI00044608 | Isoform 1 of Protein KIAA1881 | KIAA1881 |
| IPI00444138 | CDNA FLJ46074 fis, clone TESTI2001915, highly similar to Homo sapiens actin | AFAP1 |
| | filament associated protein | |
| IPI00807406 | hypothetical protein isoform 1 | LOC285346 |
| IPI00645729 | 62 kDa protein | C1QTNF8 |
| IPI00789856 | tubulin tyrosine ligase-like family, member 3 | TTLL3 |
| IPI00032680 | Apolipoprotein-L5 | APOL5 |
| IPI00298214 | Isoform 1 of ATP-binding cassette sub-family G member 2 | ABCG2 |
| IPI00030945 | Urocortin-2 precursor | UCN2 |
| IPI00333503 | Uncharacterized protein ENSP00000315571 (Fragment) | — |
| IPI00005567 | Isoform 3A of Voltage-dependent L-type calcium channel subunit beta-3 | CACNB3 |
| IPI00185038 | Isoform 1 of Dual oxidase 1 precursor | DUOX1 |
| IPI00005537 | 39S ribosomal protein L12, mitochondrial precursor | MRPL12 |
| IPI00795575 | 39 kDa protein | — |
| IPI00221240 | Isoform 2 of Leucyl-cystinyl aminopeptidase | LNPEP |
| IPI00655865 | Isoform 2 of Protein diaphanous homolog 3 | DIAPH3 |
| IPI00019270 | Tankyrase-2 | TNKS2 |
| IPI00302614 | V-set domain containing T cell activation inhibitor 1 | VTCN1 |
| IPI00176976 | Novel protein similar to mitochondrial ribosome recycling factor isoform 1 | RP1-57A13.2 |
| IPI00786931 | similar to fatty acid amide hydrolase | LOC729037 |
| IPI00063878 | Isoform 1 of Multidrug resistance-associated protein 9 | ABCC12 |
| IPI00029473 | Protein Njmu-R1 | C17orf75 |
| IPI00884076 | ENV polyprotein (coat polyprotein) family protein | — |
| IPI00449923 | Isoform 1 of Retinoic acid-induced protein 1 | RAI1 |
| IPI00294187 | Protein-arginine deiminase type-2 | PADI2 |
| IPI00045473 | Similar to C. Elegans protein F17C8.5 | LOC339123 |
| IPI00167433 | CDNA FLJ39703 fis, clone SMINT2012195 | FDX1L |
| IPI00183572 | Isoform 2 of Dedicator of cytokinesis protein 7 | DOCK7 |
| IPI00001835 | Zinc fmger and BTB domain-containing protein 4 | ZBTB4 |
| IPI00148768 | TRIO and F-actin binding protein isoform 1 | TRIOBP |
| IPI00170548 | Isoform 1 of ATPase family AAA domain-containing protein 2 | ATAD2 |
| IPI00220257 | Isoform 3 of Probable tubulin polyglutamylase | TTLL1 |
| IPI00298058 | Isoform 1 of Transcription elongation factor SPT5 | SUPT5H |
| IPI00102281 | Retroviral-like aspartic protease 1 precursor | SASP |
| IPI00030355 | Protein phosphatase 1 regulatory subunit 11 | PPP1R11 |
| IPI00217915 | Isoform 1 of Transmembrane protein 136 | TMEM136 |
| IPI00220986 | Isoform 3 of ADAMTS-9 precursor | ADAMTS9 |
| IPI00175169 | Isoform 1 of ADP-ribosylation factor GTPase-activating protein 1 | ARFGAP1 |
| IPI00550862 | hypothetical protein LOC387911 | LOC387911 |
| IPI00746459 | CDNA FLJ34517 fis, clone HLUNG2006781 | — |
| IPI00444234 | CDNA FLJ45743 fis, clone KIDNE2016464 | FLJ45743 |
| IPI00002534 | Isoform 1 of Purine-rich element-binding protein gamma | PURG |
| IPI00556253 | Isoform 1 of Coiled-coil domain-containing protein C6orf199 | C6orf199 |
| IPI00017592 | Leucine zipper-EF-hand-containing transmembrane protein 1, mitochondrial | LETM1 |
| | precursor | |
| IPI00016007 | Myosin-Vc | MYO5C |
| IPI00744927 | gastrotropin isoform 1 | FABP6 |
| IPI00024319 | Forkhead box protein J1 | FOXJ1 |
| IPI00339297 | Polypeptide N-acetylgalactosaminyltransferase 4 | GALNT4 |
| IPI00254408 | bromodomain PHD finger transcription factor isoform 1 | BPTF |
| IPI00304324 | Histone deacetylase 11 | HDAC11 |
| IPI00477693 | fatty acid binding protein | LOC646486 |
| IPI00025310 | Zinc finger protein 217 | ZNF217 |
| IPI00427808 | Isoform 1 of Uncharacterized protein C10orf71 | C10orf71 |
| IPI00735313 | similar to Peptidyl-prolyl cis-trans isomerase NIMA-interacting 4 | — |
| IPI00384013 | Polypeptide N-acetylgalactosaminyltransferase-like protein 2 | GALNTL2 |
| IPI00382495 | Ig heavy chain V-III region ZAP | — |
| IPI00477820 | Isoform 1 of FRAS1-related extracellular matrix protein 1 precursor | FREM1 |
| IPI00024776 | Calmegin precursor | CLGN |
| IPI00023110 | Potassium channel subfamily K member 3 | KCNK3 |
| IPI00182833 | Coiled-coil alpha-helical rod protein 1 | CCHCR1 |
| IPI00394994 | Isoform 1 of Nesprin-3 | C14orf49 |
| IPI00419992 | Isoform 1 of Acyl-CoA synthetase family member 4 | AASDH |
| IPI00004445 | Gamma-2-syntrophin | SNTG2 |
| IPI00878015 | 12 kDa protein | — |
| IPI00328094 | Isoform 1 of Forkhead box protein P3 | FOXP3 |
| IPI00873348 | 63 kDa protein | A1CF |
| IPI00640240 | 59 kDa protein | SPTLC3 |
| IPI00002824 | Cysteine and glycine-rich protein 2 | CSRP2 |
| IPI00398272 | Isoform 1 of Collagen alpha-1(XVII) chain | COL17A1 |
| IPI00061009 | Isoform 1 of WD repeat-containing protein 67 | WDR67 |
| IPI00299301 | Isoform 1 of Desmuslin | DMN |
| IPI00152011 | PTPL1-associated RhoGAP 1 | ARHGAP29 |
| IPI00847790 | Protein of unknown function DUF1725 domain containing protein | — |
| IPI00065121 | Isoform 2 of Coiled-coil domain-containing protein 36 | CCDC36 |
| IPI00373867 | glycolipid transfer protein domain containing 2 | GLTPD2 |
| IPI00872354 | armadillo repeat containing 2 | ARMC2 |
| IPI00152975 | Isoform 1 of Coiled-coil domain-containing protein 90A, mitochondrial precursor | CCDC90A |
| IPI00374804 | Isoform 1 of Diacylglycerol kinase delta | DGKD |
| IPI00292894 | Pre-rRNA-processing protein TSR1 homolog | TSR1 |
| IPI00028232 | Putative uncharacterized protein | FGF4 |
| IPI00027269 | E3 ubiquitin-protein ligase CBL | CBL |
| IPI00797567 | 9 kDa protein | GNAI2 |
| IPI00032958 | Isoform 2 of Actin-binding protein anillin | ANLN |
| IPI00057815 | intestinal facilitative glucose transporter 7 | SLC2A7 |
| IPI00014367 | Isoform 1 of Non-SMC element 4 homolog A | NSMCE4A |
| IPI00884022 | Similar to Immunglobulin heavy chain variable region | — |
| IPI00465319 | Chondroitin sulfate synthase 2 | CHPF |
| IPI00549844 | Isoform 1 of Protein FAM134B | FAM134B |
| IPI00457114 | Isoform 1 of IQ motif and Sec7 domain-containing protein 1 | IQSEC1 |
| IPI00002773 | Isoform 2 of Tyrosine-protein kinase JAK3 | JAK3 |
| IPI00005667 | NEDD4-binding protein 1 | N4BP1 |
| IPI00300594 | CDNA FLJ35435 fis, clone SMINT2002620 | SPATA13 |
| IPI00103142 | NudC domain-containing protein 2 | NUDCD2 |
| IPI00290785 | Isoform 1 of SET domain-containing protein 6 | SETD6 |
| IPI00297763 | Retinal-specific ATP-binding cassette transporter | ABCA4 |
| IPI00000861 | Isoform 1 of LIM and SH3 domain protein 1 | LASP1 |
| IPI00168255 | hypothetical protein LOC400451 | LOC400451 |
| IPI00015990 | Isoform 1 of Eyes absent homolog 3 | EYA3 |
| IPI00171500 | Uncharacterized protein C14orf45 | C14orf45 |
| IPI00170765 | Isoform 3 of Alanine--glyoxylate aminotransferase 2-like 2 | AGXT2L2 |
| IPI00099977 | platelet-derived growth factor C precursor | PDGFC |
| IPI00292953 | Isoform 2 of Ankycorbin | RAI14 |
| IPI00159072 | 57 kDa protein | ROD1 |
| IPI00305692 | Thioredoxin-like protein 1 | TXNL1 |
| IPI00031630 | Melanocyte protein Pmel 17 precursor | SILV |
| IPI00167446 | Isoform 2 of Uncharacterized protein C17orf78 | C17orf78 |
| IPI00479625 | Isoform 1 of ELAV-like protein 4 | ELAVL4 |
| IPI00745395 | Conserved hypothetical protein | KIAA0508 |
| IPI00043069 | Ankyrin repeat domain-containing protein 30A | ANKRD30A |
| IPI00014316 | Cyclin-dependent kinase 5 activator 1 precursor | CDK5R1 |
| IPI00301726 | Isoform 1 of Uncharacterized protein C3orf20 | C3orf20 |
| IPI00185361 | ATP-dependent RNA helicase DDX55 | DDX55 |
| IPI00293867 | D-dopachrome decarboxylase | DDT |
| IPI00794779 | DNA polymerase theta | POLQ |
| IPI00478354 | Putative uncharacterized protein CXorf31 | CXorf31 |
| IPI00166414 | Coiled-coil domain-containing protein 96 | CCDC96 |
| IPI00300053 | Keratin type II cuticular Hb2 | KRT82 |
| IPI00749062 | Conserved hypothetical protein | — |
| IPI00452161 | Isoform 1 of Mucolipin-1 | MCOLN1 |
| IPI00384546 | CDNA FLJ25883 fis, clone CBR02735 | — |
| IPI00412272 | SH3 domain-binding glutamic acid-rich-like protein 2 | SH3BGRL2 |
| IPI00006298 | Isoform 1 of Peptidyl-prolyl cis-trans isomerase G | PPIG |
| IPI00031497 | Conserved hypothetical protein | C7orf49 |
| IPI00022061 | Sodium/hydrogen exchanger 6 | SLC9A6 |
| IPI00217418 | Isoform 2 of Rho GTPase-activating protein 12 | ARHGAP12 |
| IPI00743545 | Conserved hypothetical protein | — |
| IPI00328156 | Amine oxidase [flavin-containing] B | MAOB |
| IPI00644785 | Protein | PLXNB3 |
| IPI00654693 | Protein FAM44C | FAM44C |
| IPI00880011 | 116 kDa protein | — |
| IPI00018583 | Isoform 1 of Intracellular hyaluronan-binding protein 4 | HABP4 |
| IPI00030986 | Isoform Long of Kelch repeat and BTB domain-containing protein 10 | KBTBD10 |
| IPI00045503 | Isoform 2 of Protein phosphatase 1 regulatory subunit 14A | PPP1R14A |
| IPI00793408 | 6 kDa protein | ADAMTS9 |
| IPI00024255 | G patch domain and KOW motifs-containing protein | GPKOW |
| IPI00028262 | 67 kDa protein | KIAA1754 |
| IPI00027212 | Insulin receptor-related protein precursor | INSRR |
| IPI00009724 | Isoform 1 of EF-hand calcium-binding domain-containing protein 6 | EFCAB6 |
| IPI00868835 | Similar to Heterogeneous nuclear ribonucleoproteins C1/C2 (hnRNP C1/hnRNP | — |
| | C2). Isoform 4 | |
| IPI00396658 | Isoform 2 of Protein ITFG3 | ITFG3 |
| IPI00742127 | Similar to Heterogeneous nuclear ribonucleoprotein A1 (Helix-destabilizing | — |
| | protein) (Single-strand RNA-binding protein) (hnRNP core protein A1). Isoform | |
| | A1-A | |
| IPI00398586 | hypothetical protein LOC199800 | LOC199800 |
| IPI00418164 | Novel protein | RP4-692D3.1; LOC731440 |
| IPI00033019 | Potassium voltage-gated channel subfamily B member 1 | KCNB1 |
| IPI00032598 | Isoform 1 of ETS translocation variant 3 | ETV3 |
| IPI00217494 | Isoform 1 of Protein SMG7 | SMG7 |
| IPI00000837 | Metabotropic glutamate receptor 3 precursor | GRM3 |
| IPI00296535 | 2-hydroxyacyl-CoA lyase 1 | HACL1 |
| IPI00062882 | ATP-binding domain-containing protein 3 | ATPBD3 |
| IPI00445167 | CDNA FLJ44499 fis, clone UTERU3000665, highly similar to Homo sapiens | SRCAP |
| | Snf2-related CBP activator protein | |
| IPI00442582 | CDNA FLJ26893 fis, clone RCT00305 | — |
| IPI00011274 | Isoform 1 of Heterogeneous nuclear ribonucleoprotein D-like | HNRPDL |
| IPI00002791 | Isoform Wnt-16b of Protein Wnt-16 precursor | WNT16 |
| IPI00290432 | Natural resistance-associated macrophage protein 1 | SLC11A1 |
| IPI00012540 | Prominin-1 precursor | PROM1 |
| IPI00477396 | Uncharacterized protein ZNF324B (Fragment) | ZNF584; ZNF324B |
| IPI00853149 | RW1 protein | TMEM131 |
| IPI00168977 | Olfactory receptor 6J1 | OR6J1 |
| IPI00165955 | Isoform 1 of Mitogen-activated protein kinase 15 | MAPK15 |
| IPI00377211 | Isoform 3 of Cadherin-like protein 26 precursor | CDH26 |
| IPI00736558 | THAP domain containing 4 | THAP4 |
| IPI00470518 | Mitotic spindle assembly checkpoint protein MAD1 | MAD1L1 |
| IPI00005087 | Tropomodulin-3 | TMOD3 |
| IPI00029227 | LOC93622 protein | LOC93622 |
| IPI00218839 | Cytochrome P450 1A1 | CYP1A1 |
| IPI00006356 | Isoform 1 of Patatin-like phospholipase domain-containing protein 5 | PNPLA5 |
| IPI00005616 | Isoform 1 of Trace amine-associated receptor 2 | TAAR2 |
| IPI00789494 | 8 kDa protein | TUSC4 |
| IPI00745346 | Isoform 11 of Peroxisomal N(1)-acetyl-spermine/spermidine oxidase | PAOX |
| IPI00027596 | Secreted frizzled-related protein 2 precursor | SFRP2 |
| IPI00008455 | Isoform 2 of Myosin-VI | MYO6 |
| IPI00217850 | Isoform 1 of GRAM domain-containing protein 1C | GRAMD1C |
| IPI00020771 | Isoform 3 of Ensconsin | MAP7 |
| IPI00170800 | Isoform 1 of Otoancorin precursor | OTOA |
| IPI00063181 | CDKN2A interacting protein N-terminal like | CDKN2AIPNL |
| IPI00328225 | Isoform 1 of T-box transcription factor TBX20 | TBX20 |
| IPI00166331 | Ankyrin repeat domain-containing protein 35 | ANKRD35 |
| IPI00294386 | Myosin-Ia | MYOIA |
| IPI00061680 | SAGA-associated factor 29 homolog | CCDC101 |
| IPI00220901 | TBC1D4 protein | TBC1D4 |
| IPI00059139 | Vacuolar proton pump subunit E 2 | ATP6V1E2 |
| IPI00010893 | Left-right determination factor 2 precursor | LEFTY2 |
| IPI00746222 | ATP-binding cassette, sub-family B (MDR/TAP), member 5 | ABCB5 |
| IPI00010843 | Isoform 1 of Amyloid beta A4 precursor protein-binding family B member 1 | APBB1 |
| IPI00012353 | Mitochondrial 39S ribosomal protein L3 | MRPL3 |
| IPI00385065 | Isoform 1 of Coiled-coil domain-containing protein 46 | CCDC46 |
| IPI00413930 | Elongation of very long chain fatty acids protein 2 | ELOVL2 |
| IPI00006715 | Double-strand-break repair protein rad21 homolog | RAD21 |
| IPI00787189 | similar to thioredoxin domain containing 4 | LOC727931 |
| IPI00215965 | Isoform A1-B of Heterogeneous nuclear ribonucleoprotein A1 | HNRNPA1 |
| IPI00030237 | Isoform 1 of Uncharacterized protein C1orf49 | C1orf49 |
| IPI00088953 | Phosphoinositide 3-kinase regulatory subunit 6 | C17orf38 |
| IPI00031490 | Isoform 1 of Collectin-11 precursor | COLEC11 |
| IPI00430079 | Isoform 2 of PHD finger protein 8 | PHF8 |
| IPI00296942 | Cadherin-12 precursor | CDH12 |
| IPI00852688 | Isoform 1 of Low density lipoprotein receptor-related protein 5-like protein | LRP5L |
| IPI00060440 | Zinc finger protein GLI4 | GLI4; ZFP41 |
| IPI00845299 | Putative uncharacterized protein LOC285733 | LOC285733 |
| IPI00003897 | Isoform 1 of Protocadherin gamma B6 precursor | PCDHGB6 |
| IPI00152257 | Enkurin | C10orf63 |
| IPI00043526 | Uncharacterized protein C14orf44 | C14orf44 |
| IPI00013373 | Orexin precursor | HCRT |
| IPI00829925 | Uncharacterized protein ENSP00000365090 | ELA2A |
| IPI00009333 | Transmembrane protein 9 precursor | TMEM9 |
| IPI00470917 | Isoform 2 of Uncharacterized protein KIAA1529 | KIAA1529 |
| IPI00013269 | Isoform 1 of Uncharacterized protein C6orf106 | C6orf106 |
| IPI00022433 | Heat-shock protein beta-6 | HSPB6 |
| IPI00179694 | Beta-klotho | KLB |
| IPI00027642 | Isoform 1 of JmjC domain-containing histone demethylation protein 3C | JMJD2C |
| IPI00797832 | Isoform 3 of Acyl-CoA dehydrogenase family member 10 | ACAD10 |
| IPI00410464 | Isoform 1 of Zinc finger protein 324B | ZNF324B; ZNF584 |
| IPI00166863 | Isoform 2 of Coiled-coil domain-containing protein 123, mitochondrial precursor | CCDC123 |
| IPI00008832 | Growth arrest-specific protein 1 precursor | GAS1 |
| IPI00014958 | Isoform 1 of Serum paraoxonase/arylesterase 2 | PON2 |
| IPI00719669 | Myosin regulatory light chain | MRLC2 |
| IPI00299084 | Transmembrane protein 33 | TMEM33 |
| IPI00029737 | Isoform Long of Long-chain-fatty-acid--CoA ligase 4 | ACSL4 |
| IPI00005661 | Isoform Gamma of Max-like protein X | MLX |
| IPI00217683 | A-kinase anchor protein 12 isoform 2 | AKAP12 |
| IPI00418966 | hypothetical protein LOC123688 isoform 2 | LOC123688 |
| IPI00328409 | Armadillo repeat-containing protein 4 | ARMC4 |
| IPI00065457 | OTTHUMP00000018353 | FLJ32682 |
| IPI00444502 | CDNA FLJ45482 fis, clone BRTHA2001953 | FLJ45482 |
| IPI00001780 | Ubiquitin carboxyl-terminal hydrolase 16 | USP16 |
| IPI00552840 | Probable G-protein coupled receptor 112 | GPR112 |
| IPI00016373 | Ras-related protein Rab-13 | RAB13 |
| IPI00552434 | Myosin, heavy chain 7B, cardiac muscle, beta | MYH7B |
| IPI00876962 | Isoform 2 of Inverted formin-2 | INF2 |
| IPI00026512 | Isoform HERA-A of GTP-binding protein era homolog | ERAL1 |
| IPI00180375 | Isoform I of Beta-arrestin-2 | ARRB2 |
| IPI00297407 | Solute carrier family 45 member 4 | SLC45A4 |
| IPI00152879 | Monocarboxylate transporter 10 | SLC16A10 |
| IPI00290155 | 66 kDa protein | ZNF148 |
| IPI00747017 | Putative uncharacterized protein DKFZp686D06121 | NEK1 |
| IPI00793836 | 11 kDa protein | ARHGDIB |
| IPI00382422 | Ig lambda chain V-I region NEWM | — |
| IPI00328842 | ARHGAP4 protein | ARHGAP4 |
| IPI00008632 | Isoform 1 of Putative homeodomain transcription factor 1 | PHTF1 |
| IPI00065253 | Uncharacterized protein C3orf30 | C3orf30 |
| IPI00883751 | RNA-directed DNA polymerase (Reverse transcriptase), related domain | — |
| | containing protein | |
| IPI00216137 | Synaptonemal complex protein 1 | SYCP1 |
| IPI00005621 | 7h3 protein (Fragment) | SYDE1 |
| IPI00001933 | Coiled-coil domain-containing protein 113 | CCDC113 |
| IPI00383294 | Putative uncharacterized protein pp7518 | MNT |
| IPI00016861 | 102 kDa protein | GTF3C2 |
| IPI00020903 | Isoform 1 of AF4/FMR2 family member 2 | AFF2 |
| IPI00465247 | Isoform 1 of Stimulated by retinoic acid gene 6 protein homolog | STRA6 |
| IPI00382394 | Isoform 5 of Cytosolic phospholipase A2 beta | PLA2G4B |
| IPI00167913 | Isoform 1 of Spermatid-associated protein | SPERT |
| IPI00874276 | Putative uncharacterized protein DKFZp761N1814 | EPB41L4B |
| IPI00446767 | Isoform 3 of Transducin-like enhancer protein 4 | TLE4 |
| IPI00020754 | PRO1751 | — |
| IPI00300060 | WD repeat-containing protein 70 | WDR70 |
| IPI00021979 | Peroxisomal membrane protein 11A | PEX11A |
| IPI00031768 | Hook homolog 3 | HOOK3 |
| IPI00216986 | Isoform C of Complement receptor type 2 precursor | CR2 |
| IPI00470771 | SAMD4B protein | SAMD4B |
| IPI00021143 | Class B basic helix-loop-helix protein 2 | BHLHB2 |
| IPI00297931 | Isoform 1 of AP1 subunit gamma-binding protein 1 | AP1GBP1 |
| IPI00018691 | 28S ribosomal protein S18a, mitochondrial precursor | MRPS18A |
| IPI00010604 | Isoform 1 of 1-phosphatidylinositol-4,5-bisphosphate phosphodiesterase epsilon-1 | PLCE1 |
| IPI00022438 | Isoform Long of Transcription factor Maf | MAF |
| IPI00396527 | Isoform 1 of Coiled-coil domain-containing protein 128 | CCDC128 |
| IPI00291840 | Isoform 1 of Zinc finger protein 90 homolog | ZFP90 |
| IPI00646410 | Isoform 2 of Flavin-containing amine oxidase domain-containing protein 1 | AOF1 |
| IPI00451413 | Intersectin 1 isoform 7 | ITSN1 |
| IPI00787410 | similar to Polypeptide N-acetylgalactosaminyltransferase 9 | GALNT9 |
| IPI00552601 | Putative uncharacterized protein DKFZp686M12165 (Fragment) | — |
| IPI00166833 | LAG1 longevity assurance homolog 5 | LASS5 |
| IPI00183425 | CDKN1A interacting zinc finger protein 1 | CIZ1 |
| IPI00027228 | Probable glutamyl-tRNA(Gln) amidotransferase subunit B, mitochondrial | PET112L |
| | precursor | |
| IPI00007917 | Collectin-10 precursor | COLEC10 |
| IPI00552787 | similar to Matrin-3 | RBM20 |
| IPI00065484 | CDNA FLJ32471 fis, clone SKNMC2000322, weakly similar to MAJOR | PTRH2 |
| | CENTROMERE AUTOANTIGEN B | |
| IPI00384447 | Sarcoma antigen NY-SAR-22 (Fragment) | NEXN |
| IPI00830073 | Isoform 1 of RING finger protein 207 | RNF207 |
| IPI00012411 | Zinc finger protein 32 | ZNF32 |
| IPI00760993 | IQ motif and Sec7 domain 2 isoform 2 | IQSEC2 |
| IPI00004527 | Uncharacterized protein KIAA0355 | KIAA0355 |
| IPI00018956 | ALX homeobox protein 1 | ALX1 |
| IPI00154553 | Centrosomal protein of 76 kDa | CEP76 |
| IPI00444208 | CDNA FLJ45771 fis, clone NETRP2003103 | RNF44 |
| IPI00001641 | Origin recognition complex subunit 6 | ORC6L |
| IPI00216484 | Isoform 2 of LisH domain-containing protein ARMC9 | ARMC9 |
| IPI00170961 | Isoform 2 of WD repeat-containing protein 16 | WDR16 |
| IPI00786880 | Myosin-XVB | — |
| IPI00152722 | Protein CCDC35 | CCDC35 |
| IPI00298447 | Probable methyltransferase TARBP1 | TARBP1 |
| IPI00293200 | OTTHUMP00000016423 | PARC |
| IPI00550272 | Integrator complex subunit 10 | INTS10 |
| IPI00514197 | similar to Nonhistone chromosomal protein HMG-17 | LOC648822 |
| IPI00156804 | cDNA FLJ77550, highly similar to Homo sapiens zinc finger protein 665 | ZNF665 |
| | (ZNF665), mRNA | |
| IPI00384140 | Full-length cDNA clone CS0DA005YA22 of Neuroblastoma of Homo sapiens | ESR2 |
| | (Fragment) | |
| IPI00410564 | CDNA FLJ45248 fis, clone BRHIP2006819 | FLJ45248 |
| IPI00023586 | Isoform 1 of Sentrin-specific protease 6 | SENP6 |
| IPI00028579 | Transcription initiation factor TFIID subunit 6 | TAF6 |
| IPI00253323 | Ankyrin repeat domain-containing protein 57 | ANKRD57 |
| IPI00166361 | Putative uncharacterized protein FLJ33590 | FLJ33590 |
| IPI00294810 | CDNA FLJ10824 fis, clone NT2RP4001086 (Fragment) | FAM21A |
| IPI00418408 | Cytochrome P450 | CYP2D7P1 |
| IPI00168627 | Uncharacterized protein CXorf20 | CXorf20 |
| IPI00026952 | Plakophilin-3 | PKP3 |
| IPI00014852 | Isoform 1 of Phosphoglucomutase-like protein 5 | PGM5 |
| IPI00332628 | Otopetrin-3 | OTOP3 |
| IPI00296830 | Isoform 1 of Leucine zipper protein 1 | LUZP1 |
| IPI00306127 | THUMP domain-containing protein 3 | THUMPD3 |
| IPI00022881 | Isoform 1 of Clathrin heavy chain 2 | CLTCLI |
| IPI00397015 | A.T hook DNA-binding motif-containing protein 1 | AHDC1 |
| IPI00029184 | Hyaluronan and proteoglycan link protein 2 precursor | HAPLN2 |
| IPI00795917 | 126 kDa protein | CXorf45 |
| IPI00815976 | Transmembrane protein 120B | TMEM120B |
| IPI00382671 | Isoform 6 of C-type lectin domain family 4 member M | CLEC4M |
| IPI00018303 | Isoform 2 of Protocadherin-11 X-linked precursor | PCDH11X |
| IPI00176210 | hypothetical protein LOC133558 | FLJ40243 |
| IPI00166153 | Protein KIAA0082 | KIAA0082 |
| IPI00300621 | Isoform 2B2 of Synaptojanin-2 | SYNJ2 |
| IPI00306166 | germ cell associated 1 isoform 2 | GSG1 |
| IPI00401002 | Zinc finger protein 740 | ZNF740 |
| IPI00607829 | Isoform 1 of Ret fmger protein-like 2 | RFPL2 |
| IPI00402280 | Isoform 1 of Small leucine-rich proteoglycan family member LOC150356 | CHADL |
| | precursor | |
| IPI00006800 | Isoform Long of Autophagy protein 5 | ATG5 |
| IPI00218559 | Gamma-sarcoglycan | SGCG |
| IPI00479897 | Chromosome 9 open reading frame 75 | C9orf75 |
| IPI00216003 | Cullin-5 | CUL5 |
| IPI00445796 | CDNA FLJ43383 fis, clone OCBBF2006058, highly similar to Homo sapiens | ACAD8 |
| | acyl-Coenzyme A dehydrogenase-8 mRNA | |
| IPI00742114 | similar to tripartite motif protein 11 | LOC653978 |
| IPI00023164 | Potassium/sodium hyperpolarization-activated cyclic nucleotide-gated channel 4 | HCN4 |
| IPI00012480 | FKSG42 | RACGAP1P |
| IPI00004538 | similar to nascent polypeptide-associated complex alpha polypeptide isoform 1 NACAD | |
| IPI00297462 | Uncharacterized protein C1orf65 | C1orf65 |
| IPI00793454 | 7 kDa protein | USP19 |
| IPI00028392 | specifically androgen-regulated protein isoform 1 | C1orf116 |
| IPI00385495 | Transmembrane protein 153 | LMF2 |
| IPI00017726 | Isoform 1 of 3-hydroxyacyl-CoA dehydrogenase type-2 | HSD17B10 |
| IPI00791509 | Neural precursor cell expressed, developmentally down-regulated 1 | NEDD1 |
| IPI00289931 | Isoform 1 of Mucosal addressin cell adhesion molecule 1 precursor | MADCAM1 |
| IPI00442601 | CDNA FLJ26817 fis, clone PRS06446 | — |
| IPI00012891 | Phosphorylase b kinase gamma catalytic chain, testis/liver isoform | PHKG2 |
| IPI00021338 | Dihydrolipoyllysine-residue acetyltransferase component of pyruvate | DLAT |
| | dehydrogenase complex, mitochondrial precursor | |
| IPI00455083 | Uncharacterized protein ENSP00000371558 | — |
| IPI00168877 | helicase (DNA) B | HELB |
| IPI00641214 | 177 kDa protein | ADCY10 |
| IPI00290837 | Isoform 2 of F-box/WD repeat-containing protein 10 | FBXW10 |
| IPI00009213 | Isoform 1 of B-cell lymphoma/leukemia 11B | BCL11B |
| IPI00003421 | T-brain-1 protein | TBR1 |
| IPI00396314 | Isoform 3 of CCR4-NOT transcription complex subunit 10 | CNOT10 |
| IPI00000106 | Signal transducer and activator of transcription 4 | STAT4 |
| IPI00339217 | Ovochymase-1 precursor | OVCH1 |
| IPI00395631 | Isoform 1 of E3 ubiquitin-protein ligase TRAF7 | TRAF7 |
| IPI00455397 | DMWD protein | DMWD |
| IPI00744557 | kazrin isoform E | RPI-21O18.1 |
| IPI00152692 | D-tyrosyl-tRNA(Tyr) deacylase 1 | DTD1 |
| IPI00397526 | Isoform 1 of Myosin-10 | MYH10 |
| IPI00640654 | RAP1 GTPase activating protein | RAP1GAP |
| IPI00791831 | 10 kDa protein | ADRBK I |
| IPI00479904 | Uncharacterized protein COL27A1 | COL27A1 |
| IPI00013256 | Isoform 1 of Cleavage stimulation factor 64 kDa subunit | CSTF2 |
| IPI00059279 | Exocyst complex component 4 | EXOC4 |
| IPI00299402 | Pyruvate carboxylase, mitochondrial precursor | PC |
| IPI00167639 | Vacuolar proton pump subunit d 2 | ATP6V0D2 |
| IPI00170855 | tetratricopeptide repeat domain 19 | TTC19 |
| IPI00445563 | Putative uncharacterized protein MGC21675/FLJ43787 | C4orf42 |
| IPI00884334 | Conserved hypothetical protein | LOC729968 |
| IPI00007910 | Isoform 1 of Sodium-dependent phosphate transport protein 2B | SLC34A2 |
| IPI00083708 | BAT2-iso | BAT2D1 |
| IPI00328715 | Protein LYRIC | MTDH |
| IPI00290035 | Isoform 1 of Protocadherin-15 precursor | PCDH15 |
| IPI00385511 | Uncharacterized protein TNRC6B | TNRC6B |
| IPI00006561 | Isoform 1 of Protein KIAA0317 | KIAA0317 |
| IPI00375294 | Laminin subunit alpha-1 precursor | LAMA1 |
| IPI00375325 | Keratin-associated protein 10-10 | KRTAP10-10 |
| IPI00640293 | 23 kDa protein | TIMP3 |
| IPI00235003 | Tumor necrosis factor receptor superfamily, member 6 isoform 1 variant | FAS |
| | (Fragment) | |
| IPI00216702 | Isoform 1 of ATP-binding cassette sub-family A member 9 | ABCA9 |
| IPI00167930 | ring finger protein 190 | MARCH10 |
| IPI00470657 | Anti-colorectal carcinoma heavy chain | — |
| IPI00413868 | hypothetical protein LOC79632 isoform 1 | C6orf60 |
| IPI00066367 | Endothelial cells scavenger receptor precursor | SCARF1 |
| IPI00397740 | zinc finger protein 749 | ZNF749 |
| IPI00300426 | Isoform 1 of Tetratricopeptide repeat protein 29 | TTC29 |
| IPI00307259 | DnaJ homolog subfamily C member 13 | DNAJCI3 |
| IPI00386113 | CDNA: FLJ23412 fis, clone HEP20516 | C20orf59 |
| IPI00153060 | Angiopoietin-related protein 4 precursor | ANGPTL4 |
| IPI00020354 | Interleukin-13 receptor alpha-1 chain precursor | IL13RAI |
| IPI00177888 | Isoform 1 of Leukocyte receptor cluster member 8 | LENG8 |
| IPI00101267 | Isoform 1 of Uncharacterized protein C19orf60 | C19orf60 |
| IPI00739927 | similar to vasoactive intestinal peptide receptor 2 | LOC645464 |
| IPI00782965 | huntingtin interacting protein 1 | HIP1 |
| IPI00444375 | Rh blood group, CcEe antigens | RHCE |
| IPI00794880 | Isoform 1 of Chromodomain-helicase-DNA-binding protein 7 | CHD7 |
| IPI00419791 | Isoform 1 of Arginine/serine-rich coiled-coil protein 2 | RSRC2 |
| IPI00100106 | resistance to inhibitors of cholinesterase 8 homolog A | RIC8A |
| IPI00216593 | Isoform NOH-1LV of NADPH oxidase homolog 1 | NOX1 |
| IPI00395667 | Interferon-related IFRD2 (PC4-B) protein | NAT6; IFRD2 |
| IPI00008708 | Ribosomal L1 domain-containing protein 1 | RSL1D1 |
| IPI00748649 | 25 kDa protein | — |
| IPI00100362 | erythrocyte membrane protein band 4.1 like 4B isoform 1 | EPB41L4B |
| IPI00219898 | Isoform 2 of Nephrin precursor | NPHS1; PRODH2 |
| IPI00872098 | Zinc finger protein Pegasus | IKZF5 |
| IPI00043294 | Isoform 1 of Uncharacterized protein C7orf26 | C7orf26 |
| IPI00152535 | Chromodomain-helicase-DNA-binding protein 5 | CHD5 |
| IPI00217507 | Neurofilament medium polypeptide | NEFM |
| IPI00216356 | Isoform 2 of RasGAP-activating-like protein 1 | RASAL1 |
| IPI00290562 | Nuclear factor interleukin-3-regulated protein | NFIL3 |
| IPI00006003 | CD83 antigen precursor | CD83 |
| IPI00166528 | Isoform 3 of Rapamycin-insensitive companion of mTOR | RICTOR |
| IPI00221325 | E3 SUMO-protein ligase RanBP2 | RANBP2 |
| IPI00084684 | similar to Zinc finger protein 469 | ZNF469 |
| IPI00643809 | Protein | CNTNAP3B |
| IPI00164776 | Similar to RIKEN cDNA A230078105 gene | TMEM198 |
| IPI00023410 | Integrin alpha-8 precursor | ITGA8 |
| IPI00787593 | similar to CG33300-PA | LOC730407 |
| IPI00180781 | Isoform 1 of Mixed lineage kinase domain-like protein | MLKL |
| IPI00339309 | Isoform 1 of Proto-oncogene DBL | MCF2 |
| IPI00843923 | Phosphodiesterase PDE7B2 | PDE7B |
| IPI00640865 | Integrin beta-like protein 1 precursor | ITGBLI |
| IPI00029144 | Isoform PR130 of Serine/threonine-protein phosphatase 2A regulatory subunit B″ | PPP2R3A |
| | subunit alpha | |
| IPI00175439 | F-box only protein 43 | FBXO43 |
| IPI00646485 | Protein | EFCAB2 |
| IPI00023105 | Peptidylglycine alpha-amidating monooxygenase COOH-terminal interactor | PAMCI |
| IPI00301144 | Isoform 1 of Sulfatase-modifying factor 1 precursor | SUMF1 |
| IPI00439935 | UDP-glucuronosyltransferase 1-4 precursor | UGT1A4; UGT1A6 |
| IPI00001753 | Myosin-4 | MYH4 |
| IPI00401829 | Putative uncharacterized protein ENST00000281581 | — |
| IPI00056324 | Isoform 1 of Hermansky-Pudlak syndrome 3 protein | HPS3 |
| IPI00220493 | U6 snRNA-associated Sm-like protein LSm5 | LSM5 |
| IPI00386390 | Olfactory receptor 6N2 | OR6N2 |
| IPI00044761 | Pseudouridylate synthase 7 homolog | PUS7 |
| IPI00032342 | TRIP12 protein | TRIP12 |
| IPI00307536 | Isoform 1 of Testis-expressed sequence 11 protein | TEX11 |
| IPI00244812 | Tetratricopeptide repeat protein 6 | TTC6 |
| IPI00030487 | CDNA FLJ30993 fis, clone HLUNG1000064, weakly similar to KARYOGAMY | KIAA1627 |
| | PROTEIN KAR4 | |
| IPI00787443 | similar to Collagen alpha-2(XI) chain precursor | LOC730037 |
| IPI00013371 | Protein sprouty homolog 3 | SPRY3 |
| IPI00414259 | Isoform 2 of Pregnancy-specific beta-1-glycoprotein 4 precursor | PSG4 |
| IPI00410590 | Isoform 2 of LSM14 protein homolog A | LSM14A |
| IPI00023330 | Isoform 2 of LisH domain and HEAT repeat-containing protein KIAA1468 | KIAA1468 |
| IPI00645614 | Isoform 2 of Cadherin-3 precursor | CDH3 |
| IPI00744574 | Similar to Tropomyosin alpha-4 chain | — |
| IPI00737735 | similar to cell division cycle 10 isoform 1 | LOC646913 |
| IPI00065415 | Isoform 1 of Coiled-coil domain-containing protein 138 | CCDC138 |
| IPI00376206 | Isoform 2 of 17-beta hydroxysteroid dehydrogenase 13 precursor | HSD17B13 |
| IPI00011589 | Isoform A of Septin-4 | SEPT4 |
| IPI00032158 | Isoform 2 of NMDA receptor-regulated protein 1 | NARG1 |
| IPI00013468 | Mitotic checkpoint protein BUB3 | BUB3 |
| IPI00745955 | EBNA1 binding protein 2 | EBNA1BP2 |
| IPI00157237 | Isoform 1 of MAGUK p55 subfamily member 4 | MPP4 |
| IPI00009040 | Ornithine decarboxylase antizyme 3 | OAZ3 |
| IPI00383774 | Mih1/Tx protein | CASP4 |
| IPI00182774 | SH3YL1 protein | SH3YL1 |
| IPI00217691 | Isoform 1 of EF-hand domain-containing protein C17orf57 | C17orf57 |
| IPI00514843 | KIAA0082 | KIAA0082 |
| IPI00216346 | Isoform 2 of Nucleolar protein 3 | NOL3 |
| IPI00644968 | Similar to Heterogeneous nuclear ribonucleoprotein A1 | — |
| IPI00414122 | coiled-coil domain containing 141 | CCDC141 |
| IPI00884216 | RNA-directed DNA polymerase (Reverse transcriptase), related domain | — |
| | containing protein | |
| IPI00397759 | similar to ribosomal protein L21 | LOC388621 |
| IPI00156871 | Isoform 2 of Ubiquitin carboxyl-terminal hydrolase 49 | USP49 |
| IPI00168692 | Isoform 2 of Zinc finger protein 318 | ZNF318 |
| IPI00187082 | Isoform 3 of Multiple C2 and transmembrane domain-containing protein 1 | MCTP1 |
| IPI00018805 | Uncharacterized protein C14orf105 | C14orf105 |
| IPI00446685 | CDNA FLJ41345 fis, clone BRAWH2002761 | — |
| IPI00470515 | Isoform 1 of Uncharacterized protein C1orf173 | C1orf173 |
| IPI00001317 | Rho-related BTB domain-containing protein 1 | RHOBTB1 |
| IPI00024523 | Isoform A of DnaJ homolog subfamily B member 6 | DNAJB6 |
| IPI00030814 | Fibroblast growth factor 22 precursor | FGF22 |
| IPI00441064 | Isoform 1 of Uncharacterized protein C15orf26 | C15orf26 |
| IPI00867744 | Isoform 3 of Pleckstrin homology domain-containing family H member 2 | PLEKHH2 |
| IPI00784027 | Rod cGMP-specific 3′,5′-cyclic phosphodiesterase subunit beta precursor | PDE6B |
| IPI00879277 | 436 kDa protein | TRRAP |
| IPI00011631 | Centromere/kinetochore protein zw10 homolog | ZW10 |
| IPI00014186 | Isoform A of Zinc forger homeobox protein 3 | ZFHX3 |
| IPI00852699 | 4 kDa protein | — |
| IPI00400834 | Mediator of RNA polymerase II transcription subunit 13-like | MED13L |
| IPI00018203 | Isoform SRP55-2 of Splicing factor, arginine/serine-rich 6 | SFRS6 |
| IPI00335259 | deleted in liver cancer 1 isoform 1 | DLC1 |
| IPI00239405 | Isoform 1 of Nesprin-2 | SYNE2 |
| IPI00788010 | similar to 60S ribosomal protein L21 | RPL21; LOC728501 |
| IPI00027487 | Creatine kinase M-type | CKM |
| IPI00029662 | Isoform 1 of Potassium voltage-gated channel subfamily H member 2 | KCNH2 |
| IPI00651669 | Putative uncharacterized protein | — |
| IPI00029446 | Isoform 1 of Myotubularin-related protein 5 | SBF1 |
| IPI00883929 | Conserved hypothetical protein | — |
| IPI00641155 | 20 kDa protein | HIRIP3 |
| IPI00030307 | Solute carrier family 41 member 2 | SLC41A2 |
| IPI00398162 | Isoform 2 of Nebulin-related-anchoring protein | NRAP |
| IPI00158506 | Isoform 3 of Zinc finger protein 688 | ZNF688 |
| IPI00003768 | Isoform 1 of Pescadillo homolog 1 | PES1 |
| IPI00100910 | Isoform 2 of Armadillo repeat-containing protein 5 | ARMC5 |
| IPI00445894 | CDNA FLJ42978 fis, clone BRTHA2004821 | — |
| IPI00022325 | Isoform 4 of Pro-neuregulin-2, membrane-bound isoform precursor | NRG2 |
| IPI00747271 | zinc finger protein SBZF3 | ZNF695 |
| IPI00006178 | Y6 encoding protein | NPY6R |
| IPI00796745 | 22 kDa protein | PPP1R14D |
| IPI00299880 | Isoform 2 of Calcium-activated potassium channel subunit beta-3 | KCNMB3 |
| IPI00328885 | eEF1A2 binding protein | DKFZp434B1231 |
| IPI00009305 | Glucosamine-6-phosphate isomerase | GNPDA1 |
| IPI00465054 | Putative uncharacterized protein DKFZp686C1054 | THUMPD1 |
| IPI00456676 | Isoform 1 of F-box only protein 38 | FBXO38 |
| IPI00328089 | Kappa B and V(D)J recombination signal sequences binding protein | HIVEP3 |
| IPI00002506 | Dolichyl-phosphate beta-glucosyltransferase | ALG5 |
| IPI00410325 | Low-density lipoprotein receptor class A domain-containing protein 3 precursor | LDLRAD3 |
| IPI00450358 | Putative uncharacterized protein | TOP |
| IPI00004472 | Isoform 1 of Serine/threonine-protein kinase WNK1 | WNK1 |
| IPI00428677 | Putative olfactory receptor (Fragment) | — |
| IPI00788014 | similar to Opioid growth factor receptor | LOC729015 |
| IPI00442208 | CDNA FLJ16344 fis, clone TESTI2032643, moderately similar to | ADCY4 |
| | ADENYLATE CYCLASE, TYPE IV | |
| IPI00019932 | ubiquitin-conjugating enzyme E2D 2 isoform 2 | UBE2D2 |
| IPI00869224 | Pseudogene candidate | — |
| IPI00000102 | Progonadoliberin-1 precursor | GNRH1 |
| IPI00470896 | Isoform 2 of Zinc finger FYVE domain-containing protein 26 | ZFYVE26 |
| IPI00329488 | Isoform IB of Tyrosine-protein kinase ABL2 | ABL2 |
| IPI00219604 | Dual specificity mitogen-activated protein kinase kinase 1 | MAP2K1 |
| IPI00000030 | Isoform Delta-1 of Serine/threonine-protein phosphatase 2A 56 kDa regulatory | PPP2R5D |
| | subunit delta isoform | |
| IPI00017538 | Isoform 2 of Separin | ESPL1 |
| IPI00787099 | similar to SR protein related family member | LOC728676 |
| IPI00297235 | cDNA FLJ77204 | CCPG1 |
| IPI00002405 | 2′-5′-oligoadenylate synthetase 3 | OAS3 |
| IPI00013205 | Isoform 1 of JmjC domain-containing histone demethylation protein 3B | JMJD2B |
| IPI00218715 | Isoform 2 of ADAM 33 precursor | ADAM33 |
| IPI00410666 | Isoform 3 of Protein LAP4 | SCRIB |
| IPI00827815 | Light chain Fab (Fragment) | — |
| IPI00013651 | Isoform 3 of RNA-binding protein 33 | RBM33 |
| IPI00002344 | steroid 5 alpha-reductase 3 | SRD5A3 |
| IPI00444408 | CDNA FLJ45600 fis, clone BRTHA3020369 | — |
| IPI00005013 | poly(rC) binding protein 4 isoform b | PCBP4 |
| IPI00294728 | DmX-like protein 1 | DMXL1 |
| IPI00004288 | Isoform 1 of Sialic acid-binding Ig-like lectin 7 precursor | SIGLEC7 |
| IPI00855960 | Integral membrane protein GPR137C | GPR137C |
| IPI00003571 | Isoform S-CaBP1 of Calcium-binding protein 1 | CABP1 |
| IPI00021751 | Neurofilament heavy polypeptide | NEFH |
| IPI00174347 | Inactive dual specificity phosphatase 27 | DUSP27 |
| IPI00787774 | similar to collagen, type I, alpha 1 | LOC730756 |
| IPI00419903 | Putative L-aspartate dehydrogenase | LOC554235 |
| IPI00165357 | Isoform 1 of Metastasis-associated protein MTA3 | MTA3 |
| IPI00299406 | Pituitary adenylate cyclase-activating polypeptide type I receptor precursor | ADCYAP1R1 |
| IPI00401789 | Isoform 2 of Highly divergent homeobox | HDX |
| IPI00303732 | Uncharacterized protein C20orf106 precursor | C20orf106 |
| IPI00016425 | Isoform 2 of Synaptopodin 2-like protein | SYNPO2L |
| IPI00017640 | Isoform 1 of Slit homolog 3 protein precursor | SLIT3 |
| IPI00014577 | Ras-related protein Rab-18 | RAB18 |
| IPI00023856 | Peregrin | BRPF1 |
| IPI00294241 | Transcription factor IIIA | GTF3A |
| IPI00025717 | Metaxin-2 | MTX2 |
| IPI00023095 | Myeloid leukemia factor 2 | MLF2 |
| IPI00009645 | Ran-binding protein 17 | RANBP17 |
| IPI00022629 | Isoform 1 of Protein regulator of cytokinesis 1 | PRC1 |
| IPI00031082 | Putative uncharacterized protein DKFZp761O17121 | LDOC1L |
| IPI00788120 | similar to CG14182-PA | LOC730094 |
| IPI00396063 | C-myc promoter-binding protein | DENND4A |
| IPI00013296 | 40S ribosomal protein S18 | RPS18 |
| IPI00004362 | MORC family CW-type zinc finger protein 1 | MORC1 |
| IPI00787625 | similar to 60S ribosomal protein L23a | LOC389101 |
| IPI00385020 | Putative uncharacterized protein DKFZp686D159 (Fragment) | CAPRIN2 |
| IPI00374223 | hypothetical protein LOC389119 | C3orf54 |
| IPI00039626 | Isoform D of UPF0318 protein FAM120A | FAM120A |
| IPI00295253 | Isoform 1 of Cell surface glycoprotein OX2 receptor precursor | CD200R1 |
| IPI00299040 | Polycystin-2 | PKD2 |
| IPI00307165 | Tripartite motif-containing protein 47 | TRIM47 |
| IPI00152685 | Tetratricopeptide repeat protein 15 | TTC15 |
| IPI00013789 | SET and MYND domain-containing protein 5 | SMYD5 |
| IPI00396025 | hypothetical protein LOC254122 | FLJ30934 |
| IPI00014232 | ADP-ribosylation factor-like protein 6-interacting protein 1 | ARL6IP1 |
| IPI00021715 | Isoform 1 of Collagen alpha-5(IV) chain precursor | COL4A5 |
| IPI00026305 | Hook homolog 1 | HOOK1 |
| IPI00184160 | Isoform 2 of RING finger protein 180 | RNF180 |
| IPI00216308 | Voltage-dependent anion-selective channel protein 1 | VDAC1 |
| IPI00651633 | CASKIN1 protein (Fragment) | CASKIN1 |
| IPI00247063 | Neprilysin | MME |
| IPI00398779 | Isoform 4 of Plectin-1 | PLEC1 |
| IPI00168418 | Putative uncharacterized protein C11orf64 | C11orf64 |
| IPI00167764 | Transmembrane protein 102 | TMEM102 |
| IPI00171716 | OTTHUMP00000028696 | TTC28 |
| IPI00394818 | Isoform 1 of Ankyrin repeat domain-containing protein 47 | KANK3 |
| IPI00830137 | Uncharacterized protein ENSP00000374864 | — |
| IPI00030874 | Uncharacterized gastric protein YA42P (Fragment) | — |
| IPI00455675 | Isoform 2 of Centrosomal protein of 192 kDa | CEP192 |
| IPI00789889 | Hypothetical protein | — |
| IPI00829625 | Uncharacterized protein FAM91A1 | FAM91A1 |
| IPI00000873 | Valyl-tRNA synthetase | VARS |
| IPI00386185 | FLJ00370 protein (Fragment) | — |
| IPI00829697 | Uncharacterized protein ENSP00000375030 | — |
| IPI00000690 | Isoform 1 of Apoptosis-inducing factor 1, mitochondrial precursor | AIFM1 |
| IPI00016988 | WD repeat-containing protein 13 | WDR13 |
| IPI00478058 | Isoform 2 of NMDA receptor-regulated 1-like protein | NARG1L |
| IPI00782960 | Isoform 1 of Leucine-rich repeat and IQ domain-containing protein 3 | LRRC44 |
| IPI00103373 | Cytoglobin | CYGB |
| IPI00646831 | 34 kDa protein | — |
| IPI00384164 | Full-length cDNA clone CS0DI075YC18 of Placenta of Homo sapiens | PGF |
| | (Fragment) | |
| IPI00739611 | similar to golgi autoantigen, golgin subfamily a, 8A isoform 3 | LOC727909 |
| IPI00012451 | Guanine nucleotide-binding protein subunit beta-4 | GNB4 |
| IPI00394960 | Isoform 1 of Probable G-protein coupled receptor 133 precursor | GPR133 |
| IPI00555595 | Ubiquitin carboxyl-terminal esterase L1 (Ubiquitin thiolesterase) variant | — |
| | (Fragment) | |
| IPI00431697 | Cancer/testis antigen 45-5 | RP13-36C9.6 |
| IPI00028066 | class IV alcohol dehydrogenase 7 mu or sigma subunit | ADH7 |
| IPI00152960 | RNA polymerase I-specific transcription initiation factor RRN3 | RRN3 |
| IPI00792370 | 6 kDa protein | GNAT1 |
| IPI00167762 | Isoform 1 of Uncharacterized protein C19orf47 | C19orf47 |
| IPI00385321 | Isoform 2 of Protein kinase C-binding protein 1 | ZMYND8 |
| IPI00385153 | HUMEEP | ATXN10 |
| IPI00337348 | Growth differentiation factor 7 | GDF7 |
| IPI00060627 | Coiled-coil domain-containing protein 124 | CCDC124 |
| IPI00401190 | LOC155006 protein | LOC155006 |
| IPI00025039 | rRNA 2′-O-methyltransferase fibrillarin | FBL |
| IPI00479578 | similar to Nonhistone chromosomal protein HMG-14 | hCG_1659830 |
| IPI00018245 | Isoform 3 of DNA-directed RNA polymerase III subunit RPC5 | POLR3E |
| IPI00020565 | PRO1598 | — |
| IPI00446711 | CDNA FLJ41261 fis, clone BRAMY2034920 | — |
| IPI00787990 | similar to ribosomal protein S14 | LOC644119 |
| IPI00218743 | Isoform 2 of Bestrophin-1 | BEST1 |
| IPI00736826 | similar to Temporarily Assigned Gene name family member | LOC402217 |
| IPI00412024 | Zinc finger protein 485 | ZNF485 |
| IPI00297559 | Isoform 1 of USH1C-binding protein 1 | USHBP1 |
| IPI00396147 | Protocadherin-20 precursor | PCDH20 |
| IPI00478572 | Collagen alpha-4(IV) chain precursor | COL4A4 |
| IPI00032785 | Isoform II of Amyloid beta A4 precursor protein-binding family B member 3 | APBB3 |
| IPI00009982 | Isoform 1 of Tudor and KH domain-containing protein | TDRKH |
| IPI00216164 | Peroxisomal bifunctional enzyme | EHHADH |
| IPI00419912 | YY2 transcription factor | YY2 |
| IPI00735531 | proline-rich synapse-associated protein 2 isoform 4 | SHANK3 |
| IPI00005104 | Inhibitor of nuclear factor kappa-B kinase subunit alpha | CHUK |
| IPI00008837 | Tyrosine-protein phosphatase non-receptor type 5 | PTPN5 |
| IPI00017800 | ATP-binding cassette sub-family A member 3 | ABCA3 |
| IPI00737972 | Transcription factor 24 (Fragment) | TCF24 |
| IPI00374074 | similar to CG40449-PA.3 | LOC388795 |
| IPI00220533 | Isoform D of Transmembrane protease, serine 3 | TMPRSS3 |
| IPI00302458 | Exportin-7 | XPO7 |
| IPI00646826 | Putative uncharacterized protein FLJ32658 | FLJ32658 |
| IPI00016859 | Isoform 1 of Transcription factor IIIB 90 kDa subunit | BRF1 |
| IPI00031386 | Phosphatidylinositol-4,5-bisphosphate 3-kinase catalytic subunit alpha isoform | PIK3CA |
| IPI00065547 | OTTHUMP00000031495 | RP11-93B14.6 |
| IPI00043370 | WD repeat-containing protein 52 | WDR52 |
| IPI00064767 | Isoform 1 of Rho GTPase-activating protein 17 | ARHGAP17 |
| IPI00219689 | Isoform Beta of Protein vav-3 | VAV3 |
| IPI00328291 | Cytoskeleton-associated protein 2-like | CKAP2L |
| IPI00647897 | RING finger protein 113B | RNF113B |
| IPI00024285 | similar to testicular serine protease 2 | LOC138652 |
| IPI00783200 | 38 kDa protein | RAPGEF3 |
| IPI00014877 | Isoform 2 of Glutaminyl-peptide cyclotransferase-like protein | QPCTL |
| IPI00186554 | Uncharacterized protein KIAA1853 | KIAA1853 |
| IPI00418336 | Isoform 2 of Integrator complex subunit 3 | INTS3 |
| IPI00872730 | Macrophin 1 isoform | MACF1 |
| IPI00303207 | ATP-binding cassette sub-family E member 1 | ABCE1 |
| IPI00301609 | Serine/threonine-protein kinase Nek9 | NEK9 |
| IPI00744527 | Conserved hypothetical protein | — |
| IPI00302351 | Isoform 1 of Serine/threonine-protein kinase 33 | STK33 |
| IPI00878483 | 30 kDa protein | — |
| IPI00744179 | Uncharacterized protein ENSP00000370856 (Fragment) | — |
| IPI00396279 | Isoform 1 of CLIP-associating protein 1 | CLASP1 |
| IPI00013378 | Isoform Long of DNA topoisomerase 3-alpha | TOP3A |
| IPI00429190 | Ras-related protein Rab-11A | RAB11A |
| IPI00464994 | Isoform 1 of tRNA modification GTPase GTPBP3, mitochondrial precursor | GTPBP3 |
| IPI00008449 | Isoform 3 of Pre-mRNA 3′-end-processing factor FIP1 | FIP1L1 |
| IPI00414402 | Olfactory receptor 2T27 | OR2T27 |
| IPI00168913 | Isoform 2 of Limbin | EVC2 |
| IPI00009315 | Golgi resident protein GCP60 | ACBD3 |
| IPI00871625 | cDNA FLJ77072, highly similar to Homo sapiens prostaglandin E receptor 3 | PTGER3 |
| | (subtype EP3) (PTGER3), transcript variant 9, mRNA | |
| IPI00479296 | Isoform 1 of ATP-binding cassette sub-family A member 8 | ABCA8 |
| IPI00300221 | Putative uncharacterized protein DKFZp43411916 | ELL |
| IPI00026036 | Isoform 5 of Dipeptidyl peptidase 8 | DPP8 |
| IPI00647205 | Uncharacterized protein C6orf174 precursor | C6orf174 |
| IPI00006932 | Isoform 1 of Putative RNA-binding protein Luc7-like 2 | LUC7L2 |
| IPI00017819 | Bile acid-CoA:amino acid N-acyltransferase | BAAT |
| IPI00145121 | GPI mannosyltransferase 3 | PIGB |
| IPI00744357 | Conserved hypothetical protein | — |
| IPI00410320 | Sodium-dependent glucose transporter | SLC5A11 |
| IPI00065085 | CDNA FLJ25371 fis, clone TST01885 | FLJ25371 |
| IPI00289914 | MutS protein homolog 4 | MSH4 |
| IPI00012438 | Isoform Long of 52 kDa repressor of the inhibitor of the protein kinase | PRKRIR |
| IPI00217895 | rai-like protein | SHC4 |
| IPI00294618 | Proline-rich protein PRCC | PRCC |
| IPI00000784 | Isoform MEF2DAB of Myocyte-specific enhancer factor 2D | MEF2D |
| IPI00848180 | Conserved hypothetical protein | — |
| IPI00045208 | Zinc finger protein 300-B | ZNF300 |
| IPI00871452 | Isoform 1 of PWWP domain-containing protein 2A | PWWP2A |
| IPI00102820 | Isoform 3 of Mitochondrial tumor suppressor 1 | MTUS1 |
| IPI00215786 | 3′,5′-cyclic nucleotide phosphodiesterase 10A2 | PDE10A |
| IPI00845401 | Isoform 6 of Neuron navigator 1 | NAV1 |
| IPI00217979 | Myeloid/lymphoid or mixed-lineage leukemia 5 | MLL5 |
| IPI00440764 | Isoform 1 of Gamma-glutamyltransferase 6 precursor | GGT6 |
| IPI00374068 | Isoform 1 of ADAMTS-like protein 4 precursor | ADAMTSL4 |
| IPI00186439 | Zinc finger protein 643 | ZNF643 |
| IPI00062467 | Isoform 1 of Zinc fmger protein 274 | ZNF274 |
| IPI00000890 | Isoform 1 of Growth hormone variant precursor | GH2 |
| IPI00065351 | Zinc finger protein 354B | ZNF354B |
| IPI00552455 | 12 kDa protein | INPP5A |
| IPI00395473 | Zinc finger protein 135 | ZNF135 |
| IPI00003842 | Isoform 1 of Microtubule-associated protein 2 | MAP2 |
| IPI00655760 | Envelope polyprotein | — |
| IPI00384767 | FLJ00332 protein (Fragment) | TBC1D10C |
| IPI00028050 | Selenocysteine-specific elongation factor | EEFSEC |
| IPI00152033 | Isoform 1 of Transient receptor potential cation channel subfamily M member 8 | TRPM8 |
| IPI00789564 | Isoform 3 of Metallophosphoesterase 1 | MPPE1 |
| IPI00011253 | 40S ribosomal protein S3 | RPS3 |
| IPI00454620 | HERV-K_11q22.1 provirus ancestral Pol protein | — |
| IPI00337335 | Isoform 1 of Myosin-14 | MYH14 |
| IPI00103426 | Isoform 1 of Solute carrier family 13 member 3 | SLC13A3 |
| IPI00291839 | Isoform 2 of Rap guanine nucleotide exchange factor 6 | RAPGEF6 |

TABLE 6c
CSF Proteins Absent in Chronic Fatigue
but Present in Neurologic Lyme or Normals

| CleanIPI | Protein | Gene |
|---|---|---|
| IPI00160369 | PRKCA-binding protein | PICK1 |
| IPI00289862 | Secernin-1 | SCRN1 |
| IPI00002478 | Isoform B of Endothelin-converting enzyme 1 | ECE1 |
| IPI00027438 | Flotillin-1 | FLOT1 |
| IPI00852669 | Zinc finger protein 516 | ZNF516 |
| IPI00017297 | Matrin-3 | MATR3 |
| IPI00642645 | Methylenetetrahydrofolate reductase | MTHFR |
| IPI00470812 | Putative uncharacterized protein DKFZp781E21107 | LOC339977 |
| IPI00556381 | Putative uncharacterized protein (Fragment) | TRIM16 |
| IPI00004569 | Isoform MZF1A of Myeloid zinc finger 1 | MZF1 |
| IPI00240059 | Transmembrane and coiled-coil domains protein 3 | TMCC3 |
| IPI00022361 | Band 3 anion transport protein | SLC4A1 |
| IPI00017964 | Small nuclear ribonucleoprotein Sm D3 | SNRPD3 |
| IPI00478640 | Isoform 1 of Transmembrane protein C17orf87 | C17orf87 |
| IPI00013290 | hepatoma-derived growth factor-related protein 2 isoform 1 | HDGF2 |
| IPI00011051 | T-cell leukemia homeobox protein 1 | TLX1 |
| IPI00004838 | Isoform Crk-II of Proto-oncogene C-crk | CRK |
| IPI00154858 | Platelet endothelial aggregation receptor 1 precursor | PEAR1 |
| IPI00748733 | Similar to Slit homolog 3 protein precursor | — |
| IPI00023919 | 26S protease regulatory subunit 8 | PSMC5 |
| IPI00830051 | Similar to Immunolgoobulin heavy chain | LOC90925 |
| IPI00007423 | Isoform 1 of Acidic leucine-rich nuclear phosphoprotein 32 | ANP32B |
| | family member B | |
| IPI00553215 | V1-5 protein | IGLV2-18 |
| IPI00020005 | Arylsulfatase E precursor | ARSE |
| IPI00550720 | Isoform 1 of Uncharacterized protein C19orf57 | C19orf57 |
| IPI00103510 | Relaxin receptor 2 | RXFP2 |
| IPI00373872 | polycystin 1-like 2 isoform a | PKD1L2 |
| IPI00015181 | Integrin alpha-9 precursor | ITGA9 |
| IPI00013945 | Isoform 1 of Uromodulin precursor | UMOD |
| IPI00295172 | Ninjurin-1 | NINJ1 |
| IPI00328522 | KTEL motif-containing protein 1 | KTELC1 |
| IPI00000811 | Proteasome subunit beta type-6 precursor | PSMB6 |
| IPI00847415 | Isoform 4 of Dual specificity protein kinase CLK3 | CLK3 |
| IPI00251596 | Isoform 1 of Collagen alpha-1(XXIII) chain | COL23A1 |
| IPI00550906 | Cleavage stimulation factor 64 kDa subunit, tau variant | CSTF2T |
| IPI00550263 | Isoform 5 of Serine/threonine-protein kinase MRCK alpha | CDC42BPA |
| IPI00045219 | Sorting nexin-18 | SNAG1 |
| IPI00063245 | Uncharacterized protein FUBP3 | FUBP3 |
| IPI00221235 | nucleoporin 160 kDa | NUP160 |
| IPI00167560 | PAP-associated domain-containing protein 4 | PAPD4 |
| IPI00005719 | Isoform 1 of Ras-related protein Rab-1A | RAB1A |
| IPI00013933 | Isoform DPI of Desmoplakin | DSP |
| IPI00098902 | 2-oxoglutarate dehydrogenase E1 component, | OGDH |
| | mitochondrial precursor | |
| IPI00012079 | Eukaryotic translation initiation factor 4B | EIF4B |
| IPI00005793 | AP-3 complex subunit beta-2 | AP3B2 |
| IPI00289271 | Liprin-alpha-2 | PPFIA2 |
| IPI00216470 | Isoform 1 of Phosphatidylinositol-5-phosphate 4-kinase | PIP4K2B |
| | type-2 beta | |
| IPI00410391 | Isoform 1 of Major facilitator superfamily domain- | MFSD2 |
| | containing protein 2 | |
| IPI00441952 | Isoform 1 of Ninein | NIN |
| IPI00744825 | Conserved hypothetical protein | — |
| IPI00016940 | Phospholipid scramblase 2 | PLSCR2 |
| IPI00217264 | Isoform 3 of MAP7 domain-containing protein 3 | MAP7D3 |
| IPI00005675 | NF-kappa-B-repressing factor | NKRF |
| IPI00218628 | Isoform 2 of Integrin alpha-IIb precursor | ITGA2B |
| IPI00217989 | Isoform 1 of Protein-associating with the carboxyl-terminal | SCYL3 |
| | domain of ezrin | |
| IPI00217405 | Isoform 1 of E3 ubiquitin-protein ligase UBR1 | UBR1 |
| IPI00829915 | similar to PR domain zinc finger protein 16 | LOC647868 |
| IPI00455731 | Protein FAM26F | FAM26F |
| IPI00411656 | Isoform 1 of Protein piccolo | PCLO |
| IPI00217258 | CCDC100 protein | CCDC100 |
| IPI00604430 | Isoform 2 of Receptor expression-enhancing protein 2 | REEP2 |
| IPI00456670 | Isoform 13 of Peroxisomal N(1)-acetyl- | PAOX |
| | spermine/spermidine oxidase | |
| IPI00414927 | Transmembrane and immunoglobulin domain-containing | C1orf32 |
| | protein C1orf32 | |
| IPI00871458 | 39 kDa protein | C10orf4 |
| IPI00101299 | C1GALT1-specific chaperone 1 | C1GALT1C1 |
| IPI00186621 | Orofacial clefting chromosomal breakpoint region 1 | OFCC1 |
| IPI00295098 | Signal recognition particle receptor subunit beta | SRPRB |
| IPI00071824 | Isoform 1 of Cytoskeleton-associated protein 2 | CKAP2 |
| IPI00747494 | Glutamate receptor delta-2 subunit precursor | GRID2 |
| IPI00879409 | 28 kDa protein | — |
| IPI00760941 | Uncharacterized protein ENSP00000371687 | DKFZp434J1015 |
| IPI00169259 | Small VCP/p97-interacting protein | SVIP |
| IPI00514966 | Recombining binding protein suppressor of hairless | RBPJL |
| IPI00106506 | Isoform 1 of Evolutionarily conserved signaling | ECSIT |
| | intermediate in Toll pathway, mitochondrial precursor | |
| IPI00006900 | Something about silencing protein 10 | UTP3 |
| IPI00021772 | S-adenosylmethionine synthetase isoform type-1 | MAT1A |
| IPI00472332 | similar to polyhomeotic 1-like isoform 4 | LOC653441 |
| IPI00240909 | similar to eukaryotic translation initiation factor 3, subunit 5 | hCG_15200 |
| IPI00004671 | Golgin subfamily B member 1 | GOLGB1 |
| IPI00789245 | Isoform 2 of Probable organic cation transporter protein | SLC22A23 |
| | C6orf85 | |
| IPI00291922 | Proteasome subunit alpha type-5 | PSMA5 |
| IPI00217899 | RING finger protein 168 | RNF168 |
| IPI00242905 | Uncharacterized protein ENSP00000344689 (Fragment) | — |
| IPI00152145 | Protein odd-skipped-related 1 | OSR1 |
| IPI00152462 | Ciliary dynein heavy chain 3 | DNAH3 |
| IPI00021770 | Isoform 1 of 3-hydroxy-3-methylglutaryl-coenzyme A | HMGCR |
| | reductase | |
| IPI00465045 | DIP2 disco-interacting protein 2 homolog B | DIP2B |
| IPI00004509 | Leukotriene N synthase | LTC4S |
| IPI00018803 | homeobox D12 | HOXD12 |
| IPI00884371 | Uncharacterized protein ENSP00000383215 | — |
| IPI00002881 | Bcl-2-related protein A1 | BCL2A1 |
| IPI00021594 | Isoform 1 of Glycosylphosphatidylinositol anchor | GPAA1 |
| | attachment 1 protein | |
| IPI00010360 | Isoform 1 of Collagen alpha-3(IV) chain precursor | COL4A3 |
| IPI00022890 | Ig lambda chain V region 4A precursor | IGLV7-43 |
| IPI00219468 | Isoform IIa of Profilin-2 | PFN2 |
| IPI00065352 | Isoform 1 of Uncharacterized serine/threonine-protein | FLJ25006 |
| | kinase SgK494 | |
| IPI00000388 | Zinc finger protein 175 | ZNF175 |
| IPI00017163 | Isoform 1 of E3 ubiquitin-protein ligase HECW2 | HECW2 |
| IPI00465044 | Protein RCC2 | RCC2 |
| IPI00013847 | Cytochrome b-c1 complex subunit 1, mitochondrial | UQCRC1 |
| | precursor | |
| IPI00394807 | Regenerating islet-derived protein 3 gamma precursor | REG3G |
| IPI00024248 | Sodium/iodide cotransporter | SLC5A5 |
| IPI00022830 | Isoform 2 of NSFL1 cofactor p47 | NSFL1C |
| IPI00026665 | Glutaminyl-tRNA synthetase | QARS |
| IPI00180386 | Isoform GN-1L of Glycogenin-1 | GYG1 |
| IPI00465345 | Isoform 3 of Pre-mRNA-processing factor 40 homolog B | PRPF40B |
| IPI00065388 | Isoform 1 of Coiled-coil domain-containing protein 17 | CCDC17 |
| IPI00289540 | Isoform 2 of Usherin precursor | USH2A |
| IPI00748216 | CDNA FLJ20187 fis, clone COLF0433 | GTF3C5 |
| IPI00167638 | Isoform 1 of GTP-binding protein 10 | GTPBP10 |
| IPI00294713 | Isoform 1 of Mannan-binding lectin serine protease 2 | MASP2 |
| | precursor | |
| IPI00022143 | Isoform 1 of Extended-synaptotagmin-1 | FAM62A |
| IPI00023663 | Putative transcription factor Ovo-like 1 | OVOL1 |
| IPI00003406 | Isoform 1 of Drebrin | DBN1 |
| IPI00015602 | Mitochondrial precursor proteins import receptor | TOMM70A |
| IPI00072377 | Isoform 1 of Protein SET | SET |
| IPI00554752 | cAMP-dependent protein kinase type II-beta regulatory | PRKAR2B |
| | subunit | |
| IPI00011604 | Glycine cleavage system H protein, mitochondrial | GCSH; LOC730107 |
| | precursor | |
| IPI00401586 | Chromosome 18 open reading frame 62 | C18orf62 |
| IPI00026492 | 2-amino-3-ketobutyrate coenzyme A ligase, mitochondrial | GCAT |
| | precursor | |
| IPI00384861 | Isoform 1 of ARF GTPase-activating protein GIT1 | GIT1 |
| IPI00644529 | Isoform 2 of Zinc finger protein 615 | ZNF615 |
| IPI00028880 | Putative DNA polymerase | POLN |
| IPI00020008 | NEDD8 precursor | NEDD8 |
| IPI00180707 | Isoform 1 of FMS1-related extracellular matrix protein 2 | FREM2 |
| | precursor | |
| IPI00014878 | Uncharacterized protein PRKD1 | PRKD1 |
| IPI00794791 | Isoform 2 of IQ domain-containing protein D | IQCD |
| IPI00009853 | Vomeronasal type-1 receptor 1 | VN1R1 |
| IPI00022250 | Lymphocyte antigen 96 precursor | LY96 |
| IPI00332371 | Isoform 1 of 6-phosphofructokinase, liver type | PFKL |
| IPI00004337 | Zinc finger and BTB domain-Containing protein 11 | ZBTB11 |
| IPI00478860 | Glycoprotein endo-alpha-1,2-mannosidase | MANEA |
| IPI00005708 | Heparan sulfate glucosamine 3-O-sulfotransferase 3B1 | HS3ST3B1 |
| IPI00169430 | Isoform 1 of Spermatid perinuclear RNA-binding protein | STRBP |
| IPI00007057 | Rabenosyn-5 | ZFYVE20 |
| IPI00386822 | Putative uncharacterized protein (Fragment) | TGFBI |
| IPI00477539 | Uncharacterized protein C14orf102 | C14orf102 |
| IPI00296219 | Glutaminase liver isoform, mitochondrial precursor | GLS2 |
| IPI00005564 | Stanniocalcin-1 precursor | STC1 |
| IPI00376087 | putative binding protein 7a5 | 7A5 |
| IPI00413344 | Cofilin-2 | CFL2 |
| IPI00006648 | IDN4-GGTR14 protein | RABGAP1L |
| IPI00302176 | Isoform 1 of H/ACA ribonucleoprotein complex subunit 1 | NOLA1 |
| IPI00014850 | Astrocytic phosphoprotein PEA-15 | PEA15 |
| IPI00794679 | Major histocompatibility complex, class I, B | MICA; HLA-B; HLA- |
| | | A; LOC730410; HLA- |
| | | C; HLA-A29.1 |
| IPI00008282 | Isoform 1 of Calcium/calmodulin-dependent 3′,5′-cyclic | PDE1A |
| | nucleotide phosphodiesterase 1A | |
| IPI00007277 | Isoform 1 of Leucine-rich repeat flightless-interacting | LRRFIP2 |
| | protein 2 | |
| IPI00294519 | Isoform 1 of Telomerase protein component 1 | TEP1 |
| IPI00478586 | Isoform 2 of Vacuolar protein sorting-associated protein | VPS13A |
| | 13A | |
| IPI00060439 | KIAA0226 protein | KIAA0226 |
| IPI00303343 | Splicing factor, arginine/serine-rich 19 | SCAF1 |
| IPI00179589 | Myotrophin | MTPN |
| IPI00744811 | Low-density lipoprotein receptor-related protein 5 | LRP5 |
| | precursor | |
| IPI00031485 | Mitochondrial ribosomal protein 63 | MRP63 |
| IPI00784044 | Isoform 1 of Methylcrotonoyl-CoA carboxylase beta chain, | MCCC2 |
| | mitochondrial precursor | |
| IPI00869070 | Ankyrin repeat containing protein | — |
| IPI00783186 | Isoform 2 of Ankyrin repeat domain-containing protein 17 | ANKRD17 |
| IPI00303152 | collagen, type XXII, alpha I | COL22A1 |
| IPI00429191 | Eukaryotic peptide chain release factor subunit 1 | ETF1 |
| IPI00514594 | Isoform 1 of Protein FAM5B precursor | FAM5B |
| IPI00853376 | additional sex combs like 3 | ASXL3 |
| IPI00216914 | Vitelline membrane outer layer protein 1 homolog | VMO1 |
| | precursor | |
| IPI00423379 | Putative uncharacterized protein DKFZp686G24192 | C20orf96 |
| IPI00025308 | Homeobox protein prophet of Pit-1 | PROP1 |
| IPI00219358 | Isoform 1 of Mannose-6-phosphate isomerase | MPI |
| IPI00400967 | KIAA1843 protein (Fragment) | KIAA1843 |
| IPI00166010 | Isoform 1 of CCR4-NOT transcription complex subunit 1 | CNOT1 |
| IPI00793576 | 7 kDa protein | PRH2; PRB4; PRH1 |
| IPI00307757 | Isoform 1 of Membrane-associated phosphatidylinositol | PITPNM3 |
| | transfer protein 3 | |
| IPI00166738 | Zinc-binding alcohol dehydrogenase domain-containing | ZADH2 |
| | protein 2 | |
| IPI00399252 | Isoform 1 of Protein Jade-1 | PHF17 |
| IPI00412977 | Prothymosin alpha | — |
| IPI00396077 | Isoform 1 of E3 ubiquitin-protein ligase Topors | TOPORS |
| IPI00144243 | Human immunodeficiency virus type I enhancer-binding | HIVEP2 |
| | protein 2 | |
| IPI00446355 | CDNA FLJ41803 fis, clone NHNPC2002749 | IER5L |
| IPI00167861 | Protein SMG5 | SMG5 |
| IPI00005530 | FMRFamide-related peptides precursor | NPVF |
| IPI00186004 | hypothetical protein LOC57730 | KIAA1641 |
| IPI00465178 | Isoform 1 of Vacuolar proton translocating ATPase 116 kDa | ATP6V0A1 |
| | subunit a isoform 1 | |
| IPI00291463 | Radical S-adenosyl methionine domain-containing protein 2 | RSAD2 |
| IPI00472171 | RPL7 protein | RPL7 |
| IPI00187091 | Isoform 2 of MAP kinase-interacting serine/threonine- | MKNK1 |
| | protein kinase 1 | |
| IPI00398421 | Isoform 1 of Potassium channel subfamily T member 2 | KCNT2 |
| IPI00007040 | Zinc finger protein 222 | ZNF222 |
| IPI00220578 | Guanine nucleotide-binding protein G | GNAI3 |
| IPI00740191 | similar to Forkhead box protein L1 | LOC651986 |
| IPI00044607 | Protein phosphatase inhibitor 2-like protein 1 | PPP1R2P1 |
| IPI00301923 | Isoform 1 of Cell division protein kinase 9 | CDK9 |
| IPI00255145 | hypothetical protein | LOC342346 |
| IPI00022228 | Vigilin | HDLBP |
| IPI00010737 | Thrombomodulin precursor | THBD |
| IPI00059395 | Kinesin-like protein KIFC2 | KIFC2 |
| IPI00030319 | Forkhead box protein F2 | FOXF2 |
| IPI00168806 | Isoform 1 of Myeloid/lymphoid or mixed-lineage leukemia | MLL3 |
| | protein 3 homolog | |
| IPI00018275 | Prion-like protein doppel precursor | PRND |
| IPI00216704 | Isoform 2 of Spectrin beta chain, erythrocyte | SPTB |
| IPI00034277 | Isoform A of Probable cation-transporting ATPase 13A1 | ATP13A1 |
| IPI00015285 | Ethanolamine-phosphate cytidylyltransferase | PCYT2 |
| IPI00152853 | Isoform 1 of Phostensin | KIAA1949 |
| IPI00302850 | Small nuclear ribonucleoprotein Sm DI | SNRPDI |
| IPI00478124 | 61 kDa protein | UCKL1 |
| IPI00013681 | N-terminally extended type 3 canonical transient receptor | TRPC3 |
| | potential channel | |
| IPI00446785 | CDNA FLJ41116 fis, clone BRACE1000572 | — |
| IPI00784272 | Putative uncharacterized protein | — |
| IPI00293460 | ATP-binding cassette sub-family A member 1 | ABCA1 |
| IPI00019208 | Similar to 60S ribosomal protein L29 | — |
| IPI00304596 | Non-POU domain-containing octamer-binding protein | NONO |
| IPI00411452 | Uncharacterized protein DOCK11 | DOCK11 |
| IPI00444395 | CDNA FLJ45615 fis, clone BRTHA3026180 | — |
| IPI00019278 | Thiamine transporter 2 | SLC19A3 |
| IPI00012895 | Isoform 1 of Carbonic anhydrase 12 precursor | CA12 |
| IPI00299627 | Dual oxidase 2 precursor | DUOX2 |
| IPI00216132 | Isoform Alpha 1 of Ribosomal protein S6 kinase beta-1 | RPS6KB1 |
| IPI00023513 | Isoform 1 of E3 ubiquitin-protein ligase CHFR | CHFR |
| IPI00063827 | Isoform 1 of Abhydrolase domain-containing protein 14B | ABHD14B |
| IPI00025019 | Proteasome subunit beta type-1 precursor | PSMB1 |
| IPI00328115 | Isoform 3 of 5′-3′ exoribonuclease 1 | XRN1 |
| IPI00022048 | Prostaglandin F2 receptor negative regulator precursor | PTGFRN |
| IPI00103055 | Isoform 1 of Solute carrier family 35 member F5 | SLC35F5 |
| IPI00167194 | CDNA FLJ25694 fis, clone TST04471 | FLJ25694 |
| IPI00002966 | Heat shock 70 kDa protein 4 | HSPA4 |
| IPI00152536 | Isoform 1 of Transmembrane channel-like protein 2 | TMC2 |
| IPI00783271 | Leucine-rich PPR motif-containing protein, mitochondrial | LRPPRC |
| | precursor | |
| IPI00298476 | Isoform 1 of Gremlin-1 precursor | GREM1 |
| IPI00150057 | Isoform 2 of SWI/SNF-related matrix-associated actin- | SMARCC2 |
| | dependent regulator of chromatin subfamily C member 2 | |
| IPI00456996 | Conserved hypothetical protein | PVT1 |
| IPI00232891 | Isoform 3 of Fibronectin type III domain-containing protein 1 | FNDC1 |
| IPI00018402 | Tubulin-specific chaperone E | TBCE |
| IPI00166817 | Zinc finger protein 561 | ZNF561 |
| IPI00375174 | Ankyrin repeat and sterile alpha motif domain-containing | ANKS1B |
| | protein 1B | |
| IPI00030404 | Transcriptional repressor NF-X1 | NFX1 |
| IPI00296866 | interphotoreceptor matrix proteoglycan 2 | IMPG2 |
| IPI00219661 | Isoform 1 of Myelin proteolipid protein | PLPI |
| IPI00040730 | protocadherin 21 precursor | PCDH21 |
| IPI00013508 | Alpha-actinin-1 | ACTN1 |
| IPI00186853 | pecanex-like 2 | PCNXL2 |
| IPI00001120 | CDNA F1131810 fis, clone NT2RI2009289, weakly | LINGO2 |
| | similar to CARBOXYPEPTIDASE N 83 KD CHAIN | |
| IPI00395376 | Isoform 1 of Semaphorin-5B | SEMA5B |
| IPI00555621 | p21-activated kinase 2 variant (Fragment) | — |
| IPI00746232 | coiled-coil domain containing 112 isoform 2 | CCDC112 |
| IPI00022020 | Type II inositol-3,4-bisphosphate 4-phosphatase | INPP4B |
| IPI00644231 | Isoform 1 of Cytoplasmic FMR1-interacting protein 1 | CYFIP1 |
| IPI00007193 | Isoform 2 of Ankyrin repeat domain-containing protein 26 | ANKRD26 |
| IPI00418236 | Factor in the germline alpha | FIGLA |
| IPI00328762 | Isoform 1 of ATP-binding cassette sub-family A member | ABCA13 |
| | 13 | |
| IPI00748342 | Beclin-1 | BECN1 |
| IPI00739423 | similar to c11.1 CG12132-PA isoform 11 | LOC377711 |
| IPI00026970 | FACT complex subunit SPT16 | SUPT16H |
| IPI00013466 | Arsenical pump-driving ATPase | ASNA1 |
| IPI00016605 | Uncharacterized protein C1orf123 | C1orf123 |
| IPI00872861 | PTD016 protein | LOC51136 |
| IPI00010130 | Glutamine synthetase | GLUL |
| IPI00166619 | Isoform 2 of Putative transporter SVOPL | SVOPL |
| IPI00549384 | Isoform 1 of Bromodomain-containing protein 9 | BRD9 |
| IPI00479893 | Isoform 2 of Uncharacterized protein C10orf18 | C10orf18 |
| IPI00410585 | Isoform 1 of Crumbs homolog 2 precursor | CRB2 |
| IPI00876910 | Isoform 1 of Ankyrin repeat and SOCS box-containing | ASB18 |
| | protein 18 | |
| IPI00008202 | Headcase protein homolog | HECA |
| IPI00477766 | Isoform 3 of Protein PTHB1 | BBS9 |
| IPI00029722 | Kinesin heavy chain isoform 5A | KIF5A |
| IPI00301503 | Isoform 1 of Splicing factor, arginine/serine-rich 10 | SFRS10 |
| IPI00031696 | FAST kinase domain-containing protein 3 | FASTKD3 |
| IPI00425688 | Isoform I of RUN and SH3 domain-containing protein 1 | RUSC1 |
| IPI00787427 | ankyrin repeat domain 36 | ANKRD36 |
| IPI00796647 | HIG1 domain family, member 1C | HIGD1C |
| IPI00852758 | Similar to Ankyrin repeat domain-containing protein 26. | ANKRD18B |
| | Isoform 2 | |
| IPI00023087 | Ubiquitin-conjugating enzyme E2 T | UBE2T |
| IPI00167241 | Zinc finger protein 283 | ZNF283 |
| IPI00220325 | Isoform Short of Insulin receptor precursor | INSR |
| IPI00465322 | Uncharacterized protein BOC | BOC |
| IPI00161119 | Isoform 1 of NF-kappa-B inhibitor beta | NFKBIB |
| IPI00016577 | CDNA: FLJ22814 fis, clone KAIA3004 | CD22 |
| IPI00032541 | Keratin type II cuticular Hb5 | KRT85 |
| IPI00852633 | 16 kDa protein | RABL2B |
| IPI00163391 | Isoform 1 of Putative methyltransferase METT10D | METT10D |
| IPI00299679 | Isoform B of Ral guanine nucleotide dissociation stimulator | RGL1 |
| | like 1 | |
| IPI00797699 | 20 kDa protein | — |
| IPI00384874 | Isoform 1 of ATP-binding cassette sub-family A member | ABCA10 |
| | 10 | |
| IPI00019690 | death-inducing-protein | DIP |
| IPI00794119 | 13 kDa protein | ABCC8 |
| IPI00027723 | Elastase-2B precursor | ELA2B |
| IPI00816274 | Chemokine-like factor superfamily 1 transcript variant 26 | CMTM1 |
| IPI00015737 | 15 kDa protein | DCAKD |
| IPI00641954 | 22 kDa protein | TMED5 |
| IPI00739676 | Isoform 2 of Protein DDX26B | DDX26B |
| IPI00038378 | Isoform 1 of Enolase-phosphatase E1 | ENOPH1 |
| IPI00171737 | Isoform 2 of Leucine-rich repeat and death domain- | LRDD |
| | containing protein | |
| IPI00385003 | Putative transposase | TIGD1 |
| IPI00477763 | Serine/threonine-protein kinase MRCK beta | CDC42BPB |
| IPI00029123 | Isoform A of Endothelin B receptor precursor | EDNRB |
| IPI00158992 | snRNA-activating protein complex subunit 4 | SNAPC4 |
| IPI00402006 | Similar to DNA-binding protein | — |
| IPI00306576 | Arylsulfatase B precursor | ARSB |
| IPI00184997 | cDNA FLJ78771, highly similar to Homo sapiens discs, | DLG7 |
| | large homolog 7 (Drosophila), mRNA | |
| IPI00477949 | Isoform 1 of Zinc finger MYM-type protein 4 | ZMYM4 |
| IPI00001869 | Pappalysin-1 precursor | PAPPA |
| IPI00027834 | heterogeneous nuclear ribonucleoprotein L isoform a | HNRNPL |
| IPI00062730 | Uncharacterized protein C16orf45 | C16orf45 |
| IPI00737448 | similar to heat shock 70 kD protein binding protein | LOC338805 |
| IPI00298258 | UNC13B protein | UNC13B |
| IPI00383261 | Peroxin Pex6p | PEX6 |
| IPI00022735 | Synaptotagmin-4 | SYT4 |
| IPI00003391 | Teneurin-1 | ODZ1 |
| IPI00744366 | Conserved hypothetical protein | — |
| IPI00000792 | Quinone oxidoreductase | CRYZ |
| IPI00008905 | UDP-glucuronosyltransferase 2B15 precursor | UGT2B15 |
| IPI00031411 | Cadherin-related tumor suppressor homolog precursor | FAT |
| IPI00026058 | Isoform 1 of Leucine zipper putative tumor suppressor 1 | LZTS1 |
| IPI00377077 | Isoform 3 of Astrotactin-2 precursor | ASTN2 |
| IPI00479789 | Isoform 1 of Uncharacterized protein C1orf103 | C1orf103 |
| IPI00186581 | amplified in osteosarcoma isoform 2 precursor | OS9 |
| IPI00445364 | CDNA FLJ44171 fis, clone THYMU2036058 | — |
| IPI00411637 | Galactose-3-O-sulfotransferase 4 | GAL3ST4 |
| IPI00154834 | Isoform 1 of Neighbor of punc e11 precursor | NOPE |
| IPI00216697 | Isoform Er1 of Ankyrin-1 | ANK1 |
| IPI00021985 | transmembrane 9 superfamily protein member 4 | TM9SF4 |
| IPI00166071 | B-cell CLL/lymphoma 6 member B protein | BCL6B |
| IPI00007360 | Zinc finger protein 238 | ZNF238 |
| IPI00217023 | MMAA protein | MMAA |
| IPI00218130 | Glycogen phosphorylase, muscle form | PYGM |
| IPI00384280 | Prenylcysteine oxidase 1 precursor | PCYOX1 |
| IPI00152733 | FLJ00237 protein (Fragment) | OLFML2A |
| IPI00034006 | Tyrosine-protein phosphatase non-receptor type 23 | PTPN23 |
| IPI00793585 | 57 kDa protein | OC90 |
| IPI00419253 | Isoform 1 of Nck-associated protein 5 | NAP5 |
| IPI00029133 | ATP synthase subunit b, mitochondrial precursor | ATP5F1 |
| IPI00178727 | Novel protein | C10orf79 |
| IPI00218271 | Isoform 3 of MAGUK p55 subfamily member 2 | MPP2 |
| IPI00003949 | Ubiquitin-conjugating enzyme E2 N | UBE2N |
| IPI00292393 | Sodium channel protein type 4 subunit alpha | SCN4A |
| IPI00304992 | Isoform 1 of Zinc finger protein 503 | ZNF503 |
| IPI00029012 | Eukaryotic translation initiation factor 3 subunit A | EIF3A |
| IPI00043201 | Centromere protein J | CENPJ |
| IPI00022891 | ADP/ATP translocase 1 | SLC25A4 |
| IPI00006612 | Isoform 1 of Clathrin coat assembly protein AP180 | SNAP91 |
| IPI00385042 | Nucleolar GTP-binding protein 1 | GTPBP4 |
| IPI00642305 | MCM8 protein | MCM8 |
| IPI00402144 | Isoform 1 of Zinc finger protein 555 | ZNF555 |
| IPI00478986 | Similar to 40S ribosomal protein S4 | LOC126235 |
| IPI00029623 | Proteasome subunit alpha type-6 | PSMA6 |
| IPI00026320 | E3 ubiquitin-protein ligase UBR5 | UBR5 |
| IPI00010142 | Ubiquitin-conjugating enzyme E2 G2 | UBE2G2 |
| IPI00478521 | Isoform 1 of UPF0475 protein | RILPL1 |
| IPI00029769 | Isoform p59-HCK of Tyrosine-protein kinase HCK | HCK |
| IPI00446138 | CDNA FLJ42768 fis, clone BRAWH3003522 | — |
| IPI00046057 | lsoform 2 of Syntaxin-binding protein 1 | STXBP1 |
| IPI00449071 | Isoform 5a of Paired box protein Pax-6 | PAX6 |
| IPI00328268 | EIF4G3 protein | EIF4G3 |
| IPI00305477 | Cystatin-SN precursor | CST1 |
| IPI00432893 | INPP5F protein (Fragment) | INPP5F |
| IPI00220039 | Isoform 1 of Transmembrane protein 16K | TMEM16K |
| IPI00021347 | Ubiquitin-conjugating enzyme E2 L3 | UBE2L3 |
| IPI00025700 | Isoform CD6A of T-cell differentiation antigen CD6 | CD6 |
| | precursor | |
| IPI00061507 | Isoform 3 of Ester hydrolase C11orf54 | C11orf54 |
| IPI00000270 | Ribonuclease 7 precursor | RNASE7 |
| IPI00479430 | Isoform 2 of Complement C1q tumor necrosis factor- | CIQTNF9 |
| | related protein 9 precursor | |
| IPI00386754 | Isoform 2 of Cysteine-rich with EGF-like domain protein 2 | CRELD2 |
| | precursor | |
| IPI00016949 | Isoform 4 of Electrogenic sodium bicarbonate cotransporter 1 | SLC4A4 |
| IPI00031547 | Desmoglein-3 precursor | DSG3 |
| IPI00382756 | Isoform 2 of Pleiotropic regulator 1 | PLRG1 |
| IPI00604599 | Transmembrane emp24 domain-containing protein 3 | TMED3 |
| | precursor | |
| IPI00296798 | Isoform 1 of Fibrinogen C domain-containing protein 1 | FIBCD1 |
| IPI00743284 | Methionine synthase | MTR |
| IPI00021363 | Histone demethylase JARID1A | JARID1A |
| IPI00749440 | Uncharacterized protein ENSP00000368180 | — |
| IPI00216683 | M-phase inducer phosphatase 3 | CDC25C |
| IPI00737969 | microtubule associated monoxygenase, calponin and LIM | MICAL3 |
| | domain containing 3 | |
| IPI00015522 | Growth/differentiation factor 5 precursor | GDF5 |
| IPI00877084 | Isoform 1 of Coiled-coil domain-containing protein 144C | CCDC144C |
| IPI00060265 | Zinc finger protein 775 | ZNF775 |
| IPI00171199 | Isoform 2 of Proteasome subunit alpha type-3 | PSMA3 |
| IPI00003483 | Isoform 1 of Neuralized-like protein 1 | NEURL |
| IPI00024802 | TATA-binding protein-associated factor 172 | BTAF1 |
| IPI00374039 | Conserved hypothetical protein | C1orf189 |
| IPI00419575 | Protein of unknown function DUF410 family protein | C7orf20 |
| IPI00019090 | Collagen alpha-1 | COL19A1 |
| IPI00011756 | Homeobox protein Meis1 | MEIS1 |
| IPI00021834 | Isoform Alpha of Tissue factor pathway inhibitor precursor | TFPI |
| IPI00167254 | Isoform 4 of Inactive phospholipase D5 | PLD5 |
| IPI00442121 | delta-aminolevulinic acid dehydratase isoform a | ALAD |
| IPI00013455 | CLIP1 protein | CLIP1 |
| IPI00026299 | Isoform Glycophorin C of Glycophorin-C | GYPC |
| IPI00012687 | 27 kDa protein | C1orf83 |
| IPI00845508 | BAH domain and coiled-coil containing 1 | BAHCC1 |
| IPI00332106 | Isoform 1 of Pre-B-cell leukemia transcription factor- | PBXIP1 |
| | interacting protein 1 | |
| IPI00418426 | Metal transporter CNNM4 | CNNM4 |
| IPI00002524 | ATP-sensitive inward rectifier potassium channel 14 | KCNJ14 |
| IPI00745300 | 31 kDa protein | NAT11 |
| IPI00000110 | Zinc finger protein 337 | ZNF337 |
| IPI00009148 | Diphosphoinositol polyphosphate phosphohydrolase 1 | NUDT3 |
| IPI00297767 | Casein kinase I isoform gamma-2 | CSNK1G2 |
| IPI00186826 | Ephrin receptor | EPHB4 |
| IPI00787932 | similar to zinc finger protein 10 | hCG_1646157 |
| IPI00007207 | Isoform 1 of Lysosomal acid lipase/cholesteryl ester | LIPA |
| | hydrolase precursor | |
| IPI00023868 | Canalicular multispecific organic anion transporter 1 | ABCC2 |
| IPI00166161 | Protein SIX6OS1 | C14orf39 |
| IPI00180426 | Isoform 3 of G protein-coupled receptor kinase 4 | GRK4 |
| IPI00031522 | Trifunctional enzyme subunit alpha, mitochondrial | HADHA |
| | precursor | |
| IPI00013219 | Integrin-linked protein kinase | ILK |
| IPI00070070 | Novel protein | FLJ32784 |
| IPI00329142 | Glutamate-rich protein 1 | ERICH1 |
| IPI00169253 | Olfactory receptor OR1-45 | OR5AT1 |
| IPI00045801 | Flt3-interacting zinc finger protein 1 | FIZ1 |
| IPI00017802 | Isoform 1 of Methylglutaconyl-CoA hydratase, | AUH |
| | mitochondrial precursor | |
| IPI00796906 | 8 kDa protein | ABCF3 |
| IPI00384549 | TTC31 protein (Fragment) | TTC31 |
| IPI00028932 | Microtubule-associated serine/threonine-protein kinase 3 | MAST3 |
| IPI00012756 | Interferon-induced protein with tetratricopeptide repeats 5 | IFIT5 |
| IPI00297985 | Uncharacterized protein C6orf103 | C6orf103 |
| IPI00514394 | Family with sequence similarity 120A opposite strand | FAM120AOS |
| IPI00034088 | Interleukin-27 beta chain precursor | EBI3 |
| IPI00305856 | Homeobox protein Hox-D11 | HOXD11 |
| IPI00387159 | Isoform 1 of Inhibitor of growth protein 3 | ING3 |
| IPI00737429 | Teneurin-4 | ODZ4 |
| IPI00645089 | Kv channel interacting protein 1 isoform 3 | KCNIP1 |
| IPI00797859 | 10 kDa protein | LOC643711 |
| IPI00385737 | similar to Nonhistone chromosomal protein HMG-17 | LOC646853 |
| IPI00015195 | Cleavage stimulation factor 77 kDa subunit | CSTF3 |
| IPI00006158 | Lymphoid-restricted membrane protein | LRMP |
| IPI00008554 | Angiogenin precursor | ANG |
| IPI00412216 | vacuolar protein sorting 13C protein isoform 2B | VPS13C |
| IPI00028158 | Neurotensin/neuromedin N precursor | NTS |
| IPI00020884 | Plexin-A3 precursor | PLXNA3 |
| IPI00374862 | Isoform 1 of Kelch-like protein 5 | KLHL5 |
| IPI00384393 | Myosin-reactive immunoglobulin heavy chain variable | — |
| | region (Fragment) | |
| IPI00009377 | HSPC212 | C3orf19 |
| IPI00005969 | F-actin-capping protein subunit alpha-1 | CAPZA1 |
| IPI00021129 | Isoform 1 of AP-3 complex subunit beta-1 | AP3B1 |
| IPI00013871 | Ribonucleoside-diphosphate reductase large subunit | RRM1 |
| IPI00020039 | Cylicin-1 | CYLC1 |
| IPI00300244 | zinc finger, CW type with PWWP domain 1 | ZCWPW1 |
| IPI00060308 | Isoform 6 of PDZ and LIM domain protein 7 | PDLIM7 |
| IPI00333410 | Isoform 1 of Ubiquitin-conjugating enzyme E2 Q1 | UBE2Q1 |
| IPI00737712 | CCDC125 protein | CCDC125 |
| IPI00219036 | dicer1 | DICER1 |
| IPI00168862 | Conserved hypothetical protein | PXT1 |
| IPI00025647 | Isoform 1 of F-box only protein 21 | FBXO21 |
| IPI00218075 | Protein FAM9B | FAM9B |
| IPI00738499 | Ferritin light chain | FTL |
| IPI00297212 | Paladin | KIAA1274 |
| IPI00252845 | SYT9 protein | SYT9 |
| IPI00477361 | 10 kDa protein | SDHALP1 |
| IPI00418185 | Putative uncharacterized protein FLJ20097 | CCDC132 |
| IPI00423562 | Isoform 1 of Ubiquitin carboxyl-terminal hydrolase 6 | USP6 |
| IPI00397578 | 135 kDa protein | PPFIA4 |
| IPI00004101 | Betaine--homocysteine S-methyltransferase 1 | BHMT |
| IPI00017659 | Protein kinase substrate CapZIP | RCSD1 |
| IPI00025094 | CDNA: FLJ22037 fis, clone HEP08868 (Fragment) | MYH16 |
| IPI00008475 | Hydroxymethylglutaryl-CoA synthase, cytoplasmic | HMGCS1 |
| IPI00023315 | Bone morphogenetic protein 3b precursor | GDF10 |
| IPI00412107 | FLJ43980 protein | FLJ43980 |
| IPI00328793 | Sterol regulatory element-binding protein 2 | SREBF2 |
| IPI00216963 | Isoform 9 of CASP8 and FADD-like apoptosis regulator | CFLAR |
| | precursor | |
| IPI00010895 | Tubby-related protein 2 | TULP2 |
| IPI00739106 | similar to ribosomal protein L5 isoform 1 | LOC647436 |
| IPI00022606 | Isoform 1 of Proline-serine-threonine phosphatase- | PSTPIP1 |
| | interacting protein 1 | |
| IPI00004563 | Protein FAM105A | FAM105A |
| IPI00550232 | cardiomyopathy associated 3 isoform 1 | XIRP2 |
| IPI00299147 | Small ubiquitin-related modifier 3 precursor | SUMO3 |
| IPI00295502 | Isoform 1 of Protein Wiz | WIZ |
| IPI00435925 | PP14214 | IGFBP3 |
| IPI00216106 | Isoform 3 of Obg-like ATPase 1 | OLA1 |
| IPI00013257 | Single-stranded DNA-binding protein 4 | SSBP4 |
| IPI00002707 | Isoform 1 of Spastin | SPAST |
| IPI00010118 | Isoform 1 of Prostate tumor overexpressed gene 1 protein | PTOV1 |
| IPI00158804 | basic, immunoglobulin-like variable motif containing | BIVM |
| IPI00166606 | KIAA0913 protein (Fragment) | KIAA0913 |
| IPI00329014 | Isoform 1 of Ankyrin repeat domain-containing protein 46 | ANKRD46 |
| IPI00300990 | Isoform 1 of Uncharacterized protein C1orf77 | C1orf77 |
| IPI00741780 | similar to CG4845-PA | LOC652559 |
| IPI00395603 | Isoform 3 of Uncharacterized protein C18orf34 | C18orf34 |
| IPI00454858 | similar to alpha 3 type VI collagen isoform 1 precursor | LOC344875 |
| IPI00399328 | similar to jumonji domain containing 2D | LOC390245 |
| IPI00852619 | Isoform A of Probable phospholipid-transporting ATPase | ATP10B |
| | VB | |
| IPI00303980 | Isoform 1 of Uncharacterized protein C14orf93 precursor | C14orf93 |
| IPI00433499 | rhomboid, veinlet-like 6 isoform 1 | RHBDF2 |
| IPI00006196 | Isoform 2 of Nuclear mitotic apparatus protein 1 | NUMA1 |
| IPI00004534 | Phosphoribosylformylglycinamidine synthase | PFAS |
| IPI00010575 | KIAA1466 protein | KIAA1466 |
| IPI00004446 | Sushi repeat-containing protein SRPX2 precursor | SRPX2 |
| IPI00785015 | Isoform 1 of Uncharacterized protein KIAA2030 | FLJ25778 |
| IPI00247295 | Isoform 4 of Nesprin-1 | SYNE1 |
| IPI00187143 | Isoform 2 of Ras-related protein Rab-4B | RAB4B |
| IPI00010448 | Isoform 2 of Rho GTPase-activating protein 24 | ARHGAP24 |
| IPI00103630 | Isoform 2 of Protein phosphatase 1E | PPM1E |
| 12 | | |
| IPI00479669 | Isoform 1 of Uncharacterized protein KIAA0701 | UHRF1BP1L |
| IPI00737392 | similar to proline-rich protein BstNI subfamily 3 precursor | LOC646352 |
| IPI00017940 | LMBR1 domain-containing protein 2 | LMBRD2 |
| IPI00219503 | Isoform 1 of F-box only protein 24 | FBXO24 |
| IPI00414591 | Flavoprotein oxidoreductase | MICAL2 |
| IPI00018240 | Isoform 1 of Protein SDA1 homolog | SDAD1 |
| IPI00001872 | Isoform 1 of Protocadherin gamma C3 precursor | PCDHGC3; PCDHGA12 |
| IPI00657892 | Factor VIII | F8 |
| IPI00216508 | Isoform 2 of Sorting nexin-3 | SNX3 |
| IPI00011400 | T-lymphoma invasion and metastasis-inducing protein 1 | TIAM1 |
| IPI00060146 | Isoform 1 of Smith-Magenis syndrome chromosome region | SMCR7 |
| | candidate gene 7 protein | |
| IPI00175989 | Rho family guanine-nucleotide exchange factor | MCF2L2 |
| IPI00386651 | LOC404266 protein | LOC404266 |
| IPI00022989 | Isoform Beta-1 of Retinoic acid receptor beta | RARB |
| IPI00166933 | Uncharacterized protein C10orf12 | C10orf12 |
| IPI00005254 | Isoform 1 of E3 ubiquitin-protein ligase parkin | PARK2 |
| IPI00152072 | hypothetical protein LOC387758 | FIBIN |
| IPI00060546 | Uncharacterized protein C10orf35 | C10orf35 |
| IPI00005563 | Isoform 1 of Tubulointerstitial nephritis antigen-like | TINAGL1 |
| | precursor | |
| IPI00043978 | Isoform 1 of Partitioning-defective 3 homolog B | PARD3B |
| IPI00440221 | Putative uncharacterized protein (Fragment) | CDC2L5 |
| IPI00011564 | Syndecan-4 precursor | SDC4 |
| IPI00423683 | Isoform 2 of EMI domain-containing protein 1 precursor | EMID1 |
| IPI00026612 | Isoform Beta-1 of Protein phosphatase 1B | PPM1B |
| IPI00065378 | Isoform 1 of DC-STAMP domain-containing protein 1 | DCST1 |
| IPI00163718 | Isoform 4 of Vacuolar protein sorting-associated protein 54 | VPS54 |
| IPI00167137 | Isoform 3 of SLAM family member 7 precursor | SLAMF7 |
| IPI00300623 | Pro-MCH precursor | PMCH |
| IPI00155447 | MMP28 protein | MMP28 |
| IPI00414819 | Helicase SKI2W | SKIV2L |
| IPI00001434 | Protocadherin beta 14 precursor | PCDHB14 |
| IPI00041588 | Ral guanine nucleotide exchange factor RaIGPSIA | RALGPSI |
| IPI00100787 | RANBP2-like and GRIP domain-containing protein 7 | RGPD5; RGPD7 |
| IPI00011865 | Isoform 2 of Platelet-derived growth factor D precursor | PDGFD |
| IPI00043731 | CDNA FLJ30671 fis, clone FCBBF1000687, moderately | RUNDC3B |
| | similar to Mus musculus Rap2 interacting protein 8 | |
| | (RPIP8) mRNA | |
| IPI00746388 | Ezrin | EZR |
| IPI00873759 | Uncharacterized protein ENSP00000381675 (Fragment) | — |
| IPI00012390 | Homeobox protein Hox-D4 | HOXD4 |
| IPI00645814 | Isoform 2 of MAP7 domain-containing protein 1 | MAP7D1 |
| IPI00027694 | Isoform 3 of Homeobox protein Hox-A1 | HOXA1 |
| IPI00514551 | OTTHUMP00000016936 | SCML4 |
| IPI00747264 | Gamma-aminobutyric-acid receptor subunit rho-1 precursor | GABRR1 |
| IPI00003353 | Neuronal protein 3.1 | C5orf13 |
| IPI00007402 | Importin-7 | IPO7 |
| IPI00337544 | Isoform 2 of Myomegalin | PDE4DIP |
| IPI00152470 | Prokineticin receptor 1 | PROKR1 |
| IPI00294619 | Protein TFG | TFG |
| IPI00020795 | R33083_1 (Fragment) | DPP9 |
| IPI00010740 | Isoform Long of Splicing factor, proline- and glutamine- | SFPQ |
| | rich | |
| IPI00006094 | Regulating synaptic membrane exocytosis protein 3 | RIMS3 |
| IPI00100154 | Toll-interacting protein | TOLLIP |
| IPI00019243 | Isoform Long of Matrix metalloproteinase-16 precursor | MMP16 |
| IPI00000203 | Low-density lipoprotein receptor-related protein 6 | LRP6 |
| | precursor | |
| IPI00749338 | Hypothetical gene supported by BC063892 | hCG_1651889 |
| IPI00221009 | Isoform 7 of Transcription factor 7-like 2 | TCF7L2 |
| IPI00101927 | Leucine zipper putative tumor suppressor 2 | LZTS2 |
| IPI00010346 | Neurolysin, mitochondrial precursor | NLN |
| IPI00398725 | Isoform 1 of Zinc finger protein 644 | ZNF644 |
| IPI00745832 | IQ calmodulin-binding region domain containing protein | — |
| IPI00167228 | CDNA FLJ40966 fis, clone UTERU2012407 | CHCHD7 |
| IPI00216572 | BarH-like homeobox 2 | BARX2 |
| IPI00007067 | Golgi-associated plant pathogenesis-related protein 1 | C9orf19 |
| IPI00410616 | Isoform 1 of Endonuclease VIII-like 2 | NEIL2 |
| IPI00644025 | Isoform 1 of Synaptic vesicle glycoprotein 2A | SV2A |
| IPI00005978 | Splicing factor, arginine/serine-rich 2 | SFRS2 |
| IPI00171647 | Isoform 1 of Sialic acid-binding Ig-like lectin 8 precursor | SIGLEC8 |
| IPI00020058 | Isoform I of Copper-transporting ATPase 2 | ATP7B |
| IPI00844512 | RHOV protein (Fragment) | RHOV |
| IPI00026570 | Cytochrome c oxidase polypeptide VIIa-liver/heart, | COX7A2 |
| | mitochondrial precursor | |
| IPI00021997 | Protein CREG1 precursor | CREG1 |
| IPI00879389 | 20 kDa protein | ZMYND11 |
| IPI00329251 | Isoform 1 of Transmembrane protein 146 precursor | TMEM146 |
| IPI00027726 | Isoform 1 of Krueppel-like factor 3 | KLF3 |
| IPI00064241 | Isoform 1 of Zinc finger protein Eos | IKZF4 |
| IPI00295503 | Isoform 2 of Probable ATP-dependent RNA helicase | DDX58 |
| | DDX58 | |
| IPI00465087 | Ubiquitin carboxyl-terminal hydrolase BAP1 | BAP1 |
| IPI00290292 | Rhomboid 5 homolog 1 | RHBDF1 |
| IPI00251507 | Isoform IB of Synapsin-1 | SYN1 |
| IPI00103812 | PWWP domain-containing protein 2B | PWWP2B |
| IPI00008580 | Antileukoproteinase precursor | SLPI |
| IPI00010953 | CDNA FLJ11251 fis, clone PLACE1008813 | EXOC6 |
| IPI00639924 | 97 kDa protein | LOC374920 |
| IPI00003348 | Guanine nucleotide-binding protein G(I)/G(S)/G(T) subunit | GNB2 |
| | beta-2 | |
| IPI00029533 | Integrin beta-8 precursor | ITGB8 |
| IPI00815786 | Hexokinase 1 (Fragment) | HK1 |
| IPI00292579 | Stabilin-2 precursor | STAB2 |
| IPI00067920 | FXYD domain-containing ion transport regulator 7 | FXYD7 |
| IPI00879162 | Uncharacterized protein ENSP00000374866 | — |
| IPI00008998 | Protein tyrosine phosphatase-like protein PTPLAD1 | PTPLAD1 |
| IPI00658112 | 32 kDa protein | SPEG |
| IPI00009804 | DNA-binding protein SATB1 | SATB1 |
| IPI00479361 | Isoform 1 of UDP-GlcNAc:betaGal beta-1,3-N- | B3GNT4 |
| | acetylglucosaminyltransferase 4 | |
| IPI00448465 | Isoform 1 of Ankyrin repeat domain-containing protein 12 | ANKRD12 |
| IPI00215925 | Glycine N-methyltransferase | GNMT |
| IPI00006675 | Multidrug resistance-associated protein 4 | ABCC4 |
| IPI00745122 | Conserved hypothetical protein | MGC33894 |
| IPI00442551 | CDNA FLJ26989 fis, clone SLV03395 | ATP9B |
| IPI00027436 | Tumor necrosis factor receptor superfamily member 16 | NGFR |
| | precursor | |
| IPI00005531 | Isoform 1 of Probable DNA dC->dU-editing enzyme | APOBEC3B |
| | APOBEC-3B | |
| IPI00023542 | transmembrane emp24 protein transport domain containing 9 | TMED9 |
| IPI00783313 | Glycogen phosphorylase, liver form | PYGL |
| IPI00166190 | Isoform 1 of Uncharacterized protein C19orf19 | C19orf19 |
| IPI00383832 | Protein kinase C-binding protein RACK8 | DVL3 |
| IPI00024976 | Mitochondrial import receptor subunit TOM22 homolog | TOMM22 |
| IPI00154567 | Isoform 2 of Spermatogenesis-associated protein 20 | SPATA20 |
| | precursor | |
| IPI00027685 | C-C chemokine receptor type 1 | CCR1 |
| IPI00009335 | Brain protein 16 | C8orf30A |
| IPI00008495 | NADH-ubiquinone oxidoreductase chain 4 | MT-ND4 |
| IPI00179172 | Isoform 2 of Liprin-beta-1 | PPFIBP1 |
| IPI00012315 | Nucleoside diphosphate kinase 3 | NME3 |
| IPI00432755 | PPRR6495 | FAM124A |
| IPI00550949 | Bone morphogenetic protein 7 precursor | BMP7 |
| IPI00796777 | 17 kDa protein | CRYAA |
| IPI00872928 | Several ankyrin repeat protein transcript variant 2 | ANKRD42 |
| | (Fragment) | |
| IPI00412647 | CDNA: FLJ21792 fis, clone HEP00441 | THADA |
| IPI00032929 | Isoform 1 of Thrombospondin type-1 domain-containing | THSD1 |
| | protein 1 precursor | |
| IPI00008894 | Carboxypeptidase A4 precursor | CPA4 |
| IPI00154451 | MMS19 nucleotide excision repair homolog | MMS19 |
| IPI00186145 | Isoform 1 of Protein phosphatase 1L | PPM1L |
| IPI00847759 | DENN domain-containing protein 4B | DENND4B |
| IPI00008315 | Isoform 1 of Ephrin type-B receptor 1 precursor | EPHB1 |
| IPI00386364 | Olfactory receptor 2L2 | OR2L2 |
| IPI00031005 | Protein kinase-like protein SgK196 | FLJ23356 |
| IPI00787414 | Uncharacterized protein ENSP00000381388 | MGC34829 |
| IPI00003527 | Ezrin-radixin-moesin-binding phosphoprotein 50 | SLC9A3R1 |
| IPI00218823 | Isoform 1 of WW domain-binding protein 7 | MLL4 |
| IPI00019988 | N-sulphoglucosamine sulphohydrolase precursor | SGSH |
| IPI00059164 | Galactose-3-O-sulfotransferase 3 | GAL3ST3 |
| IPI00873740 | Uncharacterized protein ENSP00000383832 (Fragment) | — |
| IPI00060379 | Integrator complex subunit 12 | INTS12 |
| IPI00032416 | Isoform Long of Protein jagged-2 precursor | JAG2 |
| IPI00444706 | CDNA FLJ45007 fis, clone BRAWH3012005 | KALRN |
| IPI00216710 | Isoform 1 of Nuclear receptor ROR-gamma | RORC |
| IPI00335589 | RNA methyltransferase-like protein 1 | RNMTL1 |
| IPI00411690 | Isoform 3 of La-related protein 1 | LARP1 |
| IPI00295519 | Isoform 1 of Uncharacterized protein C3orf17 | C3orf17 |
| IPI00514153 | transmembrahe and tetratricopeptide repeat containing 4 | TMTC4 |
| | isoform 1 | |
| IPI00398728 | Isoform 1 of Retinitis pigmentosa 1-like 1 protein | RP1L1 |
| IPI00843819 | Similar to Dual specificity protein kinase CLK2 | — |
| IPI00065276 | Isoform 2 of Tether containing UBX domain for GLUT4 | ASPSCR1 |
| IPI00021473 | 59 kDa protein | CBFA2T3 |
| IPI00745775 | Similar to PR domain containing 4 | — |
| IPI00016703 | 24-dehydrocholesterol reductase precursor | DHCR24 |
| IPI00013880 | Semaphorin-5A precursor | SEMA5A |
| IPI00010157 | S-adenosylmethionine synthetase isoform type-2 | MAT2A |
| IPI00794307 | hypothetical protein | LOC729505 |
| IPI00015782 | UPF0171 protein C16orf35 | C16orf35 |
| IPI00011652 | Isoform Efs1 of Embryonal Fyn-associated substrate | EFS |
| IPI00644522 | PNKP protein | PNKP |
| IPI00045511 | Isoform 1 of Chloride channel CLIC-like protein 1 | CLCC1 |
| | precursor | |
| IPI00028881 | NADH dehydrogenase [ubiquinone] 1 alpha subcomplex | NDUFA3 |
| | subunit 3 | |
| IPI00604763 | Transmembrane protein 66 precursor | TMEM66 |
| IPI00235647 | similar to fibrillarin | LOC345630 |
| IPI00879950 | 15 kDa protein | — |
| IPI00413385 | Inter-alpha-trypsin inhibitor heavy chain H5-like protein | ITIH5L |
| | precursor | |
| IPI00294910 | Protein PARM-1 precursor | DKFZP564O0823 |
| IPI00022334 | Ornithine aminotransferase, mitochondrial precursor | OAT |
| IPI00023617 | Zinc finger protein 197 | ZNF197 |
| IPI00008433 | 40S ribosomal protein S5 | RPS5 |
| IPI00642798 | Nucleolar autoantigen No55 | SC65 |
| IPI00470805 | Isoform 2 of Mediator of DNA damage checkpoint protein 1 | MDC1 |
| IPI00167089 | Isoform 2 of Activated CDC42 kinase 1 | TNK2 |
| IPI00102575 | ATPase family, AAA domain containing 5 | ATAD5 |
| IPI00783753 | UPF0235 protein C15orf40 | C15orf40 |
| IPI00383895 | CDNA FLJ40559 fis, clone THYMU2002910 | PRDM11 |
| IPI00152157 | Isoform 1 of Zinc finger protein 509 | ZNF509 |
| IPI00296803 | 30 kDa protein | TCP11L2 |
| IPI00020501 | Myosin-11 | MYH11 |
| IPI00168056 | Zinc finger and BTB domain-containing protein 38 | ZBTB38 |
| IPI00030919 | Mitogen-activated protein kinase kinase 1-interacting | MAP2K1IP1 |
| | protein 1 | |
| IPI00827745 | Isoform 1 of RNA-binding protein 24 | RBM24 |
| IPI00005776 | Nucleotide-binding oligomerization domain-containing | NOD1 |
| | protein 1 | |
| IPI00026105 | Isoform SCPx of Non-specific lipid-transfer protein | SCP2 |
| IPI00385255 | Ig lambda chain V-II region NIG-84 | — |
| IPI00328178 | Isoform 3 of TOM1-like protein 2 | TOM1L2 |
| IPI00394879 | Leucine-rich repeat-containing protein 9 | LRRC9 |
| IPI00401852 | Conserved hypothetical protein | DKFZP434L187 |
| IPI00442865 | CDNA FLJ26488 fis, clone KDN05770, highly similar to | SLC12A1 |
| | Bumetanide-sensitive sodium-(potassium)-chloride | |
| | cotransporter 2 | |
| IPI00748891 | hypothetical protein LOC283635 isoform 1 | C14orf24 |
| IPI00414320 | Annexin A11 | ANXA11 |
| IPI00874023 | Uncharacterized protein ENSP00000379699 | — |
| IPI00027144 | Cytochrome b561 | CYB561 |
| IPI00827584 | similar to kinesin family member 27 | KIF27 |
| IPI00217688 | Uncharacterized protein C12orf60 | C12orf60 |
| IPI00020199 | Alpha-2,8-sialyltransferase 8B | ST8SIA2 |
| IPI00003799 | Isoform 2 of Heme-binding protein 2 | HEBP2 |
| IPI00220126 | Isoform EP2B of Sperm-associated antigen 11B precursor | SPAG11B |
| IPI00007757 | Isoform 1 of Zinc finger protein Helios | IKZF2 |
| IPI00791513 | CDNA FLJ16614 fis, clone TESTI4013365 | CTA-216E10.6 |
| IPI00008756 | Isoform 1 of Bullous pemphigoid antigen 1, isoforms | DST |
| | 1/2/3/4/5/8 (Fragment) | |
| IPI00328400 | Zygote arrest protein 1 | ZAR1 |
| IPI00020201 | CMP-N-acetylneuraminate-poly-alpha-2,8-sialyltransferase | ST8SIA4 |
| IPI00465147 | Isoform 1 of Uncharacterized protein C9orf97 | C9orf97 |
| IPI00002884 | CDNA: FLJ22222 fis, clone HRC01658 | FLJ22222 |
| IPI00217465 | Histone H1.2 | HIST1H1C |
| IPI00025092 | Myosin-binding protein C, slow-type | MYBPC1 |
| IPI00003363 | Isoform 1 of Protein phosphatase 1 regulatory subunit 1B | PPP1R1B |
| IPI00333016 | Isoform 3 of DnaJ homolog subfamily C member 11 | DNAJC11 |
| IPI00005705 | Isoform Gamma-1 of Serine/threonine-protein phosphatase | PPP1CC |
| | PP1-gamma catalytic subunit | |
| IPI00014897 | Isoform 2 of 1-phosphatidylinositol-4,5-bisphosphate | PLCB4 |
| | phosphodiesterase beta-4 | |
| IPI00181279 | hypothetical protein | LRRC37A4; LOC728863 |
| IPI00022078 | Protein NDRG1 | NDRG1 |
| IPI00011894 | MRNA clone with similarity to L-glycerol-3- | — |
| | phosphate:NAD oxidoreductase and albumin gene | |
| | sequences | |
| IPI00794248 | 47 kDa protein | — |
| IPI00002511 | Cyclic AMP-dependent transcription factor ATF-6 alpha | ATF6 |
| IPI00164949 | Isoform NELF-C of Negative elongation factor C/D | TH1L |
| IPI00029647 | Zymogen granule membrane protein 16 precursor | ZG16 |
| IPI00414481 | GTF3C1 protein | GTF3C1 |
| IPI00472754 | Polycystic kidney disease 1-related protein | KIAA0319L |
| IPI00640818 | Isoform 3 of Neuropathy target esterase | PNPLA6 |
| IPI00514774 | Isoform 1 of PAB-dependent poly(A)-specific ribonuclease | PAN3 |
| | subunit 3 | |
| IPI00307733 | Isoform 1 of Histone-lysine N-methyltransferase SETD2 | SETD2 |
| IPI00154528 | Isoform 1 of Structural maintenance of chromosomes | SMC6 |
| | protein 6 | |
| IPI00164861 | Isoform 3 of Kinesin-like protein KIF13A | KIF13A |
| IPI00218896 | Alcohol dehydrogenase 1A | ADH1A |
| IPI00411674 | Isoform 1 of Zinc finger protein 254 | ZNF254 |
| IPI00414973 | FAST kinase domain-containing protein 5 | FASTKD5 |
| IPI00002441 | Syndecan-1 precursor | SDC1 |
| IPI00413272 | Isoform 3 of Mediator of RNA polymerase II transcription | MED23 |
| | subunit 23 | |
| IPI00249982 | Isoform 1 of Death-inducer obliterator 1 | DIDO1 |
| IPI00299853 | Protocadherin beta 16 precursor | PCDHB16 |
| IPI00328270 | Neuronal PAS domain-containing protein 2 | NPAS2 |
| IPI00442544 | CDNA FLJ27034 fis, clone SLV07984 | — |
| IPI00175019 | similar to Temporarily Assigned Gene name family | LOC643677 |
| | member | |
| IPI00027898 | Isoform A of Uncharacterized protein C21 orf70 | C21orf70 |
| IPI00023340 | Histone acetyltransferase MYST3 | MYST3 |
| IPI00735857 | similar to ATP-binding cassette sub-family D member 1 | LOC651123 |
| IPI00300052 | Keratin type II cuticular Hb4 | KRT84 |
| IPI00454695 | Histone H2B type 2-C | HIST2H2BC |
| IPI00647254 | 7 kDa protein | — |
| IPI00217467 | Histone H1.4 | HIST1H1E |
| IPI00328118 | Sperm-associated antigen 5 | SPAG5 |
| IPI00094740 | Isoform 1 of RING finger protein 31 | RNF31 |
| IPI00019158 | ADAM metallopeptidase domain 8 precursor | ADAM8 |
| IPI00383318 | PRO2272 | — |
| IPI00045928 | Sodium/hydrogen exchanger 7 | SLC9A7 |
| IPI00292470 | dapper, antagonist of beta-catenin, homolog 2 | DACT2 |
| IPI00009532 | 4-aminobutyrate aminotransferase, mitochondrial precursor | ABAT |
| IPI00215884 | Isoform ASF-1 of Splicing factor, arginine/serine-rich 1 | SFRS1 |
| IPI00306332 | 60S ribosomal protein L24 | RPL24 |
| IPI00013488 | Isoform Alpha-2 of N-chimaerin | CHN1 |
| IPI00218687 | Isoform 10 of Voltage-dependent T-type calcium channel | CACNA1G |
| | subunit alpha-1G | |
| IPI00015047 | 8D6 antigen (Fragment) | CD320 |
| IPI00298994 | Talin-1 | TLN1 |
| IPI00643937 | Methylenetetrahydrofolate dehydrogenase (NADP+ | MTHFD1L |
| | dependent) 1-like | |
| IPI00030909 | BB1 family protein | — |
| IPI00552213 | Isoform 6 of Disks large homolog 1 | DLG1 |
| IPI00001786 | Isoform 2 of Ubiquitin carboxyl-terminal hydrolase 36 | USP36 |
| IPI00397358 | Similar to ribosomal protein S27 | — |
| IPI00745103 | similar to melanoma associated antigen (mutated) 1-like 1 | LOC728307 |
| IPI00030876 | diaphanous 1 isoform 2 | DIAPH1 |
| IPI00021786 | RAF proto-oncogene serine/threonine-protein kinase | RAF1 |
| IPI00003438 | DnaJ homolog subfamily C member 8 | DNAJC8 |
| IPI00398900 | Homeobox protein HMX2 | HMX2 |
| IPI00031960 | polymerase (RNA) I polypeptide A, 194 kDa | POLR1A |
| IPI00298870 | Transmembrane protein 1 | TMEM1 |
| IPI00385791 | Serologically defined breast cancer antigen NY-BR-87 | MRPS26 |
| | (Fragment) | |
| IPI00017991 | jumonji domain containing 2D | JMJD2D |
| IPI00151366 | Isoform TRPM6a of Transient receptor potential cation | TRPM6 |
| | channel subfamily M member 6 | |
| IPI00031627 | DNA-directed RNA polymerase II subunit RPB1 | POLR2A |
| IPI00004488 | Vacuolar proton pump subunit F | ATP6V1F |
| IPI00145805 | Isoform 1 of TRAF2 and NCK-interacting protein kinase | TNIK |
| IPI00296374 | Zinc finger protein-like 1 | ZFPL1 |
| IPI00328825 | Isoform 1 of NEDD4-binding protein 2 | N4BP2 |
| IPI00184884 | Non-structural maintenance of chromosomes element 1 | NSMCE1 |
| | homolog | |
| IPI00011488 | Isoform 1 of Serine/threonine-protein kinase 4 | STK4 |
| IPI00103874 | Isoform 1 of Zinc finger FYVE domain-containing protein 1 | ZFYVE1 |
| IPI00737920 | similar to dynein, axonemal, heavy polypeptide 1 | DNAH3 |
| IPI00243221 | nardilysin (N-arginine dibasic convertase) isoform a | NRD1 |
| IPI00060569 | Isoform 2 of Abhydrolase domain-containing protein 12 | ABHD12 |
| IPI00101405 | Farnesyl diphosphate synthase | FDPS |
| IPI00015983 | Sphingosine 1-phosphate receptor Edg-3 | EDG3 |
| IPI00075013 | Complement C1q tumor necrosis factor-related protein 1 | C1QTNF1 |
| | precursor | |
| IPI00289965 | Potassium voltage-gated channel subfamily C member 3 | KCNC3 |
| IPI00786867 | Dual Intracellular Von Willebrand factor domain A | DIVA |
| IPI00374129 | NLR family, pyrin domain containing 3 isoform b | NLRP3 |
| IPI00006904 | Cell death regulator Aven | AVEN |
| IPI00827486 | Rheumatoid factor light chain variable region precursor | — |
| | (Fragment) | |
| IPI00853079 | Isoform 1 of Uncharacterized protein C14orf166B | C14orf166B |
| IPI00456578 | LOC441054 protein | LOC441054 |
| IPI00376213 | Isoform 1 of G-protein coupled receptor 120 | GPR120 |
| IPI00376587 | Uncharacterized protein ENSP00000345065 | LOC728780 |
| IPI00333197 | Isoform 2 of GRIP and coiled-coil domain-containing | GCC2 |
| | protein 2 | |
| IPI00010420 | ADP/ATP translocase 4 | SLC25A31 |
| IPI00004346 | C-C chemokine receptor type 10 | CCR10 |
| IPI00297251 | Isoform 2 of Probable E3 ubiquitin-protein ligase MGRN1 | MGRN1 |
| IPI00004419 | Zinc finger protein 646 | ZNF646 |
| IPI00847409 | hypothetical protein | LOC440338 |
| IPI00218131 | Protein S100-A12 | S100A12 |
| IPI00005024 | Isoform 1 of Myb-binding protein 1A | MYBBP1A |
| IPI00177890 | 54 kDa protein | SMARCD2 |
| IPI00438170 | Isoform 1 of Sorting nexin-12 | SNX12 |
| IPI00373823 | Cytochrome P450 26C1 | CYP26C1 |
| IPI00550917 | Twinfilin-2 | TWF2 |
| IPI00023635 | Isoform 1 of Inositol monophosphatase 2 | IMPA2 |
| IPI00014516 | Isoform 1 of Caldesmon | CALD1 |
| IPI00008403 | Carbohydrate sulfotransferase 7 | CHST7 |
| IPI00412298 | Isoform I of Serine/threonine-protein kinase ATR | ATR |
| IPI00021951 | Uncharacterized protein KIAA0247 precursor | KIAA0247 |
| IPI00815893 | Isoform 1 of Chromodomain-helicase-DNA-binding | CHD2 |
| | protein 2 | |
| IPI00009439 | Synaptotagmin-1 | SYT1 |
| IPI00002243 | Isoform 1 of Gamma-glutamyltransferase 5 precursor | GGTLA1 |
| IPI00159049 | SET-binding protein | SETBP1 |
| IPI00005837 | Angiopoietin-related protein 1 srecursor | ANGPTL1 |
| IPI00297277 | Isoform 1 of RING finger protein 150 precursor | RNF150 |
| IPI00304885 | Centromere protein C 1 | CENPC1 |
| IPI00784013 | janus kinase 1 | JAK1 |
| IPI00216139 | Isoform I of Septin-6 | SEPT6 |
| IPI00289954 | Heparan sulfate glucosamine 3-O-sulfotransferase 6 | HS3ST6 |
| IPI00018134 | Isoform 4 of Protein SOLO | FLJ10357 |
| IPI00855742 | sterile alpha motif domain containing 12 isoform a | SAMD12 |
| IPI00415037 | Isoform 12L of ADAM 12 precursor | ADAM12 |
| IPI00411298 | Connector enhancer of kinase suppressor of ras 3 | CNKSR3 |
| IPI00016685 | Enamelin precursor | ENAM |
| IPI00002191 | Putative uncharacterized protein FLJ12684 | — |
| IPI00007928 | Pre-mRNA-processing-splicing factor 8 | PRPF8 |
| IPI00031288 | Claudin-14 | CLDN14 |
| IPI00028053 | Gap junction alpha-9 protein | GJA9 |
| IPI00867582 | Similar to SCAN domain-containing protein 2. Isoform 2 | ZNF496 |
| IPI00418885 | Epididymal-specific lipocalin-10 precursor | LCN10 |
| IPI00166465 | hypothetical protein LOC221416 | C6orf223 |
| IPI00795992 | Hypothetical short protein | — |
| IPI00847373 | hypothetical protein LOC54627 | KIAA1383 |
| IPI00030070 | Gap junction beta-5 protein | GJB5 |
| IPI00293095 | Isoform 1 of Coiled-coil domain-containing protein 83 | CCDC83 |
| IPI00418238 | Isoform 1 of HIV-1 Rev-binding protein-like protein | HRBL |
| IPI00008504 | Carbonic anhydrase 14 precursor | CA14 |
| IPI00293409 | Zinc finger protein 81 | ZNF81 |
| IPI00003927 | 40 kDa peptidyl-prolyl cis-trans isomerase | PPID |
| IPI00747142 | Centaurin-gamma-like family member 6 | CTGLF6 |
| IPI00024801 | E3 ubiquitin-protein ligase NRDP1 | RNF41 |
| IPI00040900 | Isoform 2 of Heparan sulfate 2-O-sulfotransferase 1 | HS2ST1 |
| IPI00103356 | Uncharacterized protein ITGB2 | ITGB2 |
| IPI00604798 | Bitter taste receptor T2R2 (Fragment) | — |
| IPI00011515 | Protein kinase C and casein kinase substrate in neurons | PACSIN1 |
| | protein 1 | |
| IPI00164066 | Isoform 4 of Coiled-coil domain-containing protein 136 | CCDC136 |
| IPI00307702 | H53_GS1 (Fragment) | — |
| IPI00013887 | Beta-galactoside alpha-2,6-sialyltransferase 1 | ST6GAL1 |
| IPI00025084 | Calpain small subunit 1 | CAPNS1 |
| IPI00014899 | CDNA FLJ20744 fis, clone HEP06585 | BRPF3 |
| IPI00307611 | Isoform 1 of Microtubule-associated serine/threonine- | MAST4 |
| | protein kinase 4 | |
| IPI00014444 | Isoform 1 of Protein SERAC1 | SERAC1 |
| IPI00479217 | Isoform Short of Heterogeneous nuclear ribonucleoprotein U | HNRNPU |
| IPI00384971 | hypothetical protein LOC643155 | DKFZP686E2158 |
| IPI00025721 | COP9 signalosome complex subunit 3 | COPS3 |
| IPI00017334 | Prohibitin | PHB |
| IPI00183206 | Isoform 1 of RIM-binding protein 2 | RIMBP2 |
| IPI00220070 | 6-phosphofructo-2-kinase/fructose-2,6-biphosphatase 4 | PFKFB4 |
| IPI00023322 | Zinc finger protein ubi-d4 | DPF2 |
| IPI00216699 | Isoform 2 of Unc-112-related protein 2 | FERMT3 |
| IPI00855747 | hypothetical protein LOC57481 | RP13-347D8.3 |
| IPI00445054 | CDNA FLJ44715 fis, clone BRACE3021430 | FLJ44715 |
| IPI00303318 | Protein FAM49B | FAM49B |
| IPI00030360 | RUN domain-containing protein 2A | RUNDC2A |
| IPI00220503 | dynactin 2 | DCTN2 |
| IPI00784739 | Uncharacterized protein C14orf43 | C14orf43 |
| IPI00328159 | Isoform 1 of Uncharacterized protein C1orf88 | C1orf88 |
| IPI00003881 | Heterogeneous nuclear ribonucleoprotein F | HNRPF |
| IPI00003990 | Isoform 2 of Valacyclovir hydrolase precursor | BPHL |
| IPI00847485 | Similar to PFIV protein | — |
| IPI00738920 | similar to CG3104-PA, isoform A | LOC642574 |
| IPI00056314 | Pre-rRNA-processing protein TSR2 homolog | TSR2 |
| IPI00007300 | BTB/POZ domain-containing protein 3 | BTBD3 |
| IPI00019517 | Wilms tumor 1 isoform D | WT1 |
| IPI00797694 | 3 kDa protein | — |
| IPI00294210 | DNA-binding protein inhibitor ID-2 | ID2 |
| IPI00217490 | Isoform 1 of Fibronectin type III domain-containing protein | FNDC3B |
| | 3B | |
| IPI00848330 | Conserved hypothetical protein | LOC285144 |
| IPI00000787 | Isoform LMP2.L of Proteasome subunit beta type-9 | PSMB9 |
| | precursor | |
| IPI00004379 | Homeobox protein HMX1 | HMX1 |
| IPI00004373 | Mannose-binding protein C precursor | MBL2 |
| IPI00787020 | similar to Dynamin-1 | LOC644153 |
| IPI00375820 | Interferon-induced transmembrane protein 5 | IFITM5 |
| IPI00411635 | 180 kDa protein | — |
| IPI00795566 | 6 kDa protein | — |
| IPI00217117 | Isoform 1 of Glycerate kinase | GLYCTK |
| IPI00167941 | Midasin | MDN1 |
| IPI00300086 | Nicotinate-nucleotide pyrophosphorylase | QPRT |
| IPI00016701 | P2Y purinoceptor 14 | P2RY14 |
| IPI00007010 | Lysozyme-like protein 6 precursor | LYZL6 |
| IPI00235167 | Profilin-3 | PFN3 |
| IPI00026126 | Mammaglobin-B precursor | SCGB2A1 |
| IPI00009899 | Uncharacterized protein C5orf5 | C5orf5 |
| IPI00220167 | Isoform 1 of Putative methyltransferase NSUN7 | NSUN7 |
| IPI00155168 | Leukocyte common antigen precursor (EC 3.1.3.48) (L- | PTPRC |
| | CA) (T200) (CD45 antigen). Isoform 2 | |
| IPI00217948 | FRMD4B protein | FRMD4B |
| IPI00384268 | XRCC6BP1 protein | XRCC6BP1 |
| IPI00027457 | C1q-related factor precursor | C1QL1 |
| IPI00002993 | Transcription initiation factor TFIID subunit 9 | TAF9 |
| IPI00216348 | Isoform 2C of Cytoplasmic dynein 1 intermediate chain 2 | DYNC1I2 |
| IPI00299507 | Condensin complex subunit 2 | NCAPH |
| IPI00555600 | Solute carrier family 26, member 1 isoform a variant | IDUA |
| | (Fragment) | |
| IPI00419221 | Membrane-bound O-acyltransferase domain-containing | MBOAT2 |
| | protein 2 | |
| IPI00445643 | CDNA FLJ43684 fis, clone TBAES2001492 | — |
| IPI00165591 | Isoform 4 of Transmembrane channel-like protein 6 | TMC6 |
| IPI00010334 | Isoform 3 of Inactive phospholipase C-like protein 2 | PLCL2 |
| IPI00021634 | Kinesin light chain 2 | KLC2 |
| IPI00451429 | NIF3L1 isoform gamma | NIF3L1 |
| IPI00375803 | Isoform 1 of GON-4-like protein | GON4L |
| IPI00023184 | Isoform 1 of Poly [ADP-ribose] polymerase 3 | PARP3 |
| IPI00872739 | Uncharacterized protein C18orf2 | C18orf2 |
| IPI00241409 | hypothetical protein LOC55747 | FAM21B |
| IPI00152149 | CENP-B, N-terminal DNA-binding domain containing | — |
| | protein | |
| IPI00090764 | Toll-like receptor 1 precursor | TLR1 |
| IPI00024253 | Isoform 1 of Fibroblast growth factor 14 | FGF14 |
| IPI00029643 | Isoform 2 of Mitogen-activated protein kinase kinase | ZAK |
| | kinase MLT | |
| IPI00218637 | Major histocompatibility complex, class II, DQ beta 2 | HLA-DQB2 |
| IPI00000875 | Elongation factor 1-gamma | EEF1G |
| IPI00215948 | Isoform 1 of Catenin alpha-1 | CTNNA1 |
| IPI00022918 | Isoform 2 of Protein FAM125B | FAM125B |
| IPI00787765 | similar to solute carrier family 35, member F4 | SLC35F4 |
| IPI00216921 | Isoform 2 of Stathmin-4 | STMN4 |
| IPI00011416 | Delta(3,5)-Delta(2,4)-dienoyl-CoA isomerase, | ECH1 |
| | mitochondrial precursor | |
| IPI00007208 | Probable ATP-dependent RNA helicase DDX41 | DDX41 |
| IPI00260755 | similar to Rho GTPase activating protein 18 | C20orf95 |
| IPI00337385 | Isoform 1 of Pre-mRNA-processing factor 40 homolog A | PRPF40A |
| IPI00304527 | Protein FAM83B | FAM83B |
| IPI00000459 | Transmembrane gamma-carboxyglutamic acid protein 1 | PRRG1 |
| | precursor | |
| IPI00030741 | Uncharacterized protein C21orf13 | LCA5L |
| IPI00012007 | Adenosylhomocysteinase | AHCY |
| IPI00855922 | Isoform 4 of DENN domain-containing protein 3 | DENND3 |
| IPI00399296 | hypothetical protein LOC390110 | LOC390110 |
| IPI00233358 | islet cell autoantigen 1, 69 kDa-like isoform 2 | ICA1L |
| IPI00879842 | 6 kDa protein | — |
| IPI00328826 | Uncharacterized protein CADPS2 | CADPS2 |
| IPI00021476 | Eukaryotic translation initiation factor 4E-binding protein 3 | EIF4EBP3 |
| IPI00028383 | Uncharacterized protein C16orf24 | C16orf24 |
| IPI00007074 | Tyrosyl-tRNA synthetase, cytoplasmic | YARS |
| IPI00031056 | Zinc finger MYND domain-containing protein 15 | ZMYND15 |
| IPI00641614 | 47 kDa protein | AKAP8L |
| IPI00002134 | 26S proteasome non-ATPase regulatory subunit 5 | PSMD5 |
| IPI00056040 | Neuritin 1-like | NRN1L |
| IPI00012948 | Proheparin-binding EGF-like growth factor precursor | HBEGF |
| IPI00856045 | AHNAK nucleoprotein 2 | AHNAK2 |
| IPI00009329 | Utrophin | UTRN |
| IPI00298337 | cDNA FLJ77671 | SLC14A1 |
| IPI00218235 | dehydrogenase/reductase member 2 isoform 2 | DHRS2 |
| IPI00297315 | Alpha-2B adrenergic receptor | ADRA2B |
| IPI00219005 | FK506-binding protein 4 | FKBP4 |
| IPI00044842 | Isoform 2 of RAB3A-interacting protein | RAB3IP |
| IPI00107855 | Hephaestin precursor | HEPH |
| IPI00217662 | Uncharacterized protein C11orf35 | C11orf35 |
| IPI00386119 | Isoform 5 of Splicing factor 1 | SF1 |
| IPI00025489 | Serine/threonine-protein kinase MAK | MAK |
| IPI00013004 | Isoform 1 of Pyridoxal kinase | PDXK |
| IPI00022055 | Histone acetyltransferase PCAF | PCAF |
| IPI00328260 | Protein FAN | NSMAF |
| IPI00807418 | Isoform 9 of Lymphoid-specific helicase | HELLS |
| IPI00027774 | THAP domain-containing protein 2 | THAP2 |
| IPI00183002 | Isoform 1 of Protein phosphatase 1 regulatory subunit 12A | PPPIRI2A |
| IPI00027415 | Isoform 1 of Probable ATP-dependent RNA helicase | DHX36 |
| | DHX36 | |
| IPI00163601 | Putative uncharacterized protein FLJ10213 | FLJ10213 |
| IPI00428741 | LP2477 | FLJ35348 |
| IPI00006252 | Multisynthetase complex auxiliary component p43 | SCYE1 |
| IPI00021907 | Isoform 1 of Myelin basic protein | MBP |
| IPI00176125 | Collagen XXIX alpha 1 | COL29A1 |
| IPI00007321 | Isoform I of Acyl-protein thioesterase 1 | LYPLA1 |
| IPI00217617 | palmitoylated membrane protein 7 | MPP7 |
| IPI00008303 | Isoform 1 of N-acetylglucosamine-1-phosphodiester alpha- | NAGPA |
| | N-acetylglucosaminidase precursor | |
| IPI00418614 | 67 kDa protein | — |
| IPI00016046 | Protein MGR2 homolog | C20orf52 |
| IPI00024662 | Chromobox protein homolog 5 | CBX5 |
| IPI00550364 | Phosphoglucomutase-2 | PGM2 |
| IPI00169331 | Phosphatidylcholine:ceramide cholinephosphotransferase 2 | SGMS2 |
| IPI00028450 | Isoform 1 of Sodium/calcium exchanger 1 precursor | SLC8A1 |
| IPI00783464 | dynein heavy chain domain 3 | DNAH2 |
| IPI00002841 | Sodium/hydrogen exchanger 2 | SLC9A2 |
| IPI00472712 | Coiled-coil domain containing 7 | CCDC7 |
| IPI00061355 | Isoform 3 of Protein ZNF673 | ZNF673 |
| IPI00445804 | Uncharacterized protein ENSP00000374264 | DKFZp761B107 |
| IPI00790813 | 8 kDa protein | RAD51AP1 |
| IPI00010405 | Isoform Long of Tyrosine-protein kinase transmembrane | ROR1 |
| | receptor ROR1 precursor | |
| IPI00061780 | Isoform 1 of E3 ubiquitin-protein ligase Itchy homolog | ITCH |
| IPI00873774 | Uncharacterized protein ENSP00000383488 (Fragment) | — |
| IPI00847335 | FLJ45422 protein | — |
| IPI00030099 | Adenylate cyclase type 9 | ADCY9 |
| IPI00217740 | C20orf12 protein | C20orf12 |
| IPI00396961 | Leucine-rich repeat and fibronectin type-III domain- | LRFN5 |
| | containing protein 5 precursor | |
| IPI00514533 | Uncharacterized protein SYCP2L | SYCP2L |
| IPI00016095 | Transcription termination factor, mitochondrial precursor | MTERF |
| IPI00024689 | Aquaporin-1 | AQP1 |
| IPI00877800 | 32 kDa protein | — |
| IPI00008497 | Ornithine decarboxylase | ODC1 |
| IPI00741608 | similar to eukaryotic translation initiation factor 5A | EIF5AL3 |
| IPI00010808 | Interferon-gamma receptor alpha chain precursor | IFNGR1 |
| IPI00872550 | Uncharacterized protein PRDM2 | PRDM2 |
| IPI00004521 | Ewing's tumor-associated antigen 1 | ETAA1 |
| IPI00171525 | Sentrin-specific protease 3 | SENP3 |
| IPI00514622 | Ran-binding protein 6 | RANBP6 |
| IPI00373824 | Testis serine protease 1 precursor | TESSP1 |
| IPI00017231 | Neurensin-2 | NRSN2 |
| IPI00009203 | Sorting nexin-7 | SNX7 |
| IPI00790021 | Zinc finger protein 652 | ZNF652 |
| IPI00299729 | Transcobalamin-1 precursor | TCN1 |
| IPI00018860 | NKG2D ligand 2 precursor | ULBP2 |
| IPI00022290 | Beta-defensin 1 precursor | DEFB1 |
| IPI00007444 | Epiphycan precursor | EPYC |
| IPI00855918 | mucin 5, subtype B, tracheobronchial | MUC5B |
| IPI00167014 | EID2 protein (Fragment) | EID2 |
| IPI00180384 | dynein, axonemal, heavy chain 7 | DNAH7 |
| IPI00007327 | Isoform 1 of Tapasin precursor | TAPBP |
| IPI00244346 | Troponin 1, cardiac muscle | TNNI3 |
| IPI00009066 | similar to protein tyrosine phosphatase, receptor type, Q | PTPRQ |
| | isoform 1 precursor | |
| IPI00303195 | Syncoilin-1 | SYNC1 |
| IPI00008404 | Isoform Long of Segment polarity protein dishevelled | DVL1L1 |
| | homolog DVL-1-like | |
| IPI00658025 | Putative novel transcript | — |
| IPI00022958 | PRO0149 | C16orf72 |
| IPI00032313 | Protein S100-A4 | S100A4 |
| IPI00024094 | Rhesus blood group-associated glycoprotein | RHAG |
| IPI00027626 | T-complex protein 1 subunit zeta | CCT6A |
| IPI00031812 | Nuclease sensitive element-binding protein 1 | YBX1 |
| IPI00018854 | Coiled-coil domain-containing protein 68 | CCDC68 |
| IPI00027009 | Isoform 1 of Protein kinase C and casein kinase substrate in | PACSIN2 |
| | neurons protein 2 | |
| IPI00084582 | Isoform 7 of Collagen alpha-1(XIII) chain | COL13A1 |
| IPI00023162 | UDP-N-acetylglucosamine 2-epimerase/N- | GNE |
| | acetylmannosamine kinase | |
| IPI00216815 | Isoform 2 of DNA topoisomerase 3-beta-1 | TOP3B |
| IPI00062599 | 71 kDa protein | — |
| IPI00789181 | 115 kDa protein | PLCL1 |
| IPI00060549 | G-protein coupled receptor-associated sorting protein 1 | GPRASP1 |
| IPI00795943 | 7 kDa protein | — |
| IPI00005132 | Guanine nucleotide-binding protein-like 3-like protein | GNL3L |
| IPI00642204 | Thioredoxin domain-containing protein 16 precursor | TXNDC16 |
| IPI00646199 | Zinc finger, MYM-type 2 | ZMYM2 |
| IPI00742725 | Conserved hypothetical protein | LOC388564 |
| IPI00791447 | Pseudogene candidate | LOC339929 |
| IPI00878755 | 43 kDa protein | — |
| IPI00152050 | ataxin 2-binding protein 1 isoform 3 | A2BP1 |
| IPI00385480 | Caskin-1 | CASKIN1 |
| IPI00745433 | eukaryotic translation initiation factor 2C, 2 | EIF2C2 |
| IPI00465123 | KIAA0415 gene product | KIAA0415 |
| IPI00398992 | Isoform 1 of Chromodomain-helicase-DNA-binding | CHD8 |
| | protein 8 | |
| IPI00026031 | Uncharacterized protein C6orf72 precursor | C6orf72 |
| IPI00103090 | Isoform 1 of RNA polymerase-associated protein LEO1 | LEO1 |
| IPI00012441 | Isoform 1 of Synaptojanin-1 | SYNJ1 |
| IPI00006054 | Syntaphilin | SNPH |
| IPI00215746 | Fatty acid-binding protein, adipocyte | FABP4 |
| IPI00216780 | Cartilage intermediate layer protein 2 precursor | CILP2 |
| IPI00101186 | Isoform 1 of RRP12-like protein | RRP12 |
| IPI00013404 | Mothers against decapentaplegic homolog 4 | SMAD4 |
| IPI00385918 | CDNA FLJ90582 fis, clone PLACE1000442, moderately | ZNF627 |
| | similar to ZINC FINGER PROTEIN ZFP-36 | |
| IPI00219067 | Glutathione S-transferase Mu 2 | GSTM2 |
| IPI00008438 | 40S ribosomal protein S10 | RPS10 |
| IPI00152849 | Isoform 1 of G2/mitotic-specific cyclin-B3 | CCNB3 |
| IPI00220038 | Isoform B of Arsenite-resistance protein 2 | ARS2 |
| IPI00029556 | Uncharacterized protein C1orf105 | C1orf105 |
| IPI00385683 | Ig heavy chain V-III region GAR | — |
| IPI00441344 | Beta-galactosidase precursor | GLB1 |
| IPI00217012 | pleckstrin and Sec7 domain containing | PSD |
| IPI00006030 | Dynein intermediate chain 1, axonemal | DNAI1 |
| IPI00215914 | ADP-ribosylation factor 1 | ARF1 |
| IPI00024714 | Isoform 1 of Regulator of G-protein signaling 12 | RGS12 |
| IPI00015980 | Isoform 2 of Multiple PDZ domain protein | MPDZ |
| IPI00103853 | Isoform 1 of Putative ribosome-binding factor A, | C18orf22 |
| | mitochondrial precursor | |
| IPI00102678 | Isoform 1 of Pecanex-like protein 1 | PCNX |
| IPI00020356 | 331 kDa protein | MAP1A |
| IPI00259671 | Taste receptor type 2 member 44 | TAS2R44 |
| IPI00008226 | 73 kDa protein | THSD3 |
| IPI00551062 | Isoform 1 of Protein canopy homolog 3 precursor | CNPY3 |
| IPI00219622 | Proteasome subunit alpha type-2 | PSMA2 |
| IPI00010903 | Dopey family member 1 | DOPEY1 |
| IPI00220741 | spectrin, alpha, erythrocytic 1 | SPTA1 |
| IPI00735934 | similar to capicua homolog | LOC646070 |
| IPI00335581 | Isoform 1 of E3 ubiquitin-protein ligase UBR3 | UBR3 |
| IPI00031086 | Insulin-like growth factor-binding protein 1 precursor | IGFBP1 |
| IPI00004416 | Charged multivesicular body protein 2a | CHMP2A |
| IPI00465234 | Cytokine receptor common beta chain precursor | CSF2RB |
| IPI00010303 | Serpin B4 | SERPINB4 |
| IPI00028828 | Isoform Beta of Zinc finger protein 202 | ZNF202 |
| IPI00514795 | Isoform 1 of Tubulin--tyrosine ligase-like protein 7 | TTLL7 |
| IPI00008091 | Putative DNA helicase INO80 complex homolog 1 | INOC1 |
| IPI00016112 | peroxidasin homolog | PXDN |
| IPI00010193 | Isoform 1 of Interferon-alpha/beta receptor beta chain | IFNAR2 |
| | precursor | |
| IPI00101987 | Uncharacterized protein C19orf62 | C19orf62 |
| IPI00853224 | START domain containing 7 | STARD7 |
| IPI00329791 | cDNA FLJ78679, highly similar to Homo sapiens DEAD | DDX46 |
| | (Asp-Glu-Ala-Asp) box polypeptide 46 (DDX46), mRNA | |
| IPI00001141 | Mitochondrial import inner membrane translocase subunit | TIMM22 |
| | Tim22 | |
| IPI00152344 | Pyridoxal phosphate phosphatase PHOSPHO2 | PHOSPHO2 |
| IPI00465363 | Histone H2B type 1-A | HIST1H2BA |
| IPI00028601 | Putative metallothionein C20orf127 | MT1P3 |
| IPI00514975 | Chromosome 1 open reading frame 133 | C1orf133 |
| IPI00744029 | Similar to Zinc finger, FYVE domain containing 21 | — |
| IPI00399320 | Transmembrane protein 59 precursor | TMEM59 |
| IPI00479125 | SLIT-ROBO Rho GTPase-activating protein 2 | SRGAP2 |
| IPI00455852 | Isoform 1 of Rho guanine nucleotide exchange factor 15 | ARHGEF15 |
| IPI00019812 | Serine/threonine-protein phosphatase 5 | PPP5C |
| IPI00550876 | Protein maestro | MRO |
| IPI00217687 | Isoform 1 of Coiled-coil domain-containing protein 135 | CCDC135 |
| IPI00829745 | Cellular titin isoform PEVK variant 2 (Fragment) | — |
| IPI00216544 | Isoform 2 of Protein hairless | HR |
| IPI00064296 | PRO0633 | — |
| IPI00216651 | Isoform 1 of Interleukin-28 receptor alpha chain precursor | IL28RA |
| IPI00792945 | 38 kDa protein | CHFR |
| IPI00646323 | Isoform 1 of Uncharacterized protein C10orf76 | C10orf76 |
| IPI00149375 | Isoform 2 of Uncharacterized protein C11orf56 | C11orf56 |
| IPI00386797 | Isoform 1 of Folliculin-interacting protein 1 | FNIP1 |
| IPI00304589 | Isoform 1 of 182 kDa tankyrase 1-binding protein | TNKS1BP1 |
| IPI00015913 | 5,6-dihydroxyindole-2-carboxylic acid oxidase precursor | TYRP1 |
| IPI00002857 | Isoform CSBP2 of Mitogen-activated protein kinase 14 | MAPK14 |
| IPI00399180 | Serine/threonine-protein kinase SBK1 | SBK1 |
| IPI00007512 | Glutathione transferase omega-2 | GSTO2 |
| IPI00016576 | Isoform 1 of Grainyhead-like protein 2 homolog | GRHL2 |
| IPI00290308 | Tribbles homolog 1 | TRIB1 |
| IPI00017567 | Isoform Long of Endoglin precursor | ENG |
| IPI00005859 | Keratin, type II cytoskeletal 75 | KRT75 |
| IPI00017630 | Nuclear fragile × mental retardation-interacting protein 1 | NUFIP1 |
| IPI00019992 | Myb-related protein A | MYBL1 |
| IPI00025622 | AN1-type zinc finger protein 5 | ZFAND5 |
| IPI00168848 | UDP-GlcNAc:betaGal beta-1,3-N- | B3GNT7 |
| | acetylglucosaminyltransferase 7 | |
| IPI00019901 | Isoform 1 of Alpha-adducin | ADD1 |
| IPI00006510 | Tubulin beta-1 chain | TUBB1 |
| IPI00102808 | Isoform 1 of Dual specificity protein phosphatase 19 | DUSP19 |
| IPI00023942 | Isoform 2 of Syndecan-3 | SDC3 |
| IPI00031506 | Potassium/sodium hyperpolarization-activated cyclic | HCN1 |
| | nucleotide-gated channel 1 | |
| IPI00018208 | Tetratricopeptide repeat protein 33 | TTC33 |
| IPI00166865 | CDGSH iron sulfur domain-containing protein 2 | CISD2 |
| IPI00305833 | Smu-1 suppressor of mec-8 and unc-52 protein homolog | SMU1 |
| IPI00219129 | Ribosyldihydronicotinamide dehydrogenase | NQO2 |
| IPI00004533 | Kinesin-like protein KIF3B | KIF3B |
| IPI00028614 | erythrocyte membrane protein band 4.2 isoform 2 | EPB42 |
| IPI00216917 | Isoform 1 of Uncharacterized protein C22orf9 | C22orf9 |
| IPI00043499 | Probable urocanate hydratase | UROC1 |
| IPI00477468 | RNA polymerase-associated protein CTR9 homolog | CTR9 |
| IPI00005668 | Aldo-keto reductase family 1 member C2 | AKR1C2 |
| IPI00018098 | Isoform 1 of Pre-mRNA-splicing factor 38B | PRPF38B |
| IPI00026241 | Bone marrow stromal antigen 2 precursor | BST2 |
| IPI00056507 | Isoform 1 of F-box/WD repeat-containing protein 5 | FBXW5 |
| IPI00017451 | Splicing factor 3 subunit 1 | SF3A1 |
| IPI00243338 | 24 kDa protein | KRT23 |
| IPI00786937 | similar to deleted in malignant brain tumors 1 isoform b | LOC731940 |
| | precursor | |
| IPI00465273 | UHRF1-binding protein 1 | UHRF1BP1 |
| IPI00550746 | Nuclear migration protein nudC | NUDC |
| IPI00738999 | similar to ubiquitin-conjugating enzyme E2 variant 1 | LOC643227 |
| IPI00218918 | Annex in A1 | ANXA1 |
| IPI00031047 | CDNA: FLJ23571 fis, clone LNG12303 | IQCG |
| IPI00295577 | Receptor-type tyrosine-protein phosphatase beta precursor | PTPRB |
| IPI00718806 | arylhydrocarbon receptor repressor | AHRR; PDCD6 |
| IPI00029372 | Uncharacterized protein C4orf15 | C4orf15 |
| IPI00022256 | AP-2 complex subunit mu-1 | AP2M1 |
| IPI00744226 | Conserved hypothetical protein | — |
| IPI00299116 | Podocalyxin-like protein 1 precursor | PODXL |
| IPI00792229 | 20 kDa protein | TATDN1 |
| IPI00151141 | Isoform 1 of Serine/threonine-protein kinase WNK4 | WNK4 |
| IPI00220271 | Alcohol dehydrogenase | AKR1A1 |
| IPI00786893 | similar to LYRIC/3D3 | LOC730296; LOC728998 |
| IPI00027232 | Insulin-like growth factor 1 receptor precursor | IGF1R |
| IPI00028387 | Isoform 1 of Uncharacterized protein C20orf116 precursor | C20orf116 |
| IPI00015954 | GTP-binding protein SAR1a | SAR1A |
| IPI00397949 | G protein-coupled receptor 56 isoform b | GPR56 |
| IPI00328748 | Protein ARMET precursor | ARMET |
| IPI00027547 | Dermcidin precursor | DCD |
| IPI00009396 | Isoform 1 of Cannabinoid receptor 1 | CNR1 |
| IPI00306853 | Carbohydrate sulfotransferase 3 | CHST3 |
| IPI00418735 | hypothetical protein LOC400566 | LOC400566 |
| IPI00553006 | Probable prolyl-tRNA synthetase, mitochondrial precursor | PARS2 |
| IPI00102803 | Ankyrin repeat domain-containing protein 22 | ANKRD22 |
| IPI00306413 | Tubulin polymerization-promoting protein family member 3 | TPPP3 |
| IPI00018755 | High mobility group protein 1-like 10 | HMG1L10 |
| IPI00030153 | Tudor domain-containing protein 6 | TDRD6 |
| IPI00010212 | U6 snRNA-specific terminal uridylyltransferase 1 | TUT1 |
| IPI00844501 | MG44 protein | SUV39H1 |
| IPI00385543 | Isoform 3 of UPF0469 protein KIAA0907 | KIAA0907 |
| IPI00456060 | Protein | — |
| IPI00299076 | Receptor-binding cancer antigen expressed on SiSo cells | EBAG9 |
| | (Fragment) | |
| IPI00386393 | CDNA FLJ13729 fis, clone PLACE3000121, weakly | EXOC6B |
| | similar to VESICULAR TRAFFIC CONTROL PROTEIN | |
| | SEC15 | |
| IPI00016480 | Isoform 1 of NACHT, LRR and PYD domains-containing | NLRP2 |
| | protein 2 | |
| IPI00166043 | Isoform 2 of Progestin and adipoQ receptor family member 6 | PAQR6 |
| IPI00878511 | 45 kDa protein | — |
| IPI00411545 | Isoform 3 of Thrombopoietin precursor | THPO |
| IPI00442564 | CDNA FLJ26948 fis, clone RCT08241 | — |
| IPI00257508 | Dihydropyrimidinase-related protein 2 | DPYSL2 |
| IPI00398918 | Putative uncharacterized protein DKFZp686I21167 | DKFZp686O24166 |
| IPI00412408 | Breast cancer type 2 susceptibility protein | BRCA2 |
| IPI00005614 | Isoform Long of Spectrin beta chain, brain 1 | SPTBN1 |
| IPI00168239 | Zinc finger protein GLIS1 | GLIS1 |
| IPI00165990 | Isoform 4 of Transcription factor RFX4 | RFX4 |
| IPI00021831 | cAMP-dependent protein kinase type I-alpha regulatory | PRKAR1A |
| | subunit | |
| IPI00749489 | fibrocystin L | PKHD1L1 |
| IPI00552578 | Serum amyloid A protein precursor | SAA1; SAA2 |
| IPI00008422 | Isoform 2 of SWI/SNF-related matrix-associated actin- | SMARCAD1 |
| | dependent regulator of chromatin subfamily A containing | |
| | DEAD/H box 1 | |
| IPI00018953 | Dipeptidyl peptidase 4 | DPP4 |
| IPI00413778 | Peptidyl-prolyl cis-trans isomerase | FKBP1A |
| IPI00007682 | Vacuolar ATP synthase catalytic subunit A | ATP6V1A |
| IPI00024502 | Ubiquilin-4 | UBQLN4 |
| IPI00059144 | Protein FAM46B | FAM46B |
| IPI00550792 | Isoform 1 of Bridging integrator 2 | BIN2 |
| IPI00029175 | Strumpellin | KIAA0196 |
| IPI00045360 | Capicua-like protein/double homeodomain 4 fusion protein | CIC |
| IPI00787697 | similar to Oligophrenin 1 isoform 4 | FLJ32810 |
| IPI00749171 | Conserved hypothetical protein | LOC340184 |
| IPI00033419 | Protein Fem-1 homolog b | FEM1B |
| IPI00043237 | CDNA FLJ32095 fis, clone OCBBF2000998 | LOC730193; STX6 |
| IPI00298738 | DNA-directed RNA polymerase, mitochondrial precursor | POLRMT |
| IPI00028641 | Isoform 4 of Protein phosphatase 1 regulatory subunit 12B | PPP1R12B |
| IPI00554711 | Junction plakoglobin | JUP |
| IPI00879159 | 9 kDa protein | — |
| IPI00445278 | CDNA FLJ44033 fis, clone TESTI4028062 | — |
| IPI00335541 | Isoform 1 of Protein timeless homolog | TIMELESS |
| IPI00644840 | Hypothetical protein | LOC255783 |
| IPI00009771 | Lamin-B2 | LMNB2 |
| IPI00444842 | CDNA FLJ45125 fis, clone BRAWH3036561 | — |
| IPI00007800 | Angiopoietin-related protein 2 precursor | ANGPTL2 |
| IPI00181743 | Isoform 1 of BAI1-associated protein 3 | BAIAP3 |
| IPI00018099 | E3 ubiquitin-protein ligase FANCL | FANCL |
| IPI00455076 | Double C2-like domain-containing protein alpha | DOC2A |
| IPI00023152 | Isoform 1 of N-acetylated-alpha-linked acidic dipeptidase- | NAALADL1 |
| | like protein | |
| IPI00296727 | Kinesin-like protein KIF2B | KIF2B |
| IPI00005347 | Zinc finger Ran-binding domain-containing protein 1 | ZRANB1 |
| IPI00295940 | Protein unc-84 homolog B | UNC84B |
| IPI00470913 | RANBP2-like and GRIP domain containing 1 | RGPD1; RGPD2 |
| IPI00444331 | Isoform 4 of Histone-lysine N-methyltransferase NSD3 | WHSC1L1 |
| IPI00023407 | Nck-associated protein 1-like | NCKAP1L |
| IPI00306046 | Isoform 1 of EGF-like repeat and discoidin I-like domain-c [symbol] | EDIL3 |
| IPI00426727 | Isoform 1 of Methyl-CpG-binding domain protein 4 | MBD4 |
| IPI00002970 | Beta-sarcoglycan | SGCB |
| IPI00878142 | 68 kDa protein | — |
| IPI00554488 | cAMP-dependent protein kinase type I-beta regulatory | PRKAR1B |
| | subunit | |
| IPI00301288 | polydom | SVEP1 |
| IPI00030279 | Isoform 1 of Zinc finger Ran-binding domain-containing | ZRANB3 |
| | protein 3 | |
| IPI00074656 | Isoform 1 of Coiled-coil domain-containing protein | C6orf65 |
| | C6orf65 | |
| IPI00031907 | Isoform 1 of Transmembrane protein 108 precursor | TMEM108 |
| IPI00456635 | Isoform 1 of Protein unc-13 homolog D | UNC13D |
| IPI00445315 | Protein FAM47C | FAM47C |
| IPI00065931 | Isoform 2 of A-kinase anchor protein 13 | AKAP13 |
| IPI00020230 | Frizzled-9 precursor | FZD9 |
| IPI00149044 | Isoform 2 of Suppressor of hairy wing homolog 4 | ZNF280D |
| IPI00014903 | Isoform 1 of FGFR1 oncogene partner 2 | FGFR1OP2 |
| IPI00019502 | Myosin-9 | MYH9 |
| IPI00007834 | Isoform 1 of Ankyrin-2 | ANK2 |
| IPI00011961 | Isoform 1 of Sialoadhesin precursor | SIGLEC1 |
| IPI00746681 | Similar to Bcl-2-related ovarian killer protein | — |
| IPI00000977 | Mitogen-activated protein kinase kinase kinase 11 | MAP3K11 |
| IPI00007047 | Protein S100-A8 | S100A8 |
| IPI00884399 | RNA-directed DNA polymerase (Reverse transcriptase), rel [symbol] | — |
| IPI00871556 | 107 kDa protein | ZFYVE28 |
| IPI00746666 | hypothetical protein | LOC728262 |
| IPI00018879 | Alpha-L-iduronidase precursor | IDUA |
| IPI00170770 | Isoform 1 of PHD finger protein 3 | PHF3 |
| IPI00000057 | Conserved oligomeric Golgi complex component 2 | COG2 |
| IPI00003814 | Isoform 1 of Dual specificity mitogen-activated protein | MAP2K6 |
| | kinase kinase 6 | |
| IPI00290854 | A-kinase anchor protein 3 | AKAP3 |
| IPI00301058 | Vasodilator-stimulated phosphoprotein | VASP |
| IPI00245940 | immunoglobulin superfamily 5 like | IGSF5 |
| IPI00657699 | Protein | MUC19 |
| IPI00435928 | PP13187 | RASGRF1 |
| IPI00290094 | Splicing factor, arginine/serine-rich 8 | SFRS8 |
| IPI00030706 | Activator of 90 kDa heat shock protein ATPase homolog 1 | AHSA1 |
| IPI00013721 | Serine/threonine-protein kinase PRP4 homolog | PRPF4B |
| IPI00853516 | dynein, axonemal, heavy chain 17 | DNAH17 |
| IPI00410093 | coiled-coil domain containing 69 | CCDC69 |
| IPI00748607 | Conserved hypothetical protein | — |
| IPI00168404 | Zinc finger and BTB domain containing 34 | ZBTB34 |
| IPI00021266 | 60S ribosomal protein L23a | RPL23A; hCG_16001 |
| IPI00644766 | cDNA FLJ78048, highly similar to Homo sapiens torsin A i [symbol] | TOR1AIP1 |
| IPI00167154 | Uncharacterized protein MAPKBP1 | MAPKBP1 |
| IPI00031228 | Isoform 2 of Pleckstrin homology domain-containing | PLEKHB2 |
| | family B member 2 | |
| IPI00871533 | Uncharacterized protein C3orf48 (Fragment) | C3orf48 |
| IPI00007812 | Vacuolar ATP synthase subunit B, brain isoform | ATP6V1B2 |
| IPI00877615 | 15 kDa protein | — |
| IPI00030352 | Isoform 2 of Growth inhibition and differentiation-related | C10orf28 |
| | protein 88 | |
| IPI00384051 | Uncharacterized protein PSME2 | PSME2 |
| IPI00337415 | Guanine nucleotide-binding protein G(i), alpha-1 subunit | GNAI1 |
| IPI00003377 | Isoform 1 of Splicing factor, arginine/serine-rich 7 | SFRS7 |
| IPI00006146 | serum amyloid A2 | SAA1; SAA2 |
| IPI00017551 | Isoform 1 of Regucalcin | RGN |
| IPI00032338 | kelch-like 20 | KLHL20 |
| IPI00293679 | Isoform 1 of Potassium voltage-gated channel subfamily | KCNQ4 |
| | KQT member 4 | |
| IPI00745396 | Stromal RNA regulating factor | HNRPLL |
| IPI00604752 | Isoform 3 of Sterile alpha motif domain-containing protein | SAMD13 |
| | 13 | |
| IPI00382534 | Ig heavy chain V-II region OU | — |
| IPI00748682 | Pheromone shutdown-related, TraB family protein | — |
| IPI00008444 | Isoform 1 of Cytidine and dCMP deaminase domain- | CDADC1 |
| | containing protein 1 | |
| IPI00792933 | zinc finger protein 462 | ZNF462 |
| IPI00019888 | Succinate-semialdehyde dehydrogenase, mitochondrial | ALDH5A1 |
| | precursor | |
| IPI00032597 | RNA-binding motif protein, X-linked 2 | RBMX2 |
| IPI00402005 | Similar to DNA-binding protein | ZNF528 |
| IPI00020036 | Neuronal acetylcholine receptor subunit alpha-4 precursor | CHRNA4 |
| IPI00783855 | neighbor of BRCA1 gene 1 | NBR1 |
| IPI00329054 | Osteopetrosis-associated transmembrane protein 1 | OSTM1 |
| | precursor | |
| IPI00028864 | Neurexophilin-3 precursor | NXPH3 |
| IPI00023757 | Isoform 1 of X-linked retinitis pigmentosa GTPase | RPGR |
| | regulator | |
| IPI00746987 | Ribosomal protein S1 family protein | — |
| IPI00295469 | Copine-6 | CPNE6 |
| IPI00000663 | Isoform Mitochondrial of Malonyl-CoA decarboxylase, | MLYCD |
| | mitochondrial precursor | |
| IPI00788258 | similar to lysyl oxidase-like 1 preproprotein | LOXL1 |
| IPI00739387 | hypothetical protein LOC401478 | FLJ45872 |
| IPI00456599 | hypothetical protein LOC84792 | MGC12966 |
| IPI00607831 | PRAME family member 3 | PRAMEF3 |
| IPI00399254 | Isoform 1 of OTU domain-containing protein 4 | OTUD4 |
| IPI00164352 | zinc finger protein 292 | ZNF292 |
| IPI00719505 | RABL2A protein | RABL2A |
| IPI00008913 | Alpha-N-acetylgalactosaminide alpha-2,6-sialyltransferase 5 | ST6GALNAC5 |
| IPI00020454 | Deoxycytidine kinase | DCK |
| IPI00784880 | Cancer/testis antigen 75 | LOC440934 |
| IPI00514028 | 21 kDa protein | RNF207 |
| IPI00884353 | Ets-1 transcript variant ets-1 delta | — |
| IPI00852725 | Isoform 7 of Prolactin receptor precursor | PRLR |
| IPI00024292 | Low-density lipoprotein receptor-related protein 2 | LRP2 |
| | precursor | |
| IPI00005600 | Isoform 1 of Bifunctional heparan sulfate N-deacetylase/N- | NDST2 |
| | sulfotransferase 2 | |
| IPI00028357 | Exportin-4 | XPO4 |
| IPI00438355 | Fibrillin-3 precursor | FBN3 |
| IPI00009043 | Apoptotic-related protein PCAR (Fragment) | — |
| IPI00791593 | 8 kDa protein | GLYCAM1 |
| IPI00024346 | snRNA-activating protein complex subunit 3 | SNAPC3 |
| IPI00293396 | adaptor-related protein complex 1, gamma 1 subunit | AP1G1 |
| | isoform a | |
| IPI00010859 | cortistatin preproprotein | CORT; APITD1 |
| IPI00220412 | S100 calcium binding protein A1 | S100A1 |
| IPI00018914 | Tyrosine-protein phosphatase non-receptor type 14 | PTPN14 |
| IPI00027442 | Alanyl-tRNA synthetase, cytoplasmic | AARS |
| IPI00553138 | Vesicle-associated membrane protein 2 | VAMP2 |
| IPI00296913 | ADP-sugar pyrophosphatase | NUDT5 |
| IPI00014845 | Isoform 1 of Ciliary dynein heavy chain 8 | DNAH8 |
| IPI00185146 | Importin-9 | IPO9 |
| IPI00385687 | similar to Ubiquinol-cytochrome c reductase complex 14 kDa | — |
| | protein | |
| IPI00143753 | Isoform 1 of U2-associated protein SR140 | SR140 |
| IPI00219682 | Erythrocyte band 7 integral membrane protein | STOM |
| IPI00377214 | Isoform 2 of NLR family member X1 | NLRX1 |
| IPI00054521 | Frizzled-1 precursor | FZD1 |
| IPI00217791 | Coiled-coil domain-containing protein 105 | CCDC105 |
| IPI00329538 | Prostasin precursor | PRSS8 |
| IPI00794829 | 16 kDa protein | ACADS |
| IPI00167006 | Uncharacterized protein C13orf26 | C13orf26 |
| IPI00028481 | Ras-related protein Rab-8A | RAB8A |
| IPI00294215 | Uncharacterized protein KIAA0232 | KIAA0232 |
| IPI00027806 | Cysteine-rich secretory protein LCCL domain-containing 1 | CRISPLD1 |
| | precursor | |
| IPI00216423 | Isoform 4 of Intersectin-2 | ITSN2 |
| IPI00303870 | Seven transmembrane helix receptor | — |
| IPI00071185 | Isoform SV1 of PITSLRE serine/threonine-protein kinase | CDC2L1 |
| | CDC2L1 | |
| IPI00848229 | EGF, latrophilin and seven transmembrane domain- | ELTD1 |
| | containing protein 1 precursor | |
| IPI00166776 | Protein CREG2 precursor | CREG2 |
| IPI00456969 | Dynein heavy chain, cytosolic | DYNC1H1 |
| IPI00293857 | Isoform 1A of Beta-arrestin-1 | ARRB1 |
| IPI00001796 | Tumor necrosis factor receptor superfamily, member 18 | TNFRSF18 |
| | (Fragment) | |
| IPI00549972 | LIM domain-containing protein 2 | LIMD2 |
| IPI00604551 | Isoform 1 of Cell division cycle-associated protein 7 | CDCA7 |
| IPI00293565 | fms-related tyrosine kinase 4 isoform 1 | FLT4 |
| IPI00022471 | minor histocompatibility antigen HA-1 | HMHA1 |
| IPI00029050 | Isoform 1 of Glycosyltransferase-like protein LARGE1 | LARGE |
| IPI00848252 | hypothetical protein LOC285501 | LOC285501 |
| IPI00288939 | Isoform 3 of Coiled-coil domain-containing protein 40 | CCDC40 |
| IPI00029107 | Werner syndrome ATP-dependent helicase | WRN |
| IPI00065057 | Putative uncharacterized protein CXorf58 | CXorf58 |
| IPI00073769 | 48 kDa protein | PALM2 |
| IPI00065520 | Isoform 1 of Protein phosphatase 1M | PPM1M |
| IPI00470468 | Isoform 3 of Protein EFR3 homolog A | EFR3A |
| IPI00436518 | Benzodiazepine receptor ligand | — |
| IPI00382515 | CDNA FLJ30384 fis, clone BRACE2008114 | — |
| IPI00749514 | attractin-like 1 | ATRNL1 |
| IPI00853312 | Uncharacterized protein ENSP00000324580 | — |
| IPI00217871 | Delta-1-pyrroline-5-carboxylate dehydrogenase, | ALDH4A1 |
| | mitochondrial precursor | |
| IPI00015345 | Cadherin EGF LAG seven-pass G-type receptor 3 | CELSR3; SLC26A6 |
| | precursor | |
| IPI00644191 | 70 kDa protein | ZNF90 |
| IPI00847391 | Isoform 1 of Zinc finger protein 276 | ZNF276 |
| IPI00001654 | Isoform 3 of Pericentriolar material 1 protein | PCM1 |
| IPI00470490 | Isoform 1 of Nuclear receptor coactivator 1 | NCOA1 |
| IPI00410488 | Isoform 1 of CD276 antigen precursor | CD276 |
| IPI00001433 | Protocadherin beta 15 precursor | PCDHB15 |
| IPI00220156 | Isoform B of Transforming growth factor beta-2 precursor | TGFB2 |
| IPI00024818 | Isoform 1 of Ubiquitin-specific peptidase-like protein 1 | USPL1 |
| IPI00335437 | Ankyrin repeat and zinc finger domain-containing protein 1 | ANKZF1 |
| IPI00000076 | Beta-nerve growth factor precursor | NGFB |
| IPI00100980 | EH domain-containing protein 2 | EHD2 |
| IPI00215610 | 55 kDa erythrocyte membrane protein | MPP1 |
| IPI00015148 | Ras-related protein Rap-1b precursor | RAP1B |
| IPI00028493 | Isoform 1 of Tuberin | TSC2 |
| IPI00410657 | Isoform 2 of mRNA cap guanine-N7 methyltransferase | RNMT |
| IPI00328361 | Seryl-tRNA synthetase, mitochondrial precursor | SARS2 |
| IPI00029700 | Isoform Long of Down syndrome cell adhesion molecule | DSCAM |
| | precursor | |
| IPI00332583 | chorionic somatomammotropin hormone 1 isoform 2 | CSH1; CSH2; GH1 |
| IPI00552591 | V1-20 protein | IGLV10-54 |
| IPI00022277 | Coiled-coil domain-containing protein 56 | CCDC56 |
| IPI00301844 | Zinc fingers and homeoboxes protein 3 | ZHX3 |
| IPI00013788 | HIV Tat-specific factor 1 | HTATSF1 |
| IPI00216288 | Isoform 3 of Lethal | L3MBTL |
| IPI00025499 | Isoform Tau-F of Microtubule-associated protein tau | MAPT |
| IPI00001151 | Isoform 2 of Uncharacterized MFS-type transporter | C20orf59 |
| | C20orf59 | |
| IPI00787083 | similar to peptidylprolyl isomerase A isoform 1 | LOC256374 |
| IPI00856012 | collagen type VI alpha 6 | COL6A6 |
| IPI00001712 | Isoform 1 of Catenin alpha-3 | CTNNA3 |
| IPI00847237 | Conserved hypothetical protein | — |
| IPI00295999 | Isoform 2 of Collagen alpha-1(IX) chain precursor | COL9A1 |
| IPI00217831 | Ankyrin repeat domain-containing protein 13A | ANKRD13A |
| IPI00296196 | Dimethylglycine dehydrogenase, mitochondrial precursor | DMGDH |
| IPI00396145 | FYVE finger-containing phosphoinositide kinase | PIP5K3 |
| IPI00478110 | Probable G-protein coupled receptor 135 | GPR135 |
| IPI00335168 | Isoform Non-muscle of Myosin light polypeptide 6 | MYL6 |
| IPI00030037 | Agouti-signaling protein precursor | ASIP |
| IPI00013749 | Protein kinase C zeta type | PRKCZ |
| IPI00002491 | Isoform 9 of Sorbin and SH3 domain-containing protein 1 | SORBS1 |
| IPI00240793 | Probable phospholipid-transporting ATPase IF | ATP11B |
| IPI00168995 | Olfactory receptor 4D9 | OR4D9 |
| IPI00024704 | Uronyl 2-sulfotransferase | UST |
| IPI00031195 | Isoform 1 of Vang-like protein 1 | VANGL1 |
| IPI00059190 | Isoform 1 of SLAM family member 9 precursor | SLAMF9 |
| IPI00043467 | Tigger transposable element-derived protein 1 | TIGD1 |
| IPI00376237 | Isoform 2 of Transcription factor LBX2 | LBX2 |
| IPI00235842 | Zinc finger protein 483 | ZNF483 |
| IPI00220473 | Isoform 2 of Calcium-transporting ATPase type 2C | ATP2C1 |
| | member 1 | |
| IPI00169115 | Olfactory receptor OR9-8 | OR13C3 |
| IPI00020966 | Isoform 1 of Phosphatidylinositol N- | PIGA |
| | acetylglucosaminyltransferase subunit A | |
| IPI00456827 | Protein FAM22G precursor | FAM22G |
| IPI00382831 | Cerebral protein-13 | BAP1 |
| IPI00477692 | Uncharacterized protein ENSP00000330808 | LOC131149 |
| IPI00747657 | Similar to Rod cGMP-specific 3′,5′-cyclic | PDE6B |
| | phosphodiesterase subunit beta precursor | |
| IPI00301583 | Isoform 1 of Zinc finger protein 691 | ZNF691 |
| IPI00790447 | 12 kDa protein | — |
| IPI00829904 | Uncharacterized protein ENSP00000374814 | — |
| IPI00215777 | Isoform B of Phosphate carrier protein, mitochondrial | SLC25A3 |
| | precursor | |
| IPI00028912 | zinc finger protein 161 | VEZF1 |
| IPI00387161 | Ig lambda chain V-I region VOR | — |
| IPI00830107 | V4-2 protein | IGLV5-45 |
| IPI00440493 | ATP synthase subunit alpha, mitochondrial precursor | ATP5A1 |
| IPI00020533 | Protein BEX1 | BEX1 |
| IPI00019242 | Matrix metalloproteinase-15 precursor | MMP15 |
| IPI00376436 | Isoform 4 of Vacuolar protein sorting-associated protein | VPS13B |
| | 13B | |
| IPI00029162 | Isoform 2 of Cell division cycle 2-like protein kinase 5 | CDC2L5 |
| IPI00419237 | Isoform 1 of Cytosol aminopeptidase | LAP3 |
| IPI00065500 | BRO1 domain-containing protein BROX | C1orf58 |
| IPI00740336 | formin 1 | FMN1 |
| IPI00017203 | Protein RMI1 homolog | RMI1 |
| IPI00024664 | Isoform Long of Ubiquitin carboxyl-terminal hydrolase 5 | USP5 |
| IPI00786946 | similar to Tektin-3 | LOC642249 |
| IPI00166257 | CDNA FLJ37614 fis, clone BRCOC2011769 | — |
| IPI00012877 | Isoform 1 of Interferon-alpha/beta receptor alpha chain | IFNAR1 |
| | precursor | |
| IPI00021199 | Stathmin-3 | STMN3 |
| IPI00293431 | WUGSC:H_DJ0855D21.2 protein | MGC70863 |
| IPI00219563 | Isoform A of 1-phosphatidylinositol-4,5-bisphosphate | PLCB1 |
| | phosphodiesterase beta-1 | |
| IPI00442745 | CDNA FLJ26780 fis, clone PRS03837 | — |
| IPI00295400 | Tryptophanyl-tRNA synthetase, cytoplasmic | WARS |
| IPI00444330 | CDNA FLJ45693 fis, clone FEBRA2012625 | — |
| IPI00170509 | Isoform 3 of Calcium/calmodulin-dependent protein kinase | CAMKK2 |
| | kinase 2 | |
| IPI00017256 | Ras suppressor protein 1 | RSU1 |
| IPI00374301 | hypothetical protein | — |
| IPI00023217 | Isoform 1 of Ryanodine receptor 2 | RYR2 |
| IPI00017533 | Cytochrome c oxidase subunit 3 | MT-CO3 |
| IPI00419922 | Isoform 1 of IQ domain-containing protein E | IQCE |
| IPI00029997 | 6-phosphogluconolactonase | PGLS |
| IPI00024920 | ATP synthase subunit delta, mitochondrial precursor | ATP5D |
| IPI00005710 | Heparan sulfate glucosamine 3-O-sulfotransferase 2 | HS3ST2 |
| IPI00026262 | Isoform 1 of Ras GTPase-activating protein 1 | RASA1 |
| IPI00744872 | Isoform 1 of Uncharacterized protein KIAA0423 | KIAA0423 |
| IPI00787412 | similar to Protein C11orf2 | LOC441616 |
| IPI00168607 | tetratricopeptide repeat domain 21A isoform 1 | TTC21A |
| IPI00021091 | Isoform 1 of Leucine-rich glioma-inactivated protein 1 | LGI1 |
| | precursor | |
| IPI00419385 | olfactory receptor, family 52, subfamily E, member 8 | OR52E8 |
| IPI00018843 | Isoform 1 of D(3) dopamine receptor | DRD3 |
| IPI00477616 | Protein phosphatase 2A activator, regulatory subunit 4 | PPP2R4 |
| IPI00021695 | Isoform D of Plasma membrane calcium-transporting | ATP2B1 |
| | ATPase 1 | |
| IPI00027782 | Stromelysin-1 precursor | MMP3 |
| IPI00030009 | Isoform A of Bifunctional 3′-phosphoadenosine 5′- | PAPSS2 |
| | phosphosulfate synthetase 2 | |
| IPI00479722 | Proteasome activator complex subunit 1 | PSME1 |
| IPI00006236 | Platelet-derived growth factor receptor-like protein | PDGFRL |
| | precursor | |
| IPI00177878 | Isoform 3 of Transmembrane protein 16D | TMEM16D |
| IPI00291796 | BTB (POZ) domain containing 12 | BTBD12 |
| IPI00104907 | Isoform 1 of Uncharacterized potential DNA-binding | C14orf106 |
| | protein C14orf106 | |
| IPI00400795 | 54 kDa protein | — |
| IPI00303622 | Olfactory receptor OR9-23 | OR1N2 |
| IPI00031554 | ATP-dependent RNA helicase DDX50 | DDX50 |
| IPI00005107 | Niemann-Pick C1 protein precursor | NPC1 |
| IPI00023860 | Nucleosome assembly protein 1-like 1 | NAP1L1 |
| IPI00028520 | Isoform 1 of NADH dehydrogenase [ubiquinone] | NDUFV1 |
| | flavoprotein 1, mitochondrial precursor | |
| IPI00009070 | Isoform 1 of HBS1-like protein | HBS1L |
| IPI00375225 | Olfactory receptor 2AE1 | OR2AE1 |
| IPI00235622 | Isoform 3 of CUB domain-containing protein 1 precursor | CDCP1 |
| IPI00742031 | similar to golgi autoantigen, golgin subfamily a, 8G | LOC645137 |
| IPI00480159 | inositol polyphosphate-5-phosphatase F | INPP5F |
| IPI00221372 | Isoform 2 of 1-acyl-sn-glycerol-3-phosphate acyltransferase | AGPAT2 |
| | beta | |
| IPI00386576 | Ig lambda chain V-IV region MOL | — |
| IPI00022295 | Platelet factor 4 variant precursor | PF4V1 |
| IPI00747420 | Melanoma-derived protein (Fragment) | — |
| IPI00432226 | AVLL5809 | — |
| IPI00796972 | 62 kDa protein | ETV5 |
| IPI00171874 | Ras guanyl-releasing protein 3 | RASGRP3 |
| IPI00010807 | Frizzled-8 precursor | FZD8 |
| IPI00006746 | Ermin | ERMN |
| IPI00178894 | Zinc finger and BTB domain-containing protein 20 | ZBTB20 |
| IPI00878436 | 24 kDa protein | SLC2A11 |
| IPI00640810 | 6 kDa protein | CTDP1 |
| IPI00003921 | Isoform 1 of Protein 4.1 | EPB41 |
| IPI00031410 | FKBP12-rapamycin complex-associated protein | FRAP1 |
| IPI00019848 | Isoform 1 of Host cell factor | HCFC1 |
| IPI00306549 | CDNA FLJ11065 fis, clone PLACE1004868, weakly | MLSTD1 |
| | similar to MALE STERILITY PROTEIN 2 | |
| IPI00306611 | TRAF-interacting protein | TRAIP |
| IPI00003392 | Transmembrane protein 5 | TMEM5 |
| IPI00153031 | Isoform 1 of Uncharacterized protein C17orf80 | C17orf80 |
| IPI00289097 | Isoform 1 of Ras-GEF domain-containing family member | RASGEF1B |
| | 1B | |
| IPI00425404 | Isoform 1 of Kinesin-like protein KIF21A | KIF21A |
| IPI00293361 | Isoform 2 of Small G protein signaling modulator 2 | SGSM2 |
| IPI00477262 | Uncharacterized protein TRIM41 (Fragment) | TRIM41 |
| IPI00021753 | Kinesin-like protein KIF13B | KIF13B |
| IPI00030703 | Motor neuron and pancreas homeobox protein 1 | MNX1 |
| IPI00025974 | Charged multivesicular body protein 4b | CHMP4B |
| IPI00413655 | ankyrin repeat domain 55 isoform 1 | ANKRD55 |
| IPI00253281 | Isoform 2 of Epidermal growth factor receptor kinase | EPS8L1 |
| | substrate 8-like protein 1 | |
| IPI00005607 | Isoform 1 of Deleted in bladder cancer protein 1 precursor | DBC1 |
| IPI00027984 | Putative uncharacterized protein | DNAJC11 |
| IPI00026497 | Isoform 1 of Poly [ADP-ribose] polymerase 2 | PARP2 |
| IPI00296441 | Adenosine deaminase | ADA |
| IPI00329605 | DNA mismatch repair protein Msh3 | MSH3 |
| IPI00032425 | Receptor activity-modifying protein 3 precursor | RAMP3 |
| IPI00061206 | Polyadenylate-binding protein 5 | PABPC5 |
| IPI00289758 | Calpain-2 catalytic subunit precursor | CAPN2 |
| IPI00001568 | Vacuolar proton pump subunit D | ATP6V1D |
| IPI00465177 | Transforming growth factor-alpha variant I | TGFA |
| IPI00020470 | Isoform 1 of Glycosyltransferase 8 domain-containing | GLT8D1 |
| | protein 1 | |
| IPI00718821 | Isoform 1 of Uncharacterized protein C19orf55 | C19orf55 |
| IPI00018387 | Furin precursor | FURIN |
| IPI00398229 | similar to deubiquitinating enzyme 3 | LOC392197 |
| IPI00027248 | Tumor suppressor candidate 2 | TUSC2 |
| IPI00163851 | Isoform 1 of Eukaryotic translation initiation factor 2-alpha | EIF2AK4 |
| | kinase 4 | |
| IPI00438286 | Isoform 1 of Protein LAP2 | ERBB2IP |
| IPI00023780 | Isoform 2 of DnaJ homolog subfamily C member 5 | DNAJC5 |
| IPI00018027 | Isoform 1 of Angiogenic factor with G patch and FHA | AGGF1 |
| | domains 1 | |
| IPI00180305 | Isoform 5 of E3 ubiquitin-protein ligase UBR4 | UBR4 |
| IPI00010442 | Phospholemman precursor | FXYD1; FXYD7 |
| IPI00292657 | NADP-dependent leukotriene B4 12- | LTB4DH |
| | hydroxydehydrogenase | |
| IPI00434711 | Putative uncharacterized protein FP6679 | — |
| IPI00020329 | Potassium voltage-gated channel subfamily S member 2 | KCNS2 |
| IPI00019244 | Isoform 1 of RNA-binding protein Nova-1 | NOVA1 |
| IPI00009101 | Isoform 2 of Helicase SRCAP | SRCAP |
| IPI00184650 | Class B basic helix-loop-helix protein 4 | BHLHB4 |
| IPI00328298 | Isoform 2 of Structural maintenance of chromosomes | SMC4 |
| | protein 4 | |
| IPI00782992 | Isoform 1 of Serine/arginine repetitive matrix protein 2 | SRRM2 |
| IPI00006663 | Aldehyde dehydrogenase, mitochondrial precursor | ALDH2 |
| IPI00014340 | Isoform 1 of Protein phosphatase 1 regulatory subunit 12C | PPP1R12C |
| IPI00807675 | ZNF440 protein | ZNF440 |
| IPI00104050 | Thyroid hormone receptor-associated protein 3 | THRAP3 |
| IPI00386284 | olfactory receptor, family 2, subfamily AK, member 2 | OR2AK2 |
| IPI00411480 | hypothetical protein | LOC729461 |
| IPI00018429 | Paired mesoderm homeobox protein 2 | PRRX2 |
| IPI00646423 | Isoform 2 of Transcription initiation factor TFIID subunit | TAF4B |
| | 4B | |
| IPI00387004 | PNAS-146 | MCM7 |
| IPI00446312 | CDNA FLJ42271 fis, clone TKIDN2015788 | — |
| IPI00218667 | Stathmin-2 | STMN2 |
| IPI00179298 | 482 kDa protein | HUWE1 |
| IPI00299547 | Neutrophil gelatinase-associated lipocalin precursor | LCN2 |
| IPI00016006 | Isoform 1 of Gephyrin | GPHN |
| IPI00395630 | Putative uncharacterized protein pp6170 | SCRIB |
| IPI00009471 | WD repeat-containing protein 3 | WDR3 |
| IPI00455967 | Uncharacterized protein ENSP00000353619 (Fragment) | — |
| IPI00100067 | Isoform 1 of Tumor necrosis factor receptor superfamily | TNFRSF19 |
| | member 19 precursor | |
| IPI00299263 | ADP-ribosylation factor GTPase-activating protein 3 | ARFGAP3 |
| IPI00186966 | Isoform IIA of Myc box-dependent-interacting protein 1 | BIN1 |
| IPI00220308 | Isoform 2 of Merlin | NF2 |
| IPI00045939 | 2-aminoethanethiol dioxygenase | ADO |
| IPI00789476 | 7 kDa protein | — |
| IPI00014537 | Isoform 1 of Calumenin precursor | CALU |
| IPI00165009 | Isoform 3 of MBT domain-containing protein 1 | MBTD1 |
| IPI00306749 | Kanadaptin | SLC4A1AP |
| IPI00001922 | Suppressor of tumorigenicity protein 14 | ST14 |
| IPI00374670 | hypothetical protein isoform 2 | LOC388588 |
| IPI00005809 | Serum deprivation-response protein | SDPR |
| IPI00453473 | Histone H4 | HIST2H4A; HIST1H4E; HIST1H4H; |
| | | HIST4H4; HIST1H4C; |
| | | HIST1H4A; HIST1H4B; |
| | | HIST1H4J; HIST1H4F; |
| | | HIST1H4K; HIST1H4L; |
| | | HIST2H4B; HIST1H4I; HIST1H4D |
| IPI00464980 | Isoform 1 of Paired amphipathic helix protein Sin3b | SIN3B |
| IPI00289837 | Coiled-coil domain-containing protein 85A | CCDC85A |
| IPI00878962 | 10 kDa protein | — |
| IPI00103891 | Putative uncharacterized protein | MTMR14 |
| IPI00328709 | Gremlin-2 precursor | GREM2 |
| IPI00744932 | Conserved hypothetical protein | ZDHHC1 |
| IPI00398154 | actin filament associated protein 1 | AFAP1 |
| IPI00291939 | Structural maintenance of chromosomes protein 1A | SMC1A |
| IPI00444272 | Leukemia inhibitory factor receptor precursor | LIFR |
| IPI00641181 | MARCKS-related protein | MARCKSL1 |
| IPI00022799 | Isoform 2 of Aquaporin-4 | AQP4 |
| IPI00445750 | CDNA FLJ43142 fis, clone CTONG3007870 | — |
| IPI00006077 | Protein Jade-3 | PHF16 |
| IPI00151990 | Isoform 1 of Thioredoxin domain-containing protein 15 | TXNDC15 |
| | precursor | |
| IPI00061148 | Sodium bicarbonate cotransporter NBC4d | SLC4A5 |
| IPI00004944 | Isoform 1 of Sodium-driven chloride bicarbonate | SLC4A10 |
| | exchanger | |
| IPI00304587 | Protein dispatched homolog 2 | DISP2 |
| IPI00219703 | Parvalbumin alpha | PVALB |
| IPI00003384 | Isoform 1 of Cadherin EGF LAG seven-pass G-type | CELSR1 |
| | receptor 1 precursor | |
| IPI00007750 | Tubulin alpha-4A chain | TUBA4A |
| IPI00015988 | HLA class I histocompatibility antigen, alpha chain G | HLA-G |
| | precursor | |
| IPI00001813 | Isoform 1 of RING and PHD-finger domain-containing | KIAA1542 |
| | protein KIAA1542 | |
| IPI00171412 | Isoform 1 of Sulfatase-modifying factor 2 precursor | SUMF2 |
| IPI00005981 | Transgelin-3 | TAGLN3 |
| IPI00032826 | Hsc70-interacting protein | ST13 |
| IPI00025447 | Elongation factor 1-alpha | EEF1A1 |
| IPI00152326 | glutathione S-transferase M1 isoform 2 | GSTM1 |
| IPI00044751 | Isoform 1 of M-phase phosphoprotein 1 | MPHOSPH1 |
| IPI00028193 | 192 kDa protein | KNDC1 |
| IPI00000775 | Isoform 1 of Leucine-rich repeats and immunoglobulin-like | LRIG1 |
| IPI00329593 | Isoform 2 of ADP-dependent glucokinase | ADPGK |
| IPI00296461 | Isoform 1 of Sphingomyelin phosphodiesterase precursor | SMPD1 |
| IPI00656052 | SPANX-C | SPANXB2; SPANXB1; SPANXF1 |
| IPI00658109 | Creatine kinase, ubiquitous mitochondrial precursor | CKMT1A; CKMT1B |
| IPI00376383 | Centrosomal protein 110 kDa | CEP110 |
| IPI00019904 | Isoform 1 of Beta-adducin | ADD2 |
| IPI00413266 | Isoform 1 of IQ and AAA domain-containing protein | IQCA |
| IPI00015973 | Band 4.1-like protein 2 | EPB41L2 |
| IPI00166807 | Isoform 3 of Oxidation resistance protein 1 | OXR1 |
| IPI00472374 | zinc finger protein 98 | ZNF98 |
| IPI00032187 | nischarin | NISCH |
| IPI00420014 | Isoform 1 of U5 small nuclear ribonucleoprotein 200 kDa | ASCC3L1 |
| | helicase | |
| IPI00386314 | FLJ00064 protein (Fragment) | CNDP2 |
| IPI00021766 | Isoform 1 of Reticulon-4 | RTN4 |
| IPI00221006 | Isoform 4 of Transcription factor 7-like 2 | TCF7L2 |

[symbol]

indicates data missing or illegible when filed

TABLE 7L
Table 7L. Qualitative List of CSF Proteins Identified
(Present) in Patients with Neurologic Lyme disease and not
found in True Normals or Chronic Fatigue. All with with
immunoaffinity depletion. 692 proteins in list.

| CleanIPI | Protein | Gene |
|---|---|---|
| IPI00551062 | Isoform 1 of Protein canopy homolog 3 precursor | CNPY3 |
| IPI00017551 | Isoform 1 of Regucalcin | RGN |
| IPI00006663 | Aldehyde dehydrogenase, mitochondrial precursor | ALDH2 |
| IPI00412977 | Prothymosin alpha | — |
| IPI00384280 | Prenylcysteine oxidase 1 precursor | PCYOX1 |
| IPI00247295 | Isoform 4 of Nesprin-1 | SYNE1 |
| IPI00007800 | Angiopoietin-related protein 2 precursor | ANGPTL2 |
| IPI00553215 | V1-5 protein | IGLV2-18 |
| IPI00002707 | Isoform 1 of Spastin | SPAST |
| IPI00005563 | Isoform 1 of Tubulointerstitial nephritis antigen-like precursor | TINAGL1 |
| IPI00827486 | Rheumatoid factor light chain variable region precursor (Fragment) | — |
| IPI00004671 | Golgin subfamily B member 1 | GOLGB1 |
| IPI00465345 | Isoform 3 of Pre-mRNA-processing factor 40 homolog B | PRPF40B |
| IPI00010130 | Glutamine synthetase | GLUL |
| IPI00477763 | Serine/threonine-protein kinase MRCK beta | CDC42BPB |
| IPI00328268 | EIF4G3 protein | EIF4G3 |
| IPI00305477 | Cystatin-SN precursor | CST1 |
| IPI00070070 | Novel protein | FLJ32784 |
| IPI00852619 | Isoform A of Probable phospholipid-transporting ATPase VB | ATP10B |
| IPI00000203 | Low-density lipoprotein receptor-related protein 6 precursor | LRP6 |
| IPI00021473 | 59 kDa protein | CBFA2T3 |
| IPI00016703 | 24-dehydrocholesterol reductase precursor | DHCR24 |
| IPI00028881 | NADH dehydrogenase [ubiquinone] 1 alpha subcomplex subunit 3 | NDUFA3 |
| IPI00152157 | Isoform 1 of Zinc finger protein 509 | ZNF509 |
| IPI00328825 | Isoform 1 of NEDD4-binding protein 2 | N4BP2 |
| IPI00060569 | Isoform 2 of Abhydrolase domain-containing protein 12 | ABHD12 |
| IPI00412298 | Isoform 1 of Serine/threonine-protein kinase ATR | ATR |
| IPI00166465 | hypothetical protein LOC221416 | C6orf223 |
| IPI00019517 | Wilms tumor 1 isoform D | WT1 |
| IPI00007074 | Tyrosyl-tRNA synthetase, cytoplasmic | YARS |
| IPI00514533 | Uncharacterized protein SYCP2L | SYCP2L |
| IPI00166043 | Isoform 2 of Progestin and adipoQ receptor family member 6 | PAQR6 |
| IPI00018953 | Dipeptidyl peptidase 4 | DPP4 |
| IPI00554711 | Junction plakoglobin | JUP |
| IPI00020230 | Frizzled-9 precursor | FZD9 |
| IPI00377214 | Isoform 2 of NLR family member X1 | NLRX1 |
| IPI00054521 | Frizzled-1 precursor | FZD1 |
| IPI00387161 | Ig lambda chain V-I region VOR | — |
| IPI00219563 | Isoform A of 1-phosphatidylinositol-4,5-bisphosphate phosphodiesterase | PLCB1 |
| | beta-1 | |
| IPI00303622 | Olfactory receptor OR9-23 | OR1N2 |
| IPI00220308 | Isoform 2 of Merlin | NF2 |
| IPI00022799 | Isoform 2 of Aquaporin-4 | AQP4 |
| IPI00001813 | Isoform 1 of RING and PHD-finger domain-containing protein | KIAA1542 |
| | KIAA1542 | |
| IPI00152733 | FLJ00237 protein (Fragment) | OLFML2A |
| IPI00030360 | RUN domain-containing protein 2A | RUNDC2A |
| IPI00010334 | Isoform 3 of Inactive phospholipase C-like protein 2 | PLCL2 |
| IPI00441952 | Isoform 1 of Ninein | NIN |
| IPI00021772 | S-adenosylmethionine synthetase isoform type-1 | MAT1A |
| IPI00332371 | Isoform 1 of 6-phosphofructokinase, liver type | PFKL |
| IPI00005530 | FMRFamide-related peptides precursor | NPVF |
| IPI00784272 | Putative uncharacterized protein | — |
| IPI00004101 | Betaine--homocysteine S-methyltransferase 1 | BHMT |
| IPI00514551 | OTTHUMP00000016936 | SCML4 |
| IPI00167228 | CDNA FLJ40966 fis, clone UTERU2012407 | CHCHD7 |
| IPI00218687 | Isoform 10 of Voltage-dependent T-type calcium channel subunit alpha- | CACNA1G |
| | 1G | |
| IPI00853079 | Isoform 1 of Uncharacterized protein C14orf166B | C14orf166B |
| IPI00008403 | Carbohydrate sulfotransferase 7 | CHST7 |
| IPI00787765 | similar to solute carrier family 35, member F4 | SLC35F4 |
| IPI00386119 | Isoform 5 of Splicing factor 1 | SF1 |
| IPI00418614 | 67 kDa protein | — |
| IPI00017231 | Neurensin-2 | NRSN2 |
| IPI00022290 | Beta-defensin 1 precursor | DEFB1 |
| IPI00884399 | RNA-directed DNA polymerase (Reverse transcriptase), related domain | — |
| | containing protein | — |
| IPI00003377 | Isoform 1 of Splicing factor, arginine/serine-rich 7 | SFRS7 |
| IPI00010859 | cortistatin preproprotein | CORT; APITD1 |
| IPI00220412 | S100 calcium binding protein A1 | S100A1 |
| IPI00031195 | Isoform 1 of Vang-like protein 1 | VANGL1 |
| IPI00658109 | Creatine kinase, ubiquitous mitochondrial precursor | CKMT1A; CKMT1B |
| IPI00852669 | Zinc finger protein 516 | ZNF516 |
| IPI00017297 | Matrin-3 | MATR3 |
| IPI00470812 | Putative uncharacterized protein DKFZp781E21107 | LOC339977 |
| IPI00556381 | Putative uncharacterized protein (Fragment) | TRIM16 |
| IPI00004569 | Isoform MZF1A of Myeloid zinc finger 1 | MZF1 |
| IPI00240059 | Transmembrane and coiled-coil domains protein 3 | TMCC3 |
| IPI00004838 | Isoform Crk-II of Proto-oncogene C-crk | CRK |
| IPI00748733 | Similar to Slit homolog 3 protein precursor | — |
| IPI00023919 | 26S protease regulatory subunit 8 | PSMC5 |
| IPI00007423 | Isoform 1 of Acidic leucine-rich nuclear phosphoprotein 32 family | ANP32B |
| | member B | |
| IPI00020005 | Arylsulfatase E precursor | ARSE |
| IPI00015181 | Integrin alpha-9 precursor | ITGA9 |
| IPI00847415 | Isoform 4 of Dual specificity protein kinase CLK3 | CLK3 |
| IPI00063245 | Uncharacterized protein FUBP3 | FUBP3 |
| IPI00012079 | Eukaryotic translation initiation factor 4B | EIF4B |
| IPI00005793 | AP-3 complex subunit beta-2 | AP3B2 |
| IPI00410391 | Isoform 1 of Major facilitator superfamily domain-containing protein 2 | MFSD2 |
| IPI00016940 | Phospholipid scramblase 2 | PLSCR2 |
| IPI00829915 | similar to PR domain zinc finger protein 16 | LOC647868 |
| IPI00455731 | Protein FAM26F | FAM26F |
| IPI00414927 | Transmembrane and immunoglobulin domain-containing protein | C1orf32 |
| | C1orf32 | |
| IPI00871458 | 39 kDa protein | C10orf4 |
| IPI00101299 | C1GALT1-specific chaperone 1 | CIGALT1C1 |
| IPI00760941 | Uncharacterized protein ENSP00000371687 | DKFZp434J1015 |
| IPI00514966 | Recombining binding protein suppressor of hairless | RBPJL |
| IPI00240909 | similar to eukaryotic translation initiation factor 3, subunit 5 epsilon, | hCG_15200 |
| | 47 kDa isoform 1 | |
| IPI00217899 | RING finger protein 168 | RNF168 |
| IPI00152462 | Ciliary dynein heavy chain 3 | DNAH3 |
| IPI00004509 | Leukotriene C4 synthase | LTC4S |
| IPI00884371 | Uncharacterized protein ENSP00000383215 | — |
| IPI00002881 | Bcl-2-related protein A1 | BCL2A1 |
| IPI00065352 | Isoform 1 of Uncharacterized serine/threonine-protein kinase SgK494 | FLJ25006 |
| IPI00000388 | Zinc finger protein 175 | ZNF175 |
| IPI00394807 | Regenerating islet-derived protein 3 gamma precursor | REG3G |
| IPI00022830 | Isoform 2 of NSFL1 cofactor p47 | NSFL1C |
| IPI00065388 | Isoform 1 of Coiled-coil domain-containing protein 17 | CCDC17 |
| IPI00289540 | Isoform 2 of Usherin precursor | USH2A |
| IPI00748216 | CDNA FLJ20187 fis, clone COLF0433 | GTF3C5 |
| IPI00022143 | Isoform 1 of Extended-synaptotagmin-1 | FAM62A |
| IPI00023663 | Putative transcription factor Ovo-like 1 | OVOL1 |
| IPI00015602 | Mitochondrial precursor proteins import receptor | TOMM70A |
| IPI00072377 | Isoform 1 of Protein SET | SET |
| IPI00011604 | Glycine cleavage system H protein, mitochondrial precursor | GCSH; LOC730107 |
| IPI00401586 | Chromosome 18 open reading frame 62 | C18orf62 |
| IPI00026492 | 2-amino-3-ketobutyrate coenzyme A ligase, mitochondrial precursor | GCAT |
| IPI00644529 | Isoform 2 of Zinc finger protein 615 | ZNF615 |
| IPI00028880 | Putative DNA polymerase | POLN |
| IPI00014878 | Uncharacterized protein PRKD1 | PRKD1 |
| IPI00794791 | Isoform 2 of IQ domain-containing protein D | IQCD |
| IPI00009853 | Vomeronasal type-1 receptor 1 | VN1R1 |
| IPI00022250 | Lymphocyte antigen 96 precursor | LY96 |
| IPI00004337 | Zinc finger and BTB domain-containing protein 11 | ZBTB11 |
| IPI00005708 | Heparan sulfate glucosamine 3-O-sulfotransferase 3B1 | HS3ST3B1 |
| IPI00169430 | Isoform 1 of Spermatid perinuclear RNA-binding protein | STRBP |
| IPI00007057 | Rabenosyn-5 | ZFYVE20 |
| IPI00386822 | Putative uncharacterized protein (Fragment) | TGFBI |
| IPI00477539 | Uncharacterized protein C14orf102 | C14orf102 |
| IPI00006648 | IDN4-GGTR14 protein | RABGAP1L |
| IPI00302176 | Isoform 1 of H/ACA ribonucleoprotein complex subunit 1 | NOLA1 |
| IPI00478586 | Isoform 2 of Vacuolar protein sorting-associated protein 13A | VPS13A |
| IPI00060439 | KIAA0226 protein | KIAA0226 |
| IPI00303343 | Splicing factor, arginine/serine-rich 19 | SCAF1 |
| IPI00869070 | Ankyrin repeat containing protein | — |
| IPI00783186 | Isoform 2 of Ankyrin repeat domain-containing protein 17 | ANKRD17 |
| IPI00303152 | collagen, type XXII, alpha 1 | COL22A1 |
| IPI00423379 | Putative uncharacterized protein DKFZp686G24192 | C20orf96 |
| IPI00025308 | Homeobox protein prophet of Pit-1 | PROP1 |
| IPI00219358 | Isoform 1 of Mannose-6-phosphate isomerase | MPI |
| IPI00307757 | Isoform 1 of Membrane-associated phosphatidylinositol transfer protein 3 | PITPNM3 |
| IPI00166738 | Zinc-binding alcohol dehydrogenase domain-containing protein 2 | ZADH2 |
| IPI00446355 | CDNA FLJ41803 fis, clone NHNPC2002749 | IER5L |
| IPI00167861 | Protein SMG5 | SMG5 |
| IPI00472171 | RPL7 protein | RPL7 |
| IPI00187091 | Isoform 2 of MAP kinase-interacting serine/threonine-protein kinase 1 | MKNK1 |
| IPI00398421 | Isoform 1 of Potassium channel subfamily T member 2 | KCNT2 |
| IPI00022228 | Vigilin | HDLBP |
| IPI00034277 | Isoform A of Probable cation-transporting ATPase 13A1 | ATP13A1 |
| IPI00152853 | Isoform 1 of Phostensin | KIAA1949 |
| IPI00446785 | CDNA FLJ41116 fis, clone BRACE1000572 | |
| IPI00411452 | Uncharacterized protein DOCK11 | DOCKI1 |
| IPI00444395 | CDNA FLJ45615 fis, clone BRTHA3026180 | — |
| IPI00019278 | Thiamine transporter 2 | SLC19A3 |
| IPI00216132 | Isoform Alpha I of Ribosomal protein S6 kinase beta-1 | RPS6KB1 |
| IPI00328115 | Isoform 3 of 5′-3′ exoribonuclease 1 | XRN1 |
| IPI00022048 | Prostaglandin F2 receptor negative regulator precursor | PTGFRN |
| IPI00103055 | Isoform 1 of Solute carrier family 35 member F5 | SLC35F5 |
| IPI00167194 | CDNA FLJ25694 fis, clone TST04471 | FLJ25694 |
| IPI00152536 | Isoform 1 of Transmembrane channel-like protein 2 | TMC2 |
| IPI00783271 | Leucine-rich PPR motif-containing protein, mitochondrial precursor | LRPPRC |
| IPI00150057 | Isoform 2 of SWI/SNF-related matrix-associated actin-dependent | SMARCC2 |
| | regulator of chromatin subfamily C member 2 | |
| IPI00456996 | Conserved hypothetical protein | PVT1 |
| IPI00232891 | Isoform 3 of Fibronectin type III domain-containing protein 1 | FNDC1 |
| IPI00018402 | Tubulin-specific chaperone E | TBCE |
| IPI00030404 | Transcriptional repressor NF-X1 | NFX1 |
| IPI00219661 | Isoform 1 of Myelin proteolipid protein | PLP1 |
| IPI00186853 | pecanex-like 2 | PCNXL2 |
| IPI00395376 | Isoform 1 of Semaphorin-5B | SEMA5B |
| IPI00555621 | p21-activated kinase 2 variant (Fragment) | — |
| IPI00746232 | coiled-coil domain containing 112 isoform 2 | CCDC112 |
| IPI00022020 | Type II inositol-3,4-bisphosphate 4-phosphatase | INPP4B |
| IPI00418236 | Factor in the germline alpha | FIGLA |
| IPI00328762 | Isoform 1 of ATP-binding cassette sub-family A member 13 | ABCA13 |
| IPI00748342 | Beclin-1 | BECN1 |
| IPI00739423 | similar to c11.1 CG12132-PA isoform 11 | LOC377711 |
| IPI00026970 | FACT complex subunit SPT16 | SUPT16H |
| IPI00549384 | Isoform 1 of Bromodomain-containing protein 9 | BRD9 |
| IPI00479893 | Isoform 2 of Uncharacterized protein C10orf18 | C10orf18 |
| IPI00876910 | Isoform 1 of Ankyrin repeat and SOCS box-containing protein 18 | ASB18 |
| IPI00477766 | Isoform 3 of Protein PTHB1 | BBS9 |
| IPI00301503 | Isoform 1 of Splicing factor, arginine/serine-rich 10 | SFRS10 |
| IPI00425688 | Isoform 1 of RUN and SH3 domain-containing protein 1 | RUSC1 |
| IPI00787427 | ankyrin repeat domain 36 | ANKRD36 |
| IPI00167241 | Zinc finger protein 283 | ZNF283 |
| IPI00220325 | Isoform Short of Insulin receptor precursor | INSR |
| IPI00032541 | Keratin type II cuticular Hb5 | KRT85 |
| IPI00384874 | Isoform 1 of ATP-binding cassette sub-family A member 10 | ABCA10 |
| IPI00019690 | death-inducing-protein | DIP |
| IPI00027723 | Elastase-2B precursor | ELA2B |
| IPI00015737 | 15 kDa protein | DCAKD |
| IPI00641954 | 22 kDa protein | TMED5 |
| IPI00739676 | Isoform 2 of Protein DDX26B | DDX26B |
| IPI00402006 | Similar to DNA-binding protein | — |
| IPI00477949 | Isoform 1 of Zinc finger MYM-type protein 4 | ZMYM4 |
| IPI00737448 | similar to heat shock 70 kD protein binding protein | LOC338805 |
| IPI00383261 | Peroxin Pex6p | PEX6 |
| IPI00022735 | Synaptotagmin-4 | SYT4 |
| IPI00026058 | Isoform 1 of Leucine zipper putative tumor suppressor 1 | LZTS1 |
| IPI00479789 | Isoform 1 of Uncharacterized protein C1orf103 | C1orf103 |
| IPI00411637 | Galactose-3-O-sulfotransferase 4 | GAL3ST4 |
| IPI00154834 | Isoform 1 of Neighbor of punc e11 precursor | NOPE |
| IPI00007360 | Zinc finger protein 238 | ZNF238 |
| IPI00793585 | 57 kDa protein | OC90 |
| IPI00029133 | ATP synthase subunit b, mitochondrial precursor | ATP5F1 |
| IPI00218271 | Isoform 3 of MAGUK p55 subfamily member 2 | MPP2 |
| IPI00003949 | Ubiquitin-conjugating enzyme E2 N | UBE2N |
| IPI00304992 | Isoform 1 of Zinc finger protein 503 | ZNF503 |
| IPI00006612 | Isoform 1 of Clathrin coat assembly protein AP180 | SNAP91 |
| IPI00642305 | MCM8 protein | MCM8 |
| IPI00026320 | E3 ubiquitin-protein ligase UBR5 | UBR5 |
| IPI00010142 | Ubiquitin-conjugating enzyme E2 G2 | UBE2G2 |
| IPI00029769 | Isoform p59-HCK of Tyrosine-protein kinase HCK | HCK |
| IPI00446138 | CDNA FLJ42768 fis, clone BRAWH3003522 | — |
| IPI00449071 | Isoform 5a of Paired box protein Pax-6 | PAX6 |
| IPI00432893 | INPP5F protein (Fragment) | INPP5F |
| IPI00220039 | Isoform 1 of Transmembrane protein 16K | TMEM16K |
| IPI00000270 | Ribonuclease 7 precursor | RNASE7 |
| IPI00479430 | Isoform 2 of Complement C1q tumor necrosis factor-related protein 9 | C1QTNF9 |
| | precursor | |
| IPI00296798 | Isoform 1 of Fibrinogen C domain-containing protein 1 | FIBCD1 |
| IPI00003483 | Isoform 1 of Neuralized-like protein 1 | NEURL |
| IPI00419575 | Protein of unknown function DUF410 family protein | C7orf20 |
| IPI00019090 | Collagen alpha-1 | COL19A1 |
| IPI00011756 | Homeobox protein Meis1 | MEIS1 |
| IPI00012687 | 27 kDa protein | C1orf83 |
| IPI00332106 | Isoform 1 of Pre-B-cell leukemia transcription factor-interacting protein 1 | PBXIP1 |
| IPI00418426 | Metal transporter CNNM4 | CNNM4 |
| IPI00002524 | ATP-sensitive inward rectifier potassium channel 14 | KCNJ14 |
| IPI00000110 | Zinc finger protein 337 | ZNF337 |
| IPI00297767 | Casein kinase I isoform gamma-2 | CSNK1G2 |
| IPI00007207 | Isoform 1 of Lysosomal acid lipase/cholesteryl ester hydrolase precursor | LIPA |
| IPI00023868 | Canalicular multispecific organic anion transporter 1 | ABCC2 |
| IPI00031522 | Trifunctional enzyme subunit alpha, mitochondrial precursor | HADHA |
| IPI00329142 | Glutamate-rich protein 1 | ERICH1 |
| IPI00169253 | Olfactory receptor OR1-45 | OR5AT1 |
| IPI00045801 | Flt3-interacting zinc finger protein 1 | FIZ1 |
| IPI00017802 | Isoform 1 of Methylglutaconyl-CoA hydratase, mitochondrial precursor | AUH |
| IPI00384549 | TTC31 protein (Fragment) | TTC31 |
| IPI00012756 | Interferon-induced protein with tetratricopeptide repeats 5 | IFIT5 |
| IPI00297985 | Uncharacterized protein C6orf103 | C6orf103 |
| IPI00514394 | Family with sequence similarity 120A opposite strand | FAM120AOS |
| IPI00034088 | Interleukin-27 beta chain precursor | EBI3 |
| IPI00305856 | Homeobox protein Hox-D11 | HOXD11 |
| IPI00797859 | 10 kDa protein | LOC643711 |
| IPI00385737 | similar to Nonhistone chromosomal protein HMG-17 | LOC646853 |
| IPI00015195 | Cleavage stimulation factor 77 kDa subunit | CSTF3 |
| IPI00006158 | Lymphoid-restricted membrane protein | LRMP |
| IPI00008554 | Angiogenin precursor | ANG |
| IPI00028158 | Neurotensin/neuromedin N precursor | NTS |
| IPI00384393 | Myosin-reactive immunoglobulin heavy chain variable region | — |
| | (Fragment) | |
| IPI00021129 | Isoform 1 of AP-3 complex subunit beta-1 | AP3B1 |
| IPI00013871 | Ribonucleoside-diphosphate reductase large subunit | RRM1 |
| IPI00020039 | Cylicin-1 | CYLC1 |
| IPI00737712 | CCDC125 protein | CCDC125 |
| IPI00219036 | dicer1 | DICER1 |
| IPI00297212 | Paladin | KIAA1274 |
| IPI00418185 | Putative uncharacterized protein FLJ20097 | CCDC132 |
| IPI00423562 | Isoform 1 of Ubiquitin carboxyl-terminal hydrolase 6 | USP6 |
| IPI00008475 | Hydroxymethylglutaryl-CoA synthase, cytoplasmic | HMGCS1 |
| IPI00412107 | FLJ43980 protein | FLJ43980 |
| IPI00328793 | Sterol regulatory element-binding protein 2 | SREBF2 |
| IPI00004563 | Protein FAM105A | FAM105A |
| IPI00013257 | Single-stranded DNA-binding protein 4 | SSBP4 |
| IPI00158804 | basic, immunoglobulin-like variable motif containing | BIVM |
| IPI00166606 | KIAA0913 protein (Fragment) | KIAA0913 |
| IPI00329014 | Isoform 1 of Ankyrin repeat domain-containing protein 46 | ANKRD46 |
| IPI00395603 | Isoform 3 of Uncharacterized protein C18orf34 | C18orf34 |
| IPI00303980 | Isoform 1 of Uncharacterized protein C14orf93 precursor | C14orf93 |
| IPI00433499 | rhomboid, veinlet-like 6 isoform 1 | RHBDF2 |
| IPI00006196 | Isoform 2 of Nuclear mitotic apparatus protein 1 | NUMA1 |
| IPI00004446 | Sushi repeat-containing protein SRPX2 precursor | SRPX2 |
| IPI00010448 | Isoform 2 of Rho GTPase-activating protein 24 | ARHGAP24 |
| IPI00737392 | similar to proline-rich protein BstNI subfamily 3 precursor | LOC646352 |
| IPI00219503 | Isoform 1 of F-box only protein 24 | FBXO24 |
| IPI00414591 | Flavoprotein oxidoreductase | MICAL2 |
| IPI00018240 | Isoform 1 of Protein SDA1 homolog | SDAD1 |
| IPI00657892 | Factor VIII | F8 |
| IPI00386651 | LOC404266 protein | LOC404266 |
| IPI00166933 | Uncharacterized protein C10orf12 | C10orf12 |
| IPI00005254 | Isoform 1 of E3 ubiquitin-protein ligase parkin | PARK2 |
| IPI00065378 | Isoform 1 of DC-STAMP domain-containing protein 1 | DCST1 |
| IPI00163718 | Isoform 4 of Vacuolar protein sorting-associated protein 54 | VPS54 |
| IPI00414819 | Helicase SKI2W | SKIV2L |
| IPI00041588 | Ral guanine nucleotide exchange factor RalGPS1A | RALGPS1 |
| IPI00100787 | RANBP2-like and GRIP domain-containing protein 7 | RGPD5; RGPD7 |
| IPI00873759 | Uncharacterized protein ENSP00000381675 (Fragment) | — |
| IPI00012390 | Homeobox protein Hox-D4 | HOXD4 |
| IPI00027694 | Isoform 3 of Homeobox protein Hox-A1 | HOXA1 |
| IPI00747264 | Gamma-aminobutyric-acid receptor subunit rho-1 precursor | GABRR1 |
| IPI00337544 | Isoform 2 of Myomegalin | PDE4DIP |
| IPI00020795 | R33083_1 (Fragment) | DPP9 |
| IPI00010740 | Isoform Long of Splicing factor, proline- and glutamine-rich | SFPQ |
| IPI00019243 | Isoform Long of Matrix metalloproteinase-16 precursor | MMP16 |
| IPI00749338 | Hypothetical gene supported by BC063892 | hCG_1651889 |
| IPI00221009 | Isoform 7 of Transcription factor 7-like 2 | TCF7L2 |
| IPI00398725 | Isoform 1 of Zinc finger protein 644 | ZNF644 |
| IPI00745832 | IQ calmodulin-binding region domain containing protein | — |
| IPI00007067 | Golgi-associated plant pathogenesis-related protein 1 | C9orf19 |
| IPI00410616 | Isoform 1 of Endonuclease VIII-like 2 | NEIL2 |
| IPI00005978 | Splicing factor, arginine/serine-rich 2 | SFRS2 |
| IPI00844512 | RHOV protein (Fragment) | RHOV |
| IPI00879389 | 20 kDa protein | ZMYND11 |
| IPI00329251 | Isoform 1 of Transmembrane protein 146 precursor | TMEM146 |
| IPI00465087 | Ubiquitin carboxyl-terminal hydrolase BAP1 | BAP1 |
| IPI00103812 | PWWP domain-containing protein 2B | PWWP2B |
| IPI00010953 | CDNA FLJ11251 fis, clone PLACE1008813 | EXOC6 |
| IPI00639924 | 97 kDa protein | LOC374920 |
| IPI00292579 | Stabilin-2 precursor | STAB2 |
| IPI00067920 | FXYD domain-containing ion transport regulator 7 | FXYD7 |
| IPI00879162 | Uncharacterized protein ENSP00000374866 | — |
| IPI00009804 | DNA-binding protein SATB1 | SATB1 |
| IPI00215925 | Glycine N-methyltransferase | GNMT |
| IPI00006675 | Multidrug resistance-associated protein 4 | ABCC4 |
| IPI00442551 | CDNA FLJ26989 fis, clone SLV03395 | ATP9B |
| IPI00027436 | Tumor necrosis factor receptor superfamily member 16 precursor | NGFR |
| IPI00166190 | Isoform 1 of Uncharacterized protein C19orf19 | C19orf19 |
| IPI00154567 | Isoform 2 of Spermatogenesis-associated protein 20 precursor | SPATA20 |
| IPI00008495 | NADH-ubiquinone oxidoreductase chain 4 | MT-ND4 |
| IPI00179172 | Isoform 2 of Liprin-beta-1 | PPFIBP1 |
| IPI00872928 | Several ankyrin repeat protein transcript variant 2 (Fragment) | ANKRD42 |
| IPI00412647 | CDNA: FLJ21792 fis, clone HEP00441 | THADA |
| IPI00032929 | Isoform 1 of Thrombospondin type-1 domain-containing protein 1 | THSD1 |
| | precursor | |
| IPI00154451 | MMS19 nucleotide excision repair homolog | MMS19 |
| IPI00186145 | Isoform 1 of Protein phosphatase 1L | PPM1L |
| IPI00386364 | Olfactory receptor 2L2 | OR2L2 |
| IPI00218823 | Isoform 1 of WW domain-binding protein 7 | MLL4 |
| IPI00060379 | Integrator complex subunit 12 | INTS12 |
| IPI00444706 | CDNA FLJ45007 fis, clone BRAWH3012005 | KALRN |
| IPI00216710 | Isoform 1 of Nuclear receptor ROR-gamma | RORC |
| IPI00411690 | Isoform 3 of La-related protein 1 | LARP1 |
| IPI00295519 | Isoform 1 of Uncharacterized protein C3orf17 | C3orf17 |
| IPI00514153 | transmembrane and tetratricopeptide repeat containing 4 isoform 1 | TMTC4 |
| IPI00398728 | Isoform 1 of Retinitis pigmentosa 1-like 1 protein | RP1L1 |
| IPI00745775 | Similar to PR domain containing 4 | — |
| IPI00013880 | Semaphorin-5A precursor | SEMA5A |
| IPI00010157 | S-adenosylmethionine synthetase isoform type-2 | MAT2A |
| IPI00794307 | hypothetical protein | LOC729505 |
| IPI00015782 | UPF0171 protein C16orf35 | C16orf35 |
| IPI00011652 | Isoform Efs1 of Embryonal Fyn-associated substrate | EFS |
| IPI00413385 | Inter-alpha-trypsin inhibitor heavy chain H5-like protein precursor | ITIH5L |
| IPI00022334 | Ornithine aminotransferase, mitochondrial precursor | OAT |
| IPI00023617 | Zinc finger protein 197 | ZNF197 |
| IPI00642798 | Nucleolar autoantigen No55 | SC65 |
| IPI00383895 | CDNA FLJ40559 fis, clone THYMU2002910 | PRDM11 |
| IPI00296803 | 30 kDa protein | TCP11L2 |
| IPI00168056 | Zinc finger and BTB domain-containing protein 38 | ZBTB38 |
| IPI00026105 | Isoform SCPx of Non-specific lipid-transfer protein | SCP2 |
| IPI00385255 | Ig lambda chain V-II region NIG-84 | — |
| IPI00328178 | Isoform 3 of TOM1-like protein 2 | TOM1L2 |
| IPI00027144 | Cytochrome b561 | CYB561 |
| IPI00217688 | Uncharacterized protein C12orf60 | C12orf60 |
| IPI00220126 | Isoform EP2B of Sperm-associated antigen 11B precursor | SPAG11B |
| IPI00007757 | Isoform 1 of Zinc finger protein Helios | IKZF2 |
| IPI00328400 | Zygote arrest protein 1 | ZAR1 |
| IPI00465147 | Isoform 1 of Uncharacterized protein C9orf97 | C9orf97 |
| IPI00333016 | Isoform 3 of DnaJ homolog subfamily C member 11 | DNAJC11 |
| IPI00014897 | Isoform 2 of 1-phosphatidylinositol-4,5-bisphosphate phosphodiesterase | PLCB4 |
| | beta-4 | |
| IPI00181279 | hypothetical protein | LRRC37A4; LOC728863 |
| IPI00011894 | MRNA clone with similarity to L-glycerol-3-phosphate:NAD | — |
| | oxidoreductase and albumin gene sequences | |
| IPI00794248 | 47 kDa protein | — |
| IPI00514774 | Isoform 1 of PAB-dependent poly(A)-specific ribonuclease subunit 3 | PAN3 |
| IPI00307733 | Isoform 1 of Histone-lysine N-methyltransferase SETD2 | SETD2 |
| IPI00218896 | Alcohol dehydrogenase 1A | ADH1A |
| IPI00414973 | FAST kinase domain-containing protein 5 | FASTKD5 |
| IPI00002441 | Syndecan-1 precursor | SDC1 |
| IPI00413272 | Isoform 3 of Mediator of RNA polymerase II transcription subunit 23 | MED23 |
| IPI00299853 | Protocadherin beta 16 precursor | PCDHB16 |
| IPI00735857 | similar to ATP-binding cassette sub-family D member 1 | LOC651123 |
| IPI00647254 | 7 kDa protein | — |
| IPI00328118 | Sperm-associated antigen 5 | SPAG5 |
| IPI00094740 | Isoform 1 of RING finger protein 31 | RNF31 |
| IPI00383318 | PRO2272 | — |
| IPI00292470 | dapper, antagonist of beta-catenin, homolog 2 | DACT2 |
| IPI00215884 | Isoform ASF-1 of Splicing factor, arginine/serine-rich 1 | SFRS1 |
| IPI00013488 | Isoform Alpha-2 of N-chimaerin | CHN1 |
| IPI00030909 | BB1 family protein | — |
| IPI00552213 | Isoform 6 of Disks large homolog 1 | DLG1 |
| IPI00397358 | Similar to ribosomal protein S27 | — |
| IPI00021786 | RAF proto-oncogene serine/threonine-protein kinase | RAF1 |
| IPI00003438 | DnaJ homolog subfamily C member 8 | DNAJC8 |
| IPI00398900 | Homeobox protein HMX2 | HMX2 |
| IPI00031960 | polymerase (RNA) 1 polypeptide A, 194 kDa | POLR1A |
| IPI00298870 | Transmembrane protein 1 | TMEM1 |
| IPI00017991 | jumonji domain containing 2D | JMJD2D |
| IPI00151366 | Isoform TRPM6a of Transient receptor potential cation channel | TRPM6 |
| | subfamily M member 6 | |
| IPI00011488 | Isoform 1 of Serine/threonine-protein kinase 4 | STK4 |
| IPI00101405 | Farnesyl diphosphate synthase | FDPS |
| IPI00786867 | Dual Intracellular Von Willebrand factor domain A | DIVA |
| IPI00006904 | Cell death regulator Aven | AVEN |
| IPI00376213 | Isoform 1 of G-protein coupled receptor 120 | GPR120 |
| IPI00010420 | ADP/ATP translocase 4 | SLC25A31 |
| IPI00004419 | Zinc finger protein 646 | ZNF646 |
| IPI00847409 | hypothetical protein | LOC440338 |
| IPI00005024 | Isoform 1 of Myb-binding protein 1A | MYBBP1A |
| IPI00177890 | 54 kDa protein | SMARCD2 |
| IPI00023635 | Isoform 1 of Inositol monophosphatase 2 | IMPA2 |
| IPI00304885 | Centromere protein C 1 | CENPC1 |
| IPI00784013 | Janus kinase 1 | JAK1 |
| IPI00216139 | Isoform I of Septin-6 | SEPT6 |
| IPI00289954 | Heparan sulfate glucosamine 3-O-sulfotransferase 6 | HS3ST6 |
| IPI00018134 | Isoform 4 of Protein SOLO | FLJ10357 |
| IPI00855742 | sterile alpha motif domain containing 12 isoform a | SAMD12 |
| IPI00415037 | Isoform 12L of ADAM 12 precursor | ADAM12 |
| IPI00411298 | Connector enhancer of kinase suppressor of ras 3 | CNKSR3 |
| IPI00031288 | Claudin-14 | CLDN14 |
| IPI00867582 | Similar to SCAN domain-containing protein 2. Isoform 2 | ZNF496 |
| IPI00418885 | Epididymal-specific lipocalin-10 precursor | LCN10 |
| IPI00795992 | Hypothetical short protein | — |
| IPI00847373 | hypothetical protein LOC54627 | KIAA1383 |
| IPI00030070 | Gap junction beta-5 protein | GJB5 |
| IPI00418238 | Isoform 1 of HIV-1 Rev-binding protein-like protein | HRBL |
| IPI00293409 | Zinc finger protein 81 | ZNF81 |
| IPI00003927 | 40 kDa peptidyl-prolyl cis-trans isomerase | PPID |
| IPI00024801 | E3 ubiquitin-protein ligase NRDP1 | RNF41 |
| IPI00103356 | Uncharacterized protein ITGB2 | ITGB2 |
| IPI00604798 | Bitter taste receptor T2R2 (Fragment) | — |
| IPI00013887 | Beta-galactoside alpha-2,6-sialyltransferase 1 | ST6GAL1 |
| IPI00384971 | hypothetical protein LOC643155 | DKFZP686E2158 |
| IPI00025721 | COP9 signalosome complex subunit 3 | COPS3 |
| IPI00855747 | hypothetical protein LOC57481 | RP13- |
| | | 347D8.3 |
| IPI00445054 | CDNA FLJ44715 fis, clone BRACE3021430 | FLJ44715 |
| IPI00220503 | dynactin 2 | DCTN2 |
| IPI00328159 | Isoform 1 of Uncharacterized protein C1orf88 | C1orf88 |
| IPI00003881 | Heterogeneous nuclear ribonucleoprotein F | HNRPF |
| IPI00003990 | Isoform 2 of Valacyclovir hydrolase precursor | BPHL |
| IPI00847485 | Similar to PFIV protein | — |
| IPI00007300 | BTB/POZ domain-containing protein 3 | BTBD3 |
| IPI00217490 | Isoform 1 of Fibronectin type III domain-containing protein 3B | FNDC3B |
| IPI00848330 | Conserved hypothetical protein | LOC285144 |
| IPI00000787 | Isoform LMP2.L of Proteasome subunit beta type-9 precursor | PSMB9 |
| IPI00004379 | Homeobox protein HMX1 | HMX1 |
| IPI00375820 | Interferon-induced transmembrane protein 5 | IFITM5 |
| IPI00411635 | 180 kDa protein | — |
| IPI00795566 | 6 kDa protein | — |
| IPI00217117 | Isoform 1 of Glycerate kinase | GLYCTK |
| IPI00300086 | Nicotinate-nucleotide pyrophosphorylase | QPRT |
| IPI00235167 | Profilin-3 | PFN3 |
| IPI00026126 | Mammaglobin-B precursor | SCGB2A1 |
| IPI00220167 | Isoform 1 of Putative methyltransferase NSUN7 | NSUN7 |
| IPI00155168 | Leukocyte common antigen precursor (EC 3.1.3.48) (L-CA) (T200) | PTPRC |
| | (CD45 antigen). Isoform 2 | |
| IPI00384268 | XRCC6BP1 protein | XRCC6BP1 |
| IPI00299507 | Condensin complex subunit 2 | NCAPH |
| IPI00445643 | CDNA FLJ43684 fis, clone TBAES2001492 | — |
| IPI00165591 | Isoform 4 of Transmembrane channel-like protein 6 | TMC6 |
| IPI00152149 | CENP-B, N-terminal DNA-binding domain containing protein | — |
| IPI00029643 | Isoform 2 of Mitogen-activated srotein kinase kinase kinase MLT | ZAK |
| IPI00000875 | Elongation factor 1-gamma | EEF1G |
| IPI00215948 | Isoform 1 of Catenin alpha-1 | CTNNA1 |
| IPI00022918 | Isoform 2 of Protein FAM125B | FAM125B |
| IPI00007208 | Probable ATP-dependent RNA helicase DDX41 | DDX41 |
| IPI00855922 | Isoform 4 of DENN domain-containing protein 3 | DENND3 |
| IPI00031056 | Zinc finger MYND domain-containing protein 15 | ZMYND15 |
| IPI00641614 | 47 kDa protein | AKAP8L |
| IPI00002134 | 26S proteasome non-ATPase regulatory subunit 5 | PSMD5 |
| IPI00056040 | Neuritin 1-like | NRN1L |
| IPI00856045 | AHNAK nucleoprotein 2 | AHNAK2 |
| IPI00009329 | Utrophin | UTRN |
| IPI00218235 | dehydrogenase/reductase member 2 isoform 2 | DHRS2 |
| IPI00297315 | Alpha-2B adrenergic receptor | ADRA2B |
| IPI00107855 | Hephaestin precursor | HEPH |
| IPI00217662 | Uncharacterized protein C11orf35 | C11orf35 |
| IPI00025489 | Serine/threonine-protein kinase MAK | MAK |
| IPI00027774 | THAP domain-containing protein 2 | THAP2 |
| IPI00027415 | Isoform 1 of Probable ATP-dependent RNA helicase DHX36 | DHX36 |
| IPI00176125 | Collagen XXIX alpha 1 | COL29A1 |
| IPI00016046 | Protein MGR2 homolog | C20orf52 |
| IPI00002841 | Sodium/hydrogen exchanger 2 | SLC9A2 |
| IPI00472712 | Coiled-coil domain containing 7 | CCDC7 |
| IPI00061355 | Isoform 3 of Protein ZNF673 | ZNF673 |
| IPI00445804 | Uncharacterized protein ENSP00000374264 | DKFZp761B107 |
| IPI00790813 | 8 kDa protein | RAD51AP1 |
| IPI00061780 | Isoform 1 of E3 ubiquitin-protein ligase Itchy homolog | ITCH |
| IPI00030099 | Adenylate cyclase type 9 | ADCY9 |
| IPI00016095 | Transcription termination factor, mitochondrial precursor | MTERF |
| IPI00004521 | Ewing's tumor-associated antigen 1 | ETAA1 |
| IPI00171525 | Sentrin-specific protease 3 | SENP3 |
| IPI00373824 | Testis serine protease 1 precursor | TESSP1 |
| IPI00299729 | Transcobalamin-1 precursor | TCN1 |
| IPI00007444 | Epiphycan precursor | EPYC |
| IPI00167014 | EID2 protein (Fragment) | EID2 |
| IPI00007327 | Isoform 1 of Tapasin precursor | TAPBP |
| IPI00244346 | Troponin I, cardiac muscle | TNNI3 |
| IPI00009066 | similar to protein tyrosine phosphatase, receptor type, Q isoform 1 | PTPRQ |
| | precursor | |
| IPI00303195 | Syncoilin-1 | SYNC1 |
| IPI00031812 | Nuclease sensitive element-binding protein 1 | YBX1 |
| IPI00018854 | Coiled-coil domain-containing protein 68 | CCDC68 |
| IPI00084582 | Isoform 7 of Collagen alpha-1(XIII) chain | COL13A1 |
| IPI00216815 | Isoform 2 of DNA topoisomerase 3-beta-1 | TOP3B |
| IPI00062599 | 71 kDa protein | — |
| IPI00060549 | G-protein coupled receptor-associated sorting protein 1 | GPRASP1 |
| IPI00795943 | 7 kDa protein | — |
| IPI00642204 | Thioredoxin domain-containing protein 16 precursor | TXNDC16 |
| IPI00646199 | Zinc finger, MYM-type 2 | ZMYM2 |
| IPI00791447 | Pseudogene candidate | LOC339929 |
| IPI00745433 | eukaryotic translation initiation factor 2C, 2 | EIF2C2 |
| IPI00103090 | Isoform 1 of RNA polymerase-associated protein LEO1 | LEO1 |
| IPI00101186 | Isoform 1 of RRP12-like protein | RRP12 |
| IPI00013404 | Mothers against decapentaplegic homolog 4 | SMAD4 |
| IPI00220038 | Isoform B of Arsenite-resistance protein 2 | ARS2 |
| IPI00385683 | Ig heavy chain V-III region GAR | — |
| IPI00006030 | Dynein intermediate chain 1, axonemal | DNAI1 |
| IPI00024714 | Isoform 1 of Regulator of G-protein signaling 12 | RGS12 |
| IPI00259671 | Taste receptor type 2 member 44 | TAS2R44 |
| IPI00335581 | Isoform 1 of E3 ubiquitin-protein ligase UBR3 | UBR3 |
| IPI00004416 | Charged multivesicular body protein 2a | CHMP2A |
| IPI00028828 | Isoform Beta of Zinc finger protein 202 | ZNF202 |
| IPI00514795 | Isoform 1 of Tubulin--tyrosine ligase-like protein 7 | TTLL7 |
| IPI00101987 | Uncharacterized protein C19orf62 | C19orf62 |
| IPI00853224 | START domain containing 7 | STARD7 |
| IPI00001141 | Mitochondrial import inner membrane translocase subunit Tim22 | TIMM22 |
| IPI00514975 | Chromosome 1 open reading frame 133 | C1orf133 |
| IPI00744029 | Similar to Zinc finger, FYVE domain containing 21 | — |
| IPI00399320 | Transmembrane protein 59 precursor | TMEM59 |
| IPI00217687 | Isoform 1 of Coiled-coil domain-containing protein 135 | CCDC135 |
| IPI00829745 | Cellular titin isoform PEVK variant 2 (Fragment) | — |
| IPI00216544 | Isoform 2 of Protein hairless | HR |
| IPI00646323 | Isoform 1 of Uncharacterized protein C10orf76 | C10orf76 |
| IPI00386797 | Isoform 1 of Folliculin-interacting protein 1 | FNIP1 |
| IPI00304589 | Isoform 1 of 182 kDa tankyrase 1-binding protein | TNKS1BP1 |
| IPI00002857 | Isoform CSBP2 of Mitogen-activated protein kinase 14 | MAPK14 |
| IPI00017630 | Nuclear fragile X mental retardation-interacting protein 1 | NUFIP1 |
| IPI00019992 | Myb-related protein A | MYBL1 |
| IPI00168848 | UDP-GlcNAc:betaGal beta-1,3-N-acetylglucosaminyltransferase 7 | B3GNT7 |
| IPI00216917 | Isoform 1 of Uncharacterized protein C22orf9 | C22orf9 |
| IPI00043499 | Probable urocanate hydratase | UROC1 |
| IPI00056507 | Isoform 1 of F-box/WD repeat-containing protein 5 | FBXW5 |
| IPI00017451 | Splicing factor 3 subunit 1 | SF3A1 |
| IPI00465273 | UHRF1-binding protein 1 | UHRF1BP1 |
| IPI00738999 | similar to ubiquitin-conjugating enzyme E2 variant 1 | LOC643227 |
| IPI00031047 | CDNA: FLJ23571 fis, clone LNG12303 | IQCG |
| IPI00022256 | AP-2 complex subunit mu-1 | AP2M1 |
| IPI00151141 | Isoform 1 of Serine/threonine-protein kinase WNK4 | WNK4 |
| IPI00027232 | Insulin-like growth factor 1 receptor precursor | IGF1R |
| IPI00328748 | Protein ARMET precursor | ARMET |
| IPI00553006 | Probable prolyl-tRNA synthetase, mitochondrial precursor | PARS2 |
| IPI00102803 | Ankyrin repeat domain-containing protein 22 | ANKRD22 |
| IPI00030153 | Tudor domain-containing protein 6 | TDRD6 |
| IPI00010212 | U6 snRNA-specific terminal uridylyltransferase 1 | TUT1 |
| IPI00844501 | MG44 protein | SUV39H1 |
| IPI00456060 | Protein | — |
| IPI00016480 | Isoform 1 of NACHT, LRR and PYD domains-containing protein 2 | NLRP2 |
| IPI00411545 | Isoform 3 of Thrombopoietin precursor | THPO |
| IPI00168239 | Zinc finger protein GLIS1 | GLIS1 |
| IPI00165990 | Isoform 4 of Transcription factor RFX4 | RFX4 |
| IPI00749489 | fibrocystin L | PKHD1L1 |
| IPI00059144 | Protein FAM46B | FAM46B |
| IPI00787697 | similar to Oligophrenin 1 isoform 4 | FLJ32810 |
| IPI00043237 | CDNA FLJ32095 fis, clone OCBBF2000998 | LOC730193; STX6 |
| IPI00028641 | Isoform 4 of Protein phosphatase 1 regulatory subunit 12B | PPP1R12B |
| IPI00879159 | 9 kDa protein | — |
| IPI00018099 | E3 ubiquitin-protein ligase FANCL | FANCL |
| IPI00455076 | Double C2-like domain-containing protein alpha | DOC2A |
| IPI00295940 | Protein unc-84 homolog B | UNC84B |
| IPI00002970 | Beta-sarcoglycan | SGCB |
| IPI00878142 | 68 kDa protein | — |
| IPI00554488 | cAMP-dependent protein kinase type I-beta regulatory subunit | PRKAR1B |
| IPI00030279 | Isoform 1 of Zinc finger Ran-binding domain-containing protein 3 | ZRANB3 |
| IPI00074656 | Isoform 1 of Coiled-coil domain-containing protein C6orf65 | C6orf65 |
| IPI00014903 | Isoform 1 of FGFR1 oncogene partner 2 | FGFR1OP2 |
| IPI00011961 | Isoform 1 of Sialoadhesin precursor | SIGLEC1 |
| IPI00170770 | Isoform 1 of PHD finger protein 3 | PHF3 |
| IPI00000057 | Conserved oligomeric Golgi complex component 2 | COG2 |
| IPI00435928 | PP13187 | RASGRF1 |
| IPI00013721 | Serine/threonine-protein kinase PRP4 homolog | PRPF4B |
| IPI00748607 | Conserved hypothetical protein | — |
| IPI00021266 | 60S ribosomal protein L23a | RPL23A; hCG_16001 |
| IPI00031228 | Isoform 2 of Pleckstrin homology domain-containing family B member 2 | PLEKHB2 |
| IPI00337415 | Guanine nucleotide-binding protein G(i), alpha-1 subunit | GNAI1 |
| IPI00745396 | Stromal RNA regulating factor | HNRPLL |
| IPI00604752 | Isoform 3 of Sterile alpha motif domain-containing protein 13 | SAMD13 |
| IPI00382534 | Ig heavy chain V-II region OU | — |
| IPI00008444 | Isoform 1 of Cytidine and dCMP deaminase domain-containing protein 1 | CDADC1 |
| IPI00792933 | zinc finger protein 462 | ZNF462 |
| IPI00402005 | Similar to DNA-binding protein | ZNF528 |
| IPI00020036 | Neuronal acetylcholine receptor subunit alpha-4 precursor | CHRNA4 |
| IPI00329054 | Osteopetrosis-associated transmembrane protein 1 precursor | OSTM1 |
| IPI00028864 | Neurexophilin-3 precursor | NXPH3 |
| IPI00023757 | Isoform 1 of X-linked retinitis pigmentosa GTPase regulator | RPGR |
| IPI00000663 | Isoform Mitochondrial of Malonyl-CoA decarboxylase, mitochondrial | |
| | precursor | MLYCD |
| IPI00739387 | hypothetical protein LOC401478 | FLJ45872 |
| IPI00164352 | zinc finger protein 292 | ZNF292 |
| IPI00008913 | Alpha-N-acetylgalactosaminide alpha-2,6-sialyltransferase 5 | ST6GALNAC 5 |
| IPI00020454 | Deoxycytidine kinase | DCK |
| IPI00514028 | 21 kDa protein | RNF207 |
| IPI00024292 | Low-density lipoprotein receptor-related protein 2 precursor | LRP2 |
| IPI00028357 | Exportin-4 | XPO4 |
| IPI00438355 | Fibrillin-3 precursor | FBN3 |
| IPI00009043 | Apoptotic-related protein PCAR (Fragment) | — |
| IPI00027442 | Alanyl-tRNA synthetase, cytoplasmic | AARS |
| IPI00014845 | Isoform 1 of Ciliary dynein heavy chain 8 | DNAH8 |
| IPI00385687 | similar to Ubiquinol-cytochrome c reductase complex 14 kDa protein | — |
| IPI00794829 | 16 kDa protein | ACADS |
| IPI00216423 | Isoform 4 of Intersectin-2 | ITSN2 |
| IPI00303870 | Seven transmembrane helix receptor | — |
| IPI00848229 | EGF, latrophilin and seven transmembrane domain-containing protein 1 | ELTD1 |
| | precursor | |
| IPI00293857 | Isoform 1A of Beta-arrestin-1 | ARRB1 |
| IPI00293565 | fms-related tyrosine kinase 4 isoform 1 | FLT4 |
| IPI00022471 | minor histocompatibility antigen HA-1 | HMHA1 |
| IPI00848252 | hypothetical protein LOC285501 | LOC285501 |
| IPI00288939 | Isoform 3 of Coiled-coil domain-containing protein 40 | CCDC40 |
| IPI00065057 | Putative uncharacterized protein CXorf58 | CXorf58 |
| IPI00073769 | 48 kDa protein | PALM2 |
| IPI00065520 | Isoform 1 of Protein phosphatase 1M | PPM1M |
| IPI00436518 | Benzodiazepine receptor ligand | — |
| IPI00015345 | Cadherin EGF LAG seven-pass G-type receptor 3 precursor | CELSR3; SLC26A6 |
| IPI00847391 | Isoform 1 of Zinc finger protein 276 | ZNF276 |
| IPI00001654 | Isoform 3 of Pericentriolar material 1 protein | PCM1 |
| IPI00028493 | Isoform 1 of Tuberin | TSC2 |
| IPI00332583 | chorionic somatomammotropin hormone 1 isoform 2 | CSH1; CSH2; |
| | | GH1 |
| IPI00301844 | Zinc fingers and homeoboxes protein 3 | ZHX3 |
| IPI00013788 | HIV Tat-specific factor 1 | HTATSF1 |
| IPI00001151 | Isoform 2 of Uncharacterized MFS-type transporter C20orf59 | C20orf59 |
| IPI00847237 | Conserved hypothetical protein | — |
| IPI00295999 | Isoform 2 of Collagen alpha-1(IX) chain precursor | COL9A1 |
| IPI00296196 | Dimethylglycine dehydrogenase, mitochondrial precursor | DMGDH |
| IPI00396145 | FYVE finger-containing phosphoinositide kinase | PIP5K3 |
| IPI00478110 | Probable G-protein coupled receptor 135 | GPR135 |
| IPI00168995 | Olfactory receptor 4D9 | OR4D9 |
| IPI00059190 | Isoform 1 of SLAM family member 9 precursor | SLAMF9 |
| IPI00043467 | Tigger transposable element-derived protein 1 | TIGD1 |
| IPI00235842 | Zinc finger protein 483 | ZNF483 |
| IPI00220473 | Isoform 2 of Calcium-transporting ATPase type 2C member 1 | ATP2C1 |
| IPI00382831 | Cerebral protein-13 | BAP1 |
| IPI00477692 | Uncharacterized protein ENSP00000330808 | LOC131149 |
| IPI00301583 | Isoform 1 of Zinc finger protein 691 | ZNF691 |
| IPI00790447 | 12 kDa protein | — |
| IPI00829904 | Uncharacterized protein ENSP00000374814 | — |
| IPI00020533 | Protein BEX1 | BEX1 |
| IPI00376436 | Isoform 4 of Vacuolar protein sorting-associated protein 13B | VPS13B |
| IPI00029162 | Isoform 2 of Cell division cycle 2-like protein kinase 5 | CDC2L5 |
| IPI00065500 | BRO1 domain-containing protein BROX | C1orf58 |
| IPI00740336 | formin 1 | FMN1 |
| IPI00017203 | Protein RMI1 homolog | RMI1 |
| IPI00293431 | WUGSC:H_DJ0855D21.2 protein | MGC70863 |
| IPI00295400 | Tryptophanyl-tRNA synthetase, cytoplasmic | WARS |
| IPI00444330 | CDNA FLJ45693 fis, clone FEBRA2012625 | — |
| IPI00170509 | Isoform 3 of Calcium/calmodulin-dependent protein kinase kinase 2 | CAMKK2 |
| IPI00017533 | Cytochrome c oxidase subunit 3 | MT-CO3 |
| IPI00419922 | Isoform 1 of IQ domain-containing protein E | IQCE |
| IPI00005710 | Heparan sulfate glucosamine 3-O-sulfotransferase 2 | HS3ST2 |
| IPI00744872 | Isoform 1 of Uncharacterized protein KIAA0423 | KIAA0423 |
| IPI00787412 | similar to Protein C11orf2 | LOC441616 |
| IPI00168607 | tetratricopeptide repeat domain 21A isoform 1 | TTC21A |
| IPI00419385 | olfactory receptor, family 52, subfamily E, member 8 | OR52E8 |
| IPI00006236 | Platelet-derived growth factor receptor-like protein precursor | PDGFRL |
| IPI00291796 | BTB (POZ) domain containing 12 | BTBD12 |
| IPI00400795 | 54 kDa protein | — |
| IPI00031554 | ATP-dependent RNA helicase DDX50 | DDX50 |
| IPI00023860 | Nucleosome assembly protein 1-like 1 | NAP1L1 |
| IPI00375225 | Olfactory receptor 2AE1 | OR2AE1 |
| IPI00235622 | Isoform 3 of CUB domain-containing protein 1 precursor | CDCP1 |
| IPI00742031 | similar to golgi autoantigen, golgin subfamily a, 8G | LOC645137 |
| IPI00221372 | Isoform 2 of 1-acyl-sn-glycerol-3-phosphate acyltransferase beta | AGPAT2 |
| IPI00796972 | 62 kDa protein | ETV5 |
| IPI00010807 | Frizzled-8 precursor | FZD8 |
| IPI00031410 | FKBP12-rapamycin complex-associated protein | FRAP1 |
| IPI00019848 | Isoform 1 of Host cell factor | HCFC1 |
| IPI00306611 | TRAF-interacting protein | TRAIP |
| IPI00153031 | Isoform 1 of Uncharacterized protein C17orf80 | C17orf80 |
| IPI00289097 | Isoform 1 of Ras-GEF domain-containing family member 1B | RASGEF1B |
| IPI00425404 | Isoform 1 of Kinesin-like protein KIF21A | KIF21A |
| IPI00477262 | Uncharacterized protein TRIM41 (Fragment) | TRIM41 |
| IPI00030703 | Motor neuron and pancreas homeobox protein 1 | MNX1 |
| IPI00025974 | Charged multivesicular body protein 4b | CHMP4B |
| IPI00413655 | ankyrin repeat domain 55 isoform 1 | ANKRD55 |
| IPI00026497 | Isoform 1 of Poly [ADP-ribose] polymerase 2 | PARP2 |
| IPI00061206 | Polyadenylate-binding protein 5 | PABPC5 |
| IPI00289758 | Calpain-2 catalytic subunit precursor | CAPN2 |
| IPI00465177 | Transforming growth factor-alpha variant I | TGFA |
| IPI00180305 | Isoform 5 of E3 ubiquitin-protein ligase UBR4 | UBR4 |
| IPI00019244 | Isoform 1 of RNA-binding protein Nova-1 | NOVA1 |
| IPI00009101 | Isoform 2 of Helicase SRCAP | SRCAP |
| IPI00782992 | Isoform 1 of Serine/arginine repetitive matrix protein 2 | SRRM2 |
| IPI00807675 | ZNF440 protein | ZNF440 |
| IPI00104050 | Thyroid hormone receptor-associated protein 3 | THRAP3 |
| IPI00411480 | hypothetical protein | LOC729461 |
| IPI00646423 | Isoform 2 of Transcription initiation factor TFIID subunit 4B | TAF4B |
| IPI00446312 | CDNA FLJ42271 fis, clone TKIDN2015788 | — |
| IPI00179298 | 482 kDa protein | HUWE1 |
| IPI00016006 | Isoform 1 of Gephyrin | GPHN |
| IPI00395630 | Putative uncharacterized protein pp6170 | SCRIB |
| IPI00100067 | Isoform 1 of Tumor necrosis factor receptor superfamily member 19 | TNFRSF19 |
| | precursor | |
| IPI00789476 | 7 kDa protein | — |
| IPI00306749 | Kanadaptin | SLC4A1AP |
| IPI00001922 | Suppressor of tumorigenicity protein 14 | ST14 |
| IPI00464980 | Isoform 1 of Paired amphipathic helix protein Sin3b | SIN3B |
| IPI00744932 | Conserved hypothetical protein | ZDHHC1 |
| IPI00445750 | CDNA FLJ43142 fis, clone CTONG3007870 | — |
| IPI00006077 | Protein Jade-3 | PHF16 |
| IPI00061148 | Sodium bicarbonate cotransporter NBC4d | SLC4A5 |
| IPI00004944 | Isoform 1 of Sodium-driven chloride bicarbonate exchanger | SLC4A10 |
| IPI00304587 | Protein dispatched homolog 2 | DISP2 |
| IPI00219703 | Parvalbumin alpha | PVALB |
| IPI00656052 | SPANX-C | SPANXB2; SPANXB1; |
| | | SPANXF1 |
| IPI00413266 | Isoform 1 of IQ and AAA domain-containing protein | IQCA |
| IPI00472374 | zinc finger protein 98 | ZNF98 |

TABLE 8l
Table 8l. Proteins Absent in Neurologic Lyme disease but
Present in Normals or Chronic Fatigue. 1597 Proteins

| IPI | Protein | Gene |
|---|---|---|
| IPI00216704 | Isoform 2 of Spectrin beta chain, erythrocyte | SPTB |
| IPI00022361 | Band 3 anion transport protein | SLC4A1 |
| IPI00003921 | Isoform 1 of Protein 4.1 | EPB41 |
| IPI00014592 | Chondroadherin precursor | CHAD |
| IPI00028614 | erythrocyte membrane protein band 4.2 isoform 2 | EPB42 |
| IPI00246058 | PDCD6IP protein | PDCD6IP |
| IPI00027462 | Protein S100-A9 | S100A9 |
| IPI00019502 | Myosin-9 | MYH9 |
| IPI00005981 | Transgelin-3 | TAGLN3 |
| IPI00024094 | Rhesus blood group-associated glycoprotein | RHAG |
| IPI00251507 | Isoform IB of Synapsin-1 | SYN1 |
| IPI00218345 | Isoform 2 of Tubulin alpha-3C/D chain | TUBA3C; TUBA3D; LOC654264 |
| IPI00023576 | Leucine-rich repeat transmembrane neuronal protein 2 precursor | LRRTM2 |
| IPI00871227 | Isoform 1 of Hemicentin-1 precursor | HMCN1 |
| IPI00005652 | Isoform 1 of WSC domain-containing protein 2 | WSCD2 |
| IPI00217466 | Histone H1.3 | HIST1H1D |
| IPI00031789 | Isoform 1 of Interleukin-1 receptor accessory protein precursor | IL1RAP |
| IPI00007102 | Uncharacterized protein C17orf25 | GLOD4 |
| IPI00257508 | Dihydropyrimidinase-related protein 2 | DPYSL2 |
| IPI00168520 | Isoform 2 of Matrilin-2 precursor | MATN2 |
| IPI00410585 | Isoform 1 of Crumbs homolog 2 precursor | CRB2 |
| IPI00217465 | Histone H1.2 | HIST1H1C |
| IPI00217467 | Histone H1.4 | HIST1H1E |
| IPI00007709 | Isoform 1 of ADAM 28 precursor | ADAM28 |
| IPI00219930 | Cellular retinoic acid-binding protein 1 | CRABP1 |
| IPI00302944 | Isoform 4 of Collagen alpha-1(XII) chain precursor | COL12A1 |
| IPI00017567 | Isoform Long of Endoglin precursor | ENG |
| IPI00005809 | Serum deprivation-response protein | SDPR |
| IPI00022774 | Transitional endoplasmic reticulum ATPase | VCP |
| IPI00028082 | Reversion-inducing cysteine-rich protein with Kazal motifs precursor | RECK |
| IPI00219910 | 22 kDa protein | — |
| IPI00012510 | EMILIN-2 precursor | EMILIN2 |
| IPI00029928 | Elastin | ELN |
| IPI00472754 | Polycystic kidney disease 1-related protein | KIAA0319L |
| IPI00441344 | Beta-galactosidase precursor | GLB1 |
| IPI00396378 | Isoform B1 of Heterogeneous nuclear ribonucleoproteins A2/B1 | HNRNPA2B1 |
| IPI00411656 | Isoform 1 of Protein piccolo | PCLO |
| IPI00021347 | Ubiquitin-conjugating enzyme E2 L3 | UBE2L3 |
| IPI00007047 | Protein S100-A8 | S100A8 |
| IPI00024689 | Aquaporin-1 | AQP1 |
| IPI00025812 | Carbonic anhydrase-related protein 11 precursor | CA11 |
| IPI00646689 | Thioredoxin domain-containing protein 17 | TXNDC17 |
| IPI00220281 | Guanine nucleotide-binding protein G(o) subunit alpha 1 | GNAO1 |
| IPI00024067 | Isoform 1 of Clathrin heavy chain 1 | CLTC |
| IPI00217005 | Ankyrin repeat domain-containing protein 18A | ANKRD18A |
| IPI00294713 | Isoform 1 of Mannan-binding lectin serine protease 2 precursor | MASP2 |
| IPI00004488 | Vacuolar proton pump subunit F | ATP6V1F |
| IPI00007812 | Vacuolar ATP synthase subunit B, brain isoform | ATP6V1B2 |
| IPI00219806 | Protein S100-A7 | S100A7 |
| IPI00011865 | Isoform 2 of Platelet-derived growth factor D precursor | PDGFD |
| IPI00009439 | Synaptotagmin-1 | SYT1 |
| IPI00219682 | Erythrocyte band 7 integral membrane protein | STOM |
| IPI00004047 | Isoform 1 of Exostosin-2 | EXT2 |
| IPI00384016 | Full-length cDNA 5-PRIME end of clone CS0DJ009YL13 of T cells | DLST |
| | (Jurkat cell line) of Homo sapiens (Fragment) | |
| IPI00152524 | Isoform 3 of Neuropilin and tolloid-like protein 1 precursor | NETO1 |
| IPI00552943 | V1-11 protein | IGLV1-36 |
| IPI00031718 | Isoform 1 of Ectonucleoside triphosphate diphosphohydrolase 4 | ENTPD4 |
| IPI00300623 | Pro-MCH precursor | PMCH |
| IPI00008756 | Isoform 1 of Bullous pemphigoid antigen 1, isoforms 1/2/3/4/5/8 | DST |
| | (Fragment) | |
| IPI00002511 | Cyclic AMP-dependent transcription factor ATF-6 alpha | ATF6 |
| IPI00216699 | Isoform 2 of Unc-112-related protein 2 | FERMT3 |
| IPI00023942 | Isoform 2 of Syndecan-3 | SDC3 |
| IPI00009396 | Isoform 1 of Cannabinoid receptor 1 | CNR1 |
| IPI00426727 | Isoform 1 of Methyl-CpG-binding domain protein 4 | MBD4 |
| IPI00018879 | Alpha-L-iduronidase precursor | IDUA |
| IPI00788258 | similar to lysyl oxidase-like 1 preproprotein | LOXL1 |
| IPI00019904 | Isoform 1 of Beta-adducin | ADD2 |
| IPI00020407 | Alpha-1,6-mannosylglycoprotein 6-beta-N- | MGAT5 |
| | acetylglucosaminyltransferase A | |
| IPI00295767 | Noelin-2 precursor | OLFM2 |
| IPI00216694 | plastin 3 | PLS3 |
| IPI00306844 | Corticotropin-releasing factor-binding protein precursor | CRHBP |
| IPI00024580 | Methylcrotonoyl-CoA carboxylase subunit alpha, mitochondrial | MCCC1 |
| | precursor | |
| IPI00023191 | Target of myb1 | TOM1 |
| IPI00005690 | Matrilin-3 precursor | MATN3 |
| IPI00465261 | Isoform 1 of Endoplasmic reticulum aminopeptidase 2 | ERAP2 |
| IPI00026546 | Platelet-activating factor acetylhydrolase IB subunit beta | PAFAH1B2 |
| IPI00061354 | Isoform 2 of Bromodomain adjacent to zinc finger domain protein 2B | BAZ2B |
| IPI00297779 | T-complex protein 1 subunit beta | CCT2 |
| IPI00168920 | collagen, type XXIV, alpha 1 | COL24A1 |
| IPI00003441 | Isoform 1 of Protein C1orf9 precursor | C1orf9 |
| IPI00020692 | Isoform 1 of Sodium channel protein type 3 subunit alpha | SCN3A |
| IPI00289862 | Secernin-1 | SCRN1 |
| IPI00013945 | Isoform 1 of Uromodulin precursor | UMOD |
| IPI00242905 | Uncharacterized protein ENSP00000344689 (Fragment) | — |
| IPI00554752 | cAMP-dependent protein kinase type II-beta regulatory subunit | PRKAR2B |
| IPI00293460 | ATP-binding cassette sub-family A member 1 | ABCA1 |
| IPI00002966 | Heat shock 70 kDa protein 4 | HSPA4 |
| IPI00298476 | Isoform 1 of Gremlin-1 precursor | GREM1 |
| IPI00008202 | Headcase protein homolog | HECA |
| IPI00306576 | Arylsulfatase B precursor | ARSB |
| IPI00003391 | Teneurin-1 | ODZ1 |
| IPI00178727 | Novel protein | C10orf79 |
| IPI00029623 | Proteasome subunit alpha type-6 | PSMA6 |
| IPI00171199 | Isoform 2 of Proteasome subunit alpha type-3 | PSMA3 |
| IPI00020884 | Plexin-A3 precursor | PLXNA3 |
| IPI00001872 | Isoform 1 of Protocadherin gamma C3 precursor | PCDHGC3; PCDHGA12 |
| IPI00175989 | Rho family guanine-nucleotide exchange factor | MCF2L2 |
| IPI00294619 | Protein TFG | TFG |
| IPI00100154 | Toll-interacting protein | TOLLIP |
| IPI00024976 | Mitochondrial import receptor subunit TOM22 homolog | TOMM22 |
| IPI00012315 | Nucleoside diphosphate kinase 3 | NME3 |
| IPI00879950 | 15 kDa protein | — |
| IPI00454695 | Histone H2B type 2-C | HIST2H2BC |
| IPI00216348 | Isoform 2C of Cytoplasmic dynein 1 intermediate chain 2 | DYNC1I2 |
| IPI00328826 | Uncharacterized protein CADPS2 | CADPS2 |
| IPI00396961 | Leucine-rich repeat and fibronectin type-III domain-containing | LRFN5 |
| | protein 5 precursor | |
| IPI00026031 | Uncharacterized protein C6orf72 precursor | C6orf72 |
| IPI00305833 | Smu-1 suppressor of mec-8 and unc-52 protein homolog | SMU1 |
| IPI00550746 | Nuclear migration protein nudC | NUDC |
| IPI00299116 | Podocalyxin-like protein 1 precursor | PODXL |
| IPI00181743 | Isoform 1 of BAI1-associated protein 3 | BAIAP3 |
| IPI00031907 | Isoform 1 of Transmembrane protein 108 precursor | TMEM108 |
| IPI00749514 | attractin-like 1 | ATRNL1 |
| IPI00024704 | Uronyl 2-sulfotransferase | UST |
| IPI00419237 | Isoform 1 of Cytosol aminopeptidase | LAP3 |
| IPI00021695 | Isoform D of Plasma membrane calcium-transporting ATPase 1 | ATP2B1 |
| IPI00480159 | inositol polyphosphate-5-phosphatase F | INPP5F |
| IPI00019901 | Isoform 1 of Alpha-adducin | ADD1 |
| IPI00017163 | Isoform 1 of E3 ubiquitin-protein ligase HECW2 | HECW2 |
| IPI00027464 | Calcineurin subunit B isoform 1 | PPP3R1 |
| IPI00001568 | Vacuolar proton pump subunit D | ATP6V1D |
| IPI00005776 | Nucleotide-binding oligomerization domain-containing protein 1 | NOD1 |
| IPI00432707 | Caspase-12 | CASP12 |
| IPI00106502 | Kelch-like ECH-associated protein 1 | KEAP1 |
| IPI00746177 | similar to Tubulin alpha-2 chain | LOC730222 |
| IPI00013272 | Isoform 1 of Golgin subfamily A member 4 | GOLGA4 |
| IPI00043201 | Centromere protein J | CENPJ |
| IPI00642259 | Dystonin | DST |
| IPI00879575 | 71 kDa protein | — |
| IPI00004315 | Sialic acid-binding Ig-like lectin 9 precursor | SIGLEC9 |
| IPI00005159 | Actin-related protein 2 | ACTR2 |
| IPI00001755 | Glypican-6 precursor | GPC6 |
| IPI00604430 | Isoform 2 of Receptor expression-enhancing protein 2 | REEP2 |
| IPI00023315 | Bone morphogenetic protein 3b precursor | GDF10 |
| IPI00219622 | Proteasome subunit alpha type-2 | PSMA2 |
| IPI00328361 | Seryl-tRNA synthetase, mitochondrial precursor | SARS2 |
| IPI00027248 | Tumor suppressor candidate 2 | TUSC2 |
| IPI00021364 | Properdin precursor | CFP |
| IPI00307612 | Cadherin-20 precursor | CDH20 |
| IPI00383594 | melanoma ubiquitous mutated protein | MUM1 |
| IPI00013281 | Fukutin-related protein | FKRP |
| IPI00013978 | Speckle-type POZ protein | SPOP |
| IPI00335946 | Family with sequence similarity 120B | FAM120B |
| IPI00033600 | Isoform 1 of Protein phosphatase 1 regulatory subunit 7 | PPP1R7 |
| IPI00216774 | Cerebellin-2 | CBLN2 |
| IPI00152769 | Isoform 1 of Trpc4-associated protein | TRPC4AP |
| IPI00647217 | Superkiller viralicidic activity 2-like 2 | SKIV2L2 |
| IPI00027438 | Flotillin-1 | FLOT1 |
| IPI00013933 | Isoform DPI of Desmoplakin | DSP |
| IPI00003406 | Isoform 1 of Drebrin | DBN1 |
| IPI00744811 | Low-density lipoprotein receptor-related protein 5 precursor | LRP5 |
| IPI00465178 | Isoform 1 of Vacuolar proton translocating ATPase 116 kDa subunit a | ATP6V0A1 |
| | isoform 1 | |
| IPI00018275 | Prion-like protein doppel precursor | PRND |
| IPI00046057 | Isoform 2 of Syntaxin-binding protein 1 | STXBP1 |
| IPI00008580 | Antileukoproteinase precursor | SLPI |
| IPI00003348 | Guanine nucleotide-binding protein G(I)/G(S)/G(T) subunit beta-2 | GNB2 |
| IPI00008998 | Protein tyrosine phosphatase-like protein PTPLAD1 | PTPLAD1 |
| IPI00847759 | DENN domain-containing protein 4B | DENND4B |
| IPI00294910 | Protein PARM-1 precursor | DKFZP564O0823 |
| IPI00008433 | 40S ribosomal protein S5 | RPS5 |
| IPI00827745 | Isoform 1 of RNA-binding protein 24 | RBM24 |
| IPI00009532 | 4-aminobutyrate aminotransferase, mitochondrial precursor | ABAT |
| IPI00030876 | diaphanous 1 isoform 2 | DIAPH1 |
| IPI00145805 | Isoform 1 of TRAF2 and NCK-interacting protein kinase | TNIK |
| IPI00296374 | Zinc finger protein-like 1 | ZFPL1 |
| IPI00373823 | Cytochrome P450 26C1 | CYP26C1 |
| IPI00815893 | Isoform 1 of Chromodomain-helicase-DNA-binding protein 2 | CHD2 |
| IPI00021907 | Isoform 1 of Myelin basic protein | MBP |
| IPI00027626 | T-complex protein 1 subunit zeta | CCT6A |
| IPI00398992 | Isoform 1 of Chromodomain-helicase-DNA-binding protein 8 | CHD8 |
| IPI00215746 | Fatty acid-binding protein, adipocyte | FABP4 |
| IPI00215914 | ADP-ribosylation factor 1 | ARF1 |
| IPI00399180 | Serine/threonine-protein kinase SBK1 | SBK1 |
| IPI00027547 | Dermcidin precursor | DCD |
| IPI00445315 | Protein FAM47C | FAM47C |
| IPI00746666 | hypothetical protein | LOC728262 |
| IPI00290854 | A-kinase anchor protein 3 | AKAP3 |
| IPI00604551 | Isoform 1 of Cell division cycle-associated protein 7 | CDCA7 |
| IPI00787083 | similar to peptidylprolyl isomerase A isoform 1 | LOC256374 |
| IPI00001712 | Isoform 1 of Catenin alpha-3 | CTNNA3 |
| IPI00217831 | Ankyrin repeat domain-containing protein 13A | ANKRD13A |
| IPI00024920 | ATP synthase subunit delta, mitochondrial precursor | ATP5D |
| IPI00028520 | Isoform 1 of NADH dehydrogenase [ubiquinone] flavoprotein 1, | NDUFV1 |
| | mitochondrial precursor | |
| IPI00006746 | Ermin | ERMN |
| IPI00003392 | Transmembrane protein 5 | TMEM5 |
| IPI00186966 | Isoform IIA of Myc box-dependent-interacting protein 1 | BIN1 |
| IPI00165009 | Isoform 3 of MBT domain-containing protein 1 | MBTD1 |
| IPI00641181 | MARCKS-related protein | MARCKSL1 |
| IPI00018342 | Adenylate kinase isoenzyme 1 | AK1 |
| IPI00394712 | Granulocyte inhibitory protein | — |
| IPI00141938 | H2A histone family, member V isoform 2 | H2AFV |
| IPI00028786 | Isoform 3 of Polycystin-1 precursor | PKD1 |
| IPI00299778 | Serum paraoxonase/lactonase 3 | PON3 |
| IPI00456683 | Isoform 3 of Transcription elongation factor SPT6 | SUPT6H |
| IPI00012391 | Isoform Long of Adenomatous polyposis coli protein | APC |
| IPI00185088 | immunoglobulin superfamily, member 11 isoform b | IGSF11 |
| IPI00152182 | Isoform 1 of Kelch domain-containing protein 4 | KLHDC4 |
| IPI00033030 | Protein ADRM1 | ADRM1 |
| IPI00185661 | Ubiquitin carboxyl-terminal hydrolase 32 | USP32 |
| IPI00218292 | Isoform Short of Ubiquitin fusion degradation protein 1 homolog | UFD1L |
| IPI00000104 | Isoform 1 of mRNA-capping enzyme | RNGTT |
| IPI00514893 | Disheveled-associated activator of morphogenesis 2 | DAAM2 |
| IPI00025363 | Isoform 1 of Glial fibrillary acidic protein | GFAP |
| IPI00743898 | Uncharacterized protein ENSP00000357890 (Fragment) | — |
| IPI00023359 | Isoform 1 of Malonyl CoA-acyl carrier protein transacylase, | MCAT |
| | mitochondrial precursor | |
| IPI00021274 | Ephrin type-A receptor 8 precursor | EPHA8 |
| IPI00410013 | Isoform 1 of Zinc finger CCCH domain-containing protein 3 | ZC3H3 |
| IPI00454910 | Serine/threonine-protein kinase MRCK gamma | CDC42BPG |
| IPI00302133 | Transient receptor potential cation channel subfamily V member 5 | TRPV5 |
| IPI00010207 | Ubiquitin-fold modifier 1 precursor | UFM1 |
| IPI00018708 | Isoform 2 of Centrosomal protein of 63 kDa | CEP63 |
| IPI00221034 | Transcription factor RelB | RELB |
| IPI00215979 | Bisphosphoglycerate mutase | BPGM |
| IPI00008274 | Adenylyl cyclase-associated protein 1 | CAP1 |
| IPI00479083 | Isoform 2 of Erythroid differentiation-related factor 1 | C10orf137 |
| IPI00168885 | Isoform 1 of Putative ATP-dependent RNA helicase DHX57 | DHX57 |
| IPI00748955 | platelet glycoprotein Ib alpha polypeptide precursor | GP1BA |
| IPI00024853 | Isoform 1 of Periaxin | PRX |
| IPI00307591 | Zinc finger protein 609 | ZNF609 |
| IPI00375881 | Polycystic kidney disease 1-like protein 3 | PKD1L3 |
| IPI00217652 | Isoform 1 of Glycosyltransferase 8 domain-containing protein 3 | GLT8D3 |
| IPI00219525 | 6-phosphogluconate dehydrogenase, decarboxylating | PGD |
| IPI00306884 | CDNA FLJ11867 fis, clone HEMBA1006976, weakly similar to | ST3GAL4 |
| | H. sapiens Gal-beta(1-3/1-4)GlcNAc alpha-2.3-sialyltransferase | |
| IPI00005158 | Lon protease homolog, mitochondrial precursor | LONP1 |
| IPI00013319 | Isoform 2 of 43 kDa receptor-associated protein of the synapse | RAPSN |
| IPI00290857 | Keratin, type II cytoskeletal 3 | KRT3 |
| IPI00216049 | Isoform 1 of Heterogeneous nuclear ribonucleoprotein K | HNRPK |
| IPI00014398 | Four and a half LIM domains 1 variant | FHL1 |
| IPI00001863 | Wnt inhibitory factor 1 precursor | WIF1 |
| IPI00218465 | Phospholipase A-2-activating protein | PLAA |
| IPI00045839 | Isoform 3 of Prolyl 3-hydroxylase 1 precursor | LEPRE1 |
| IPI00218730 | Rod cGMP-specific 3′,5′-cyclic phosphodiesterase subunit alpha | PDE6A |
| IPI00301465 | 14-3-3-associated AKT substrate | HJURP |
| IPI00011781 | Ankyrin repeat-containing protein C20orf86 precursor | C20orf86 |
| IPI00002459 | annexin VI isoform 2 | ANXA6 |
| IPI00025311 | Isoform 1 of Breast carcinoma-amplified sequence 1 | BCAS1 |
| IPI00021048 | Isoform 1 of Myoferlin | FER1L3 |
| IPI00027744 | Isoform 1 of Mineralocorticoid receptor | NR3C2 |
| IPI00029591 | P-selectin glycoprotein ligand 1 precursor | SELPLG |
| IPI00026230 | Heterogeneous nuclear ribonucleoprotein H2 | HNRPH2 |
| IPI00009365 | COX16-like protein C14orf112, mitochondrial precursor | C14orf112 |
| IPI00170766 | Isoform 2 of Protein CASC5 | CASC5 |
| IPI00741005 | similar to MAX-interacting protein isoform 4 | MGA |
| IPI00853400 | Isoform 1 of FK506-binding protein 15 | FKBP15 |
| IPI00867509 | Coronin-1C_i3 protein | CORO1C |
| IPI00290078 | keratin 4 | KRT4 |
| IPI00216592 | Isoform C1 of Heterogeneous nuclear ribonucleoproteins C1/C2 | HNRNPC |
| IPI00017529 | Isoform 1 of Lymphocyte function-associated antigen 3 precursor | CD58 |
| IPI00011592 | Cytoplasmic dynein 1 light intermediate chain 2 | DYNC1LI2 |
| IPI00006470 | Neuron-specific protein family member 2 | HMP19 |
| IPI00165229 | proprotein convertase subtilisin/kexin type 5 preproprotein | PCSK5 |
| IPI00845229 | Isoform 2 of DEP domain-containing protein 2 | DEPDC2 |
| IPI00221255 | Isoform 2 of Myosin light chain kinase, smooth muscle | MYLK |
| IPI00297550 | Coagulation factor XIII A chain precursor | F13A1 |
| IPI00304789 | 27 kDa protein | TTLL9 |
| IPI00792759 | 73 kDa protein | THSD4 |
| IPI00446588 | Isoform 2 of Plexin-A4 precursor | PLXNA4 |
| IPI00419836 | Isoform 1 of Discoidin, CUB and LCCL domain-containing protein 2 | DCBLD2 |
| | precursor | |
| IPI00160369 | PRKCA-binding protein | PICK1 |
| IPI00002478 | Isoform B of Endothelin-converting enzyme 1 | ECE1 |
| IPI00642645 | Methylenetetrahydrofolate reductase | MTHFR |
| IPI00017964 | Small nuclear ribonucleoprotein Sm D3 | SNRPD3 |
| IPI00478640 | Isoform 1 of Transmembrane protein C17orf87 | C17orf87 |
| IPI00013290 | hepatoma-derived growth factor-related protein 2 isoform 1 | HDGF2 |
| IPI00011051 | T-cell leukemia homeobox protein 1 | TLX1 |
| IPI00830051 | Similar to Immunolgoobulin heavy chain | LOC90925 |
| IPI00550720 | Isoform 1 of Uncharacterized protein C19orf57 | C19orf57 |
| IPI00103510 | Relaxin receptor 2 | RXFP2 |
| IPI00373872 | polycystin 1-like 2 isoform a | PKD1L2 |
| IPI00328522 | KTEL motif-containing protein 1 | KTELC1 |
| IPI00000811 | Proteasome subunit beta type-6 precursor | PSMB6 |
| IPI00251596 | Isoform 1 of Collagen alpha-1(XXIII) chain | COL23A1 |
| IPI00550906 | Cleavage stimulation factor 64 kDa subunit, tau variant | CSTF2T |
| IPI00045219 | Sorting nexin-18 | SNAG1 |
| IPI00221235 | nucleoporin 160 kDa | NUP160 |
| IPI00167560 | PAP-associated domain-containing protein 4 | PAPD4 |
| IPI00005719 | Isoform 1 of Ras-related protein Rab-1A | RAB1A |
| IPI00098902 | 2-oxoglutarate dehydrogenase E1 component, mitochondrial precursor | OGDH |
| IPI00289271 | Liprin-alpha-2 | PPFIA2 |
| IPI00216470 | Isoform 1 of Phosphatidylinositol-5-phosphate 4-kinase type-2 beta | PIP4K2B |
| IPI00744825 | Conserved hypothetical protein | — |
| IPI00005675 | NF-kappa-B-repressing factor | NKRF |
| IPI00218628 | Isoform 2 of Integrin alpha-IIb precursor | ITGA2B |
| IPI00217989 | Isoform 1 of Protein-associating with the carboxyl-terminal domain of | SCYL3 |
| | ezrin | |
| IPI00217405 | Isoform 1 of E3 ubiquitin-protein ligase UBR1 | UBR1 |
| IPI00217258 | CCDC100 protein | CCDC100 |
| IPI00456670 | Isoform 13 of Peroxisomal N(1)-acetyl-spermine/spermidine oxidase | PAOX |
| IPI00186621 | Orofacial clefting chromosomal breakpoint region 1 | OFCC1 |
| IPI00295098 | Signal recognition particle receptor subunit beta | SRPRB |
| IPI00071824 | Isoform 1 of Cytoskeleton-associated protein 2 | CKAP2 |
| IPI00747494 | Glutamate receptor delta-2 subunit precursor | GRID2 |
| IPI00879409 | 28 kDa protein | — |
| IPI00169259 | Small VCP/p97-interacting protein | SVIP |
| IPI00106506 | Isoform 1 of Evolutionarily conserved signaling intermediate in Toll | ECSIT |
| | pathway, mitochondrial precursor | |
| IPI00006900 | Something about silencing protein 10 | UTP3- |
| IPI00472332 | similar to polyhomeotic 1-like isoform 4 | LOC653441 |
| IPI00789245 | Isoform 2 of Probable organic cation transporter protein C6orf85 | SLC22A23 |
| IPI00291922 | Proteasome subunit alpha type-5 | PSMA5 |
| IPI00152145 | Protein odd-skipped-related 1 | OSR1 |
| IPI00021770 | Isoform 1 of 3-hydroxy-3-methylglutaryl-coenzyme A reductase | HMGCR |
| IPI00465045 | DIP2 disco-interacting protein 2 homolog B | DIP2B |
| IPI00018803 | homeobox D12 | HOXD12 |
| IPI00021594 | Isoform 1 of Glycosylphosphatidylinositol anchor attachment 1 | GPAA1 |
| | protein | |
| IPI00010360 | Isoform 1 of Collagen alpha-3(IV) chain precursor | COL4A3 |
| IPI00465044 | Protein RCC2 | RCC2 |
| IPI00013847 | Cytochrome b-c1 complex subunit 1, mitochondrial precursor | UQCRC1 |
| IPI00026665 | Glutaminyl-tRNA synthetase | QARS |
| IPI00180386 | Isoform GN-1L of Glycogenin-1 | GYG1 |
| IPI00167638 | Isoform 1 of GTP-binding protein 10 | GTPBP10 |
| IPI00384861 | lsoform 1 of ARF GTPase-activating protein GIT1 | GIT1 |
| IPI00296219 | Glutaminase liver isoform, mitochondrial precursor | GLS2 |
| IPI00376087 | putative binding protein 7a5 | 7A5 |
| IPI00794679 | Major histocompatibility complex, class I, B | MICA; HLA-B; HLA- |
| | | A; LOC730410; HLA-C; HLA- |
| | | A29.1 |
| IPI00008282 | Isoform 1 of Calcium/calmodulin-dependent 3′,5′-cyclic nucleotide | PDE1A |
| | phosphodiesterase 1A | |
| IPI00007277 | Isoform 1 of Leucine-rich repeat flightless-interacting protein 2 | LRRFIP2 |
| IPI00294519 | Isoform 1 of Telomerase protein component 1 | TEP1 |
| IPI00179589 | Myotrophin | MTPN |
| IPI00031485 | Mitochondrial ribosomal protein 63 | MRP63 |
| IPI00429191 | Eukaryotic peptide chain release factor subunit 1 | ETF1 |
| IPI00514594 | Isoform 1 of Protein FAM5B precursor | FAM5B |
| IPI00853376 | additional sex combs like 3 | ASXL3 |
| IPI00216914 | Vitelline membrane outer layer protein 1 homolog precursor | VMO1 |
| IPI00166010 | Isoform 1 of CCR4-NOT transcription complex subunit 1 | CNOT1 |
| IPI00399252 | Isoform 1 of Protein Jade-1 | PHF17 |
| IPI00144243 | Human immunodeficiency virus type I enhancer-binding protein 2 | HIVEP2 |
| IPI00291463 | Radical S-adenosyl methionine domain-containing protein 2 | RSAD2 |
| IPI00007040 | Zinc finger protein 222 | ZNF222 |
| IPI00220578 | Guanine nucleotide-binding protein G | GNAI3 |
| IPI00740191 | similar to Forkhead box protein L1 | LOC651986 |
| IPI00044607 | Protein phosphatase inhibitor 2-like protein 1 | PPP1R2P1 |
| IPI00301923 | Isoform 1 of Cell division protein kinase 9 | CDK9 |
| IPI00255145 | hypothetical protein | LOC342346 |
| IPI00010737 | Thrombomodulin precursor | THBD |
| IPI00030319 | Forkhead box protein F2 | FOXF2 |
| IPI00168806 | Isoform 1 of Myeloid/lymphoid or mixed-lineage leukemia protein 3 | MLL3 |
| | homolog | |
| IPI00015285 | Ethanolamine-phosphate cytidylyltransferase | PCYT2 |
| IPI00302850 | Small nuclear ribonucleoprotein Sm D1 | SNRPD1 |
| IPI00478124 | 61 kDa protein | UCKL1 |
| IPI00013681 | N-terminally extended type 3 canonical transient receptor potential | TRPC3 |
| | channel | |
| IPI00019208 | Similar to 60S ribosomal protein L29 | — |
| IPI00012895 | Isoform 1 of Carbonic anhydrase 12 precursor | CA12 |
| IPI00023513 | Isoform 1 of E3 ubiquitin-protein ligase CHFR | CHFR |
| IPI00025019 | Proteasome subunit beta type-1 precursor | PSMB1 |
| IPI00166817 | Zinc finger protein 561 | ZNF561 |
| IPI00375174 | Ankyrin repeat and sterile alpha motif domain-containing protein 1B | ANKS1B |
| IPI00296866 | interphotoreceptor matrix proteoglycan 2 | IMPG2 |
| IPI00040730 | protocadherin 21 precursor | PCDH21 |
| IPI00013508 | Alpha-actinin-1 | ACTN1 |
| IPI00644231 | Isoform 1 of Cytoplasmic FMR1-interacting protein 1 | CYFIPI |
| IPI00013466 | Arsenical pump-driving ATPase | ASNA1 |
| IPI00016605 | Uncharacterized protein C1orf123 | C1orf123 |
| IPI00872861 | PTD016 protein | LOC51136 |
| IPI00166619 | Isoform 2 of Putative transporter SVOPL | SVOPL |
| IPI00796647 | HIG1 domain family, member 1C | HIGD1C |
| IPI00852758 | Similar to Ankyrin repeat domain-containing protein 26. Isoform 2 | ANKRD18B |
| IPI00023087 | Ubiquitin-conjugating enzyme E2 T | UBE2T |
| IPI00161119 | Isoform 1 of NF-kappa-B inhibitor beta | NFKBIB |
| IPI00016577 | CDNA: FLJ22814 fis, clone KAIA3004 | CD22 |
| IPI00852633 | 16 kDa protein | RABL2B |
| IPI00163391 | Isoform 1 of Putative methyltransferase METT10D | METT10D |
| IPI00299679 | Isoform B of Ral guanine nucleotide dissociation stimulator-like 1 | RGL1 |
| IPI00797699 | 20 kDa protein | — |
| IPI00794119 | 13 kDa protein | ABCC8 |
| IPI00816274 | Chemokine-like factor superfamily 1 transcript variant 26 | CMTM1 |
| IPI00171737 | Isoform 2 of Leucine-rich repeat and death domain-containing protein | LRDD |
| IPI00385003 | Putative transposase | TIGD1 |
| IPI00029123 | Isoform A of Endothelin B receptor precursor | EDNRB |
| IPI00158992 | snRNA-activating protein complex subunit 4 | SNAPC4 |
| IPI00184997 | cDNA FLJ78771, highly similar to Homo sapiens discs, large | DLG7 |
| | homolog 7 (Drosophila), mRNA | |
| IPI00001869 | Pappalysin-1 precursor | PAPPA |
| IPI00027834 | heterogeneous nuclear ribonucleoprotein L isoform a | HNRNPL |
| IPI00062730 | Uncharacterized protein C16orf45 | C16orf45 |
| IPI00298258 | UNC13B protein | UNC13B |
| IPI00744366 | Conserved hypothetical protein | — |
| IPI00000792 | Quinone oxidoreductase | CRYZ |
| IPI00008905 | UDP-glucuronosyltransferase 2B15 precursor | UGT2B15 |
| IPI00186581 | amplified in osteosarcoma isoform 2 precursor | OS9 |
| IPI00445364 | CDNA FLJ44171 fis, clone THYMU2036058 | — |
| IPI00166071 | B-cell CLL/lymphoma 6 member B protein | BCL6B |
| IPI00218130 | Glycogen phosphorylase, muscle form | PYGM |
| IPI00034006 | Tyrosine-protein phosphatase non-receptor type 23 | PTPN23 |
| IPI00419253 | Isoform 1 of Nck-associated protein 5 | NAP5 |
| IPI00292393 | Sodium channel protein type 4 subunit alpha | SCN4A |
| IPI00029012 | Eukaryotic translation initiation factor 3 subunit A | EIF3A |
| IPI00385042 | Nucleolar GTP-binding protein 1 | GTPBP4 |
| IPI00402144 | Isoform 1 of Zinc finger protein 555 | ZNF555 |
| IPI00478986 | Similar to 40S ribosomal protein S4 | LOC126235 |
| IPI00478521 | lsoform 1 of UPF0475 protein | RILPL1 |
| IPI00025700 | Isoform CD6A of T-cell differentiation antigen CD6 precursor | CD6 |
| IPI00061507 | Isoform 3 of Ester hydrolase C11orf54 | C11orf54 |
| IPI00016949 | Isoform 4 of Electrogenic sodium bicarbonate cotransporter 1 | SLC4A4 |
| IPI00031547 | Desmoglein-3 precursor | DSG3 |
| IPI00382756 | Isoform 2 of Pleiotropic regulator 1 | PLRG1 |
| IPI00743284 | Methionine synthase | MTR |
| IPI00021363 | Histone demethylase JARID1A | JARID1A |
| IPI00216683 | M-phase inducer phosphatase 3 | CDC25C |
| IPI00737969 | microtubule associated monoxygenase, calponin and LIM domain | MICAL3 |
| | containing 3 | |
| IPI00015522 | Growth/differentiation factor 5 precursor | GDF5 |
| IPI00877084 | Isoform 1 of Coiled-coil domain-containing protein 144C | CCDC144C |
| IPI00060265 | Zinc finger protein 775 | ZNF775 |
| IPI00024802 | TATA-binding protein-associated factor 172 | BTAF1 |
| IPI00374039 | Conserved hypothetical protein | C1orf189 |
| IPI00442121 | delta-aminolevulinic acid dehydratase isoform a | ALAD |
| IPI00013455 | CLIP1 protein | CLIP1 |
| IPI00026299 | Isoform Glycophorin C of Glycophorin-C | GYPC |
| IPI00845508 | BAH domain and coiled-coil containing 1 | BAHCC1 |
| IPI00745300 | 31 kDa protein | NAT11 |
| IPI00186826 | Ephrin receptor | EPHB4 |
| IPI00787932 | similar to zinc finger protein 10 | hCG_1646157 |
| IPI00166161 | Protein SIX6OS1 | C14orf39 |
| IPI00180426 | Isoform 3 of G protein-coupled receptor kinase 4 | GRK4 |
| IPI00013219 | Integrin-linked protein kinase | ILK |
| IPI00796906 | 8 kDa protein | ABCF3 |
| IPI00028932 | Microtubule-associated serine/threonine-protein kinase 3 | MAST3 |
| IPI00387159 | Isoform 1 of Inhibitor of growth protein 3 | ING3 |
| IPI00645089 | Kv channel interacting protein 1 isoform 3 | KCNIP1 |
| IPI00412216 | vacuolar protein sorting 13C protein isoform 2B | VPS13C |
| IPI00374862 | Isoform 1 of Kelch-like protein 5 | KLHL5 |
| IPI00009377 | HSPC212 | C3orf19 |
| IPI00005969 | F-actin-capping protein subunit alpha-1 | CAPZA1 |
| IPI00300244 | zinc finger, CW type with PWWP domain 1 | ZCWPW1 |
| IPI00333410 | Isoform 1 of Ubiquitin-conjugating enzyme E2 Q1 | UBE2Q1 |
| IPI00218075 | Protein FAM9B | FAM9B |
| IPI00252845 | SYT9 protein | SYT9 |
| IPI00477361 | 10 kDa protein | SDHALP1 |
| IPI00397578 | 135 kDa protein | PPFIA4 |
| IPI00017659 | Protein kinase substrate CapZIP | RCSD1 |
| IPI00025094 | CDNA: FLJ22037 fis, clone HEP08868 (Fragment) | MYH16 |
| IPI00216963 | Isoform 9 of CASP8 and FADD-like apoptosis regulator precursor | CFLAR |
| IPI00010895 | Tubby-related protein 2 | TULP2 |
| IPI00739106 | similar to ribosomal protein L5 isoform 1 | LOC647436 |
| IPI00022606 | Isoform 1 of Proline-serine-threonine phosphatase-interacting protein 1 | PSTPIP1 |
| IPI00295502 | Isoform 1 of Protein Wiz | WIZ |
| IPI00435925 | PP14214 | IGFBP3 |
| IPI00010118 | Isoform 1 of Prostate tumor overexpressed gene 1 protein | PTOV1 |
| IPI00300990 | Isoform 1 of Uncharacterized protein C1orf77 | C1orf77 |
| IPI00741780 | similar to CG4845-PA | LOC652559 |
| IPI00454858 | similar to alpha 3 type VI collagen isoform 1 precursor | LOC344875 |
| IPI00399328 | similar to jumonji domain containing 2D | LOC390245 |
| IPI00004534 | Phosphoribosylformylglycinamidine synthase | PFAS |
| IPI00010575 | KIAA1466 protein | KIAA1466 |
| IPI00785015 | Isoform 1 of Uncharacterized protein KIAA2030 | FLJ25778 |
| IPI00103630 | Isoform 2 of Protein phosphatase 1E | PPM1E |
| IPI00479669 | Isoform 1 of Uncharacterized protein KIAA0701 | UHRF1BP1L |
| IPI00017940 | LMBR1 domain-containing protein 2 | LMBRD2 |
| IPI00216508 | Isoform 2 of Sorting nexin-3 | SNX3 |
| IPI00060146 | Isoform 1 of Smith-Magenis syndrome chromosome region candidate | SMCR7 |
| | gene 7 protein | |
| IPI00022989 | Isoform Beta-1 of Retinoic acid receptor beta | RARB |
| IPI00152072 | hypothetical protein LOC387758 | FIBIN |
| IPI00060546 | Uncharacterized protein C10orf35 | C10orf35 |
| IPI00043978 | Isoform 1 of Partitioning-defective 3 homolog B | PARD3B |
| IPI00440221 | Putative uncharacterized protein (Fragment) | CDC2L5 |
| IPI00011564 | Syndecan-4 precursor | SDC4 |
| IPI00423683 | Isoform 2 of EMI domain-containing protein 1 precursor | EMID1 |
| IPI00026612 | Isoform Beta-1 of Protein phosphatase 1B | PPM1B |
| IPI00167137 | Isoform 3 of SLAM family member 7 precursor | SLAMF7 |
| IPI00155447 | MMP28 protein | MMP28 |
| IPI00001434 | Protocadherin beta 14 precursor | PCDHB14 |
| IPI00043731 | CDNA FLJ30671 fis, clone FCBBF1000687, moderately similar to | RUNDC3B |
| | Mus musculus Rap2 interacting protein 8 (RPIP8) mRNA | |
| IPI00645814 | Isoform 2 of MAP7 domain-containing protein 1 | MAP7D1 |
| IPI00003353 | Neuronal protein 3.1 | C5orf13 |
| IPI00007402 | Importin-7 | IPO7 |
| IPI00152470 | Prokineticin receptor 1 | PROKR1 |
| IPI00006094 | Regulating synaptic membrane exocytosis protein 3 | RIMS3 |
| IPI00101927 | Leucine zipper putative tumor suppressor 2 | LZTS2 |
| IPI00010346 | Neurolysin, mitochondrial precursor | NLN |
| IPI00216572 | BarH-like homeobox 2 | BARX2 |
| IPI00171647 | Isoform 1 of Sialic acid-binding Ig-like lectin 8 precursor | SIGLEC8 |
| IPI00020058 | Isoform 1 of Copper-transporting ATPase 2 | ATP7B |
| IPI00026570 | Cytochrome c oxidase polypeptide VIIa-liver/heart, mitochondrial | COX7A2 |
| | precursor | |
| IPI00027726 | Isoform 1 of Krueppel-like factor 3 | KLF3 |
| IPI00064241 | Isoform 1 of Zinc finger protein Eos | IKZF4 |
| IPI00295503 | Isoform 2 of Probable ATP-dependent RNA helicase DDX58 | DDX58 |
| IPI00290292 | Rhomboid 5 homolog 1 | RHBDF1 |
| IPI00029533 | Integrin beta-8 precursor | ITGB8 |
| IPI00815786 | Hexokinase 1 (Fragment) | HK1 |
| IPI00658112 | 32 kDa protein | SPEG |
| IPI00479361 | Isoform 1 of UDP-GlcNAc:betaGal beta-1,3-N- | B3GNT4 |
| | acetylglucosaminyltransferase 4 | |
| IPI00448465 | Isoform 1 of Ankyrin repeat domain-containing protein 12 | ANKRD12 |
| IPI00745122 | Conserved hypothetical protein | MGC33894 |
| IPI00005531 | Isoform 1 of Probable DNA dC->dU-editing enzyme APOBEC-3B | APOBEC3B |
| IPI00383832 | Protein kinase C-binding protein RACK8 | DVL3 |
| IPI00027685 | C-C chemokine receptor type 1 | CCR1 |
| IPI00009335 | Brain protein 16 | C8orf30A |
| IPI00432755 | PPRR6495 | FAM124A |
| IPI00796777 | 17 kDa protein | CRYAA |
| IPI00008894 | Carboxypeptidase A4 precursor | CPA4 |
| IPI00031005 | Protein kinase-like protein SgK196 | FLJ23356 |
| IPI00787414 | Uncharacterized protein ENSP00000381388 | MGC34829 |
| IPI00019988 | N-sulphoglucosamine sulphohydrolase precursor | SGSH |
| IPI00873740 | Uncharacterized protein ENSP00000383832 (Fragment) | — |
| IPI00032416 | Isoform Long of Protein jagged-2 precursor | JAG2 |
| IPI00335589 | RNA methyltransferase-like protein 1 | RNMTL1 |
| IPI00843819 | Similar to Dual specificity protein kinase CLK2 | — |
| IPI00065276 | Isoform 2 of Tether containing UBX domain for GLUT4 | ASPSCR1 |
| IPI00644522 | PNKP protein | PNKP |
| IPI00045511 | Isoform 1 of Chloride channel CLIC-like protein 1 precursor | CLCC1 |
| IPI00604763 | Transmembrane protein 66 precursor | TMEM66 |
| IPI00235647 | similar to fibrillarin | LOC345630 |
| IPI00470805 | Isoform 2 of Mediator of DNA damage checkpoint protein 1 | MDC1 |
| IPI00167089 | Isoform 2 of Activated CDC42 kinase 1 | TNK2 |
| IPI00783753 | UPF0235 protein C15orf40 | C15orf40 |
| IPI00030919 | Mitogen-activated protein kinase kinase 1-interacting protein 1 | MAP2K1IP1 |
| IPI00394879 | Leucine-rich repeat-containing protein 9 | LRRC9 |
| IPI00401852 | Conserved hypothetical protein | DKFZP434L187 |
| IPI00442865 | CDNA FLJ26488 fis, clone KDN05770, highly similar to Bumetanide- | SLC12A1 |
| | sensitive sodium-(potassium)-chloride cotransporter 2 | |
| IPI00748891 | hypothetical protein LOC283635 isoform 1 | C14orf24 |
| IPI00414320 | Annexin A11 | ANXA11 |
| IPI00874023 | Uncharacterized protein ENSP00000379699 | — |
| IPI00827584 | similar to kinesin family member 27 | KIF27 |
| IPI00020199 | Alpha-2,8-sialyltransferase 8B | ST8SIA2 |
| IPI00791513 | CDNA FLJ16614 fis, clone TESTI4013365 | CTA-216E10.6 |
| IPI00002884 | CDNA: FLJ22222 fis, clone HRC01658 | FLJ22222 |
| IPI00003363 | Isoform 1 of Protein phosphatase 1 regulatory subunit 1B | PPP1R1B |
| IPI00005705 | Isoform Gamma-1 of Serine/threonine-protein phosphatase PP1- | PPP1CC |
| | gamma catalytic subunit | |
| IPI00164949 | Isoform NELF-C of Negative elongation factor C/D | TH1L |
| IPI00029647 | Zymogen granule membrane protein 16 precursor | ZG16 |
| IPI00414481 | GTF3C1 protein | GTF3C1 |
| IPI00154528 | Isoform 1 of Structural maintenance of chromosomes protein 6 | SMC6 |
| IPI00164861 | Isoform 3 of Kinesin-like protein KIF13A | KIF13A |
| IPI00411674 | Isoform 1 of Zinc finger protein 254 | ZNF254 |
| IPI00249982 | Isoform 1 of Death-inducer obliterator 1 | DIDO1 |
| IPI00328270 | Neuronal PAS domain-containing protein 2 | NPAS2 |
| IPI00442544 | CDNA FLJ27034 fis, clone SLV07984 | — |
| IPI00175019 | similar to Temporarily Assigned Gene name family member | LOC643677 |
| IPI00027898 | Isoform A of Uncharacterized protein C21orf70 | C21orf70 |
| IPI00023340 | Histone acetyltransferase MYST3 | MYST3 |
| IPI00300052 | Keratin type II cuticular Hb4 | KRT84 |
| IPI00019158 | ADAM metallopeptidase domain 8 precursor | ADAM8 |
| IPI00045928 | Sodium/hydrogen exchanger 7 | SLC9A7 |
| IPI00306332 | 60S ribosomal protein L24 | RPL24 |
| IPI00023184 | Isoform 1 of Poly [ADP-ribose] polymerase 3 | PARP3 |
| IPI00872739 | Uncharacterized protein C18orf2 | C18orf2 |
| IPI00241409 | hypothetical protein LOC55747 | FAM21B |
| IPI00090764 | Toll-like receptor 1 precursor | TLR1 |
| IPI00024253 | Isoform 1 of Fibroblast growth factor 14 | FGF14 |
| IPI00218637 | Major histocompatibility complex, class II, DQ beta 2 | HLA-DQB2 |
| IPI00216921 | Isoform 2 of Stathmin-4 | STMN4 |
| IPI00011416 | Delta(3,5)-Delta(2,4)-dienoyl-CoA isomerase, mitochondrial | ECH1 |
| | precursor | |
| IPI00260755 | similar to Rho GTPase activating protein 18 | C20orf95 |
| IPI00337385 | Isoform 1 of Pre-mRNA-processing factor 40 homolog A | PRPF40A |
| IPI00304527 | Protein FAM83B | FAM83B |
| IPI00030741 | Uncharacterized protein C21orf13 | LCA5L |
| IPI00399296 | hypothetical protein LOC390110 | LOC390110 |
| IPI00233358 | islet cell autoantigen 1, 69 kDa-like isoform 2 | ICA1L |
| IPI00879842 | 6 kDa protein | — |
| IPI00021476 | Eukaryotic translation initiation factor 4E-binding protein 3 | EIF4EBP3 |
| IPI00028383 | Uncharacterized protein C16orf24 | C16orf24 |
| IPI00298337 | cDNA FLJ77671 | SLC14A1 |
| IPI00044842 | Isoform 2 of RAB3A-interacting protein | RAB3IP |
| IPI00013004 | Isoform 1 of Pyridoxal kinase | PDXK |
| IPI00328260 | Protein FAN | NSMAF |
| IPI00807418 | Isoform 9 of Lymphoid-specific helicase | HELLS |
| IPI00183002 | Isoform 1 of Protein phosphatase 1 regulatory subunit 12A | PPP1R12A |
| IPI00163601 | Putative uncharacterized protein FLJ10213 | FLJ10213 |
| IPI00428741 | LP2477 | FLJ35348 |
| IPI00006252 | Multisynthetase complex auxiliary component p43 | SCYE1 |
| IPI00007321 | Isoform 1 of Acyl-protein thioesterase 1 | LYPLA1 |
| IPI00217617 | palmitoylated membrane protein 7 | MPP7 |
| IPI00024662 | Chromobox protein homolog 5 | CBX5 |
| IPI00550364 | Phosphoglucomutase-2 | PGM2 |
| IPI00169331 | Phosphatidylcholine:ceramide cholinephosphotransferase 2 | SGMS2 |
| IPI00028450 | Isoform 1 of Sodium/calcium exchanger 1 precursor | SLC8A1 |
| IPI00010405 | Isoform Long of Tyrosine-protein kinase transmembrane receptor | ROR1 |
| | ROR1 precursor | |
| IPI00873774 | Uncharacterized protein ENSP00000383488 (Fragment) | — |
| IPI00847335 | FLJ45422 protein | — |
| IPI00217740 | C20orf12 protein | C20orf12 |
| IPI00008497 | Ornithine decarboxylase | ODC1 |
| IPI00010808 | Interferon-gamma receptor alpha chain precursor | IFNGR1 |
| IPI00872550 | Uncharacterized protein PRDM2 | PRDM2 |
| IPI00514622 | Ran-binding protein 6 | RANBP6 |
| IPI00009203 | Sorting nexin-7 | SNX7 |
| IPI00790021 | Zinc finger protein 652 | ZNF652 |
| IPI00855918 | mucin 5, subtype B, tracheobronchial | MUC5B |
| IPI00008404 | Isoform Long of Segment polarity protein dishevelled homolog DVL- | DVL1L1 |
| | 1-like | |
| IPI00658025 | Putative novel transcript | — |
| IPI00022958 | PRO0149 | C16orf72 |
| IPI00032313 | Protein S100-A4 | S100A4 |
| IPI00027009 | Isoform 1 of Protein kinase C and casein kinase substrate in neurons | PACSIN2 |
| | protein 2 | |
| IPI00023162 | UDP-N-acetylglucosamine 2-epimerase/N-acetylmannosamine kinase | GNE |
| IPI00789181 | 115 kDa protein | PLCL1 |
| IPI00005132 | Guanine nucleotide-binding protein-like 3-like protein | GNL3L |
| IPI00742725 | Conserved hypothetical protein | LOC388564 |
| IPI00878755 | 43 kDa protein | — |
| IPI00152050 | ataxin 2-binding protein 1 isoform 3 | A2BP1 |
| IPI00385480 | Caskin-1 | CASKIN1 |
| IPI00465123 | KIAA0415 gene product | KIAA0415 |
| IPI00012441 | Isoform 1 of Synaptojanin-1 | SYNJ1 |
| IPI00006054 | Syntaphilin | SNPH |
| IPI00385918 | CDNA FLJ90582 fis, clone PLACE1000442, moderately similar to | ZNF627 |
| | ZINC FINGER PROTEIN ZFP-36 | |
| IPI00008438 | 40S ribosomal protein S10 | RPS10 |
| IPI00152849 | Isoform 1 of G2/mitotic-specific cyclin-B3 | CCNB3 |
| IPI00029556 | Uncharacterized protein C1orf105 | C1orf105 |
| IPI00217012 | pleckstrin and Sec7 domain containing | PSD |
| IPI00015980 | Isoform 2 of Multiple PDZ domain protein | MPDZ |
| IPI00102678 | Isoform 1 of Pecanex-like protein 1 | PCNX |
| IPI00020356 | 331 kDa protein | MAP1A |
| IPI00008226 | 73 kDa protein | THSD3 |
| IPI00010903 | Dopey family member 1 | DOPEY1 |
| IPI00735934 | similar to capicua homolog | LOC646070 |
| IPI00465234 | Cytokine receptor common beta chain precursor | CSF2RB |
| IPI00010303 | Serpin B4 | SERPINB4 |
| IPI00008091 | Putative DNA helicase INO80 complex homolog 1 | INOC1 |
| IPI00016112 | peroxidasin homolog | PXDN |
| IPI00329791 | cDNA FLJ78679, highly similar to Homo sapiens DEAD (Asp-Glu- | DDX46 |
| | Ala-Asp) box polypeptide 46 (DDX46), mRNA | |
| IPI00152344 | Pyridoxal phosphate phosphatase PHOSPHO2 | PHOSPHO2 |
| IPI00028601 | Putative metallothionein C20orf127 | MT1P3 |
| IPI00479125 | SLIT-ROBO Rho GTPase-activating protein 2 | SRGAP2 |
| IPI00455852 | Isoform 1 of Rho guanine nucleotide exchange factor 15 | ARHGEF15 |
| IPI00550876 | Protein maestro | MRO |
| IPI00064296 | PRO0633 | — |
| IPI00216651 | Isoform 1 of Interleukin-28 receptor alpha chain precursor | IL28RA |
| IPI00792945 | 38 kDa protein | CHFR |
| IPI00149375 | Isoform 2 of Uncharacterized protein C11orf56 | C11orf56 |
| IPI00015913 | 5,6-dihydroxyindole-2-carboxylic acid oxidase precursor | TYRP1 |
| IPI00007512 | Glutathione transferase omega-2 | GSTO2 |
| IPI00016576 | Isoform 1 of Grainyhead-like protein 2 homolog | GRHL2 |
| IPI00290308 | Tribbles homolog 1 | TRIB1 |
| IPI00025622 | AN1-type zinc finger protein 5 | ZFAND5 |
| IPI00102808 | Isoform 1 of Dual specificity protein phosphatase 19 | DUSP19 |
| IPI00018208 | Tetratricopeptide repeat protein 33 | TTC33 |
| IPI00166865 | CDGSH iron sulfur domain-containing protein 2 | CISD2 |
| IPI00004533 | Kinesin-like protein KIF3B | KIF3B |
| IPI00477468 | RNA polymerase-associated protein CTR9 homolog | CTR9 |
| IPI00005668 | Aldo-keto reductase family 1 member C2 | AKR1C2 |
| IPI00018098 | Isoform 1 of Pre-mRNA-splicing factor 38B | PRPF38B |
| IPI00026241 | Bone marrow stromal antigen 2 precursor | BST2 |
| IPI00243338 | 24 kDa protein | KRT23 |
| IPI00786937 | similar to deleted in malignant brain tumors 1 isoform b precursor | LOC731940 |
| IPI00218918 | Annexin A1 | ANXA1 |
| IPI00295577 | Receptor-type tyrosine-protein phosphatase beta precursor | PTPRB |
| IPI00029372 | Uncharacterized protein C4orf15 | C4orf15 |
| IPI00744226 | Conserved hypothetical protein | — |
| IPI00792229 | 20 kDa protein | TATDN1 |
| IPI00220271 | Alcohol dehydrogenase | AKR1A1 |
| IPI00015954 | GTP-binding protein SAR1a | SAR1A |
| IPI00418735 | hypothetical protein LOC400566 | LOC400566 |
| IPI00306413 | Tubulin polymerization-promoting protein family member 3 | TPPP3 |
| IPI00018755 | High mobility group protein 1-like 10 | HMG1L10 |
| IPI00385543 | Isoform 3 of UPF0469 protein KIAA0907 | KIAA0907 |
| IPI00299076 | Receptor-binding cancer antigen expressed on SiSo cells (Fragment) | EBAG9 |
| IPI00386393 | CDNA FLJ13729 fis, clone PLACE3000121, weakly similar to | EXOC6B |
| | VESICULAR TRAFFIC CONTROL PROTEIN SEC15 | |
| IPI00878511 | 45 kDa protein | — |
| IPI00442564 | CDNA FLJ26948 fis, clone RCT08241 | — |
| IPI00412408 | Breast cancer type 2 susceptibility protein | BRCA2 |
| IPI00005614 | Isoform Long of Spectrin beta chain, brain 1 | SPTBN1 |
| IPI00021831 | cAMP-dependent protein kinase type I-alpha regulatory subunit | PRKAR1A |
| IPI00008422 | Isoform 2 of SWI/SNF-related matrix-associated actin-dependent | SMARCAD1 |
| | regulator of chromatin subfamily A containing DEAD/H box 1 | |
| IPI00007682 | Vacuolar ATP synthase catalytic subunit A | ATP6V1A |
| IPI00024502 | Ubiquilin-4 | UBQLN4 |
| IPI00029175 | Strumpellin | KIAA0196 |
| IPI00045360 | Capicua-like protein/double homeodomain 4 fusion protein | CIC |
| IPI00749171 | Conserved hypothetical protein | LOC340184 |
| IPI00298738 | DNA-directed RNA polymerase, mitochondrial precursor | POLRMT |
| IPI00445278 | CDNA FLJ44033 fis, clone TEST14028062 | — |
| IPI00335541 | Isoform 1 of Protein timeless homolog | TIMELESS |
| IPI00444842 | CDNA FLJ45125 fis, clone BRAWH3036561 | — |
| IPI00023152 | Isoform 1 of N-acetylated-alpha-linked acidic dipeptidase-like protein | NAALADL1 |
| IPI00296727 | Kinesin-like protein KIF2B | KIF2B |
| IPI00005347 | Zinc finger Ran-binding domain-containing protein 1 | ZRANB1 |
| IPI00470913 | RANBP2-like and GRIP domain containing 1 | RGPD1; RGPD2 |
| IPI00444331 | Isoform 4 of Histone-lysine N-methyltransferase NSD3 | WHSC1L1 |
| IPI00023407 | Nck-associated protein 1-like | NCKAP1L |
| IPI00456635 | Isoform 1 of Protein unc-13 homolog D | UNC13D |
| IPI00149044 | Isoform 2 of Suppressor of hairy wing homolog 4 | ZNF280D |
| IPI00007834 | Isoform 1 of Ankyrin-2 | ANK2 |
| IPI00746681 | Similar to Bcl-2-related ovarian killer protein | — |
| IPI00000977 | Mitogen-activated protein kinase kinase kinase 11 | MAP3K11 |
| IPI00871556 | 107 kDa protein | ZFYVE28 |
| IPI00003814 | Isoform 1 of Dual specificity mitogen-activated protein kinase kinase 6 | MAP2K6 |
| IPI00301058 | Vasodilator-stimulated phosphoprotein | VASP |
| IPI00245940 | immunoglobulin superfamily 5 like | IGSF5 |
| IPI00657699 | Protein | MUC19 |
| IPI00290094 | Splicing factor, arginine/serine-rich 8 | SFRS8 |
| IPI00030706 | Activator of 90 kDa heat shock protein ATPase homolog 1 | AHSA1 |
| IPI00853516 | dynein, axonemal, heavy chain 17 | DNAH17 |
| IPI00410093 | coiled-coil domain containing 69 | CCDC69 |
| IPI00168404 | Zinc finger and BTB domain containing 34 | ZBTB34 |
| IPI00644766 | cDNA FLJ78048, highly similar to Homo sapiens torsin A interacting | TOR1AIP1 |
| | protein 1, mRNA | |
| IPI00167154 | Uncharacterized protein MAPKBP1 | MAPKBP1 |
| IPI00871533 | Uncharacterized protein C3orf48 (Fragment) | C3orf48 |
| IPI00877615 | 15 kDa protein | — |
| IPI00030352 | Isoform 2 of Growth inhibition and differentiation-related protein 88 | C10orf28 |
| IPI00293679 | Isoform 1 of Potassium voltage-gated channel subfamily KQT | KCNQ4 |
| | member 4 | |
| IPI00748682 | Pheromone shutdown-related, TraB family protein | — |
| IPI00019888 | Succinate-semialdehyde dehydrogenase, mitochondrial precursor | ALDH5A1 |
| IPI00032597 | RNA-binding motif protein, X-linked 2 | RBMX2 |
| IPI00783855 | neighbor of BRCA1 gene 1 | NBR1 |
| IPI00746987 | Ribosomal protein S1 family protein | — |
| IPI00295469 | Copine-6 | CPNE6 |
| IPI00456599 | hypothetical protein LOC84792 | MGC12966 |
| IPI00607831 | PRAME family member 3 | PRAMEF3 |
| IPI00399254 | Isoform 1 of OTU domain-containing protein 4 | OTUD4 |
| IPI00719505 | RABL2A protein | RABL2A |
| IPI00784880 | Cancer/testis antigen 75 | LOC440934 |
| IPI00884353 | Ets-1 transcript variant ets-1 delta | — |
| IPI00852725 | Isoform 7 of Prolactin receptor precursor | PRLR |
| IPI00005600 | Isoform 1 of Bifunctional heparan sulfate N-deacetylase/N- | NDST2 |
| | sulfotransferase 2 | |
| IPI00791593 | 8 kDa protein | GLYCAM1 |
| IPI00024346 | snRNA-activating protein complex subunit 3 | SNAPC3 |
| IPI00293396 | adaptor-related protein complex 1, gamma 1 subunit isoform a | AP1G1 |
| IPI00018914 | Tyrosine-protein phosphatase non-receptor type 14 | PTPN14 |
| IPI00296913 | ADP-sugar pyrophosphatase | NUDT5 |
| IPI00185146 | Importin-9 | IPO9 |
| IPI00217791 | Coiled-coil domain-containing protein 105 | CCDC105 |
| IPI00167006 | Uncharacterized protein C13orf26 | C13orf26 |
| IPI00028481 | Ras-related protein Rab-8A | RAB8A |
| IPI00294215 | Uncharacterized protein KIAA0232 | KIAA0232 |
| IPI00071185 | Isoform SV1 of PITSLRE serine/threonine-protein kinase CDC2L1 | CDC2L1 |
| IPI00166776 | Protein CREG2 precursor | CREG2 |
| IPI00001796 | Tumor necrosis factor receptor superfamily, member 18 (Fragment) | TNFRSF18 |
| IPI00549972 | LIM domain-containing protein 2 | LIMD2 |
| IPI00029107 | Werner syndrome ATP-dependent helicase | WRN |
| IPI00470468 | Isoform 3 of Protein EFR3 homolog A | EFR3A |
| IPI00382515 | CDNA FLJ30384 fis, clone BRACE2008114 | — |
| IPI00853312 | Uncharacterized protein ENSP00000324580 | — |
| IPI00217871 | Delta-1-pyrroline-5-carboxylate dehydrogenase, mitochondrial | ALDH4A1 |
| | precursor | |
| IPI00470490 | Isoform 1 of Nuclear receptor coactivator 1 | NCOA1 |
| IPI00001433 | Protocadherin beta 15 precursor | PCDHB15 |
| IPI00220156 | Isoform B of Transforming growth factor beta-2 precursor | TGFB2 |
| IPI00024818 | Isoform 1 of Ubiquitin-specific peptidase-like protein 1 | USPL1 |
| IPI00335437 | Ankyrin repeat and zinc finger domain-containing protein 1 | ANKZF1 |
| IPI00000076 | Beta-nerve growth factor precursor | NGFB |
| IPI00100980 | EH domain-containing protein 2 | EHD2 |
| IPI00215610 | 55 kDa erythrocyte membrane protein | MPP1 |
| IPI00015148 | Ras-related protein Rap-1b precursor | RAP1B |
| IPI00410657 | Isoform 2 of mRNA cap guanine-N7 methyltransferase | RNMT |
| IPI00552591 | V1-20 protein | IGLV10-54 |
| IPI00022277 | Coiled-coil domain-containing protein 56 | CCDC56 |
| IPI00216288 | Isoform 3 of Lethal | L3MBTL |
| IPI00025499 | Isoform Tau-F of Microtubule-associated protein tau | MAPT |
| IPI00856012 | collagen type VI alpha 6 | COL6A6 |
| IPI00335168 | Isoform Non-muscle of Myosin light polypeptide 6 | MYL6 |
| IPI00030037 | Agouti-signaling protein precursor | ASIP |
| IPI00013749 | Protein kinase C zeta type | PRKCZ |
| IPI00002491 | Isoform 9 of Sorbin and SH3 domain-containing protein 1 | SORBS1 |
| IPI00240793 | Probable phospholipid-transporting ATPase IF | ATP11B |
| IPI00376237 | Isoform 2 of Transcription factor LBX2 | LBX2 |
| IPI00020966 | Isoform 1 of Phosphatidylinositol N-acetylglucosaminyltransferase | PIGA |
| | subunit A | |
| IPI00456827 | Protein FAM22G precursor | FAM22G |
| IPI00747657 | Similar to Rod cGMP-specific 3′,5′-cyclic phosphodiesterase subunit | PDE6B |
| | beta precursor | |
| IPI00215777 | Isoform B of Phosphate carrier protein, mitochondrial precursor | SLC25A3 |
| IPI00028912 | zinc finger protein 161 | VEZF1 |
| IPI00019242 | Matrix metalloproteinase-15 precursor | MMP15 |
| IPI00024664 | Isoform Long of Ubiquitin carboxyl-terminal hydrolase 5 | USP5 |
| IPI00786946 | similar to Tektin-3 | LOC642249 |
| IPI00166257 | CDNA FLJ37614 fis, clone BRCOC2011769 | — |
| IPI00442745 | CDNA FLJ26780 fis, clone PRS03837 | — |
| IPI00017256 | Ras suppressor protein 1 | RSU1 |
| IPI00374301 | hypothetical protein | — |
| IPI00023217 | Isoform 1 of Ryanodine receptor 2 | RYR2 |
| IPI00018843 | Isoform 1 of D(3) dopamine receptor | DRD3 |
| IPI00027782 | Stromelysin-1 precursor | MMP3 |
| IPI00030009 | Isoform A of Bifunctional 3′-phosphoadenosine 5′-phosphosulfate | PAPSS2 |
| | synthetase 2 | |
| IPI00479722 | Proteasome activator complex subunit 1 | PSME1 |
| IPI00177878 | Isoform 3 of Transmembrane protein 16D | TMEM16D |
| IPI00104907 | Isoform 1 of Uncharacterized potential DNA-binding protein | C14orf106 |
| | C14orf106 | |
| IPI00005107 | Niemann-Pick C1 protein precursor | NPC1 |
| IPI00009070 | Isoform 1 of HBS1-like protein | HBS1L |
| IPI00386576 | Ig lambda chain V-IV region MOL | — |
| IPI00747420 | Melanoma-derived protein (Fragment) | — |
| IPI00432226 | AVLL5809 | — |
| IPI00178894 | Zinc finger and BTB domain-containing protein 20 | ZBTB20 |
| IPI00878436 | 24 kDa protein | SLC2A11 |
| IPI00640810 | 6 kDa protein | CTDP1 |
| IPI00306549 | CDNA FLJ11065 fis, clone PLACE1004868, weakly similar to | MLSTD1 |
| | MALE STERILITY PROTEIN 2 | |
| IPI00293361 | Isoform 2 of Small G protein signaling modulator 2 | SGSM2 |
| IPI00021753 | Kinesin-like protein KIF13B | KIF13B |
| IPI00253281 | Isoform 2 of Epidermal growth factor receptor kinase substrate 8-like | EPS8L1 |
| | protein 1 | |
| IPI00005607 | Isoform 1 of Deleted in bladder cancer protein 1 precursor | DBC1 |
| IPI00027984 | Putative uncharacterized protein | DNAJC11 |
| IPI00032425 | Receptor activity-modifying protein 3 precursor | RAMP3 |
| IPI00020470 | Isoform 1 of Glycosyltransferase 8 domain-containing protein 1 | GLT8D1 |
| IPI00718821 | Isoform 1 of Uncharacterized protein C19orf55 | C19orf55 |
| IPI00018387 | Furin precursor | FURIN |
| IPI00398229 | similar to deubiquitinating enzyme 3 | LOC392197 |
| IPI00163851 | Isoform 1 of Eukaryotic translation initiation factor 2-alpha kinase 4 | EIF2AK4 |
| IPI00023780 | Isoform 2 of DnaJ homolog subfamily C member 5 | DNAJC5 |
| IPI00018027 | Isoform 1 of Angiogenic factor with G patch and FHA domains 1 | AGGF1 |
| IPI00010442 | Phospholemman precursor | FXYD1; FXYD7 |
| IPI00434711 | Putative uncharacterized protein FP6679 | — |
| IPI00184650 | Class B basic helix-loop-helix protein 4 | BHLHB4 |
| IPI00328298 | Isoform 2 of Structural maintenance of chromosomes protein 4 | SMC4 |
| IPI00014340 | Isoform 1 of Protein phosphatase 1 regulatory subunit 12C | PPP1R12C |
| IPI00386284 | olfactory receptor, family 2, subfamily AK, member 2 | OR2AK2 |
| IPI00018429 | Paired mesoderm homeobox protein 2 | PRRX2 |
| IPI00387004 | PNAS-146 | MCM7 |
| IPI00009471 | WD repeat-containing protein 3 | WDR3 |
| IPI00299263 | ADP-ribosylation factor GTPase-activating protein 3 | ARFGAP3 |
| IPI00045939 | 2-aminoethanethiol dioxygenase | ADO |
| IPI00014537 | Isoform 1 of Calumenin precursor | CALU |
| IPI00374670 | hypothetical protein isoform 2 | LOC388588 |
| IPI00289837 | Coiled-coil domain-containing protein 85A | CCDC85A |
| IPI00878962 | 10 kDa protein | — |
| IPI00103891 | Putative uncharacterized protein | MTMR14 |
| IPI00398154 | actin filament associated protein 1 | AFAP1 |
| IPI00291939 | Structural maintenance of chromosomes protein 1A | SMC1A |
| IPI00151990 | Isoform 1 of Thioredoxin domain-containing protein 15 precursor | TXNDC15 |
| IPI00003384 | Isoform 1 of Cadherin EGF LAG seven-pass G-type receptor 1 | CELSR1 |
| | precursor | |
| IPI00376383 | Centrosomal protein 110 kDa | CEP110 |
| IPI00015973 | Band 4.1-like protein 2 | EPB41L2 |
| IPI00420014 | Isoform 1 of U5 small nuclear ribonucleoprotein 200 kDa helicase | ASCC3L1 |
| IPI00386314 | FLJ00064 protein (Fragment) | CNDP2 |
| IPI00021766 | Isoform 1 of Reticulon-4 | RTN4 |
| IPI00103595 | Centrosome-associated protein 350 | CEP350 |
| IPI00006173 | Isoform 1 of Cholesteryl ester transfer protein precursor | CETP |
| IPI00183526 | NCL protein | NCL |
| IPI00020406 | Beta-1,4-mannosyl-glycoprotein 4-beta-N- | MGAT3 |
| | acetylglucosaminyltransferase | |
| IPI00022479 | guanine nucleotide exchange factor p532 | HERC1 |
| IPI00179452 | Isoform 1 of Protein CBFA2T2 | CBFA2T2 |
| IPI00017480 | Sonic hedgehog protein precursor | SHH |
| IPI00853133 | DEAD (Asp-Glu-Ala-Asp) box polypeptide 60-like | DDX60L |
| IPI00387132 | CDNA FLJ32661 fis, clone TESTI1000055, weakly similar to | hCG_2007354 |
| | HOMEOBOX PROTEIN SIX1 | |
| IPI00878369 | 12 kDa protein | — |
| IPI00382492 | Ig heavy chain V-III region LAY | — |
| IPI00334492 | Isoform 3 of Coiled-coil domain-containing protein 144A | LOC731479; CCDC144A; LOC652491; |
| | | CCDC144B |
| IPI00018880 | Tumor necrosis factor receptor superfamily member 1A precursor | TNFRSF1A |
| IPI00784385 | Adenylate cyclase type 1 | ADCY1 |
| IPI00465168 | Isoform 1 of Serine/threonine-protein kinase TAO2 | TAOK2 |
| IPI00028213 | Semaphorin-3D precursor | SEMA3D |
| IPI00296421 | EH domain-binding protein 1-like protein 1 | EHBP1L1 |
| IPI00787587 | similar to Temporarily Assigned Gene name family member | LOC729549 |
| IPI00028980 | Uncharacterized protein KIAA0133 | KIAA0133 |
| IPI00430792 | Isoform 1 of Protein ERGIC-53-like precursor | LMAN1L |
| IPI00018335 | Isoform Flt1 of Vascular endothelial growth factor receptor 1 | FLT1 |
| | precursor | |
| IPI00795736 | Fer-1-like 4 | — |
| IPI00000656 | Isoform 1 of Uncharacterized protein KIAA0892 precursor | KIAA0892 |
| IPI00169377 | Isoform 1 of GAS2-like protein 2 | GAS2L2 |
| IPI00030648 | Isoform 1 of Zinc finger FYVE domain-containing protein 9 | ZFYVE9 |
| IPI00419675 | hypothetical protein LOC150763 | LOC150763 |
| IPI00003893 | Isoform 1 of Protocadherin gamma B3 precursor | PCDHGB3 |
| IPI00410127 | Transforming, acidic coiled-coil containing protein 2 | TACC2 |
| IPI00005711 | Histone deacetylase 6 | HDAC6 |
| IPI00883997 | Coiled-coil domain-containing protein 19 | CCDC19 |
| IPI00419180 | CDNA FLJ41429 fis, clone BRHIP2005354 | CC2D2B |
| IPI00335711 | Ciliary dynein heavy chain 11 | DNAH11 |
| IPI00411291 | Peroxisome biogenesis factor 1 | PEX1 |
| IPI00397622 | Isoform 1 of Transmembrane and immunoglobulin domain-containing | TMIGD1 |
| | protein 1 precursor | |
| IPI00003564 | Lymphocyte antigen 86 precursor | LY86 |
| IPI00100247 | Thioredoxin domain-containing protein 13 precursor | TXNDC13 |
| IPI00059711 | LRP11 protein (Fragment) | LRP11 |
| IPI00141933 | Mitotic checkpoint serine/threonine-protein kinase BUB1 beta | BUB1B |
| IPI00387098 | Ig kappa chain V-I region OU | — |
| IPI00299554 | Kinesin-like protein KIF14 | KIF14 |
| IPI00515034 | 87 kDa protein | KIFAP3 |
| IPI00044608 | Isoform 1 of Protein KIAA1881 | KIAA1881 |
| IPI00444138 | CDNA FLJ46074 fis, clone TESTI2001915, highly similar to Homo | AFAP1 |
| | sapiens actin filament associated protein | |
| IPI00807406 | hypothetical protein isoform 1 | LOC285346 |
| IPI00645729 | 62 kDa protein | C1QTNF8 |
| IPI00789856 | tubulin tyrosine ligase-like family, member 3 | TTLL3 |
| IPI00032680 | Apolipoprotein-L5 | APOL5 |
| IPI00298214 | Isoform 1 of ATP-binding cassette sub-family G member 2 | ABCG2 |
| IPI00030945 | Urocortin-2 precursor | UCN2 |
| IPI00333503 | Uncharacterized protein ENSP00000315571 (Fragment) | — |
| IPI00005567 | Isoform 3A of Voltage-dependent L-type calcium channel subunit | CACNB3 |
| | beta-3 | |
| IPI00185038 | Isoform 1 of Dual oxidase 1 precursor | DUOX1 |
| IPI00005537 | 39S ribosomal protein L12, mitochondrial precursor | MRPL12 |
| IPI00795575 | 39 kDa protein | — |
| IPI00221240 | Isoform 2 of Leucyl-cystinyl aminopeptidase | LNPEP |
| IPI00655865 | Isoform 2 of Protein diaphanous homolog 3 | DIAPH3 |
| IPI00019270 | Tankyrase-2 | TNKS2 |
| IPI00302614 | V-set domain containing T cell activation inhibitor 1 | VTCN1 |
| IPI00176976 | Novel protein similar to mitochondrial ribosome recycling factor | RP1-57A13.2 |
| | isoform 1 | |
| IPI00786931 | similar to fatty acid amide hydrolase | LOC729037 |
| IPI00063878 | Isoform 1 of Multidrug resistance-associated protein 9 | ABCC12 |
| IPI00029473 | Protein Njmu-R1 | C17orf75 |
| IPI00884076 | ENV polyprotein (coat polyprotein) family protein | — |
| IPI00449923 | Isoform 1 of Retinoic acid-induced protein 1 | RAI1 |
| IPI00294187 | Protein-arginine deiminase type-2 | PADI2 |
| IPI00045473 | Similar to C. Elegans protein F17C8.5 | LOC339123 |
| IPI00167433 | CDNA FLJ39703 fis, clone SMINT2012195 | FDX1L |
| IPI00183572 | Isoform 2 of Dedicator of cytokinesis protein 7 | DOCK7 |
| IPI00001835 | Zinc finger and BTB domain-containing protein 4 | ZBTB4 |
| IPI00148768 | TRIO and F-actin binding protein isoform 1 | TRIOBP |
| IPI00170548 | Isoform 1 of ATPase family AAA domain-containing protein 2 | ATAD2 |
| IPI00220257 | Isoform 3 of Probable tubulin polyglutamylase | TTLL1 |
| IPI00298058 | Isoform 1 of Transcription elongation factor SPT5 | SUPT5H |
| IPI00102281 | Retroviral-like aspartic protease 1 precursor | SASP |
| IPI00030355 | Protein phosphatase 1 regulatory subunit 11 | PPP1R11 |
| IPI00217915 | Isoform 1 of Transmembrane protein 136 | TMEM136 |
| IPI00220986 | Isoform 3 of ADAMTS-9 precursor | ADAMTS9 |
| IPI00175169 | Isoform 1 of ADP-ribosylation factor GTPase-activating protein 1 | ARFGAP1 |
| IPI00550862 | hypothetical protein LOC387911 | LOC387911 |
| IPI00746459 | CDNA FLJ34517 fis, clone HLUNG2006781 | — |
| IPI00444234 | CDNA FLJ45743 fis, clone KIDNE2016464 | FLJ45743 |
| IPI00002534 | Isoform 1 of Purine-rich element-binding protein gamma | PURG |
| IPI00556253 | Isoform 1 of Coiled-coil domain-containing protein C6orf199 | C6orf199 |
| IPI00017592 | Leucine zipper-EF-hand-containing transmembrane protein 1, | LETM1 |
| | mitochondrial precursor | |
| IPI00016007 | Myosin-Vc | MYO5C |
| IPI00744927 | gastrotropin isoform 1 | FABP6 |
| IPI00024319 | Forkhead box protein J1 | FOXJ1 |
| IPI00339297 | Polypeptide N-acetylgalactosaminyltransferase 4 | GALNT4 |
| IPI00254408 | bromodomain PHD finger transcription factor isoform 1 | BPTF |
| IPI00304324 | Histone deacetylase 11 | HDAC11 |
| IPI00477693 | fatty acid binding protein | LOC646486 |
| IPI00025310 | Zinc finger protein 217 | ZNF217 |
| IPI00427808 | Isoform 1 of Uncharacterized protein C10orf71 | C10orf71 |
| IPI00735313 | similar to Peptidyl-prolyl cis-trans isomerase NIMA-interacting 4 | — |
| IPI00384013 | Polypeptide N-acetylgalactosaminyltransferase-like protein 2 | GALNTL2 |
| IPI00382495 | Ig heavy chain V-III region ZAP | — |
| IPI00477820 | Isoform 1 of FRAS1-related extracellular matrix protein I precursor | FREM1 |
| IPI00024776 | Calmegin precursor | CLGN |
| IPI00023110 | Potassium channel subfamily K member 3 | KCNK3 |
| IPI00182833 | Coiled-coil alpha-helical rod protein 1 | CCHCR1 |
| IPI00394994 | Isoform 1 of Nesprin-3 | C14orf49 |
| IPI00419992 | Isoform 1 of Acyl-CoA synthetase family member 4 | AASDH |
| IPI00004445 | Gamma-2-syntrophin | SNTG2 |
| IPI00878015 | 12 kDa protein | — |
| IPI00328094 | Isoform 1 of Forkhead box protein P3 | FOXP3 |
| IPI00873348 | 63 kDa protein | A1CF |
| IPI00640240 | 59 kDa protein | SPTLC3 |
| IPI00002824 | Cysteine and glycine-rich protein 2 | CSRP2 |
| IPI00398272 | Isoform 1 of Collagen alpha-1(XVII) chain | COL17A1 |
| IPI00061009 | Isoform 1 of WD repeat-containing protein 67 | WDR67 |
| IPI00299301 | Isoform 1 of Desmuslin | DMN |
| IPI00152011 | PTPL1-associated RhoGAP 1 | ARHGAP29 |
| IPI00847790 | Protein of unknown function DUF1725 domain containing protein | — |
| IPI00065121 | Isoform 2 of Coiled-coil domain-containing protein 36 | CCDC36 |
| IPI00373867 | glycolipid transfer protein domain containing 2 | GLTPD2 |
| IPI00872354 | armadillo repeat containing 2 | ARMC2 |
| IPI00152975 | Isoform 1 of Coiled-coil domain-containing protein 90A, | CCDC90A |
| | mitochondrial precursor | |
| IPI00374804 | Isoform 1 of Diacylglycerol kinase delta | DGKD |
| IPI00292894 | Pre-rRNA-processing protein TSR1 homolog | TSR1 |
| IPI00028232 | Putative uncharacterized protein | FGF4 |
| IPI00027269 | E3 ubiquitin-protein ligase CBL | CBL |
| IPI00797567 | 9 kDa protein | GNAI2 |
| IPI00032958 | Isoform 2 of Actin-binding protein anillin | ANLN |
| IPI00057815 | intestinal facilitative glucose transporter 7 | SLC2A7 |
| IPI00014367 | Isoform 1 of Non-SMC element 4 homolog A | NSMCE4A |
| IPI00884022 | Similar to Immunglobulin heavy chain variable region | — |
| IPI00465319 | Chondroitin sulfate synthase 2 | CHPF |
| IPI00549844 | Isoform 1 of Protein FAM134B | FAMI34B |
| IPI00457114 | Isoform 1 of IQ motif and Sec7 domain-containing protein 1 | IQSEC1 |
| IPI00002773 | Isoform 2 of Tyrosine-protein kinase JAK3 | JAK3 |
| IPI00005667 | NEDD4-binding protein 1 | N4BP1 |
| IPI00300594 | CDNA FLJ35435 fis, clone SMINT2002620 | SPATA13 |
| IPI00103142 | NudC domain-containing protein 2 | NUDCD2 |
| IPI00290785 | Isoform 1 of SET domain-containing protein 6 | SETD6 |
| IPI00297763 | Retinal-specific ATP-binding cassette transporter | ABCA4 |
| IPI00000861 | Isoform 1 of LIM and SH3 domain protein 1 | LASP1 |
| IPI00168255 | hypothetical protein LOC400451 | LOC400451 |
| IPI00015990 | Isoform 1 of Eyes absent homolog 3 | EYA3 |
| IPI00171500 | Uncharacterized protein C14orf45 | C14orf45 |
| IPI00170765 | Isoform 3 of Alanine--glyoxylate aminotransferase 2-like 2 | AGXT2L2 |
| IPI00099977 | platelet-derived growth factor C precursor | PDGFC |
| IPI00292953 | Isoform 2 of Ankycorbin | RAI14 |
| IPI00159072 | 57 kDa protein | ROD1 |
| IPI00305692 | Thioredoxin-like protein 1 | TXNL1 |
| IPI00031630 | Melanocyte protein Pmel 17 precursor | SILV |
| IPI00167446 | Isoform 2 of Uncharacterized protein C17orf78 | C17orf78 |
| IPI00479625 | Isoform 1 of ELAV-like protein 4 | ELAVL4 |
| IPI00745395 | Conserved hypothetical protein | KIAA0508 |
| IPI00043069 | Ankyrin repeat domain-containing protein 30A | ANKRD30A |
| IPI00014316 | Cyclin-dependent kinase 5 activator 1 precursor | CDK5R1 |
| IPI00301726 | Isoform 1 of Uncharacterized protein C3orf20 | C3orf20 |
| IPI00185361 | ATP-dependent RNA helicase DDX55 | DDX55 |
| IPI00293867 | D-dopachrome decarboxylase | DDT |
| IPI00794779 | DNA polymerase theta | POLQ |
| IPI00478354 | Putative uncharacterized protein CXorf31 | CXorf31 |
| IPI00166414 | Coiled-coil domain-containing protein 96 | CCDC96 |
| IPI00300053 | Keratin type II cuticular Hb2 | KRT82 |
| IPI00749062 | Conserved hypothetical protein | — |
| IPI00452161 | Isoform 1 of Mucolipin-1 | MCOLN1 |
| IPI00384546 | CDNA FLJ25883 fis, clone CBR02735 | — |
| IPI00412272 | SH3 domain-binding glutamic acid-rich-like protein 2 | SH3BGRL2 |
| IPI00006298 | Isoform 1 of Peptidyl-prolyl cis-trans isomerase G | PPIG |
| IPI00031497 | Conserved hypothetical protein | C7orf49 |
| IPI00022061 | Sodium/hydrogen exchanger 6 | SLC9A6 |
| IPI00217418 | Isoform 2 of Rho GTPase-activating protein 12 | ARHGAP12 |
| IPI00743545 | Conserved hypothetical protein | — |
| IPI00328156 | Amine oxidase [flavin-containing] B | MAOB |
| IPI00644785 | Protein | PLXNB3 |
| IPI00654693 | Protein FAM44C | FAM44C |
| IPI00880011 | 116 kDa protein | — |
| IPI00018583 | Isoform 1 of Intracellular hyaluronan-binding protein 4 | HABP4 |
| IPI00030986 | Isoform Long of Kelch repeat and BTB domain-containing protein 10 | KBTBD10 |
| IPI00045503 | Isoform 2 of Protein phosphatase 1 regulatory subunit 14A | PPP1R14A |
| IPI00793408 | 6 kDa protein | ADAMTS9 |
| IPI00024255 | G patch domain and KOW motifs-containing protein | GPKOW |
| IPI00028262 | 67 kDa protein | KIAA1754 |
| IPI00027212 | Insulin receptor-related protein precursor | INSRR |
| IPI00009724 | Isoform 1 of EF-hand calcium-binding domain-containing protein 6 | EFCAB6 |
| IPI00868835 | Similar to Heterogeneous nuclear ribonucleoproteins C1/C2 (hnRNP | — |
| | C1/hnRNP C2). Isoform 4 | |
| IPI00396658 | Isoform 2 of Protein ITFG3 | ITFG3 |
| IPI00742127 | Similar to Heterogeneous nuclear ribonucleoprotein A1 (Helix- | — |
| | destabilizing protein) (Single-strand RNA-binding protein) (hnRNP | |
| | core protein A1). Isoform A1-A | |
| IPI00398586 | hypothetical protein LOC199800 | LOC199800 |
| IPI00418164 | Novel protein | RP4-692D3.1; LOC731440 |
| IPI00033019 | Potassium voltage-gated channel subfamily B member 1 | KCNB1 |
| IPI00032598 | Isoform 1 of ETS translocation variant 3 | ETV3 |
| IPI00217494 | Isoform 1 of Protein SMG7 | SMG7 |
| IPI00000837 | Metabotropic glutamate receptor 3 precursor | GRM3 |
| IPI00296535 | 2-hydroxyacyl-CoA lyase 1 | HACL1 |
| IPI00062882 | ATP-binding domain-containing protein 3 | ATPBD3 |
| IPI00445167 | CDNA FLJ44499 fis, clone UTERU3000665, highly similar to Homo | SRCAP |
| | sapiens Snf2-related CBP activator protein | |
| IPI00442582 | CDNA FLJ26893 fis, clone RCT00305 | — |
| IPI00011274 | Isoform 1 of Heterogeneous nuclear ribonucleoprotein D-like | HNRPDL |
| IPI00002791 | Isoform Wnt-16b of Protein Wnt-16 precursor | WNT16 |
| IPI00290432 | Natural resistance-associated macrophage protein 1 | SLC11A1 |
| IPI00012540 | Prominin-1 precursor | PROM1 |
| IPI00477396 | Uncharacterized protein ZNF324B (Fragment) | ZNF584; ZNF324B |
| IPI00853149 | RW1 protein | TMEM131 |
| IPI00168977 | Olfactory receptor 6J1 | OR6J1 |
| IPI00165955 | Isoform 1 of Mitogen-activated protein kinase 15 | MAPK15 |
| IPI00377211 | Isoform 3 of Cadherin-like protein 26 precursor | CDH26 |
| IPI00736558 | THAP domain containing 4 | THAP4 |
| IPI00470518 | Mitotic spindle assembly checkpoint protein MAD1 | MAD1L1 |
| IPI00005087 | Tropomodulin-3 | TMOD3 |
| IPI00029227 | LOC93622 protein | LOC93622 |
| IPI00218839 | Cytochrome P450 1A1 | CYP1A1 |
| IPI00006356 | Isoform 1 of Patatin-like phospholipase domain-containing protein 5 | PNPLA5 |
| IPI00005616 | Isoform 1 of Trace amine-associated receptor 2 | TAAR2 |
| IPI00789494 | 8 kDa protein | TUSC4 |
| IPI00745346 | Isoform 11 of Peroxisomal N(1)-acetyl-spermine/spermidine oxidase | PAOX |
| IPI00027596 | Secreted frizzled-related protein 2 precursor | SFRP2 |
| IPI00008455 | Isoform 2 of Myosin-VI | MYO6 |
| IPI00217850 | Isoform 1 of GRAM domain-containing protein 1C | GRAMD1C |
| IPI00020771 | Isoform 3 of Ensconsin | MAP7 |
| IPI00170800 | Isoform 1 of Otoancorin precursor | OTOA |
| IPI00063181 | CDKN2A interacting protein N-terminal like | CDKN2AIPNL |
| IPI00328225 | Isoform 1 of T-box transcription factor TBX20 | TBX20 |
| IPI00166331 | Ankyrin repeat domain-containing protein 35 | ANKRD35 |
| IPI00294386 | Myosin-Ia | MYO1A |
| IPI00061680 | SAGA-associated factor 29 homolog | CCDC101 |
| IPI00220901 | TBC1D4 protein | TBC1D4 |
| IPI00059139 | Vacuolar proton pump subunit E 2 | ATP6V1E2 |
| IPI00010893 | Left-right determination factor 2 precursor | LEFTY2 |
| IPI00746222 | ATP-binding cassette, sub-family B (MDR/TAP), member 5 | ABCB5 |
| IPI00010843 | Isoform 1 of Amyloid beta A4 precursor protein-binding family B | APBB1 |
| | member 1 | |
| IPI00012353 | Mitochondrial 39S ribosomal protein L3 | MRPL3 |
| IPI00385065 | Isoform 1 of Coiled-coil domain-containing protein 46 | CCDC46 |
| IPI00413930 | Elongation of very long chain fatty acids protein 2 | ELOVL2 |
| IPI00006715 | Double-strand-break repair protein rad21 homolog | RAD21 |
| IPI00787189 | similar to thioredoxin domain containing 4 | LOC727931 |
| IPI00215965 | Isoform A1-B of Heterogeneous nuclear ribonucleoprotein A1 | HNRNPA1 |
| IPI00030237 | Isoform 1 of Uncharacterized protein C1orf49 | C1orf49 |
| IPI00088953 | Phosphoinositide 3-kinase regulatory subunit 6 | C17orf38 |
| IPI00031490 | Isoform 1 of Collectin-11 precursor | COLEC11 |
| IPI00430079 | Isoform 2 of PHD finger protein 8 | PHF8 |
| IPI00296942 | Cadherin-12 precursor | CDH12 |
| IPI00852688 | Isoform 1 of Low density lipoprotein receptor-related protein 5-like | LRP5L |
| | protein | |
| IPI00060440 | Zinc finger protein GLI4 | GLI4; ZFP41 |
| IPI00845299 | Putative uncharacterized protein LOC285733 | LOC285733 |
| IPI00003897 | Isoform 1 of Protocadherin gamma B6 precursor | PCDHGB6 |
| IPI00152257 | Enkurin | C10orf63 |
| IPI00043526 | Uncharacterized protein C14orf44 | C14orf44 |
| IPI00013373 | Orexin precursor | HCRT |
| IPI00829925 | Uncharacterized protein ENSP00000365090 | ELA2A |
| IPI00009333 | Transmembrane protein 9 precursor | TMEM9 |
| IPI00470917 | Isoform 2 of Uncharacterized protein KIAA1529 | KIAA1529 |
| IPI00013269 | Isoform 1 of Uncharacterized protein C6orf106 | C6orf106 |
| IPI00022433 | Heat-shock protein beta-6 | HSPB6 |
| IPI00179694 | Beta-klotho | KLB |
| IPI00027642 | Isoform 1 of JmjC domain-containing histone demethylation protein | JMJD2C |
| | 3C | |
| IPI00797832 | Isoform 3 of Acyl-CoA dehydrogenase family member 10 | ACAD10 |
| IPI00410464 | Isoform 1 of Zinc finger protein 324B | ZNF324B; ZNF584 |
| IPI00166863 | Isoform 2 of Coiled-coil domain-containing protein 123, | CCDC123 |
| | mitochondrial precursor | |
| IPI00008832 | Growth arrest-specific protein 1 precursor | GAS1 |
| IPI00014958 | Isoform 1 of Serum paraoxonase/arylesterase 2 | PON2 |
| IPI00719669 | Myosin regulatory light chain | MRLC2 |
| IPI00299084 | Transmembrane protein 33 | TMEM33 |
| IPI00029737 | Isoform Long of Long-chain-fatty-acid--CoA ligase 4 | ACSL4 |
| IPI00005661 | Isoform Gamma of Max-like protein X | MLX |
| IPI00217683 | A-kinase anchor protein 12 isoform 2 | AKAP12 |
| IPI00418966 | hypothetical protein LOC123688 isoform 2 | LOC123688 |
| IPI00328409 | Armadillo repeat-containing protein 4 | ARMC4 |
| IPI00065457 | OTTHUMP00000018353 | FLJ32682 |
| IPI00444502 | CDNA FLJ45482 fis, clone BRTHA2001953 | FLJ45482 |
| IPI00001780 | Ubiquitin carboxyl-terminal hydrolase 16 | USP16 |
| IPI00552840 | Probable G-protein coupled receptor 112 | GPR112 |
| IPI00016373 | Ras-related protein Rab-13 | RAB13 |
| IPI00552434 | Myosin, heavy chain 7B, cardiac muscle, beta | MYH7B |
| IPI00876962 | Isoform 2 of Inverted formin-2 | INF2 |
| IPI00026512 | Isoform HERA-A of GTP-binding protein era homolog | ERAL1 |
| IPI00180375 | Isoform 1 of Beta-arrestin-2 | ARRB2 |
| IPI00297407 | Solute carrier family 45 member 4 | SLC45A4 |
| IPI00152879 | Monocarboxylate transporter 10 | SLC16A10 |
| IPI00290155 | 66 kDa protein | ZNF148 |
| IPI00747017 | Putative uncharacterized protein DKFZp686D06121 | NEK1 |
| IPI00793836 | 11 kDa protein | ARHGDIB |
| IPI00382422 | Ig lambda chain V-I region NEWM | — |
| IPI00328842 | ARHGAP4 protein | ARHGAP4 |
| IPI00008632 | Isoform 1 of Putative homeodomain transcription factor 1 | PHTF1 |
| IPI00065253 | Uncharacterized protein C3orf30 | C3orf30 |
| IPI00883751 | RNA-directed DNA polymerase (Reverse transcriptase), related | — |
| | domain containing protein | |
| IPI00216137 | Synaptonemal complex protein 1 | SYCP1 |
| IPI00005621 | 7h3 protein (Fragment) | SYDE1 |
| IPI00001933 | Coiled-coil domain-containing protein 113 | CCDC113 |
| IPI00383294 | Putative uncharacterized protein pp7518 | MNT |
| IPI00016861 | 102 kDa protein | GTF3C2 |
| IPI00020903 | Isoform 1 of AF4/FMR2 family member 2 | AFF2 |
| IPI00465247 | Isoform 1 of Stimulated by retinoic acid gene 6 protein homolog | STRA6 |
| IPI00382394 | Isoform 5 of Cytosolic phospholipase A2 beta | PLA2G4B |
| IPI00167913 | Isoform 1 of Spermatid-associated protein | SPERT |
| IPI00874276 | Putative uncharacterized protein DKFZp761N1814 | EPB41L4B |
| IPI00446767 | Isoform 3 of Transducin-like enhancer protein 4 | TLE4 |
| IPI00020754 | PRO1751 | — |
| IPI00300060 | WD repeat-containing protein 70 | WDR70 |
| IPI00021979 | Peroxisomal membrane protein 11A | PEX11A |
| IPI00031768 | Hook homolog 3 | HOOK3 |
| IPI00216986 | Isoform C of Complement receptor type 2 precursor | CR2 |
| IPI00470771 | SAMD4B protein | SAMD4B |
| IPI00021143 | Class B basic helix-loop-helix protein 2 | BHLHB2 |
| IPI00297931 | Isoform 1 of AP1 subunit gamma-binding protein 1 | AP1GBP1 |
| IPI00018691 | 28S ribosomal protein S18a, mitochondrial precursor | MRPS18A |
| IPI00010604 | Isoform 1 of 1-phosphatidylinositol-4,5-bisphosphate | PLCE1 |
| | phosphodiesterase epsilon-1 | |
| IPI00022438 | Isoform Long of Transcription factor Maf | MAF |
| IPI00396527 | Isoform 1 of Coiled-coil domain-containing protein 128 | CCDC128 |
| IPI00291840 | Isoform 1 of Zinc finger protein 90 homolog | ZFP90 |
| IPI00646410 | Isoform 2 of Flavin-containing amine oxidase domain-containing | AOF1 |
| | protein 1 | |
| IPI00451413 | Intersectin 1 isoform 7 | ITSN1 |
| IPI00787410 | similar to Polypeptide N-acetylgalactosaminyltransferase 9 | GALNT9 |
| IPI00552601 | Putative uncharacterized protein DKFZp686M12165 (Fragment) | — |
| IPI00166833 | LAG1 longevity assurance homolog 5 | LASS5 |
| IPI00183425 | CDKN1A interacting zinc finger protein 1 | CIZ1 |
| IPI00027228 | Probable glutamyl-tRNA(Gln) amidotransferase subunit B, | PET112L |
| | mitochondrial precursor | |
| IPI00007917 | Collectin-10 precursor | COLEC10 |
| IPI00552787 | similar to Matrin-3 | RBM20 |
| IPI00065484 | CDNA FLJ32471 fis, clone SKNMC2000322, weakly similar to | PTRH2 |
| | MAJOR CENTROMERE AUTOANTIGEN B | |
| IPI00384447 | Sarcoma antigen NY-SAR-22 (Fragment) | NEXN |
| IPI00830073 | Isoform 1 of RING finger protein 207 | RNF207 |
| IPI00012411 | Zinc finger protein 32 | ZNF32 |
| IPI00760993 | IQ motif and Sec7 domain 2 isoform 2 | IQSEC2 |
| IPI00004527 | Uncharacterized protein KIAA0355 | KIAA0355 |
| IPI00018956 | ALX homeobox protein 1 | ALX1 |
| IPI00154553 | Centrosomal protein of 76 kDa | CEP76 |
| IPI00444208 | CDNA FLJ45771 fis, clone NETRP2003103 | RNF44 |
| IPI00001641 | Origin recognition complex subunit 6 | ORC6L |
| IPI00216484 | Isoform 2 of LisH domain-containing protein ARMC9 | ARMC9 |
| IPI00170961 | Isoform 2 of WD repeat-containing protein 16 | WDR16 |
| IPI00786880 | Myosin-XVB | — |
| IPI00152722 | Protein CCDC35 | CCDC35 |
| IPI00298447 | Probable methyltransferase TARBP1 | TARBP1 |
| IPI00293200 | OTTHUMP00000016423 | PARC |
| IPI00550272 | Integrator complex subunit 10 | INTS10 |
| IPI00514197 | similar to Nonhistone chromosomal protein HMG-17 | LOC648822 |
| IPI00156804 | cDNA FLJ77550, highly similar to Homo sapiens zinc finger protein | ZNF665 |
| | 665 (ZNF665), mRNA | |
| IPI00384140 | Full-length cDNA clone CS0DA005YA22 of Neuroblastoma of Homo | ESR2 |
| | sapiens (Fragment) | |
| IPI00410564 | CDNA FLJ45248 fis, clone BRHIP2006819 | FLJ45248 |
| IPI00023586 | Isoform 1 of Sentrin-specific protease 6 | SENP6 |
| IPI00028579 | Transcription initiation factor TFIID subunit 6 | TAF6 |
| IPI00253323 | Ankyrin repeat domain-containing protein 57 | ANKRD57 |
| IPI00166361 | Putative uncharacterized protein FLJ33590 | FLJ33590 |
| IPI00294810 | CDNA FLJ10824 fis, clone NT2RP4001086 (Fragment) | FAM21A |
| IPI00418408 | Cytochrome P450 | CYP2D7P1 |
| IPI00168627 | Uncharacterized protein CXorf20 | CXorf20 |
| IPI00026952 | Plakophilin-3 | PKP3 |
| IPI00014852 | Isoform 1 of Phosphoglucomutase-like protein 5 | PGM5 |
| IPI00332628 | Otopetrin-3 | OTOP3 |
| IPI00296830 | Isoform 1 of Leucine zipper protein 1 | LUZP1 |
| IPI00306127 | THUMP domain-containing protein 3 | THUMPD3 |
| IPI00022881 | Isoform 1 of Clathrin heavy chain 2 | CLTCL1 |
| IPI00397015 | A.T hook DNA-binding motif-containing protein 1 | AHDC1 |
| IPI00029184 | Hyaluronan and proteoglycan link protein 2 precursor | HAPLN2 |
| IPI00795917 | 126 kDa protein | CXorf45 |
| IPI00815976 | Transmembrane protein 120B | TMEM120B |
| IPI00382671 | Isoform 6 of C-type lectin domain family 4 member M | CLEC4M |
| IPI00018303 | Isoform 2 of Protocadherin-11 X-linked precursor | PCDH11X |
| IPI00176210 | hypothetical protein LOC133558 | FLJ40243 |
| IPI00166153 | Protein KIAA0082 | KIAA0082 |
| IPI00300621 | Isoform 2B2 of Synaptojanin-2 | SYNJ2 |
| IPI00306166 | germ cell associated 1 isoform 2 | GSG1 |
| IPI00401002 | Zinc finger protein 740 | ZNF740 |
| IPI00607829 | Isoform 1 of Ret finger protein-like 2 | RFPL2 |
| IPI00402280 | Isoform 1 of Small leucine-rich proteoglycan family member | CHADL |
| | LOC150356 precursor | |
| IPI00006800 | Isoform Long of Autophagy protein 5 | ATG5 |
| IPI00218559 | Gamma-sarcoglycan | SGCG |
| IPI00479897 | Chromosome 9 open reading frame 75 | C9orf75 |
| IPI00216003 | Cullin-5 | CUL5 |
| IPI00445796 | CDNA FLJ43383 fis, clone OCBBF2006058, highly similar to Homo | ACAD8 |
| | sapiens acyl-Coenzyme A dehydrogenase-8 mRNA | |
| IPI00742114 | similar to tripartite motif protein 11 | LOC653978 |
| IPI00023164 | Potassium/sodium hyperpolarization-activated cyclic nucleotide-gated | HCN4 |
| | channel 4 | |
| IPI00012480 | FKSG42 | RACGAP1P |
| IPI00004538 | similar to nascent polypeptide-associated complex alpha polypeptide | NACAD |
| | isoform 1 | |
| IPI00297462 | Uncharacterized protein C1orf65 | C1orf65 |
| IPI00793454 | 7 kDa protein | USP19 |
| IPI00028392 | specifically androgen-regulated protein isoform 1 | C1orf116 |
| IPI00385495 | Transmembrane protein 153 | LMF2 |
| IPI00017726 | Isoform 1 of 3-hydroxyacyl-CoA dehydrogenase type-2 | HSD17B10 |
| IPI00791509 | Neural precursor cell expressed, developmentally down-regulated 1 | NEDD1 |
| IPI00289931 | Isoform 1 of Mucosal addressin cell adhesion molecule 1 precursor | MADCAM1 |
| IPI00442601 | CDNA FLJ26817 fis, clone PRS06446 | — |
| IPI00012891 | Phosphorylase b kinase gamma catalytic chain, testis/liver isoform | PHKG2 |
| IPI00021338 | Dihydrolipoyllysine-residue acetyltransferase component of pyruvate | DLAT |
| | dehydrogenase complex, mitochondrial precursor | |
| IPI00455083 | Uncharacterized protein ENSP00000371558 | — |
| IPI00168877 | helicase (DNA) B | HELB |
| IPI00641214 | 177 kDa protein | ADCY10 |
| IPI00290837 | Isoform 2 of F-box/WD repeat-containing protein 10 | FBXW10 |
| IPI00009213 | Isoform 1 of B-cell lymphoma/leukemia 11B | BCL11B |
| IPI00003421 | T-brain-1 protein | TBR1 |
| IPI00396314 | Isoform 3 of CCR4-NOT transcription complex subunit 10 | CNOT10 |
| IPI00000106 | Signal transducer and activator of transcription 4 | STAT4 |
| IPI00339217 | Ovochymase-1 precursor | OVCH1 |
| IPI00395631 | Isoform 1 of E3 ubiquitin-protein ligase TRAF7 | TRAF7 |
| IPI00455397 | DMWD protein | DMWD |
| IPI00744557 | kazrin isoform E | RP1-21O18.1 |
| IPI00152692 | D-tyrosyl-tRNA(Tyr) deacylase 1 | DTD1 |
| IPI00397526 | Isoform 1 of Myosin-10 | MYH10 |
| IPI00640654 | RAP1 GTPase activating protein | RAP1GAP |
| IPI00791831 | 10 kDa protein | ADRBK1 |
| IPI00479904 | Uncharacterized protein COL27A1 | COL27A1 |
| IPI00013256 | Isoform 1 of Cleavage stimulation factor 64 kDa subunit | CSTF2 |
| IPI00059279 | Exocyst complex component 4 | EXOC4 |
| IPI00299402 | Pyruvate carboxylase, mitochondrial precursor | PC |
| IPI00167639 | Vacuolar proton pump subunit d 2 | ATP6V0D2 |
| IPI00170855 | tetratricopeptide repeat domain 19 | TTC19 |
| IPI00445563 | Putative uncharacterized protein MGC21675/FLJ43787 | C4orf42 |
| IPI00884334 | Conserved hypothetical protein | LOC729968 |
| IPI00007910 | Isoform 1 of Sodium-dependent phosphate transport protein 2B | SLC34A2 |
| IPI00083708 | BAT2-iso | BAT2D1 |
| IPI00328715 | Protein LYRIC | MTDH |
| IPI00290035 | Isoform 1 of Protocadherin-15 precursor | PCDH15 |
| IPI00385511 | Uncharacterized protein TNRC6B | TNRC6B |
| IPI00006561 | Isoform 1 of Protein KIAA0317 | KIAA0317 |
| IPI00375294 | Laminin subunit alpha-1 precursor | LAMA1 |
| IPI00375325 | Keratin-associated protein 10-10 | KRTAP10-10 |
| IPI00640293 | 23 kDa protein | TIMP3 |
| IPI00235003 | Tumor necrosis factor receptor superfamily, member 6 isoform 1 | FAS |
| | variant (Fragment) | |
| IPI00216702 | Isoform 1 of ATP-binding cassette sub-family A member 9 | ABCA9 |
| IPI00167930 | ring finger protein 190 | MARCH10 |
| IPI00470657 | Anti-colorectal carcinoma heavy chain | — |
| IPI00413868 | hypothetical protein LOC79632 isoform 1 | C6orf60 |
| IPI00066367 | Endothelial cells scavenger receptor precursor | SCARF1 |
| IPI00397740 | zinc finger protein 749 | ZNF749 |
| IPI00300426 | Isoform 1 of Tetratricopeptide repeat protein 29 | TTC29 |
| IPI00307259 | DnaJ homolog subfamily C member 13 | DNAJC13 |
| IPI00386113 | CDNA: FLJ23412 fis, clone HEP20516 | C20orf59 |
| IPI00153060 | Angiopoietin-related protein 4 precursor | ANGPTL4 |
| IPI00020354 | Interleukin-13 receptor alpha-1 chain precursor | IL13RA1 |
| IPI00177888 | Isoform 1 of Leukocyte receptor cluster member 8 | LENG8 |
| IPI00101267 | Isoform 1 of Uncharacterized protein C19orf60 | C19orf60 |
| IPI00739927 | similar to vasoactive intestinal peptide receptor 2 | LOC645464 |
| IPI00782965 | huntingtin interacting protein 1 | HIP1 |
| IPI00444375 | Rh blood group, CcEe antigens | RHCE |
| IPI00794880 | Isoform 1 of Chromodomain-helicase-DNA-binding protein 7 | CHD7 |
| IPI00419791 | Isoform 1 of Arginine/serine-rich coiled-coil protein 2 | RSRC2 |
| IPI10100106 | resistance to inhibitors of cholinesterase 8 homolog A | RIC8A |
| IPI00216593 | Isoform NOH-1LV of NADPH oxidase homolog 1 | NOX1 |
| IPI00395667 | Interferon-related IFRD2 (PC4-B) protein | NAT6; IFRD2 |
| IPI00008708 | Ribosomal L1 domain-containing protein 1 | RSL1D1 |
| IPI00748649 | 25 kDa protein | — |
| IPI00100362 | erythrocyte membrane protein band 4.1 like 4B isoform 1 | EPB41L4B |
| IPI00219898 | Isoform 2 of Nephrin precursor | NPHS1; PRODH2 |
| IPI00872098 | Zinc finger protein Pegasus | IKZF5 |
| IPI00043294 | Isoform 1 of Uncharacterized protein C7orf26 | C7orf26 |
| IPI00152535 | Chromodomain-helicase-DNA-binding protein 5 | CHD5 |
| IPI00217507 | Neurofilament medium polypeptide | NEFM |
| IPI00216356 | Isoform 2 of RasGAP-activating-like protein 1 | RASAL1 |
| IPI00290562 | Nuclear factor interleukin-3-regulated protein | NFIL3 |
| IPI00006003 | CD83 antigen precursor | CD83 |
| IPI00166528 | Isoform 3 of Rapamycin-insensitive companion of mTOR | RICTOR |
| IPI00221325 | E3 SUMO-protein ligase RanBP2 | RANBP2 |
| IPI00084684 | similar to Zinc finger protein 469 | ZNF469 |
| IPI00643809 | Protein | CNTNAP3B |
| IPI00164776 | Similar to RIKEN cDNA A230078105 gene | TMEM198 |
| IPI00023410 | Integrin alpha-8 precursor | ITGA8 |
| IPI00787593 | similar to CG33300-PA | LOC730407 |
| IPI00180781 | Isoform 1 of Mixed lineage kinase domain-like protein | MLKL |
| IPI00339309 | Isoform 1 of Proto-oncogene DBL | MCF2 |
| IPI00843923 | Phosphodiesterase PDE7B2 | PDE7B |
| IPI00640865 | Integrin beta-like protein 1 precursor | ITGBL1 |
| IPI00029144 | Isoform PR130 of Serine/threonine-protein phosphatase 2A regulatory | PPP2R3A |
| | subunit B″ subunit alpha | |
| IPI00175439 | F-box only protein 43 | FBXO43 |
| IPI00646485 | Protein | EFCAB2 |
| IPI00023105 | Peptidylglycine alpha-amidating monooxygenase COOH-terminal | PAMCI |
| | interactor | |
| IPI00301144 | Isoform 1 of Sulfatase-modifying factor 1 precursor | SUMF1 |
| IPI00439935 | UDP-glucuronosyltransferase 1-4 precursor | UGT1A4; UGT1A6 |
| IPI00001753 | Myosin-4 | MYH4 |
| IPI00401829 | Putative uncharacterized protein ENST00000281581 | — |
| IPI00056324 | Isoform 1 of Hermansky-Pudlak syndrome 3 protein | HPS3 |
| IPI00220493 | U6 snRNA-associated Sm-like protein LSm5 | LSM5 |
| IPI00386390 | Olfactory receptor 6N2 | OR6N2 |
| IPI00044761 | Pseudouridylate synthase 7 homolog | PUS7 |
| IPI00032342 | TRIP12 protein | TRIP12 |
| IPI00307536 | Isoform 1 of Testis-expressed sequence 11 protein | TEX11 |
| IPI00244812 | Tetratricopeptide repeat protein 6 | TTC6 |
| IPI00030487 | CDNA FLJ30993 fis, clone HLUNG1000064, weakly similar to | KIAA1627 |
| | KARYOGAMY PROTEIN KAR4 | |
| IPI00787443 | similar to Collagen alpha-2(XI) chain precursor | LOC730037 |
| IPI00013371 | Protein sprouty homolog 3 | SPRY3 |
| IPI00414259 | Isoform 2 of Pregnancy-specific beta-1-glycoprotein 4 precursor | PSG4 |
| IPI00410590 | Isoform 2 of LSM14 protein homolog A | LSM14A |
| IPI00023330 | Isoform 2 of LisH domain and HEAT repeat-containing protein | KIAA1468 |
| | KIAA1468 | |
| IPI00645614 | Isoform 2 of Cadherin-3 precursor | CDH3 |
| IPI00744574 | Similar to Tropomyosin alpha-4 chain | — |
| IPI00737735 | similar to cell division cycle 10 isoform 1 | LOC646913 |
| IPI00065415 | Isoform 1 of Coiled-coil domain-containing protein 138 | CCDC138 |
| IPI00376206 | Isoform 2 of 17-beta hydroxysteroid dehydrogenase 13 precursor | HSD17B13 |
| IPI00011589 | Isoform A of Septin-4 | SEPT4 |
| IPI00032158 | Isoform 2 of NMDA receptor-regulated protein 1 | NARG1 |
| IPI00013468 | Mitotic checkpoint protein BUB3 | BUB3 |
| IPI00745955 | EBNA1 binding_protein 2 | EBNA1BP2 |
| IPI00157237 | Isoform 1 of MAGUK p55 subfamily member 4 | MPP4 |
| IPI00009040 | Ornithine decarboxylase antizyme 3 | OAZ3 |
| IPI00383774 | Mih1/Tx protein | CASP4 |
| IPI00182774 | SH3YL1 protein | SH3YL1 |
| IPI00217691 | Isoform 1 of EF-hand domain-containing protein C17orf57 | C17orf57 |
| IPI00514843 | KIAA0082 | KIAA0082 |
| IPI00216346 | Isoform 2 of Nucleolar protein 3 | NOL3 |
| IPI00644968 | Similar to Heterogeneous nuclear ribonucleoprotein A1 | — |
| IPI00414122 | coiled-coil domain containing 141 | CCDC141 |
| IPI00884216 | RNA-directed DNA polymerase (Reverse transcriptase), related | — |
| | domain containing protein | |
| IPI00397759 | similar to ribosomal protein L21 | LOC388621 |
| IPI00156871 | Isoform 2 of Ubiquitin carboxyl-terminal hydrolase 49 | USP49 |
| IPI00168692 | Isoform 2 of Zinc finger protein 318 | ZNF318 |
| IPI00187082 | Isoform 3 of Multiple C2 and transmembrane domain-containing | MCTP1 |
| | protein 1 | |
| IPI00018805 | Uncharacterized protein C14orf105 | C14orf105 |
| IPI00446685 | CDNA FLJ41345 fis, clone BRAWH2002761 | — |
| IPI00470515 | Isoform 1 of Uncharacterized protein C1orf173 | C1orf173 |
| IPI00001317 | Rho-related BTB domain-containing protein 1 | RHOBTB1 |
| IPI00024523 | Isoform A of DnaJ homolog subfamily B member 6 | DNAJB6 |
| IPI00030814 | Fibroblast growth factor 22 precursor | FGF22 |
| IPI00441064 | Isoform 1 of Uncharacterized protein C15orf26 | C15orf26 |
| IPI00867744 | Isoform 3 of Pleckstrin homology domain-containing family H | PLEKHH2 |
| | member 2 | |
| IPI00784027 | Rod cGMP-specific 3′,5′-cyclic phosphodiesterase subunit beta | PDE6B |
| | precursor | |
| IPI00879277 | 436 kDa protein | TRRAP |
| IPI00011631 | Centromere/kinetochore protein zw10 homolog | ZW10 |
| IPI00014186 | Isoform A of Zinc finger homeobox protein 3 | ZFHX3 |
| IPI00852699 | 4 kDa protein | — |
| IPI00400834 | Mediator of RNA polymerase II transcription subunit 13-like | MED13L |
| IPI00018203 | Isoform SRP55-2 of Splicing factor, arginine/serine-rich 6 | SFRS6 |
| IPI00335259 | deleted in liver cancer 1 isoform 1 | DLC1 |
| IPI00239405 | Isoform 1 of Nesprin-2 | SYNE2 |
| IPI00788010 | similar to 60S ribosomal protein L21 | RPL21; LOC728501 |
| IPI00027487 | Creatine kinase M-type | CKM |
| IPI00029662 | Isoform 1 of Potassium voltage-gated channel subfamily H member 2 | KCNH2 |
| IPI00651669 | Putative uncharacterized protein | — |
| IPI00029446 | Isoform 1 of Myotubularin-related protein 5 | SBF1 |
| IPI00883929 | Conserved hypothetical protein | — |
| IPI00641155 | 20 kDa protein | HIRIP3 |
| IPI00030307 | Solute carrier family 41 member 2 | SLC41A2 |
| IPI00398162 | Isoform 2 of Nebulin-related-anchoring protein | NRAP |
| IPI00158506 | Isoform 3 of Zinc finger protein 688 | ZNF688 |
| IPI00003768 | Isoform 1 of Pescadillo homolog 1 | PES1 |
| IPI00100910 | Isoform 2 of Armadillo repeat-containing protein 5 | ARMC5 |
| IPI00445894 | CDNA FLJ42978 fis, clone BRTHA2004821 | — |
| IPI00022325 | Isoform 4 of Pro-neuregulin-2, membrane-bound isoform precursor | NRG2 |
| IPI00747271 | zinc finger protein SBZF3 | ZNF695 |
| IPI00006178 | Y6 encoding protein | NPY6R |
| IPI00796745 | 22 kDa protein | PPP1R14D |
| IPI00299880 | Isoform 2 of Calcium-activated potassium channel subunit beta-3 | KCNMB3 |
| IPI00328885 | eEF1A2 binding protein | DKFZp434B1231 |
| IPI00009305 | Glucosamine-6-phosphate isomerase | GNPDA1 |
| IPI00465054 | Putative uncharacterized protein DKFZp686C1054 | THUMPD1 |
| IPI00456676 | Isoform 1 of F-box only protein 38 | FBXO38 |
| IPI00328089 | Kappa B and V(D)J recombination signal sequences binding protein | HIVEP3 |
| IPI00002506 | Dolichyl-phosphate beta-glucosyltransferase | ALG5 |
| IPI00410325 | Low-density lipoprotein receptor class A domain-containing protein 3 | LDLRAD3 |
| | precursor | |
| IPI00450358 | Putative uncharacterized protein | TOP |
| IPI00004472 | Isoform 1 of Serine/threonine-protein kinase WNK1 | WNK1 |
| IPI00428677 | Putative olfactory receptor (Fragment) | — |
| IPI00788014 | similar to Opioid growth factor receptor | LOC729015 |
| IPI00442208 | CDNA FLJ16344 fis, clone TEST12032643, moderately similar to | ADCY4 |
| | ADENYLATE CYCLASE, TYPE IV | |
| IPI00019932 | ubiquitin-conjugating enzyme E2D 2 isoform 2 | UBE2D2 |
| IPI00869224 | Pseudogene candidate | — |
| IPI00000102 | Progonadoliberin-1 precursor | GNRH1 |
| IPI00470896 | Isoform 2 of Zinc finger FYVE domain-containing protein 26 | ZFYVE26 |
| IPI00329488 | Isoform IB of Tyrosine-protein kinase ABL2 | ABL2 |
| IPI00219604 | Dual specificity mitogen-activated protein kinase kinase 1 | MAP2K1 |
| IPI00000030 | Isoform Delta-1 of Serine/threonine-protein phosphatase 2A 56 kDa | PPP2R5D |
| | regulatory subunit delta isoform | |
| IPI00017538 | Isoform 2 of Separin | ESPL1 |
| IPI00787099 | similar to SR protein related family member | LOC728676 |
| IPI00297235 | cDNA FLJ77204 | CCPG1 |
| IPI00002405 | 2′-5′-oligoadenylate synthetase 3 | OAS3 |
| IPI00013205 | Isoform 1 of JmjC domain-containing histone demethylation protein | JMJD2B |
| | 3B | |
| IPI00218715 | Isoform 2 of ADAM 33 precursor | ADAM33 |
| IPI00410666 | Isoform 3 of Protein LAP4 | SCRIB |
| IPI00827815 | Light chain Fab (Fragment) | — |
| IPI00013651 | Isoform 3 of RNA-binding protein 33 | RBM33 |
| IPI00002344 | steroid 5 alpha-reductase 3 | SRD5A3 |
| IPI00444408 | CDNA FLJ45600 fis, clone BRTHA3020369 | — |
| IPI00005013 | poly(rC) binding protein 4 isoform b | PCBP4 |
| IPI00294728 | DmX-like protein 1 | DMXL1 |
| IPI00004288 | Isoform 1 of Sialic acid-binding Ig-like lectin 7 precursor | SIGLEC7 |
| IPI00855960 | Integral membrane protein GPR137C | GPR137C |
| IPI00003571 | Isoform S-CaBP1 of Calcium-binding protein 1 | CABP1 |
| IPI00021751 | Neurofilament heavy polypeptide | NEFH |
| IPI00174347 | Inactive dual specificity phosphatase 27 | DUSP27 |
| IPI00787774 | similar to collagen, type I, alpha 1 | LOC730756 |
| IPI00419903 | Putative L-aspartate dehydrogenase | LOC554235 |
| IPI00165357 | Isoform 1 of Metastasis-associated protein MTA3 | MTA3 |
| IPI00299406 | Pituitary adenylate cyclase-activating polypeptide type I receptor | ADCYAP1R1 |
| | precursor | |
| IPI00401789 | Isoform 2 of Highly divergent homeobox | HDX |
| IPI00303732 | Uncharacterized protein C20orf106 precursor | C20orf106 |
| IPI00016425 | Isoform 2 of Synaptopodin 2-like protein | SYNPO2L |
| IPI00017640 | Isoform 1 of Slit homolog 3 protein precursor | SLIT3 |
| IPI00014577 | Ras-related protein Rab-18 | RAB18 |
| IPI00023856 | Peregrin | BRPF1 |
| IPI00294241 | Transcription factor IIIA | GTF3A |
| IPI00025717 | Metaxin-2 | MTX2 |
| IPI00023095 | Myeloid leukemia factor 2 | MLF2 |
| IPI00009645 | Ran-binding protein 17 | RANBP17 |
| IPI00022629 | Isoform 1 of Protein regulator of cytokinesis 1 | PRC1 |
| IPI00031082 | Putative uncharacterized protein DKFZp761O17121 | LDOC1L |
| IPI00788120 | similar to CG14182-PA | LOC730094 |
| IPI00396063 | C-myc promoter-binding protein | DENND4A |
| IPI00013296 | 40S ribosomal protein S18 | RPS18 |
| IPI00004362 | MORC family CW-type zinc finger protein 1 | MORC1 |
| IPI00787625 | similar to 60S ribosomal protein L23a | LOC389101 |
| IPI00385020 | Putative uncharacterized protein DKFZp686D159 (Fragment) | CAPRIN2 |
| IPI00374223 | hypothetical protein LOC389119 | C3orf54 |
| IPI00039626 | Isoform D of UPF0318 protein FAM120A | FAM120A |
| IPI00295253 | Isoform 1 of Cell surface glycoprotein OX2 receptor precursor | CD200R1 |
| IPI00299040 | Polycystin-2 | PKD2 |
| IPI00307165 | Tripartite motif-containing protein 47 | TRIM47 |
| IPI00152685 | Tetratricopeptide repeat protein 15 | TTC15 |
| IPI00013789 | SET and MYND domain-containing protein 5 | SMYD5 |
| IPI00396025 | hypothetical protein LOC254122 | FLJ30934 |
| IPI00014232 | ADP-ribosylation factor-like protein 6-interacting protein 1 | ARL6IP1 |
| IPI00021715 | Isoform 1 of Collagen alpha-5(IV) chain precursor | COL4A5 |
| IPI00026305 | Hook homolog 1 | HOOK1 |
| IPI00184160 | Isoform 2 of RING finger protein 180 | RNF180 |
| IPI00216308 | Voltage-dependent anion-selective channel protein 1 | VDAC1 |
| IPI00651633 | CASKIN1 protein (Fragment) | CASKIN1 |
| IPI00247063 | Neprilysin | MME |
| IPI00398779 | Isoform 4 of Plectin-1 | PLEC1 |
| IPI00168418 | Putative uncharacterized protein C11orf64 | C11orf64 |
| IPI00167764 | Transmembrane protein 102 | TMEM102 |
| IPI00171716 | OTTHUMP00000028696 | TTC28 |
| IPI00394818 | Isoform 1 of Ankyrin repeat domain-containing protein 47 | KANK3 |
| IPI00830137 | Uncharacterized protein ENSP00000374864 | — |
| IPI00030874 | Uncharacterized gastric protein YA42P (Fragment) | — |
| IPI00455675 | Isoform 2 of Centrosomal protein of 192 kDa | CEP192 |
| IPI00789889 | Hypothetical protein | — |
| IPI00829625 | Uncharacterized protein FAM91A1 | FAM91A1 |
| IPI00000873 | Valyl-tRNA synthetase | VARS |
| IPI00386185 | FLJ00370 protein (Fragment) | — |
| IPI00829697 | Uncharacterized protein ENSP00000375030 | — |
| IPI00000690 | Isoform 1 of Apoptosis-inducing factor 1, mitochondrial precursor | AIFM1 |
| IPI00016988 | WD repeat-containing protein 13 | WDR13 |
| IPI00478058 | Isoform 2 of NMDA receptor-regulated 1-like protein | NARG1L |
| IPI00782960 | Isoform 1 of Leucine-rich repeat and IQ domain-containing protein 3 | LRRC44 |
| IPI00103373 | Cytoglobin | CYGB |
| IPI00646831 | 34 kDa protein | — |
| IPI00384164 | Full-length cDNA clone CS0DI075YC18 of Placenta of Homo | PGF |
| | sapiens (Fragment) | |
| IPI00739611 | similar to golgi autoantigen, golgin subfamily a, 8A isoform 3 | LOC727909 |
| IPI00012451 | Guanine nucleotide-binding protein subunit beta-4 | GNB4 |
| IPI00394960 | Isoform 1 of Probable G-protein coupled receptor 133 precursor | GPR133 |
| IPI00555595 | Ubiquitin carboxyl-terminal esterase L1 (Ubiquitin thiolesterase) | — |
| | variant (Fragment) | |
| IPI00431697 | Cancer/testis antigen 45-5 | RP13-36C9.6 |
| IPI00028066 | class IV alcohol dehydrogenase 7 mu or sigma subunit | ADH7 |
| IPI00152960 | RNA polymerase 1-specific transcription initiation factor RRN3 | RRN3 |
| IPI00792370 | 6 kDa protein | GNAT1 |
| IPI00167762 | Isoform 1 of Uncharacterized protein C19orf47 | C19orf47 |
| IPI00385321 | Isoform 2 of Protein kinase C-binding protein 1 | ZMYND8 |
| IPI00385153 | HUMEEP | ATXN10 |
| IPI00337348 | Growth differentiation factor 7 | GDF7 |
| IPI00060627 | Coiled-coil domain-containing protein 124 | CCDC124 |
| IPI00401190 | LOC155006 protein | LOC155006 |
| IPI00025039 | rRNA 2′-O-methyltransferase fibrillarin | FBL |
| IPI00479578 | similar to Nonhistone chromosomal protein HMG-14 | hCG_1659830 |
| IPI00018245 | Isoform 3 of DNA-directed RNA polymerase III subunit RPC5 | POLR3E |
| IPI00020565 | PRO1598 | — |
| IPI00446711 | CDNA FLJ41261 fis, clone BRAMY2034920 | — |
| IPI00787990 | similar to ribosomal protein S14 | LOC644119 |
| IPI00218743 | Isoform 2 of Bestrophin-1 | BEST1 |
| IPI00736826 | similar to Temporarily Assigned Gene name family member | LOC402217 |
| IPI00412024 | Zinc finger protein 485 | ZNF485 |
| IPI00297559 | Isoform 1 of USH1C-binding protein 1 | USHBP1 |
| IPI00396147 | Protocadherin-20 precursor | PCDH20 |
| IPI00478572 | Collagen alpha-4(IV) chain precursor | COL4A4 |
| IPI00032785 | Isoform II of Amyloid beta A4 precursor protein-binding family B | APBB3 |
| | member 3 | |
| IPI00009982 | Isoform 1 of Tudor and KH domain-containing protein | TDRKH |
| IPI00216164 | Peroxisomal bifunctional enzyme | EHHADH |
| IPI00419912 | YY2 transcription factor | YY2 |
| IPI00735531 | proline-rich synapse-associated protein 2 isoform 4 | SHANK3 |
| IPI00005104 | Inhibitor of nuclear factor kappa-B kinase subunit alpha | CHUK |
| IPI00008837 | Tyrosine-protein phosphatase non-receptor type 5 | PTPN5 |
| IPI00017800 | ATP-binding cassette sub-family A member 3 | ABCA3 |
| IPI00737972 | Transcription factor 24 (Fragment) | TCF24 |
| IPI00374074 | similar to CG40449-PA.3 | LOC388795 |
| IPI00220533 | Isoform D of Transmembrane protease, serine 3 | TMPRSS3 |
| IPI00302458 | Exportin-7 | XPO7 |
| IPI00646826 | Putative uncharacterized protein FLJ32658 | FLJ32658 |
| IPI00016859 | Isoform 1 of Transcription factor IIIB 90 kDa subunit | BRF1 |
| IPI00031386 | Phosphatidylinositol-4,5-bisphosphate 3-kinase catalytic subunit | PIK3CA |
| | alpha isoform | |
| IPI00065547 | OTTHUMP00000031495 | RP11-93B14.6 |
| IPI00043370 | WD repeat-containing protein 52 | WDR52 |
| IPI00064767 | Isoform 1 of Rho GTPase-activating protein 17 | ARHGAP17 |
| IPI00219689 | Isoform Beta of Protein vav-3 | VAV3 |
| IPI00328291 | Cytoskeleton-associated protein 2-like | CKAP2L |
| IPI00647897 | RING finger protein 113B | RNF113B |
| IPI00024285 | similar to testicular serine protease 2 | LOC138652 |
| IPI00783200 | 38 kDa protein | RAPGEF3 |
| IPI00014877 | Isoform 2 of Glutaminyl-peptide cyclotransferase-like protein | QPCTL |
| IPI00186554 | Uncharacterized protein KIAA1853 | KIAA1853 |
| IPI00418336 | Isoform 2 of Integrator complex subunit 3 | INTS3 |
| IPI00872730 | Macrophin 1 isoform | MACF1 |
| IPI00303207 | ATP-binding cassette sub-family E member 1 | ABCE1 |
| IPI00301609 | Serine/threonine-protein kinase Nek9 | NEK9 |
| IPI00744527 | Conserved hypothetical protein | — |
| IPI00302351 | Isoform 1 of Serine/threonine-protein kinase 33 | STK33 |
| IPI00878483 | 30 kDa protein | — |
| IPI00744179 | Uncharacterized protein ENSP00000370856 (Fragment) | — |
| IPI00396279 | Isoform 1 of CLIP-associating protein 1 | CLASP1 |
| IPI00013378 | Isoform Long of DNA topoisomerase 3-alpha | TOP3A |
| IPI00429190 | Ras-related protein Rab-11A | RAB11A |
| IPI00464994 | Isoform 1 of tRNA modification GTPase GTPBP3, mitochondrial | GTPBP3 |
| | precursor | |
| IPI00008449 | Isoform 3 of Pre-mRNA 3′-end-processing factor FIP1 | FIP1L1 |
| IPI00414402 | Olfactory receptor 2T27 | OR2T27 |
| IPI00168913 | Isoform 2 of Limbin | EVC2 |
| IPI00009315 | Golgi resident protein GCP60 | ACBD3 |
| IPI00871625 | cDNA FLJ77072, highly similar to Homo sapiens prostaglandin E | PTGER3 |
| | receptor 3 (subtype EP3) (PTGER3), transcript variant 9, mRNA | |
| IPI00479296 | Isoform 1 of ATP-binding cassette sub-family A member 8 | ABCA8 |
| IPI00300221 | Putative uncharacterized protein DKFZp434I1916 | ELL |
| IPI00026036 | Isoform 5 of Dipeptidyl peptidase 8 | DPP8 |
| IPI00647205 | Uncharacterized protein C6orf174 precursor | C6orf174 |
| IPI00006932 | Isoform 1 of Putative RNA-binding protein Luc7-like 2 | LUC7L2 |
| IPI00017819 | Bile acid-CoA:amino acid N-acyltransferase | BAAT |
| IPI00145121 | GPI mannosyltransferase 3 | PIGB |
| IPI00744357 | Conserved hypothetical protein | — |
| IPI00410320 | Sodium-dependent glucose transporter | SLC5A11 |
| IPI00065085 | CDNA FLJ25371 fis, clone TST01885 | FLJ25371 |
| IPI00289914 | MutS protein homolog 4 | MSH4 |
| IPI00012438 | Isoform Long of 52 kDa repressor of the inhibitor of the protein | PRKRIR |
| | kinase | |
| IPI00217895 | rai-like protein | SHC4 |
| IPI00294618 | Proline-rich protein PRCC | PRCC |
| IPI00000784 | Isoform MEF2DAB of Myocyte-specific enhancer factor 2D | MEF2D |
| IPI00848180 | Conserved hypothetical protein | — |
| IPI00045208 | Zinc finger protein 300-B | ZNF300 |
| IPI00871452 | Isoform 1 of PWWP domain-containing protein 2A | PWWP2A |
| IPI00102820 | Isoform 3 of Mitochondrial tumor suppressor 1 | MTUS1 |
| IPI00215786 | 3′,5′-cyclic nucleotide phosphodiesterase 10A2 | PDE10A |
| IPI00845401 | Isoform 6 of Neuron navigator 1 | NAV1 |
| IPI00217979 | Myeloid/lymphoid or mixed-lineage leukemia 5 | MLL5 |
| IPI00440764 | Isoform 1 of Gamma-glutamyltransferase 6 precursor | GGT6 |
| IPI00374068 | Isoform 1 of ADAMTS-like protein 4 precursor | ADAMTSL4 |
| IPI00186439 | Zinc finger protein 643 | ZNF643 |
| IPI00062467 | Isoform 1 of Zinc finger protein 274 | ZNF274 |
| IPI00000890 | Isoform 1 of Growth hormone variant precursor | GH2 |
| IPI00065351 | Zinc finger protein 354B | ZNF354B |
| IPI00552455 | 12 kDa protein | INPP5A |
| IPI00395473 | Zinc finger protein 135 | ZNF135 |
| IPI00003842 | Isoform 1 of Microtubule-associated protein 2 | MAP2 |
| IPI00655760 | Envelope polyprotein | — |
| IPI00384767 | FLJ00332 protein (Fragment) | TBC1D10C |
| IPI00028050 | Selenocysteine-specific elongation factor | EEFSEC |
| IPI00152033 | Isoform 1 of Transient receptor potential cation channel subfamily M | TRPM8 |
| | member 8 | |
| IPI00789564 | Isoform 3 of Metallophosphoesterase 1 | MPPE1 |
| IPI00011253 | 40S ribosomal protein S3 | RPS3 |
| IPI00454620 | HERV-K_11q22.1 provirus ancestral Pol protein | — |
| IPI00337335 | Isoform 1 of Myosin-14 | MYH14 |
| IPI00103426 | Isoform 1 of Solute carrier family 13 member 3 | SLC13A3 |
| IPI00291839 | Isoform 2 of Rap guanine nucleotide exchange factor 6 | RAPGEF6 |

TABLE 9
Relative Abundance of CSF Proteins Chronic Fatigue compared to Neurologic Lyme
Table 9. Quantitative Relative Abundance of CSF Proteins in Chronic Fatigue (CF) compared to
Neurologic Lyme Disease. A ratio greater than 1 indicates there is a relative quantitative
difference in Chronic Fatigue compared to Neurologic Lyme disease (LD). Ratios near 1 suggest
equal relative abundances. Ratios less than 1 indicate the proteins is present in a greater relative
abundance in Neurologic Lyme disease compared to Chronic Fatigue. There are 168 proteins in
Chronic Fatigue that are 2 fold greater in relative abundance than those proteins Neurologic
Lyme disease. There are 1081 proteins that are relatively more abundant in Neurologic Lyme
disease compared to Chronic Fatigue with a ratio of 0.5 or less. Blank cell is close to 0 ratio.

| Clean IPI | CF to LD Ratio |
|---|---|
| IPI00217493 | 18.00 |
| IPI00783656 | 6.67 |
| IPI00465436 | 6.50 |
| IPI00161055 | 6.50 |
| IPI00445716 | 5.00 |
| IPI00384395 | 5.00 |
| IPI00020747 | 5.00 |
| IPI00291807 | 5.00 |
| IPI00827978 | 4.00 |
| IPI00827485 | 4.00 |
| IPI00024887 | 4.00 |
| IPI00419442 | 4.00 |
| IPI00008107 | 4.00 |
| IPI00022445 | 4.00 |
| IPI00002307 | 4.00 |
| IPI00465070 | 4.00 |
| IPI00023604 | 4.00 |
| IPI00395769 | 4.00 |
| IPI00220706 | 3.60 |
| IPI00382682 | 3.50 |
| IPI00830035 | 3.33 |
| IPI00465315 | 3.22 |
| IPI00384407 | 3.00 |
| IPI00296259 | 3.00 |
| IPI00400986 | 3.00 |
| IPI00010863 | 3.00 |
| IPI00005153 | 3.00 |
| IPI00020131 | 3.06 |
| IPI00176920 | 3.00 |
| IPI00176104 | 3.00 |
| IPI00019600 | 3.00 |
| IPI00299435 | 3.00 |
| IPI00008207 | 3.00 |
| IPI00830057 | 3.00 |
| IPI00028931 | 3.00 |
| IPI00006556 | 3.00 |
| IPI00022674 | 3.00 |
| IPI00043810 | 3.00 |
| IPI00166892 | 3.00 |
| IPI00398007 | 3.00 |
| IPI00016604 | 3.00 |
| IPI00003469 | 3.00 |
| IPI00827643 | 3.00 |
| IPI00741524 | 3.00 |
| IPI00179415 | 3.00 |
| IPI00442002 | 3.00 |
| IPI00387099 | 3.00 |
| IPI00021828 | 3.00 |
| IPI00410714 | 2.91 |
| IPI00383808 | 2.75 |
| IPI00182138 | 2.67 |
| IPI00473011 | 2.67 |
| IPI00442909 | 2.67 |
| IPI00023019 | 2.55 |
| IPI00021833 | 2.50 |
| IPI00883772 | 2.50 |
| IPI00293088 | 2.50 |
| IPI00216983 | 2.50 |
| IPI00410079 | 2.50 |
| IPI00215983 | 2.45 |
| IPI00220301 | 2.40 |
| IPI00024621 | 2.40 |
| IPI00029275 | 2.40 |
| IPI00008556 | 2.33 |
| IPI00009477 | 2.29 |
| IPI00023505 | 2.25 |
| IPI00654755 | 2.15 |
| IPI00418960 | 2.14 |
| IPI00010381 | 2.11 |
| IPI00003907 | 2.09 |
| IPI00002818 | 2.00 |
| IPI00002535 | 2.00 |
| IPI00030757 | 2.00 |
| IPI00217781 | 2.00 |
| IPI00382478 | 2.00 |
| IPI00018352 | 2.00 |
| IPI00470766 | 2.00 |
| IPI00007617 | 2.00 |
| IPI00301098 | 2.00 |
| IPI00000070 | 2.00 |
| IPI00827788 | 2.00 |
| IPI00003807 | 2.00 |
| IPI00010133 | 2.00 |
| IPI00099650 | 2.00 |
| IPI00166553 | 2.00 |
| IPI00221080 | 2.00 |
| IPI00099838 | 2.00 |
| IPI00744706 | 2.00 |
| IPI00291987 | 2.00 |
| IPI00031019 | 2.00 |
| IPI00387101 | 2.00 |
| IPI00004480 | 2.00 |
| IPI00021812 | 2.00 |
| IPI00008085 | 2.00 |
| IPI00027972 | 2.00 |
| IPI00455739 | 2.00 |
| IPI00883879 | 2.00 |
| IPI00297208 | 2.00 |
| IPI00151036 | 2.00 |
| IPI00218914 | 2.00 |
| IPI00328587 | 2.00 |
| IPI00005517 | 2.00 |
| IPI00029605 | 2.00 |
| IPI00009881 | 2.00 |
| IPI00030431 | 2.00 |
| IPI00384400 | 2.00 |
| IPI00008997 | 2.00 |
| IPI00019755 | 2.00 |
| IPI00383951 | 2.00 |
| IPI00000160 | 2.00 |
| IPI00012048 | 2.00 |
| IPI00010810 | 2.00 |
| IPI00020431 | 2.00 |
| IPI00297288 | 2.00 |
| IPI00218474 | 2.00 |
| IPI00719621 | 2.00 |
| IPI00387110 | 2.00 |
| IPI00329352 | 2.00 |
| IPI00399089 | 2.00 |
| IPI00027239 | 2.00 |
| IPI00783604 | 2.00 |
| IPI00008726 | 2.00 |
| IPI00299399 | 2.00 |
| IPI00168921 | 2.00 |
| IPI00383603 | 2.00 |
| IPI00179851 | 2.00 |
| IPI00219684 | 2.00 |
| IPI00644472 | 2.00 |
| IPI00220362 | 2.00 |
| IPI00006130 | 2.00 |
| IPI00550363 | 2.00 |
| IPI00420071 | 2.00 |
| IPI00103871 | 2.00 |
| IPI00218539 | 2.00 |
| IPI00018941 | 2.00 |
| IPI00432766 | 2.00 |
| IPI00746600 | 2.00 |
| IPI00419815 | 2.00 |
| IPI00376197 | 2.00 |
| IPI00293613 | 2.00 |
| IPI00796518 | 2.00 |
| IPI00007648 | 2.00 |
| IPI00409659 | 2.00 |
| IPI00220113 | 2.00 |
| IPI00015135 | 2.00 |
| IPI00306929 | 2.00 |
| IPI00011092 | 2.00 |
| IPI00293616 | 2.00 |
| IPI00643747 | 2.00 |
| IPI00465173 | 2.00 |
| IPI00011832 | 2.00 |
| IPI00185892 | 2.00 |
| IPI00152189 | 2.00 |
| IPI00176778 | 2.00 |
| IPI00829979 | 2.00 |
| IPI00004970 | 2.00 |
| IPI00003084 | 2.00 |
| IPI00025155 | 2.00 |
| IPI00401776 | 2.00 |
| IPI00009504 | 2.00 |
| IPI00015473 | 2.00 |
| IPI00304533 | 2.00 |
| IPI00031101 | 2.00 |
| IPI00402291 | 2.00 |
| IPI00385799 | 2.00 |
| IPI00829803 | 2.00 |
| IPI00033541 | 2.00 |
| IPI00027220 | 2.00 |
| IPI00014048 | 2.00 |
| IPI00329775 | 1.97 |
| IPI00022284 | 1.90 |
| IPI00165975 | 1.88 |
| IPI00745313 | 1.86 |
| IPI00016679 | 1.86 |
| IPI00016666 | 1.83 |
| IPI00307592 | 1.83 |
| IPI00006803 | 1.83 |
| IPI00382420 | 1.83 |
| IPI00001895 | 1.83 |
| IPI00012887 | 1.80 |
| IPI00397645 | 1.80 |
| IPI00873544 | 1.80 |
| IPI00479116 | 1.78 |
| IPI00829841 | 1.78 |
| IPI00010402 | 1.78 |
| IPI00025426 | 1.77 |
| IPI00025318 | 1.75 |
| IPI00019530 | 1.75 |
| IPI00020906 | 1.75 |
| IPI00375364 | 1.75 |
| IPI00418471 | 1.75 |
| IPI00013438 | 1.75 |
| IPI00443799 | 1.73 |
| IPI00219131 | 1.71 |
| IPI00059366 | 1.69 |
| IPI00003111 | 1.67 |
| IPI00465377 | 1.67 |
| IPI00419565 | 1.67 |
| IPI00025473 | 1.67 |
| IPI00001610 | 1.67 |
| IPI00007240 | 1.67 |
| IPI00009054 | 1.67 |
| IPI00465255 | 1.67 |
| IPI00010896 | 1.67 |
| IPI00099670 | 1.67 |
| IPI00419966 | 1.67 |
| IPI00031708 | 1.67 |
| IPI00419720 | 1.67 |
| IPI00382486 | 1.67 |
| IPI00337351 | 1.67 |
| IPI00008752 | 1.67 |
| IPI00027765 | 1.67 |
| IPI00854589 | 1.67 |
| IPI00294193 | 1.65 |
| IPI00218192 | 1.65 |
| IPI00784154 | 1.62 |
| IPI00844156 | 1.61 |
| IPI00743963 | 1.60 |
| IPI00018311 | 1.60 |
| IPI00152850 | 1.60 |
| IPI00816775 | 1.60 |
| IPI00440932 | 1.60 |
| IPI00470607 | 1.60 |
| IPI00428511 | 1.60 |
| IPI00743194 | 1.58 |
| IPI00743766 | 1.58 |
| IPI00029131 | 1.57 |
| IPI00382481 | 1.57 |
| IPI00829701 | 1.57 |
| IPI00292732 | 1.57 |
| IPI00827522 | 1.57 |
| IPI00012075 | 1.57 |
| IPI00298547 | 1.56 |
| IPI00001506 | 1.55 |
| IPI00027350 | 1.53 |
| IPI00169383 | 1.53 |
| IPI00008944 | 1.51 |
| IPI00854745 | 1.50 |
| IPI00024035 | 1.50 |
| IPI00394820 | 1.50 |
| IPI00301294 | 1.50 |
| IPI00014964 | 1.50 |
| IPI00412541 | 1.50 |
| IPI00064652 | 1.50 |
| IPI00883765 | 1.50 |
| IPI00002412 | 1.50 |
| IPI00552852 | 1.50 |
| IPI00736885 | 1.50 |
| IPI00789234 | 1.50 |
| IPI00165972 | 1.50 |
| IPI00297180 | 1.50 |
| IPI00008533 | 1.50 |
| IPI00302962 | 1.50 |
| IPI00045536 | 1.50 |
| IPI00005222 | 1.50 |
| IPI00215980 | 1.50 |
| IPI00641251 | 1.50 |
| IPI00015911 | 1.50 |
| IPI00306322 | 1.50 |
| IPI00301180 | 1.50 |
| IPI00217963 | 1.50 |
| IPI00293530 | 1.50 |
| IPI00018246 | 1.50 |
| IPI00184094 | 1.50 |
| IPI00165044 | 1.50 |
| IPI00030882 | 1.50 |
| IPI00008223 | 1.50 |
| IPI00169285 | 1.50 |
| IPI00291006 | 1.50 |
| IPI00021304 | 1.50 |
| IPI00167215 | 1.50 |
| IPI00295601 | 1.50 |
| IPI00306343 | 1.50 |
| IPI00387025 | 1.50 |
| IPI00064429 | 1.50 |
| IPI00001674 | 1.50 |
| IPI00217975 | 1.50 |
| IPI00291878 | 1.50 |
| IPI00012544 | 1.50 |
| IPI00064606 | 1.50 |
| IPI00001328 | 1.50 |
| IPI00000436 | 1.50 |
| IPI00009375 | 1.50 |
| IPI00015049 | 1.48 |
| IPI00296058 | 1.47 |
| IPI00001593 | 1.47 |
| IPI00015479 | 1.45 |
| IPI00783024 | 1.45 |
| IPI00301961 | 1.45 |
| IPI00829947 | 1.45 |
| IPI00024601 | 1.45 |
| IPI00455667 | 1.45 |
| IPI00009901 | 1.44 |
| IPI00299150 | 1.44 |
| IPI00024307 | 1.44 |
| IPI00297284 | 1.44 |
| IPI00022333 | 1.44 |
| IPI00293836 | 1.44 |
| IPI00032179 | 1.44 |
| IPI00006154 | 1.44 |
| IPI00787853 | 1.43 |
| IPI00550991 | 1.43 |
| IPI00026991 | 1.43 |
| IPI00442911 | 1.42 |
| IPI00289058 | 1.42 |
| IPI00029863 | 1.42 |
| IPI00867665 | 1.42 |
| IPI00018274 | 1.42 |
| IPI00854709 | 1.41 |
| IPI00743302 | 1.41 |
| IPI00003590 | 1.40 |
| IPI00020019 | 1.40 |
| IPI00011578 | 1.40 |
| IPI00829590 | 1.40 |
| IPI00299083 | 1.40 |
| IPI00002334 | 1.40 |
| IPI00884389 | 1.40 |
| IPI00003919 | 1.40 |
| IPI00026285 | 1.40 |
| IPI00783399 | 1.40 |
| IPI00031534 | 1.40 |
| IPI00019449 | 1.40 |
| IPI00005516 | 1.40 |
| IPI00747758 | 1.40 |
| IPI00291136 | 1.39 |
| IPI00019581 | 1.38 |
| IPI00166039 | 1.38 |
| IPI00001592 | 1.38 |
| IPI00025809 | 1.38 |
| IPI00010796 | 1.38 |
| IPI00183321 | 1.38 |
| IPI00218046 | 1.38 |
| IPI00032328 | 1.37 |
| IPI00465016 | 1.37 |
| IPI00334667 | 1.37 |
| IPI00026050 | 1.36 |
| IPI00477992 | 1.36 |
| IPI00032220 | 1.36 |
| IPI00217345 | 1.36 |
| IPI00472249 | 1.36 |
| IPI00297646 | 1.35 |
| IPI00328746 | 1.35 |
| IPI00794450 | 1.35 |
| IPI00026154 | 1.35 |
| IPI00514285 | 1.35 |
| IPI00646281 | 1.34 |
| IPI00019176 | 1.33 |
| IPI00387113 | 1.33 |
| IPI00011643 | 1.33 |
| IPI00005129 | 1.33 |
| IPI00657936 | 1.33 |
| IPI00387024 | 1.33 |
| IPI00298650 | 1.33 |
| IPI00884080 | 1.33 |
| IPI00418125 | 1.33 |
| IPI00789477 | 1.33 |
| IPI00021856 | 1.33 |
| IPI00789259 | 1.33 |
| IPI00741710 | 1.33 |
| IPI00000144 | 1.33 |
| IPI00219425 | 1.33 |
| IPI00783471 | 1.33 |
| IPI00171196 | 1.33 |
| IPI00160552 | 1.33 |
| IPI00883855 | 1.33 |
| IPI00006713 | 1.33 |
| IPI00011518 | 1.33 |
| IPI00293303 | 1.33 |
| IPI00031549 | 1.33 |
| IPI00296197 | 1.33 |
| IPI00554786 | 1.33 |
| IPI00000824 | 1.33 |
| IPI00018396 | 1.33 |
| IPI00165125 | 1.33 |
| IPI00787265 | 1.33 |
| IPI00299024 | 1.33 |
| IPI00299299 | 1.33 |
| IPI00009111 | 1.33 |
| IPI00300838 | 1.33 |
| IPI00027497 | 1.33 |
| IPI00006543 | 1.33 |
| IPI00142538 | 1.33 |
| IPI00015614 | 1.33 |
| IPI00786926 | 1.33 |
| IPI00009851 | 1.33 |
| IPI00013895 | 1.33 |
| IPI00019943 | 1.33 |
| IPI00220292 | 1.33 |
| IPI00027087 | 1.33 |
| IPI00654888 | 1.32 |
| IPI00023807 | 1.32 |
| IPI00783287 | 1.32 |
| IPI00018219 | 1.31 |
| IPI00007778 | 1.31 |
| IPI00022431 | 1.31 |
| IPI00013682 | 1.30 |
| IPI00456736 | 1.30 |
| IPI00103175 | 1.30 |
| IPI00025461 | 1.30 |
| IPI00169276 | 1.30 |
| IPI00394992 | 1.30 |
| IPI00163207 | 1.30 |
| IPI00152847 | 1.29 |
| IPI00005142 | 1.29 |
| IPI00299758 | 1.29 |
| IPI00022608 | 1.29 |
| IPI00294615 | 1.29 |
| IPI00827829 | 1.29 |
| IPI00854841 | 1.29 |
| IPI00008780 | 1.29 |
| IPI00006657 | 1.29 |
| IPI00003865 | 1.29 |
| IPI00027174 | 1.29 |
| IPI00003176 | 1.29 |
| IPI00220739 | 1.29 |
| IPI00217966 | 1.29 |
| IPI00879915 | 1.29 |
| IPI00328550 | 1.28 |
| IPI00383732 | 1.28 |
| IPI00292150 | 1.28 |
| IPI00289204 | 1.28 |
| IPI00162547 | 1.28 |
| IPI00062037 | 1.27 |
| IPI00012283 | 1.27 |
| IPI00382500 | 1.27 |
| IPI00514676 | 1.27 |
| IPI00220342 | 1.27 |
| IPI00029235 | 1.27 |
| IPI00477597 | 1.27 |
| IPI00029699 | 1.27 |
| IPI00301255 | 1.27 |
| IPI00432592 | 1.27 |
| IPI00218733 | 1.27 |
| IPI00795013 | 1.26 |
| IPI00395488 | 1.26 |
| IPI00783492 | 1.25 |
| IPI00015315 | 1.25 |
| IPI00016150 | 1.25 |
| IPI00816155 | 1.25 |
| IPI00884004 | 1.25 |
| IPI00552939 | 1.25 |
| IPI00783156 | 1.25 |
| IPI00021817 | 1.25 |
| IPI00017557 | 1.25 |
| IPI00000265 | 1.25 |
| IPI00002925 | 1.25 |
| IPI00382493 | 1.25 |
| IPI00033560 | 1.25 |
| IPI00414896 | 1.25 |
| IPI00028714 | 1.25 |
| IPI00477714 | 1.25 |
| IPI00401283 | 1.25 |
| IPI00030847 | 1.25 |
| IPI00006524 | 1.25 |
| IPI00168479 | 1.25 |
| IPI00293757 | 1.25 |
| IPI00024273 | 1.25 |
| IPI00646304 | 1.25 |
| IPI00015964 | 1.25 |
| IPI00001734 | 1.25 |
| IPI00430291 | 1.25 |
| IPI00382426 | 1.25 |
| IPI00328840 | 1.25 |
| IPI00009997 | 1.25 |
| IPI00009030 | 1.24 |
| IPI00032293 | 1.24 |
| IPI00556287 | 1.24 |
| IPI00854743 | 1.24 |
| IPI00022895 | 1.24 |
| IPI00019580 | 1.24 |
| IPI00299738 | 1.23 |
| IPI00220562 | 1.23 |
| IPI00166048 | 1.23 |
| IPI00000044 | 1.23 |
| IPI00787050 | 1.23 |
| IPI00002211 | 1.23 |
| IPI00292946 | 1.23 |
| IPI00215894 | 1.23 |
| IPI00007853 | 1.22 |
| IPI00376689 | 1.22 |
| IPI00220334 | 1.22 |
| IPI00739827 | 1.22 |
| IPI00744835 | 1.22 |
| IPI00465248 | 1.22 |
| IPI00829877 | 1.22 |
| IPI00011252 | 1.21 |
| IPI00028413 | 1.21 |
| IPI00298828 | 1.21 |
| IPI00004413 | 1.21 |
| IPI00795055 | 1.21 |
| IPI00012503 | 1.21 |
| IPI00027482 | 1.20 |
| IPI00029819 | 1.20 |
| IPI00387095 | 1.20 |
| IPI00013701 | 1.20 |
| IPI00029817 | 1.20 |
| IPI00827940 | 1.20 |
| IPI00219465 | 1.20 |
| IPI00749328 | 1.20 |
| IPI00215997 | 1.20 |
| IPI00013096 | 1.20 |
| IPI00478809 | 1.20 |
| IPI00028015 | 1.20 |
| IPI00290856 | 1.20 |
| IPI00218414 | 1.20 |
| IPI00410487 | 1.20 |
| IPI00029273 | 1.20 |
| IPI00386418 | 1.20 |
| IPI00303963 | 1.20 |
| IPI00022488 | 1.20 |
| IPI00291866 | 1.20 |
| IPI00552905 | 1.20 |
| IPI00009920 | 1.19 |
| IPI00171473 | 1.19 |
| IPI00022937 | 1.19 |
| IPI00292304 | 1.19 |
| IPI00852979 | 1.19 |
| IPI00465325 | 1.19 |
| IPI00830047 | 1.19 |
| IPI00827906 | 1.19 |
| IPI00386630 | 1.19 |
| IPI00294705 | 1.19 |
| IPI00376394 | 1.19 |
| IPI00183445 | 1.18 |
| IPI00031030 | 1.18 |
| IPI00301364 | 1.18 |
| IPI00167710 | 1.18 |
| IPI00292950 | 1.18 |
| IPI00413959 | 1.18 |
| IPI00456623 | 1.18 |
| IPI00010949 | 1.18 |
| IPI00007257 | 1.18 |
| IPI00295832 | 1.17 |
| IPI00292530 | 1.17 |
| IPI00297124 | 1.17 |
| IPI00022420 | 1.17 |
| IPI00064935 | 1.17 |
| IPI00023824 | 1.17 |
| IPI00167093 | 1.17 |
| IPI00004946 | 1.17 |
| IPI00063048 | 1.17 |
| IPI00022367 | 1.17 |
| IPI00414717 | 1.17 |
| IPI00328391 | 1.17 |
| IPI00218493 | 1.17 |
| IPI00329688 | 1.17 |
| IPI00013569 | 1.17 |
| IPI00032311 | 1.17 |
| IPI00009865 | 1.17 |
| IPI00002406 | 1.17 |
| IPI00827650 | 1.17 |
| IPI00032291 | 1.17 |
| IPI00816555 | 1.17 |
| IPI00027827 | 1.16 |
| IPI00000130 | 1.16 |
| IPI00816741 | 1.16 |
| IPI00303161 | 1.16 |
| IPI00745363 | 1.16 |
| IPI00022337 | 1.16 |
| IPI00012058 | 1.16 |
| IPI00027507 | 1.15 |
| IPI00385253 | 1.15 |
| IPI00217146 | 1.15 |
| IPI00385985 | 1.15 |
| IPI00216691 | 1.15 |
| IPI00296537 | 1.15 |
| IPI00017696 | 1.15 |
| IPI00305461 | 1.15 |
| IPI00879309 | 1.15 |
| IPI00853369 | 1.15 |
| IPI00843910 | 1.15 |
| IPI00798430 | 1.15 |
| IPI00555812 | 1.15 |
| IPI00791343 | 1.15 |
| IPI00301143 | 1.15 |
| IPI00016915 | 1.15 |
| IPI00013179 | 1.15 |
| IPI00642632 | 1.14 |
| IPI00001793 | 1.14 |
| IPI00291395 | 1.14 |
| IPI00219301 | 1.14 |
| IPI00021033 | 1.14 |
| IPI00023845 | 1.14 |
| IPI00418163 | 1.14 |
| IPI00021854 | 1.14 |
| IPI00742696 | 1.14 |
| IPI00019568 | 1.14 |
| IPI00032258 | 1.14 |
| IPI00247243 | 1.14 |
| IPI00328609 | 1.14 |
| IPI00004440 | 1.14 |
| IPI00419722 | 1.13 |
| IPI00176424 | 1.13 |
| IPI00022392 | 1.13 |
| IPI00879665 | 1.13 |
| IPI00296777 | 1.13 |
| IPI00022463 | 1.13 |
| IPI00296992 | 1.13 |
| IPI00020996 | 1.13 |
| IPI00816626 | 1.13 |
| IPI00005908 | 1.13 |
| IPI00032292 | 1.13 |
| IPI00032405 | 1.13 |
| IPI00152418 | 1.13 |
| IPI00021447 | 1.13 |
| IPI00182944 | 1.13 |
| IPI00816799 | 1.13 |
| IPI00025840 | 1.13 |
| IPI00007199 | 1.13 |
| IPI00019954 | 1.13 |
| IPI00219526 | 1.13 |
| IPI00026216 | 1.13 |
| IPI00021885 | 1.12 |
| IPI00064607 | 1.12 |
| IPI00000828 | 1.12 |
| IPI00784119 | 1.12 |
| IPI00289083 | 1.12 |
| IPI00176221 | 1.12 |
| IPI00029260 | 1.12 |
| IPI00296534 | 1.12 |
| IPI00298793 | 1.12 |
| IPI00299059 | 1.12 |
| IPI00783390 | 1.12 |
| IPI00607600 | 1.11 |
| IPI00299724 | 1.11 |
| IPI00011662 | 1.11 |
| IPI00289831 | 1.11 |
| IPI00032288 | 1.11 |
| IPI00299503 | 1.11 |
| IPI00001399 | 1.11 |
| IPI00376427 | 1.11 |
| IPI00298237 | 1.11 |
| IPI00477804 | 1.11 |
| IPI00020012 | 1.10 |
| IPI00442297 | 1.10 |
| IPI00607580 | 1.10 |
| IPI00442294 | 1.10 |
| IPI00218732 | 1.10 |
| IPI00553177 | 1.10 |
| IPI00218834 | 1.10 |
| IPI00020987 | 1.10 |
| IPI00060310 | 1.10 |
| IPI00168626 | 1.10 |
| IPI00297263 | 1.10 |
| IPI00219029 | 1.10 |
| IPI00003802 | 1.10 |
| IPI00006166 | 1.10 |
| IPI00022395 | 1.10 |
| IPI00017601 | 1.09 |
| IPI00019591 | 1.09 |
| IPI00024966 | 1.09 |
| IPI00552267 | 1.09 |
| IPI00639937 | 1.09 |
| IPI00011264 | 1.09 |
| IPI00020986 | 1.09 |
| IPI00291262 | 1.09 |
| IPI00478003 | 1.09 |
| IPI00451625 | 1.09 |
| IPI00006608 | 1.09 |
| IPI00785200 | 1.08 |
| IPI00012303 | 1.08 |
| IPI00010295 | 1.08 |
| IPI00295414 | 1.08 |
| IPI00289501 | 1.08 |
| IPI00069058 | 1.08 |
| IPI00828205 | 1.08 |
| IPI00027851 | 1.08 |
| IPI00005292 | 1.08 |
| IPI00003366 | 1.08 |
| IPI00854644 | 1.08 |
| IPI00550162 | 1.08 |
| IPI00794184 | 1.08 |
| IPI00845354 | 1.07 |
| IPI00241562 | 1.07 |
| IPI00003351 | 1.07 |
| IPI00102543 | 1.07 |
| IPI00171411 | 1.07 |
| IPI00219798 | 1.07 |
| IPI00298388 | 1.07 |
| IPI00299699 | 1.07 |
| IPI00854806 | 1.07 |
| IPI00829626 | 1.07 |
| IPI00001611 | 1.07 |
| IPI00301812 | 1.07 |
| IPI00026944 | 1.07 |
| IPI00384542 | 1.07 |
| IPI00375205 | 1.07 |
| IPI00015346 | 1.07 |
| IPI00382938 | 1.07 |
| IPI00104074 | 1.07 |
| IPI00027310 | 1.07 |
| IPI00016334 | 1.07 |
| IPI00785196 | 1.07 |
| IPI00827892 | 1.07 |
| IPI00410210 | 1.07 |
| IPI00026104 | 1.07 |
| IPI00293748 | 1.07 |
| IPI00784773 | 1.06 |
| IPI00154742 | 1.06 |
| IPI00328113 | 1.06 |
| IPI00877029 | 1.06 |
| IPI00023814 | 1.06 |
| IPI00472961 | 1.06 |
| IPI00301865 | 1.06 |
| IPI00009950 | 1.06 |
| IPI00853045 | 1.06 |
| IPI00784865 | 1.06 |
| IPI00746963 | 1.06 |
| IPI00784519 | 1.06 |
| IPI00784985 | 1.06 |
| IPI00430808 | 1.06 |
| IPI00440577 | 1.06 |
| IPI00301579 | 1.06 |
| IPI00044369 | 1.06 |
| IPI00306710 | 1.06 |
| IPI00387105 | 1.06 |
| IPI00019906 | 1.06 |
| IPI00854667 | 1.06 |
| IPI00294834 | 1.06 |
| IPI00000779 | 1.06 |
| IPI00028911 | 1.05 |
| IPI00006601 | 1.05 |
| IPI00028553 | 1.05 |
| IPI00791134 | 1.05 |
| IPI00783689 | 1.05 |
| IPI00221224 | 1.05 |
| IPI00550731 | 1.05 |
| IPI00853525 | 1.05 |
| IPI00794070 | 1.05 |
| IPI00012440 | 1.05 |
| IPI00011605 | 1.05 |
| IPI00292791 | 1.05 |
| IPI00219757 | 1.05 |
| IPI00152540 | 1.05 |
| IPI00426051 | 1.05 |
| IPI00479514 | 1.04 |
| IPI00289819 | 1.04 |
| IPI00178302 | 1.04 |
| IPI00374563 | 1.04 |
| IPI00220327 | 1.04 |
| IPI00419595 | 1.04 |
| IPI00026314 | 1.04 |
| IPI00025465 | 1.04 |
| IPI00021842 | 1.04 |
| IPI00218874 | 1.04 |
| IPI00027780 | 1.04 |
| IPI00878576 | 1.03 |
| IPI00156171 | 1.03 |
| IPI00022331 | 1.03 |
| IPI00788835 | 1.03 |
| IPI00022229 | 1.03 |
| IPI00470535 | 1.03 |
| IPI00218911 | 1.03 |
| IPI00166766 | 1.03 |
| IPI00216171 | 1.03 |
| IPI00022892 | 1.02 |
| IPI00028908 | 1.02 |
| IPI00022417 | 1.02 |
| IPI00306339 | 1.02 |
| IPI00333140 | 1.02 |
| IPI00022426 | 1.02 |
| IPI00303210 | 1.02 |
| IPI00385264 | 1.02 |
| IPI00218875 | 1.02 |
| IPI00218413 | 1.02 |
| IPI00166729 | 1.01 |
| IPI00022434 | 1.01 |
| IPI00007921 | 1.01 |
| IPI00647027 | 1.01 |
| IPI00878517 | 1.01 |
| IPI00298971 | 1.01 |
| IPI00296165 | 1.01 |
| IPI00021841 | 1.00 |
| IPI00000027 | 1.00 |
| IPI00830122 | 1.00 |
| IPI00383032 | 1.00 |
| IPI00297040 | 1.00 |
| IPI00031008 | 1.00 |
| IPI00218398 | 1.00 |
| IPI00073763 | 1.00 |
| IPI00167619 | 1.00 |
| IPI00004798 | 1.00 |
| IPI00305380 | 1.00 |
| IPI00827846 | 1.00 |
| IPI00025365 | 1.00 |
| IPI00455521 | 1.00 |
| IPI00307729 | 1.00 |
| IPI00644346 | 1.00 |
| IPI00829980 | 1.00 |
| IPI00219219 | 1.00 |
| IPI00748265 | 1.00 |
| IPI00418169 | 1.00 |
| IPI00171611 | 1.00 |
| IPI00827839 | 1.00 |
| IPI00030634 | 1.00 |
| IPI00017841 | 1.00 |
| IPI00011140 | 1.00 |
| IPI00290744 | 1.00 |
| IPI00028381 | 1.00 |
| IPI00748890 | 1.00 |
| IPI00816737 | 1.00 |
| IPI00440153 | 1.00 |
| IPI00298285 | 1.00 |
| IPI00030877 | 1.00 |
| IPI00024032 | 1.00 |
| IPI00028600 | 1.00 |
| IPI00024107 | 1.00 |
| IPI00026125 | 1.00 |
| IPI00029061 | 1.00 |
| IPI00006128 | 1.00 |
| IPI00216461 | 1.00 |
| IPI00827929 | 1.00 |
| IPI00830018 | 1.00 |
| IPI00550533 | 1.00 |
| IPI00022389 | 1.00 |
| IPI00382488 | 1.00 |
| IPI00854624 | 1.00 |
| IPI00477868 | 1.00 |
| IPI00827580 | 1.00 |
| IPI00328745 | 1.00 |
| IPI00025476 | 1.00 |
| IPI00021983 | 1.00 |
| IPI00413641 | 1.00 |
| IPI00394870 | 1.00 |
| IPI00024572 | 1.00 |
| IPI00018769 | 1.00 |
| IPI00024825 | 1.00 |
| IPI00022822 | 1.00 |
| IPI00023751 | 1.00 |
| IPI00289802 | 1.00 |
| IPI00031765 | 1.00 |
| IPI00004457 | 1.00 |
| IPI00024766 | 1.00 |
| IPI00301631 | 1.00 |
| IPI00478816 | 1.00 |
| IPI00171230 | 1.00 |
| IPI00478997 | 1.00 |
| IPI00306850 | 1.00 |
| IPI00218946 | 1.00 |
| IPI00014371 | 1.00 |
| IPI00305975 | 1.00 |
| IPI00221332 | 1.00 |
| IPI00016014 | 1.00 |
| IPI00413781 | 1.00 |
| IPI00874156 | 1.00 |
| IPI00248596 | 1.00 |
| IPI00296337 | 1.00 |
| IPI00300207 | 1.00 |
| IPI00333126 | 1.00 |
| IPI00010369 | 1.00 |
| IPI00300020 | 1.00 |
| IPI00002320 | 1.00 |
| IPI00103552 | 1.00 |
| IPI00022542 | 1.00 |
| IPI00174976 | 1.00 |
| IPI00382482 | 1.00 |
| IPI00554799 | 1.00 |
| IPI00032830 | 1.00 |
| IPI00219420 | 1.00 |
| IPI00217537 | 1.00 |
| IPI00829836 | 1.00 |
| IPI00000959 | 1.00 |
| IPI00004084 | 1.00 |
| IPI00006644 | 1.00 |
| IPI00170814 | 1.00 |
| IPI00382421 | 1.00 |
| IPI00827724 | 1.00 |
| IPI00375746 | 1.00 |
| IPI00009943 | 1.00 |
| IPI00797310 | 1.00 |
| IPI00026358 | 1.00 |
| IPI00003031 | 1.00 |
| IPI00175654 | 1.00 |
| IPI00243995 | 1.00 |
| IPI00018980 | 1.00 |
| IPI00374590 | 1.00 |
| IPI00163724 | 1.00 |
| IPI00828083 | 1.00 |
| IPI00016645 | 1.00 |
| IPI00645206 | 1.00 |
| IPI00103471 | 1.00 |
| IPI00009793 | 1.00 |
| IPI00014223 | 1.00 |
| IPI00019209 | 1.00 |
| IPI00289924 | 1.00 |
| IPI00883711 | 1.00 |
| IPI00004669 | 1.00 |
| IPI00019038 | 1.00 |
| IPI00023014 | 1.00 |
| IPI00181174 | 1.00 |
| IPI00012585 | 1.00 |
| IPI00025204 | 1.00 |
| IPI00013162 | 1.00 |
| IPI00026103 | 1.00 |
| IPI00011094 | 1.00 |
| IPI00000871 | 1.00 |
| IPI00004494 | 1.00 |
| IPI00182194 | 1.00 |
| IPI00304577 | 1.00 |
| IPI00030111 | 1.00 |
| IPI00029046 | 1.00 |
| IPI00027078 | 1.00 |
| IPI00418531 | 1.00 |
| IPI00296558 | 1.00 |
| IPI00304962 | 1.00 |
| IPI00061448 | 1.00 |
| IPI00025110 | 1.00 |
| IPI00021485 | 1.00 |
| IPI00304925 | 1.00 |
| IPI00297188 | 1.00 |
| IPI00217759 | 1.00 |
| IPI00880120 | 1.00 |
| IPI00171410 | 1.00 |
| IPI00005794 | 1.00 |
| IPI00007702 | 1.00 |
| IPI00024046 | 1.00 |
| IPI00293276 | 1.00 |
| IPI00297181 | 1.00 |
| IPI00006987 | 1.00 |
| IPI00873344 | 1.00 |
| IPI00027264 | 1.00 |
| IPI00027847 | 1.00 |
| IPI00032227 | 1.00 |
| IPI00012269 | 1.00 |
| IPI00004409 | 1.00 |
| IPI00221117 | 1.00 |
| IPI00554521 | 1.00 |
| IPI00218487 | 1.00 |
| IPI00795481 | 1.00 |
| IPI00807609 | 1.00 |
| IPI00065312 | 1.00 |
| IPI00259102 | 1.00 |
| IPI00289746 | 1.00 |
| IPI00002283 | 1.00 |
| IPI00005605 | 1.00 |
| IPI00003971 | 1.00 |
| IPI00295618 | 1.00 |
| IPI00019372 | 1.00 |
| IPI00514517 | 1.00 |
| IPI00412987 | 1.00 |
| IPI00016870 | 1.00 |
| IPI00412492 | 1.00 |
| IPI00015842 | 1.00 |
| IPI00021119 | 1.00 |
| IPI00025869 | 1.00 |
| IPI00006034 | 1.00 |
| IPI00400935 | 1.00 |
| IPI00176581 | 1.00 |
| IPI00061520 | 1.00 |
| IPI00645194 | 1.00 |
| IPI00155729 | 1.00 |
| IPI00216319 | 1.00 |
| IPI00018534 | 1.00 |
| IPI00293723 | 1.00 |
| IPI00006971 | 1.00 |
| IPI00009826 | 1.00 |
| IPI00480183 | 1.00 |
| IPI00328257 | 1.00 |
| IPI00293128 | 1.00 |
| IPI00005038 | 1.00 |
| IPI00297252 | 1.00 |
| IPI00101608 | 1.00 |
| IPI00005774 | 1.00 |
| IPI00008148 | 1.00 |
| IPI00217236 | 1.00 |
| IPI00000760 | 1.00 |
| IPI00018909 | 1.00 |
| IPI00300407 | 1.00 |
| IPI00005722 | 1.00 |
| IPI00165936 | 1.00 |
| IPI00018381 | 1.00 |
| IPI00749245 | 1.00 |
| IPI00010343 | 1.00 |
| IPI00289876 | 1.00 |
| IPI00008215 | 1.00 |
| IPI00337612 | 1.00 |
| IPI00292496 | 1.00 |
| IPI00019180 | 1.00 |
| IPI00386575 | 1.00 |
| IPI00025992 | 1.00 |
| IPI00005123 | 1.00 |
| IPI00002525 | 1.00 |
| IPI00043756 | 1.00 |
| IPI00292300 | 1.00 |
| IPI00102435 | 1.00 |
| IPI00402293 | 1.00 |
| IPI00432723 | 1.00 |
| IPI00243451 | 1.00 |
| IPI00456589 | 1.00 |
| IPI00007425 | 1.00 |
| IPI00328488 | 1.00 |
| IPI00413451 | 1.00 |
| IPI00290328 | 1.00 |
| IPI00556391 | 1.00 |
| IPI00290826 | 1.00 |
| IPI00012989 | 1.00 |
| IPI00478892 | 1.00 |
| IPI00171438 | 1.00 |
| IPI00431738 | 1.00 |
| IPI00019862 | 1.00 |
| IPI00643115 | 1.00 |
| IPI00479997 | 1.00 |
| IPI00002899 | 1.00 |
| IPI00426267 | 1.00 |
| IPI00306851 | 1.00 |
| IPI00374293 | 1.00 |
| IPI00060423 | 1.00 |
| IPI00005806 | 1.00 |
| IPI00008816 | 1.00 |
| IPI00334845 | 1.00 |
| IPI00240345 | 1.00 |
| IPI00447328 | 1.00 |
| IPI00456604 | 1.00 |
| IPI00220736 | 1.00 |
| IPI00410631 | 1.00 |
| IPI00007127 | 1.00 |
| IPI00719690 | 1.00 |
| IPI00554701 | 1.00 |
| IPI00008868 | 1.00 |
| IPI00024134 | 1.00 |
| IPI00387100 | 1.00 |
| IPI00012622 | 1.00 |
| IPI00032358 | 1.00 |
| IPI00442474 | 1.00 |
| IPI00005465 | 1.00 |
| IPI00830059 | 1.00 |
| IPI00217051 | 1.00 |
| IPI00414784 | 1.00 |
| IPI00847644 | 1.00 |
| IPI00412982 | 1.00 |
| IPI00782966 | 1.00 |
| IPI00216969 | 1.00 |
| IPI60451450 | 1.00 |
| IPI00788118 | 1.00 |
| IPI00250716 | 1.00 |
| IPI00296563 | 1.00 |
| IPI00020729 | 1.00 |
| IPI00816409 | 1.00 |
| IPI00376317 | 1.00 |
| IPI00382439 | 1.00 |
| IPI00152432 | 1.00 |
| IPI00022820 | 1.00 |
| IPI00744816 | 1.00 |
| IPI00647124 | 1.00 |
| IPI00010085 | 1.00 |
| IPI00604549 | 1.00 |
| IPI00012869 | 1.00 |
| IPI00028122 | 1.00 |
| IPI00001528 | 1.00 |
| IPI00829845 | 1.00 |
| IPI00290903 | 1.00 |
| IPI00385578 | 1.00 |
| IPI00003053 | 1.00 |
| IPI00006653 | 1.00 |
| IPI00016848 | 1.00 |
| IPI00872208 | 1.00 |
| IPI00032939 | 1.00 |
| IPI00743335 | 1.00 |
| IPI00783950 | 1.00 |
| IPI00001516 | 1.00 |
| IPI00012573 | 1.00 |
| IPI00292134 | 1.00 |
| IPI00658167 | 1.00 |
| IPI00157820 | 1.00 |
| IPI00395010 | 1.00 |
| IPI00021302 | 1.00 |
| IPI00023555 | 1.00 |
| IPI00176574 | 1.00 |
| IPI00027258 | 1.00 |
| IPI00395444 | 1.00 |
| IPI00020228 | 1.00 |
| IPI00291662 | 1.00 |
| IPI00219817 | 1.00 |
| IPI00010869 | 1.00 |
| IPI00877627 | 1.00 |
| IPI00619927 | 1.00 |
| IPI00006152 | 1.00 |
| IPI00026941 | 1.00 |
| IPI00016600 | 1.00 |
| IPI00303335 | 1.00 |
| IPI00031171 | 1.00 |
| IPI00003375 | 1.00 |
| IPI00387109 | 1.00 |
| IPI00217955 | 1.00 |
| IPI00220002 | 1.00 |
| IPI00009524 | 1.00 |
| IPI00003894 | 1.00 |
| IPI00175649 | 1.00 |
| IPI00307016 | 1.00 |
| IPI00455927 | 1.00 |
| IPI00167280 | 1.00 |
| IPI00398709 | 1.00 |
| IPI00027667 | 1.00 |
| IPI00012760 | 1.00 |
| IPI00795119 | 1.00 |
| IPI00023501 | 1.00 |
| IPI00743696 | 1.00 |
| IPI00009891 | 1.00 |
| IPI00002897 | 1.00 |
| IPI00292471 | 1.00 |
| IPI00216313 | 1.00 |
| IPI00419215 | 1.00 |
| IPI00015802 | 1.00 |
| IPI00171160 | 1.00 |
| IPI00029039 | 1.00 |
| IPI00159652 | 1.00 |
| IPI00232571 | 1.00 |
| IPI00060969 | 1.00 |
| IPI00293588 | 1.00 |
| IPI00386574 | 1.00 |
| IPI00470596 | 1.00 |
| IPI00005171 | 1.00 |
| IPI00054042 | 1.00 |
| IPI00025862 | 1.00 |
| IPI00100867 | 1.00 |
| IPI00219953 | 1.00 |
| IPI00217442 | 1.00 |
| IPI00873139 | 1.00 |
| IPI00288940 | 1.00 |
| IPI00024915 | 1.00 |
| IPI00166044 | 1.00 |
| IPI00470606 | 1.00 |
| IPI00007836 | 1.00 |
| IPI00106491 | 1.00 |
| IPI00021131 | 1.00 |
| IPI00010676 | 1.00 |
| IPI00743360 | 1.00 |
| IPI00737363 | 1.00 |
| IPI00300046 | 1.00 |
| IPI00000621 | 1.00 |
| IPI00017603 | 1.00 |
| IPI00410214 | 1.00 |
| IPI00103604 | 1.00 |
| IPI00153053 | 1.00 |
| IPI00296434 | 1.00 |
| IPI00411979 | 1.00 |
| IPI00186114 | 1.00 |
| IPI00216236 | 1.00 |
| IPI00816289 | 1.00 |
| IPI00060866 | 1.00 |
| IPI00013743 | 1.00 |
| IPI00829750 | 1.00 |
| IPI00472782 | 1.00 |
| IPI00022621 | 1.00 |
| IPI00012759 | 1.00 |
| IPI00166323 | 1.00 |
| IPI00742682 | 1.00 |
| IPI00175193 | 1.00 |
| IPI00002689 | 1.00 |
| IPI00103146 | 1.00 |
| IPI00011951 | 1.00 |
| IPI00292086 | 1.00 |
| IPI00413293 | 1.00 |
| IPI00382539 | 1.00 |
| IPI00024975 | 1.00 |
| IPI00027356 | 1.00 |
| IPI00472426 | 1.00 |
| IPI00792626 | 1.00 |
| IPI00789954 | 1.00 |
| IPI00011229 | 0.99 |
| IPI00748312 | 0.99 |
| IPI00021000 | 0.99 |
| IPI00296608 | 0.99 |
| IPI00384697 | 0.99 |
| IPI00159927 | 0.98 |
| IPI00027703 | 0.98 |
| IPI00290315 | 0.98 |
| IPI00018236 | 0.98 |
| IPI00294004 | 0.98 |
| IPI00396423 | 0.98 |
| IPI00018136 | 0.98 |
| IPI00002280 | 0.98 |
| IPI00029739 | 0.98 |
| IPI00290085 | 0.98 |
| IPI00333776 | 0.98 |
| IPI00746623 | 0.98 |
| IPI00883753 | 0.98 |
| IPI00515041 | 0.97 |
| IPI00414249 | 0.97 |
| IPI00219446 | 0.97 |
| IPI00415032 | 0.97 |
| IPI00029751 | 0.97 |
| IPI00003102 | 0.97 |
| IPI00000137 | 0.97 |
| IPI00006967 | 0.96 |
| IPI00023728 | 0.96 |
| IPI00216728 | 0.96 |
| IPI00297224 | 0.96 |
| IPI00064667 | 0.96 |
| IPI00297487 | 0.96 |
| IPI00328829 | 0.96 |
| IPI00760721 | 0.96 |
| IPI00784430 | 0.96 |
| IPI00829663 | 0.96 |
| IPI00103597 | 0.96 |
| IPI00011302 | 0.96 |
| IPI00001952 | 0.95 |
| IPI00027466 | 0.95 |
| IPI00024284 | 0.95 |
| IPI00387106 | 0.95 |
| IPI00294395 | 0.95 |
| IPI00641737 | 0.95 |
| IPI00001662 | 0.95 |
| IPI00827637 | 0.95 |
| IPI00022391 | 0.95 |
| IPI00855725 | 0.95 |
| IPI00739237 | 0.94 |
| IPI00387117 | 0.94 |
| IPI00010471 | 0.94 |
| IPI00296141 | 0.94 |
| IPI00072917 | 0.94 |
| IPI00643348 | 0.94 |
| IPI00607655 | 0.94 |
| IPI00009362 | 0.94 |
| IPI00783987 | 0.94 |
| IPI00022371 | 0.94 |
| IPI00394655 | 0.94 |
| IPI00655702 | 0.94 |
| IPI00034319 | 0.94 |
| IPI00829740 | 0.94 |
| IPI00019771 | 0.93 |
| IPI00031821 | 0.93 |
| IPI00044743 | 0.93 |
| IPI00030075 | 0.93 |
| IPI00000877 | 0.93 |
| IPI00334238 | 0.93 |
| IPI00219217 | 0.93 |
| IPI00215767 | 0.93 |
| IPI00220361 | 0.93 |
| IPI00029193 | 0.93 |
| IPI00023648 | 0.93 |
| IPI00334282 | 0.93 |
| IPI00414984 | 0.93 |
| IPI00020557 | 0.92 |
| IPI00024272 | 0.92 |
| IPI00387118 | 0.92 |
| IPI00328520 | 0.92 |
| IPI00107731 | 0.92 |
| IPI00384404 | 0.92 |
| IPI00827581 | 0.92 |
| IPI00056478 | 0.92 |
| IPI00025846 | 0.92 |
| IPI00017569 | 0.92 |
| IPI00157414 | 0.92 |
| IPI00827510 | 0.92 |
| IPI00784950 | 0.92 |
| IPI00166622 | 0.92 |
| IPI00003470 | 0.92 |
| IPI00300241 | 0.92 |
| IPI00015260 | 0.92 |
| IPI00217882 | 0.92 |
| IPI00002816 | 0.92 |
| IPI00024587 | 0.92 |
| IPI00008290 | 0.91 |
| IPI00784807 | 0.91 |
| IPI00872555 | 0.91 |
| IPI00292071 | 0.91 |
| IPI00006114 | 0.91 |
| IPI00004656 | 0.91 |
| IPI00216882 | 0.91 |
| IPI00257882 | 0.91 |
| IPI00550558 | 0.91 |
| IPI00788786 | 0.91 |
| IPI00028448 | 0.91 |
| IPI00829640 | 0.91 |
| IPI00026199 | 0.91 |
| IPI00015102 | 0.91 |
| IPI00032532 | 0.91 |
| IPI00784156 | 0.91 |
| IPI00386879 | 0.91 |
| IPI00176427 | 0.91 |
| IPI00643034 | 0.91 |
| IPI00827560 | 0.90 |
| IPI00025818 | 0.90 |
| IPI00292550 | 0.90 |
| IPI00784942 | 0.90 |
| IPI00387115 | 0.90 |
| IPI00784830 | 0.90 |
| IPI00216138 | 0.90 |
| IPI00747849 | 0.90 |
| IPI00000138 | 0.90 |
| IPI00301459 | 0.90 |
| IPI00016422 | 0.90 |
| IPI00785067 | 0.90 |
| IPI00162735 | 0.90 |
| IPI00019399 | 0.90 |
| IPI00022432 | 0.90 |
| IPI00784758 | 0.90 |
| IPI00384391 | 0.90 |
| IPI00027493 | 0.90 |
| IPI00008318 | 0.90 |
| IPI00010154 | 0.89 |
| IPI00019157 | 0.89 |
| IPI00788189 | 0.89 |
| IPI00852577 | 0.89 |
| IPI00745660 | 0.89 |
| IPI00784969 | 0.89 |
| IPI00242956 | 0.89 |
| IPI00785079 | 0.89 |
| IPI00002147 | 0.89 |
| IPI00020599 | 0.89 |
| IPI00027848 | 0.89 |
| IPI00871139 | 0.89 |
| IPI00007221 | 0.89 |
| IPI00017257 | 0.89 |
| IPI00021552 | 0.89 |
| IPI00021794 | 0.89 |
| IPI00829812 | 0.89 |
| IPI00015688 | 0.89 |
| IPI00784998 | 0.89 |
| IPI00027166 | 0.89 |
| IPI00106646 | 0.88 |
| IPI00011651 | 0.88 |
| IPI00790899 | 0.88 |
| IPI00030205 | 0.88 |
| IPI00815938 | 0.88 |
| IPI00795918 | 0.88 |
| IPI00439446 | 0.88 |
| IPI00166339 | 0.88 |
| IPI00015881 | 0.88 |
| IPI00026946 | 0.88 |
| IPI00025257 | 0.88 |
| IPI00061977 | 0.88 |
| IPI00465184 | 0.88 |
| IPI00744561 | 0.88 |
| IPI00430842 | 0.88 |
| IPI00295542 | 0.88 |
| IPI00413826 | 0.88 |
| IPI00021857 | 0.88 |
| IPI00013897 | 0.88 |
| IPI00465439 | 0.88 |
| IPI00009276 | 0.88 |
| IPI00478483 | 0.88 |
| IPI00402157 | 0.88 |
| IPI00784257 | 0.88 |
| IPI00384952 | 0.87 |
| IPI00163446 | 0.87 |
| IPI00011732 | 0.87 |
| IPI00166392 | 0.87 |
| IPI00877169 | 0.87 |
| IPI00410600 | 0.87 |
| IPI00029693 | 0.87 |
| IPI00216602 | 0.87 |
| IPI00647704 | 0.87 |
| IPI00375547 | 0.86 |
| IPI00301395 | 0.86 |
| IPI00385252 | 0.86 |
| IPI00029723 | 0.86 |
| IPI00022314 | 0.86 |
| IPI00828105 | 0.86 |
| IPI00032050 | 0.86 |
| IPI00012009 | 0.86 |
| IPI00029168 | 0.86 |
| IPI00026174 | 0.86 |
| IPI00382499 | 0.86 |
| IPI00075248 | 0.86 |
| IPI00302592 | 0.86 |
| IPI00791479 | 0.86 |
| IPI00008586 | 0.86 |
| IPI00002142 | 0.86 |
| IPI00027192 | 0.86 |
| IPI00423461 | 0.85 |
| IPI00023673 | 0.85 |
| IPI00829767 | 0.85 |
| IPI00002732 | 0.85 |
| IPI00477747 | 0.85 |
| IPI00872363 | 0.85 |
| IPI00829711 | 0.85 |
| IPI00031769 | 0.85 |
| IPI00027038 | 0.85 |
| IPI00011261 | 0.84 |
| IPI00290283 | 0.84 |
| IPI00295741 | 0.84 |
| IPI00642861 | 0.84 |
| IPI00004962 | 0.84 |
| IPI00176458 | 0.84 |
| IPI00879084 | 0.84 |
| IPI00220117 | 0.84 |
| IPI00472345 | 0.84 |
| IPI00784894 | 0.83 |
| IPI00382476 | 0.83 |
| IPI00179473 | 0.83 |
| IPI00030887 | 0.83 |
| IPI00478414 | 0.83 |
| IPI00218795 | 0.83 |
| IPI00783818 | 0.83 |
| IPI00003648 | 0.83 |
| IPI00745251 | 0.83 |
| IPI00007797 | 0.83 |
| IPI00217376 | 0.83 |
| IPI00023858 | 0.83 |
| IPI00010348 | 0.83 |
| IPI00011730 | 0.83 |
| IPI00168866 | 0.83 |
| IPI00182438 | 0.83 |
| IPI00163187 | 0.83 |
| IPI00019501 | 0.83 |
| IPI00328243 | 0.83 |
| IPI00219042 | 0.83 |
| IPI00784828 | 0.83 |
| IPI00004433 | 0.83 |
| IPI00383887 | 0.82 |
| IPI00009802 | 0.82 |
| IPI00761159 | 0.82 |
| IPI00020990 | 0.82 |
| IPI00787781 | 0.82 |
| IPI00107831 | 0.82 |
| IPI00177543 | 0.82 |
| IPI00735451 | 0.82 |
| IPI00410122 | 0.82 |
| IPI00550115 | 0.82 |
| IPI00026237 | 0.82 |
| IPI00024034 | 0.82 |
| IPI00294650 | 0.82 |
| IPI00154734 | 0.82 |
| IPI00014572 | 0.81 |
| IPI00165438 | 0.81 |
| IPI00419585 | 0.81 |
| IPI00021727 | 0.81 |
| IPI00337548 | 0.81 |
| IPI00008994 | 0.81 |
| IPI00815926 | 0.81 |
| IPI00784842 | 0.80 |
| IPI00448925 | 0.80 |
| IPI00031121 | 0.80 |
| IPI00785084 | 0.80 |
| IPI00423463 | 0.80 |
| IPI00298702 | 0.80 |
| IPI00002790 | 0.80 |
| IPI00830025 | 0.80 |
| IPI00019359 | 0.80 |
| IPI00398715 | 0.80 |
| IPI00006009 | 0.80 |
| IPI00219025 | 0.80 |
| IPI00414467 | 0.80 |
| IPI00017745 | 0.80 |
| IPI00374914 | 0.80 |
| IPI00440580 | 0.80 |
| IPI00428967 | 0.80 |
| IPI00026270 | 0.80 |
| IPI00304331 | 0.80 |
| IPI00303071 | 0.80 |
| IPI00011994 | 0.80 |
| IPI00017562 | 0.80 |
| IPI00005474 | 0.80 |
| IPI00643667 | 0.80 |
| IPI00477611 | 0.80 |
| IPI00178767 | 0.80 |
| IPI00423466 | 0.80 |
| IPI00011218 | 0.80 |
| IPI00006662 | 0.80 |
| IPI00029343 | 0.80 |
| IPI00163563 | 0.80 |
| IPI00784810 | 0.79 |
| IPI00168884 | 0.79 |
| IPI00419724 | 0.79 |
| IPI00829752 | 0.79 |
| IPI00045841 | 0.79 |
| IPI00165949 | 0.79 |
| IPI00026530 | 0.79 |
| IPI00432525 | 0.79 |
| IPI00186903 | 0.79 |
| IPI00783184 | 0.79 |
| IPI00291488 | 0.79 |
| IPI00012545 | 0.79 |
| IPI00552937 | 0.78 |
| IPI00873863 | 0.78 |
| IPI00375879 | 0.78 |
| IPI00555693 | 0.78 |
| IPI00304865 | 0.78 |
| IPI00854707 | 0.77 |
| IPI00412988 | 0.77 |
| IPI00013299 | 0.77 |
| IPI00015525 | 0.77 |
| IPI00328680 | 0.77 |
| IPI00783393 | 0.77 |
| IPI00216298 | 0.77 |
| IPI00012386 | 0.77 |
| IPI00855824 | 0.77 |
| IPI00383953 | 0.76 |
| IPI00003362 | 0.76 |
| IPI00029658 | 0.76 |
| IPI00022394 | 0.76 |
| IPI00790473 | 0.76 |
| IPI00465028 | 0.76 |
| IPI00000190 | 0.75 |
| IPI00552771 | 0.75 |
| IPI00003933 | 0.75 |
| IPI00884092 | 0.75 |
| IPI00011899 | 0.75 |
| IPI00744692 | 0.75 |
| IPI00411706 | 0.75 |
| IPI00828156 | 0.75 |
| IPI00005732 | 0.75 |
| IPI00027463 | 0.75 |
| IPI00020984 | 0.75 |
| IPI00252731 | 0.75 |
| IPI00395866 | 0.75 |
| IPI00029756 | 0.75 |
| IPI00018305 | 0.75 |
| IPI00329332 | 0.75 |
| IPI00385980 | 0.75 |
| IPI00000914 | 0.75 |
| IPI00017968 | 0.75 |
| IPI00299571 | 0.75 |
| IPI00293849 | 0.75 |
| IPI00010790 | 0.75 |
| IPI00009145 | 0.75 |
| IPI00031564 | 0.75 |
| IPI00295399 | 0.75 |
| IPI00299086 | 0.75 |
| IPI00004503 | 0.75 |
| IPI00026185 | 0.75 |
| IPI00065533 | 0.75 |
| IPI00002714 | 0.75 |
| IPI00022429 | 0.74 |
| IPI00180240 | 0.74 |
| IPI00220642 | 0.74 |
| IPI00250724 | 0.74 |
| IPI00328703 | 0.73 |
| IPI00021900 | 0.73 |
| IPI00081836 | 0.73 |
| IPI00791228 | 0.73 |
| IPI00004114 | 0.73 |
| IPI00027230 | 0.73 |
| IPI00298281 | 0.72 |
| IPI00304273 | 0.72 |
| IPI00000832 | 0.72 |
| IPI00302641 | 0.72 |
| IPI00020091 | 0.72 |
| IPI00830132 | 0.72 |
| IPI00847179 | 0.72 |
| IPI00790122 | 0.71 |
| IPI00023754 | 0.71 |
| IPI00016467 | 0.71 |
| IPI00030255 | 0.71 |
| IPI00387168 | 0.71 |
| IPI00009904 | 0.71 |
| IPI00409640 | 0.71 |
| IPI00013698 | 0.71 |
| IP100871326 | 0.71 |
| IPI00029606 | 0.71 |
| IPI00298497 | 0.71 |
| IPI00718977 | 0.71 |
| IPI00031461 | 0.71 |
| IPI00296176 | 0.71 |
| IPI00382606 | 0.71 |
| IPI00827876 | 0.71 |
| IPI00384392 | 0.71 |
| IPI00013303 | 0.70 |
| IPI00478890 | 0.70 |
| IPI00001960 | 0.70 |
| IPI00293925 | 0.70 |
| IPI00176193 | 0.70 |
| IPI00021903 | 0.70 |
| IPI00291005 | 0.70 |
| IPI00013976 | 0.69 |
| IPI00827482 | 0.69 |
| IPI00015756 | 0.69 |
| IPI00418446 | 0.69 |
| IPI00014439 | 0.69 |
| IPI00002745 | 0.69 |
| IPI00220644 | 0.69 |
| IPI00003269 | 0.69 |
| IPI00386133 | 0.68 |
| IPI00853454 | 0.68 |
| IPI00009028 | 0.67 |
| IPI00792115 | 0.67 |
| IPI00418262 | 0.67 |
| IPI00796279 | 0.67 |
| IPI00289334 | 0.67 |
| IPI00219077 | 0.67 |
| IPI00554474 | 0.67 |
| IPI00024012 | 0.67 |
| IPI00013860 | 0.67 |
| IPI00847723 | 0.67 |
| IPI00029236 | 0.67 |
| IPI00166613 | 0.67 |
| IPI00024036 | 0.67 |
| IPI00829810 | 0.67 |
| IPI00292218 | 0.67 |
| IPI00549330 | 0.67 |
| IPI00289926 | 0.67 |
| IPI00387096 | 0.67 |
| IPI00022462 | 0.67 |
| IPI00443909 | 0.67 |
| IPI00019485 | 0.67 |
| IPI00374732 | 0.67 |
| IPI00168847 | 0.67 |
| IPI00220791 | 0.67 |
| IPI00847652 | 0.67 |
| IPI00015351 | 0.67 |
| IPI00024105 | 0.67 |
| IPI00153049 | 0.67 |
| IPI00387119 | 0.67 |
| IPI00027721 | 0.67 |
| IPI00640292 | 0.67 |
| IPI00414909 | 0.67 |
| IPI00170692 | 0.67 |
| IPI00018146 | 0.67 |
| IPI00064377 | 0.67 |
| IPI00008164 | 0.67 |
| IPI00470625 | 0.67 |
| IPI00829827 | 0.67 |
| IPI00788824 | 0.67 |
| IPI00176398 | 0.67 |
| IPI00304840 | 0.67 |
| IPI00033466 | 0.67 |
| IPI00302181 | 0.67 |
| IPI00022959 | 0.67 |
| IPI00217435 | 0.67 |
| IPI00027377 | 0.67 |
| IPI00170635 | 0.67 |
| IPI00220766 | 0.67 |
| IPI00295386 | 0.67 |
| IPI00289870 | 0.67 |
| IPI00044707 | 0.67 |
| IPI00847670 | 0.67 |
| IPI00815662 | 0.67 |
| IPI00025861 | 0.67 |
| IPI00295461 | 0.67 |
| IPI00015343 | 0.67 |
| IPI00023283 | 0.67 |
| IPI00005721 | 0.67 |
| IPI00292130 | 0.67 |
| IPI00021458 | 0.67 |
| IPI00376210 | 0.67 |
| IPI00418573 | 0.67 |
| IPI00019576 | 0.66 |
| IPI00296922 | 0.66 |
| IPI00784258 | 0.65 |
| IPI00026197 | 0.65 |
| IPI00012102 | 0.65 |
| IPI00183487 | 0.65 |
| IPI00024570 | 0.64 |
| IPI00411680 | 0.64 |
| IPI00216457 | 0.63 |
| IPI00009123 | 0.63 |
| IPI00218407 | 0.63 |
| IPI00026259 | 0.63 |
| IPI00414676 | 0.63 |
| IPI00329482 | 0.63 |
| IPI00003021 | 0.63 |
| IPI00025276 | 0.63 |
| IPI00303476 | 0.63 |
| IPI00736860 | 0.62 |
| IPI00005126 | 0.62 |
| IPI00218725 | 0.61 |
| IPI00000816 | 0.61 |
| IPI00793166 | 0.61 |
| IPI00021263 | 0.61 |
| IPI00005707 | 0.61 |
| IPI00013216 | 0.60 |
| IPI00009890 | 0.60 |
| IPI00414294 | 0.60 |
| IPI00000087 | 0.60 |
| IPI00328431 | 0.60 |
| IPI00827939 | 0.60 |
| IPI00293971 | 0.60 |
| IPI00413912 | 0.60 |
| IPI00376131 | 0.60 |
| IPI00030739 | 0.60 |
| IPI00329685 | 0.60 |
| IPI00291175 | 0.60 |
| IPI00029628 | 0.60 |
| IPI00029631 | 0.60 |
| IPI00008787 | 0.59 |
| IPI00387097 | 0.59 |
| IPI00307276 | 0.59 |
| IPI00828191 | 0.58 |
| IPI00026800 | 0.58 |
| IPI00297444 | 0.57 |
| IPI00444605 | 0.57 |
| IPI00437751 | 0.57 |
| IPI00171928 | 0.57 |
| IPI00015877 | 0.57 |
| IPI00008603 | 0.57 |
| IPI00022296 | 0.56 |
| IPI00022810 | 0.56 |
| IPI00387120 | 0.56 |
| IPI00025252 | 0.56 |
| IPI00026240 | 0.56 |
| IPI00401264 | 0.56 |
| IPI00107886 | 0.56 |
| IPI00024048 | 0.56 |
| IPI00019533 | 0.56 |
| IPI00056357 | 0.56 |
| IPI00740545 | 0.56 |
| IPI00032063 | 0.56 |
| IPI00022792 | 0.55 |
| IPI00293539 | 0.55 |
| IPI00020977 | 0.55 |
| IPI00017704 | 0.55 |
| IPI00015902 | 0.55 |
| IPI00019190 | 0.54 |
| IPI00299652 | 0.54 |
| IPI00011454 | 0.53 |
| IPI00025864 | 0.53 |
| IPI00027422 | 0.53 |
| IPI00783665 | 0.53 |
| IPI00007236 | 0.53 |
| IPI00299485 | 0.50 |
| IPI00021327 | 0.50 |
| IPI00555614 | 0.50 |
| IPI00289329 | 0.50 |
| IPI00383016 | 0.50 |
| IPI00012044 | 0.50 |
| IPI00552874 | 0.50 |
| IPI00853073 | 0.50 |
| IPI00028561 | 0.50 |
| IPI00645078 | 0.50 |
| IPI00027429 | 0.50 |
| IPI00013991 | 0.50 |
| IPI00044326 | 0.50 |
| IPI00027875 | 0.50 |
| IPI00424119 | 0.50 |
| IPI00789398 | 0.50 |
| IPI00169426 | 0.50 |
| IPI00829759 | 0.50 |
| IPI00005128 | 0.50 |
| IPI00013302 | 0.50 |
| IPI00306959 | 0.50 |
| IPI00218820 | 0.50 |
| IPI00384722 | 0.50 |
| IPI00787936 | 0.50 |
| IPI00009294 | 0.50 |
| IPI00290358 | 0.50 |
| IPI00103755 | 0.50 |
| IPI00030871 | 0.50 |
| IPI00031131 | 0.50 |
| IPI00553092 | 0.50 |
| IPI00021733 | 0.50 |
| IPI00303882 | 0.50 |
| IPI00382474 | 0.50 |
| IPI00296099 | 0.50 |
| IPI00178352 | 0.50 |
| IPI00413587 | 0.50 |
| IPI00006444 | 0.50 |
| IPI00007960 | 0.50 |
| IPI00293464 | 0.50 |
| IPI00002236 | 0.50 |
| IPI00149097 | 0.50 |
| IPI00410588 | 0.50 |
| IPI00218319 | 0.50 |
| IPI00215899 | 0.50 |
| IPI00010706 | 0.50 |
| IPI00410675 | 0.50 |
| IPI00043215 | 0.50 |
| IPI00004573 | 0.50 |
| IPI00221178 | 0.50 |
| IPI00220748 | 0.50 |
| IPI00166075 | 0.50 |
| IPI00016862 | 0.50 |
| IPI00184019 | 0.50 |
| IPI00024466 | 0.50 |
| IPI00022649 | 0.50 |
| IPI00020672 | 0.50 |
| IPI00012792 | 0.50 |
| IPI00184851 | 0.50 |
| IPI00019907 | 0.50 |
| IPI00001893 | 0.50 |
| IPI00157454 | 0.50 |
| IPI00024129 | 0.50 |
| IPI00024929 | 0.50 |
| IPI00016621 | 0.50 |
| IPI00001633 | 0.50 |
| IPI00027341 | 0.50 |
| IPI00470484 | 0.50 |
| IPI00008494 | 0.50 |
| IPI00022640 | 0.50 |
| IPI00022039 | 0.50 |
| IPI00385007 | 0.50 |
| IPI00010470 | 0.50 |
| IPI00657742 | 0.50 |
| IPI00010148 | 0.50 |
| IPI00329801 | 0.50 |
| IPI00643920 | 0.50 |
| IPI00289849 | 0.50 |
| IPI00382424 | 0.50 |
| IPI00006705 | 0.50 |
| IPI00048230 | 0.50 |
| IPI00550571 | 0.50 |
| IPI00056309 | 0.50 |
| IPI00000949 | 0.50 |
| IPI00644752 | 0.50 |
| IPI00641920 | 0.50 |
| IPI00382483 | 0.50 |
| IPI00032876 | 0.50 |
| IPI00012402 | 0.50 |
| IPI00217787 | 0.50 |
| IPI00015159 | 0.50 |
| IPI00746034 | 0.50 |
| IPI00306402 | 0.50 |
| IPI00478921 | 0.50 |
| IPI00296063 | 0.50 |
| IPI00009286 | 0.50 |
| IPI00457109 | 0.50 |
| IPI00006631 | 0.50 |
| IPI00294398 | 0.50 |
| IPI00216256 | 0.50 |
| IPI00010277 | 0.50 |
| IPI00020017 | 0.50 |
| IPI00011773 | 0.50 |
| IPI00178150 | 0.50 |
| IPI00446753 | 0.50 |
| IPI00007729 | 0.50 |
| IPI00013260 | 0.50 |
| IPI00302647 | 0.50 |
| IPI00025753 | 0.50 |
| IPI00013418 | 0.50 |
| IPI00002649 | 0.50 |
| IPI00221101 | 0.50 |
| IPI00000874 | 0.47 |
| IPI00827891 | 0.46 |
| IPI00216318 | 0.46 |
| IPI00219365 | 0.45 |
| IPI00008087 | 0.44 |
| IPI00218570 | 0.44 |
| IPI00790775 | 0.44 |
| IPI00473033 | 0.43 |
| IPI00010182 | 0.43 |
| IPI00828037 | 0.43 |
| IPI00374065 | 0.43 |
| IPI00021275 | 0.43 |
| IPI00044600 | 0.43 |
| IPI00302840 | 0.43 |
| IPI00032904 | 0.40 |
| IPI00556643 | 0.40 |
| IPI00019146 | 0.40 |
| IPI00024919 | 0.40 |
| IPI00387116 | 0.40 |
| IPI00027223 | 0.40 |
| IPI00006451 | 0.40 |
| IPI00162329 | 0.40 |
| IPI00012011 | 0.40 |
| IPI00419908 | 0.33 |
| IPI00289861 | 0.33 |
| IPI00830044 | 0.33 |
| IPI00152216 | 0.33 |
| IPI00027444 | 0.33 |
| IPI00396383 | 0.33 |
| IPI00550677 | 0.33 |
| IPI00297714 | 0.33 |
| IPI00171678 | 0.33 |
| IPI00297655 | 0.33 |
| IPI00816794 | 0.33 |
| IPI00178926 | 0.33 |
| IPI00784368 | 0.33 |
| IPI00396930 | 0.33 |
| IPI00384402 | 0.33 |
| IPI00016371 | 0.33 |
| IPI00150881 | 0.33 |
| IPI00220827 | 0.33 |
| IPI00383680 | 0.33 |
| IPI00020396 | 0.33 |
| IPI00023643 | 0.33 |
| IPI00001477 | 0.33 |
| IPI00789847 | 0.33 |
| IPI00303894 | 0.33 |
| IPI00023598 | 0.33 |
| IPI00011654 | 0.33 |
| IPI00013475 | 0.33 |
| IPI00026646 | 0.33 |
| IPI00550069 | 0.33 |
| IPI00023843 | 0.33 |
| IPI00013079 | 0.33 |
| IPI00549189 | 0.33 |
| IPI00063523 | 0.33 |
| IPI00296286 | 0.33 |
| IPI00012902 | 0.33 |
| IPI00014230 | 0.29 |
| IPI00007798 | 0.29 |
| IPI00441498 | 0.27 |
| IPI00829834 | 0.27 |
| IPI00470838 | 0.25 |
| IPI00004367 | 0.25 |
| IPI00646291 | 0.25 |
| IPI00007249 | 0.25 |
| IPI00009867 | 0.25 |
| IPI00739099 | 0.25 |
| IPI00219575 | 0.25 |
| IPI00382442 | 0.25 |
| IPI00022977 | 0.25 |
| IPI00031510 | 0.25 |
| IPI00328872 | 0.25 |
| IPI00001580 | 0.25 |
| IPI00103749 | 0.25 |
| IPI00256974 | 0.25 |
| IPI00007461 | 0.25 |
| IPI00025295 | 0.25 |
| IPI00385143 | 0.23 |
| IPI00013495 | 0.20 |
| IPI00377045 | 0.20 |
| IP100386131 | 0.20 |
| IPI00181079 | 0.20 |
| IPI00006482 | 0.20 |
| IPI00300725 | 0.20 |
| IPI00299145 | 0.20 |
| IPI00007752 | 0.20 |
| IPI00382490 | 0.20 |
| IPI00552735 | 0.17 |
| IPI00384225 | 0.17 |
| IPI00021855 | 0.17 |
| IPI00827789 | 0.17 |
| IPI00024138 | 0.14 |
| IPI00011062 | 0.14 |
| IPI00060715 | 0.13 |
| IPI00018206 | 0.13 |
| IPI00412264 | 0.08 |
| IPI00167419 | 0.03 |
| IPI00394712 | |
| IPI00141938 | |
| IPI00018342 | |
| IPI00456683 | |
| IPI00012391 | |
| IPI00185088 | |
| IPI00028786 | |
| IPI00152182 | |
| IPI00299778 | |
| IPI00216694 | |
| IPI00020407 | |
| IPI00295767 | |
| IPI00004047 | |
| IPI00008274 | |
| IPI00001863 | |
| IPI00743898 | |
| IPI00290078 | |
| IPI00215979 | |
| IPI00025311 | |
| IPI00006470 | |
| IPI00023359 | |
| IPI00018708 | |
| IPI00011781 | |
| IPI00033030 | |
| IPI00307612 | |
| IPI00002459 | |
| IPI00221034 | |
| IPI00021274 | |
| IPI00024580 | |
| IPI00185661 | |
| IPI00479083 | |
| IPI00045839 | |
| IPI00216592 | |
| IPI00168885 | |
| IPI00642259 | |
| IPI00306844 | |
| IPI00165229 | |
| IPI00217652 | |
| IPI00741005 | |
| IPI00446588 | |
| IPI00029591 | |
| IPI00514893 | |
| IPI00023191 | |
| IPI00025363 | |
| IPI00009365 | |
| IPI00792759 | |
| IPI00219525 | |
| IPI00005690 | |
| IPI00017529 | |
| IPI00218730 | |
| IPI00216049 | |
| IPI00383594 | |
| IPI00307591 | |
| IPI00170766 | |
| IPI00302133 | |
| IPI00304789 | |
| IPI00027744 | |
| IPI00375881 | |
| IPI00297550 | |
| IPI00026230 | |
| IPI00021048 | |
| IPI00290857 | |
| IPI00845229 | |
| IPI00867509 | |
| IPI00010207 | |
| IPI00014398 | |
| IPI00218465 | |
| IPI00011592 | |
| IPI00005158 | |
| IPI00410013 | |
| IPI00218292 | |
| IPI00853400 | |
| IPI00748955 | |
| IPI00013319 | |
| IPI00301465 | |
| IPI00024853 | |
| IPI00221255 | |
| IPI00419836 | |
| IPI00000104 | |
| IPI00454910 | |
| IPI00021364 | |
| IPI00306884 | |
| IPI00007709 | |
| IPI00879575 | |
| IPI00004315 | |
| IPI00384016 | |
| IPI00005159 | |
| IPI00024067 | |
| IPI00465261 | |
| IPI00152769 | |
| IPI00432707 | |
| IPI00020692 | |
| IPI00647217 | |
| IPI00219910 | |
| IPI00013281 | |
| IPI00297779 | |
| IPI00033600 | |
| IPI00061354 | |
| IPI00012510 | |
| IPI00216774 | |
| IPI00026546 | |
| IPI00003441 | |
| IPI00335946 | |
| IPI00013978 | |
| IPI00168920 | |
| IPI00219930 | |
| IPI00168520 | |
| IPI00028082 | |
| IPI00871227 | |
| IPI00396378 | |
| IPI00031718 | |
| IPI00152524 | |
| IPI00646689 | |
| IPI00552943 | |
| IPI00001755 | |
| IPI00746177 | |
| IPI00029928 | |
| IPI00219806 | |
| IPI00023576 | |
| IPI00302944 | |
| IPI00013272 | |
| IPI00106502 | |
| IPI00218345 | |
| IPI00025812 | |
| IPI00217005 | |
| IPI00220281 | |
| IPI00007102 | |
| IPI00217466 | |
| IPI00022774 | |
| IPI00031789 | |
| IPI00005652 | |
| IPI00027464 | |
| IPI00246058 | |
| IPI00014592 | |
| IPI00027462 | |
| IPI00442582 | |
| IPI00013378 | |
| IPI00008449 | |
| IPI00062882 | |
| IPI00414402 | |
| IPI00445167 | |
| IPI00018880 | |
| IPI00028262 | |
| IPI00027212 | |
| IPI00291839 | |
| IPI00335711 | |
| IPI00024255 | |
| IPI00009315 | |
| IPI00018583 | |
| IPI00793408 | |
| IPI00045503 | |
| IPI00011274 | |
| IPI00030986 | |
| IPI00168913 | |
| IPI00871625 | |
| IPI00868835 | |
| IPI00742127 | |
| IPI00744179 | |
| IPI00410127 | |
| IPI00418164 | |
| IPI00396658 | |
| IPI00654693 | |
| IPI00429190 | |
| IPI00880011 | |
| IPI00005567 | |
| IPI00217494 | |
| IPI00032598 | |
| IPI00009724 | |
| IPI00644785 | |
| IPI00396279 | |
| IPI00464994 | |
| IPI00296535 | |
| IPI00878483 | |
| IPI00033019 | |
| IPI00398586 | |
| IPI00000837 | |
| IPI00004445 | |
| IPI00028232 | |
| IPI00103595 | |
| IPI00384767 | |
| IPI00398272 | |
| IPI00002824 | |
| IPI00640240 | |
| IPI00028050 | |
| IPI00873348 | |
| IPI00003842 | |
| IPI00878015 | |
| IPI00395473 | |
| IPI00302614 | |
| IPI00419992 | |
| IPI00176976 | |
| IPI00394994 | |
| IPI00786931 | |
| IPI00182833 | |
| IPI00023110 | |
| IPI00011253 | |
| IPI00328094 | |
| IPI00065121 | |
| IPI00057815 | |
| IPI00186439 | |
| IPI00019270 | |
| IPI00062467 | |
| IPI00374804 | |
| IPI00152975 | |
| IPI00000890 | |
| IPI00872354 | |
| IPI00655760 | |
| IPI00373867 | |
| IPI00382495 | |
| IPI00454620 | |
| IPI00847790 | |
| IPI00065351 | |
| IPI00552455 | |
| IPI00152011 | |
| IPI00299301 | |
| IPI00061009 | |
| IPI00003564 | |
| IPI00419180 | |
| IPI00030355 | |
| IPI00746459 | |
| IPI00152033 | |
| IPI00550862 | |
| IPI00175169 | |
| IPI00449923 | |
| IPI00294187 | |
| IPI00045473 | |
| IPI00220986 | |
| IPI00024776 | |
| IPI00217915 | |
| IPI00002534 | |
| IPI00102281 | |
| IPI00298058 | |
| IPI00017480 | |
| IPI00220257 | |
| IPI00170548 | |
| IPI00148768 | |
| IPI00001835 | |
| IPI00183572 | |
| IPI00167433 | |
| IPI00477693 | |
| IPI00027269 | |
| IPI00384013 | |
| IPI00063878 | |
| IPI00735313 | |
| IPI00427808 | |
| IPI00789564 | |
| IPI00029473 | |
| IPI00025310 | |
| IPI00444234 | |
| IPI00853133 | |
| IPI00005711 | |
| IPI00304324 | |
| IPI00254408 | |
| IPI00339297 | |
| IPI00024319 | |
| IPI00744927 | |
| IPI00016007 | |
| IPI00017592 | |
| IPI00556253 | |
| IPI00477820 | |
| IPI00884076 | |
| IPI00043069 | |
| IPI00292894 | |
| IPI00794779 | |
| IPI00293867 | |
| IPI00185361 | |
| IPI00185038 | |
| IPI00744357 | |
| IPI00301726 | |
| IPI00410320 | |
| IPI00017819 | |
| IPI00065085 | |
| IPI00478354 | |
| IPI00289914 | |
| IPI00745395 | |
| IPI00479625 | |
| IPI00005537 | |
| IPI00167446 | |
| IPI00012438 | |
| IPI00031630 | |
| IPI00217895 | |
| IPI00014316 | |
| IPI00412272 | |
| IPI00743545 | |
| IPI00479296 | |
| IPI00397622 | |
| IPI00300221 | |
| IPI00217418 | |
| IPI00026036 | |
| IPI00022061 | |
| IPI00647205 | |
| IPI00145121 | |
| IPI00006298 | |
| IPI00000784 | |
| IPI00030945 | |
| IPI00384546 | |
| IPI00006932 | |
| IPI00452161 | |
| IPI00020406 | |
| IPI00749062 | |
| IPI00300053 | |
| IPI00166414 | |
| IPI00031497 | |
| IPI00878369 | |
| IPI00103142 | |
| IPI00655865 | |
| IPI00845401 | |
| IPI00217979 | |
| IPI00300594 | |
| IPI00005667 | |
| IPI00002773 | |
| IPI00457114 | |
| IPI00294618 | |
| IPI00465319 | |
| IPI00297763 | |
| IPI00884022 | |
| IPI00014367 | |
| IPI00440764 | |
| IPI00387132 | |
| IPI00032958 | |
| IPI00374068 | |
| IPI00797567 | |
| IPI00337335 | |
| IPI00549844 | |
| IPI00171500 | |
| IPI00328156 | |
| IPI00103426 | |
| IPI00848180 | |
| IPI00795575 | |
| IPI00305692 | |
| IPI00159072 | |
| IPI00292953 | |
| IPI00099977 | |
| IPI00290785 | |
| IPI00045208 | |
| IPI00382492 | |
| IPI00334492 | |
| IPI00221240 | |
| IPI00015990 | |
| IPI00871452 | |
| IPI00102820 | |
| IPI00215786 | |
| IPI00168255 | |
| IPI00000861 | |
| IPI00333503 | |
| IPI00170765 | |
| IPI00299402 | |
| IPI00152879 | |
| IPI00004288 | |
| IPI00294728 | |
| IPI00005013 | |
| IPI00032680 | |
| IPI00855960 | |
| IPI00059279 | |
| IPI00479904 | |
| IPI00002344 | |
| IPI00167639 | |
| IPI00013651 | |
| IPI00827815 | |
| IPI00410666 | |
| IPI00170855 | |
| IPI00444408 | |
| IPI00152692 | |
| IPI00000106 | |
| IPI00339217 | |
| IPI00395631 | |
| IPI00044608 | |
| IPI00455397 | |
| IPI00013256 | |
| IPI00744557 | |
| IPI00013205 | |
| IPI00021751 | |
| IPI00397526 | |
| IPI00003571 | |
| IPI00640654 | |
| IPI00795736 | |
| IPI00791831 | |
| IPI00444138 | |
| IPI00019932 | |
| IPI00375325 | |
| IPI00000102 | |
| IPI00640293 | |
| IPI00235003 | |
| IPI00000656 | |
| IPI00445563 | |
| IPI00216702 | |
| IPI00219604 | |
| IPI00442208 | |
| IPI00807406 | |
| IPI00167930 | |
| IPI00470657 | |
| IPI00413868 | |
| IPI00788014 | |
| IPI00869224 | |
| IPI00297235 | |
| IPI00003421 | |
| IPI00884334 | |
| IPI00007910 | |
| IPI00083708 | |
| IPI00328715 | |
| IPI00002405 | |
| IPI00470896 | |
| IPI00385511 | |
| IPI00329488 | |
| IPI00787099 | |
| IPI00017538 | |
| IPI00000030 | |
| IPI00006561 | |
| IPI00375294 | |
| IPI00218715 | |
| IPI00290035 | |
| IPI00402280 | |
| IPI00300621 | |
| IPI00306166 | |
| IPI00025717 | |
| IPI00294241 | |
| IPI00401002 | |
| IPI00479897 | |
| IPI00607829 | |
| IPI00176210 | |
| IPI00006800 | |
| IPI00014577 | |
| IPI00218559 | |
| IPI00017640 | |
| IPI00028980 | |
| IPI00396314 | |
| IPI00023856 | |
| IPI00022629 | |
| IPI00332628 | |
| IPI00788120 | |
| IPI00296830 | |
| IPI00306127 | |
| IPI00022881 | |
| IPI00031082 | |
| IPI00023095 | |
| IPI00029184 | |
| IPI00166153 | |
| IPI00009645 | |
| IPI00795917 | |
| IPI00815976 | |
| IPI00382671 | |
| IPI00018303 | |
| IPI00216003 | |
| IPI00397015 | |
| IPI00641214 | |
| IPI00791509 | |
| IPI00289931 | |
| IPI00442601 | |
| IPI00012891 | |
| IPI00021338 | |
| IPI00016425 | |
| IPI00168877 | |
| IPI00419903 | |
| IPI00290837 | |
| IPI00006173 | |
| IPI00009213 | |
| IPI00018335 | |
| IPI00787774 | |
| IPI00397740 | |
| IPI00455083 | |
| IPI00297462 | |
| IPI00445796 | |
| IPI00303732 | |
| IPI00742114 | |
| IPI00401789 | |
| IPI00023164 | |
| IPI00299406 | |
| IPI00017726 | |
| IPI00004538 | |
| IPI00385495 | |
| IPI00515034 | |
| IPI00430792 | |
| IPI00165357 | |
| IPI00793454 | |
| IPI00028392 | |
| IPI00174347 | |
| IPI00012480 | |
| IPI00018203 | |
| IPI00030487 | |
| IPI00787443 | |
| IPI00013371 | |
| IPI00335259 | |
| IPI00414259 | |
| IPI00737735 | |
| IPI00789856 | |
| IPI00788010 | |
| IPI00400834 | |
| IPI00852699 | |
| IPI00014186 | |
| IPI00023330 | |
| IPI00645614 | |
| IPI00301144 | |
| IPI00410590 | |
| IPI00386390 | |
| IPI00066367 | |
| IPI00001753 | |
| IPI00029446 | |
| IPI00401829 | |
| IPI00651669 | |
| IPI00056324 | |
| IPI00239405 | |
| IPI00169377 | |
| IPI00244812 | |
| IPI00044761 | |
| IPI00032342 | |
| IPI00029662 | |
| IPI00027487 | |
| IPI00307536 | |
| IPI00011631 | |
| IPI00220493 | |
| IPI00168692 | |
| IPI00157237 | |
| IPI00009040 | |
| IPI00383774 | |
| IPI00419675 | |
| IPI00182774 | |
| IPI00744574 | |
| IPI00217691 | |
| IPI00470515 | |
| IPI00156871 | |
| IPI00514843 | |
| IPI00216346 | |
| IPI00644968 | |
| IPI00397759 | |
| IPI00414122 | |
| IPI00187082 | |
| IPI00867744 | |
| IPI00030648 | |
| IPI00065415 | |
| IPI00879277 | |
| IPI00376206 | |
| IPI00011589 | |
| IPI00784027 | |
| IPI00018805 | |
| IPI00013468 | |
| IPI00446685 | |
| IPI00441064 | |
| IPI00030814 | |
| IPI00745955 | |
| IPI00024523 | |
| IPI00001317 | |
| IPI00883929 | |
| IPI00032158 | |
| IPI00395667 | |
| IPI00444375 | |
| IPI00794880 | |
| IPI00419791 | |
| IPI00009305 | |
| IPI00328885 | |
| IPI00219898 | |
| IPI00216593 | |
| IPI00328089 | |
| IPI00299880 | |
| IPI00008708 | |
| IPI00796745 | |
| IPI00006178 | |
| IPI00748649 | |
| IPI00439935 | |
| IPI00100106 | |
| IPI00020354 | |
| IPI00013296 | |
| IPI00004472 | |
| IPI00300426 | |
| IPI00450358 | |
| IPI00307259 | |
| IPI00410325 | |
| IPI00465054 | |
| IPI00153060 | |
| IPI00456676 | |
| IPI00177888 | |
| IPI00101267 | |
| IPI00739927 | |
| IPI00002506 | |
| IPI00782965 | |
| IPI00872098 | |
| IPI00386113 | |
| IPI00640865 | |
| IPI00023410 | |
| IPI00787593 | |
| IPI00158506 | |
| IPI00180781 | |
| IPI00339309 | |
| IPI00100362 | |
| IPI00843923 | |
| IPI00003768 | |
| IPI00029144 | |
| IPI00030307 | |
| IPI00175439 | |
| IPI00646485 | |
| IPI00641155 | |
| IPI00023105 | |
| IPI00398162 | |
| IPI00006003 | |
| IPI00043294 | |
| IPI00152535 | |
| IPI00217507 | |
| IPI00216356 | |
| IPI00747271 | |
| IPI00290562 | |
| IPI00164776 | |
| IPI00022325 | |
| IPI00643809 | |
| IPI00166528 | |
| IPI00221325 | |
| IPI00445894 | |
| IPI00100910 | |
| IPI00084684 | |
| IPI00428677 | |
| IPI00645729 | |
| IPI00829925 | |
| IPI00003897 | |
| IPI00297559 | |
| IPI00152257 | |
| IPI00043526 | |
| IPI00412024 | |
| IPI00020565 | |
| IPI00013373 | |
| IPI00852688 | |
| IPI00218743 | |
| IPI00009333 | |
| IPI00470917 | |
| IPI00013269 | |
| IPI00787990 | |
| IPI00088953 | |
| IPI00736826 | |
| IPI00032785 | |
| IPI00552434 | |
| IPI00017800 | |
| IPI00008837 | |
| IPI00005104 | |
| IPI00735531 | |
| IPI00419912 | |
| IPI00845299 | |
| IPI00009982 | |
| IPI00060440 | |
| IPI00478572 | |
| IPI00396147 | |
| IPI00430079 | |
| IPI00141933 | |
| IPI00296942 | |
| IPI00018245 | |
| IPI00216164 | |
| IPI00328409 | |
| IPI00385153 | |
| IPI00029737 | |
| IPI00005661 | |
| IPI00217683 | |
| IPI00385321 | |
| IPI00446711 | |
| IPI00418966 | |
| IPI00465168 | |
| IPI00792370 | |
| IPI00065457 | |
| IPI00444502 | |
| IPI00001780 | |
| IPI00552840 | |
| IPI00396063 | |
| IPI00167762 | |
| IPI00166863 | |
| IPI00479578 | |
| IPI00022433 | |
| IPI00179694 | |
| IPI00027642 | |
| IPI00797832 | |
| IPI00410464 | |
| IPI00337348 | |
| IPI00401190 | |
| IPI00028213 | |
| IPI00060627 | |
| IPI00008832 | |
| IPI00014958 | |
| IPI00719669 | |
| IPI00299084 | |
| IPI00030237 | |
| IPI00025039 | |
| IPI00647897 | |
| IPI00024285 | |
| IPI00736558 | |
| IPI00470518 | |
| IPI00005087 | |
| IPI00029227 | |
| IPI00008455 | |
| IPI00006356 | |
| IPI00186554 | |
| IPI00005616 | |
| IPI00789494 | |
| IPI00745346 | |
| IPI00328291 | |
| IPI00027596 | |
| IPI00031490 | |
| IPI00218839 | |
| IPI00168977 | |
| IPI00002791 | |
| IPI00290432 | |
| IPI00012540 | |
| IPI00477396 | |
| IPI00744527 | |
| IPI00301609 | |
| IPI00783200 | |
| IPI00303207 | |
| IPI00014877 | |
| IPI00872730 | |
| IPI00165955 | |
| IPI00418336 | |
| IPI00377211 | |
| IPI00100247 | |
| IPI00217850 | |
| IPI00853149 | |
| IPI00220533 | |
| IPI00016859 | |
| IPI00746222 | |
| IPI00010843 | |
| IPI00646826 | |
| IPI00012353 | |
| IPI00219689 | |
| IPI00385065 | |
| IPI00059711 | |
| IPI00413930 | |
| IPI00006715 | |
| IPI00374074 | |
| IPI00787189 | |
| IPI00737972 | |
| IPI00215965 | |
| IPI00302458 | |
| IPI00166331 | |
| IPI00020771 | |
| IPI00170800 | |
| IPI00063181 | |
| IPI00328225 | |
| IPI00064767 | |
| IPI00784385 | |
| IPI00003893 | |
| IPI00065547 | |
| IPI00010893 | |
| IPI00294386 | |
| IPI00061680 | |
| IPI00220901 | |
| IPI00059139 | |
| IPI00031386 | |
| IPI00876962 | |
| IPI00043370 | |
| IPI00184160 | |
| IPI00298214 | |
| IPI00012411 | |
| IPI00760993 | |
| IPI00004527 | |
| IPI00018956 | |
| IPI00014232 | |
| IPI00216308 | |
| IPI00065484 | |
| IPI00444208 | |
| IPI00026305 | |
| IPI00001641 | |
| IPI00021715 | |
| IPI00216484 | |
| IPI00291840 | |
| IPI00154553 | |
| IPI00166833 | |
| IPI00016373 | |
| IPI00022479 | |
| IPI00646410 | |
| IPI00398779 | |
| IPI00451413 | |
| IPI00787410 | |
| IPI00830073 | |
| IPI00552601 | |
| IPI00384447 | |
| IPI00651633 | |
| IPI00183425 | |
| IPI00027228 | |
| IPI00007917 | |
| IPI00552787 | |
| IPI00786880 | |
| IPI00247063 | |
| IPI00787625 | |
| IPI00374223 | |
| IPI00023586 | |
| IPI00028579 | |
| IPI00385020 | |
| IPI00253323 | |
| IPI00170961 | |
| IPI00166361 | |
| IPI00787587 | |
| IPI00294810 | |
| IPI00418408 | |
| IPI00004362 | |
| IPI00168627 | |
| IPI00026952 | |
| IPI00302351 | |
| IPI00299554 | |
| IPI00307165 | |
| IPI00152722 | |
| IPI00298447 | |
| IPI00396025 | |
| IPI00293200 | |
| IPI00013789 | |
| IPI00550272 | |
| IPI00410564 | |
| IPI00152685 | |
| IPI00384140 | |
| IPI00514197 | |
| IPI00299040 | |
| IPI00295253 | |
| IPI00039626 | |
| IPI00156804 | |
| IPI00396527 | |
| IPI00183526 | |
| IPI00782960 | |
| IPI00012451 | |
| IPI00739611 | |
| IPI00384164 | |
| IPI00065253 | |
| IPI00646831 | |
| IPI00383294 | |
| IPI00103373 | |
| IPI00008632 | |
| IPI00216137 | |
| IPI00005621 | |
| IPI00478058 | |
| IPI00001933 | |
| IPI00411291 | |
| IPI00387098 | |
| IPI00883751 | |
| IPI00884216 | |
| IPI00026512 | |
| IPI00180375 | |
| IPI00152960 | |
| IPI00028066 | |
| IPI00179452 | |
| IPI00297407 | |
| IPI00296421 | |
| IPI00290155 | |
| IPI00394960 | |
| IPI00747017 | |
| IPI00793836 | |
| IPI00382422 | |
| IPI00328842 | |
| IPI00555595 | |
| IPI00016861 | |
| IPI00431697 | |
| IPI00470771 | |
| IPI00830137 | |
| IPI00031768 | |
| IPI00883997 | |
| IPI00394818 | |
| IPI00171716 | |
| IPI00016988 | |
| IPI00216986 | |
| IPI00030874 | |
| IPI00168418 | |
| IPI00021143 | |
| IPI00297931 | |
| IPI00018691 | |
| IPI00010604 | |
| IPI00022438 | |
| IPI00167764 | |
| IPI00874276 | |
| IPI00000690 | |
| IPI00829697 | |
| IPI00020903 | |
| IPI00386185 | |
| IPI00465247 | |
| IPI00382394 | |
| IPI00021979 | |
| IPI00167913 | |
| IPI00300060 | |
| IPI00829625 | |
| IPI00446767 | |
| IPI00020754 | |
| IPI00789889 | |
| IPI00455675 | |
| IPI00014852 | |
| IPI00000873 | |

TABLE 10
CSF Proteins in True Normals by IPI

| | | | | | |
|---|---|---|---|---|---|
| IPI00022434 | IPI00007257 | IPI00853525 | IPI00816741 | IPI00009920 | IPI00784950 |
| IPI00783987 | IPI00032291 | IPI00218874 | IPI00296534 | IPI00300241 | IPI00550731 |
| IPI00418163 | IPI00156171 | IPI00215894 | IPI00218725 | IPI00386879 | IPI00472249 |
| IPI00032258 | IPI00414249 | IPI00031030 | IPI00027703 | IPI00382606 | IPI00654888 |
| IPI00878517 | IPI00878576 | IPI00025276 | IPI00423463 | IPI00788835 | IPI00792626 |
| IPI00006601 | IPI00413959 | IPI00032328 | IPI00784842 | IPI00015102 | IPI00171411 |
| IPI00384697 | IPI00021842 | IPI00376427 | IPI00219029 | IPI00013976 | IPI00020996 |
| IPI00022463 | IPI00064667 | IPI00456623 | IPI00295542 | IPI00216171 | IPI00783399 |
| IPI00069058 | IPI00303210 | IPI00019943 | IPI00744835 | IPI00439446 | IPI00160552 |
| IPI00798430 | IPI00006608 | IPI00748312 | IPI00012503 | IPI00027780 | IPI00294004 |
| IPI00478003 | IPI00216728 | IPI00291866 | IPI00418262 | IPI00162735 | IPI00301865 |
| IPI00017601 | IPI00007921 | IPI00021885 | IPI00159927 | IPI00002147 | IPI00216697 |
| IPI00292071 | IPI00550991 | IPI00022395 | IPI00784810 | IPI00294395 | IPI00440577 |
| IPI00026314 | IPI00009362 | IPI00289831 | IPI00785084 | IPI00008318 | IPI00061977 |
| IPI00029751 | IPI00655702 | IPI00027310 | IPI00011229 | IPI00853454 | IPI00855725 |
| IPI00289501 | IPI00795918 | IPI00815926 | IPI00465439 | IPI00027087 | IPI00020986 |
| IPI00013179 | IPI00019591 | IPI00302641 | IPI00016334 | IPI00029863 | IPI00297646 |
| IPI00514285 | IPI00290315 | IPI00017696 | IPI00003362 | IPI00029343 | IPI00845354 |
| IPI00296777 | IPI00470535 | IPI00375547 | IPI00853369 | IPI00430842 | IPI00169383 |
| IPI00022229 | IPI00291136 | IPI00015260 | IPI00872363 | IPI00744561 | IPI00004669 |
| IPI00006114 | IPI00021841 | IPI00607580 | IPI00423466 | IPI00784998 | IPI00022426 |
| IPI00783390 | IPI00177543 | IPI00784828 | IPI00790899 | IPI00166048 | IPI00022284 |
| IPI00029739 | IPI00394655 | IPI00784807 | IPI00334282 | IPI00442294 | IPI00328609 |
| IPI00242956 | IPI00479514 | IPI00002714 | IPI00019568 | IPI00296922 | IPI00219365 |
| IPI00299059 | IPI00639937 | IPI00219798 | IPI00296165 | IPI00016915 | IPI00006662 |
| IPI00291262 | IPI00021000 | IPI00426051 | IPI00829767 | IPI00844156 | IPI00004656 |
| IPI00794184 | IPI00478809 | IPI00296537 | IPI00292950 | IPI00003351 | IPI00021891 |
| IPI00788189 | IPI00022937 | IPI00384542 | IPI00292530 | IPI00646281 | IPI00018136 |
| IPI00553177 | IPI00032179 | IPI00025465 | IPI00023673 | IPI00023845 | IPI00001662 |
| IPI00304273 | IPI00023814 | IPI00796279 | IPI00303963 | IPI00374563 | IPI00477747 |
| IPI00024284 | IPI00220741 | IPI00107831 | IPI00000828 | IPI00002280 | IPI00183445 |
| IPI00883753 | IPI00019580 | IPI00761159 | IPI00828205 | IPI00298971 | IPI00784985 |
| IPI00847179 | IPI00294193 | IPI00026944 | IPI00451625 | IPI00029260 | IPI00334667 |
| IPI00555812 | IPI00024966 | IPI00472345 | IPI00029658 | IPI00442297 | IPI00784969 |
| IPI00333776 | IPI00218192 | IPI00791343 | IPI00016150 | IPI00830132 | IPI00785067 |
| IPI00742696 | IPI00218875 | IPI00784894 | IPI00004433 | IPI00872555 | IPI00430808 |
| IPI00415032 | IPI00219042 | IPI00018219 | IPI00012545 | IPI00218733 | IPI00477597 |
| IPI00072917 | IPI00306339 | IPI00298497 | IPI00025252 | IPI00515041 | IPI00301579 |
| IPI00022488 | IPI00296608 | IPI00003802 | IPI00220117 | IPI00019157 | IPI00290085 |
| IPI00647027 | IPI00739237 | IPI00025257 | IPI00784942 | IPI00465028 | IPI00022371 |
| IPI00020557 | IPI00009997 | IPI00448925 | IPI00022895 | IPI00220562 | IPI00176221 |
| IPI00241562 | IPI00305461 | IPI00328829 | IPI00394992 | IPI00166622 | IPI00480183 |
| IPI00607600 | IPI00032293 | IPI00031121 | IPI00550558 | IPI00787050 | IPI00855824 |
| IPI00020012 | IPI00220644 | IPI00022432 | IPI00298828 | IPI00216704 | IPI00289083 |
| IPI00032220 | IPI00334238 | IPI00641737 | IPI00163207 | IPI00647704 | IPI00016422 |
| IPI00000779 | IPI00022420 | IPI00794070 | IPI00015881 | IPI00646304 | IPI00292550 |
| IPI00784830 | IPI00298793 | IPI00009802 | IPI00787781 | IPI00029693 | IPI00027497 |
| IPI00784758 | IPI00001952 | IPI00465184 | IPI00026104 | IPI00877169 | IPI00815938 |
| IPI00021854 | IPI00152540 | IPI00795013 | IPI00000816 | IPI00010949 | IPI00816555 |
| IPI00298281 | IPI00027848 | IPI00385980 | IPI00793166 | IPI00220342 | IPI00015964 |
| IPI00008787 | IPI00643034 | IPI00218046 | IPI00299738 | IPI00718977 | IPI00299299 |
| IPI00028911 | IPI00026946 | IPI00022337 | IPI00022822 | IPI00302181 | IPI00301459 |
| IPI00166729 | IPI00166766 | IPI00103175 | IPI00218413 | IPI00465325 | IPI00290856 |
| IPI00022417 | IPI00154734 | IPI00009865 | IPI00019581 | IPI00337548 | IPI00029723 |
| IPI00022431 | IPI00410210 | IPI00552905 | IPI00006967 | IPI00032532 | IPI00297487 |
| IPI00010154 | IPI00008944 | IPI00787853 | IPI00019576 | IPI00643920 | IPI00027482 |
| IPI00009028 | IPI00384952 | IPI00000130 | IPI00028413 | IPI00743766 | IPI00215983 |
| IPI00746963 | IPI00180240 | IPI00829877 | IPI00167093 | IPI00000877 | IPI00790473 |
| IPI00003590 | IPI00176458 | IPI00013698 | IPI00018236 | IPI00423461 | IPI00000137 |
| IPI00027827 | IPI00011218 | IPI00011264 | IPI00877029 | IPI00219526 | IPI00477992 |
| IPI00219217 | IPI00292946 | IPI00163563 | IPI00027166 | IPI00783665 | IPI00015756 |
| IPI00465248 | IPI00329775 | IPI00328391 | IPI00411680 | IPI00012102 | IPI00002816 |
| IPI00792115 | IPI00024046 | IPI00026216 | IPI00852979 | IPI00003921 | IPI00023014 |
| IPI00410600 | IPI00221224 | IPI00783492 | IPI00414984 | IPI00011261 | IPI00021485 |
| IPI00298237 | IPI00297124 | IPI00816626 | IPI00791228 | IPI00879084 | IPI00293088 |
| IPI00419722 | IPI00297284 | IPI00027230 | IPI00162547 | IPI00029235 | IPI00020990 |
| IPI00879665 | IPI00102543 | IPI00290283 | IPI00022608 | IPI00395488 | IPI00745660 |
| IPI00028908 | IPI00550162 | IPI00376689 | IPI00024570 | IPI00298994 | IPI00414896 |
| IPI00252731 | IPI00165949 | IPI00419724 | IPI00008994 | IPI00010471 | IPI00023019 |
| IPI00012585 | IPI00056478 | IPI00218732 | IPI00157414 | IPI00298547 | IPI00002732 |
| IPI00021304 | IPI00385264 | IPI00021263 | IPI00443799 | IPI00220642 | IPI00007221 |
| IPI00299024 | IPI00418471 | IPI00031461 | IPI00746623 | IPI00843910 | IPI00018769 |
| IPI00784773 | IPI00178302 | IPI00293836 | IPI00829711 | IPI00002211 | IPI00788786 |
| IPI00784865 | IPI00465016 | IPI00220334 | IPI00785196 | IPI00418531 | IPI00064607 |
| IPI00008603 | IPI00104074 | IPI00176427 | IPI00743302 | IPI00025426 | IPI00216691 |
| IPI00020599 | IPI00012887 | IPI00410714 | IPI00032063 | IPI00022331 | IPI00005142 |
| IPI00472961 | IPI00219446 | IPI00220327 | IPI00301395 | IPI00010796 | IPI00001611 |
| IPI00011651 | IPI00154742 | IPI00022361 | IPI00328746 | IPI00009793 | IPI00000874 |
| IPI00027851 | IPI00009950 | IPI00012989 | IPI00298388 | IPI00297263 | IPI00163446 |
| IPI00396423 | IPI00012303 | IPI00152418 | IPI00026154 | IPI00871139 | IPI00027038 |
| IPI00295741 | IPI00829626 | IPI00001593 | IPI00217966 | IPI00784156 | IPI00013682 |
| IPI00853045 | IPI00005794 | IPI00166392 | IPI00014048 | IPI00103597 | IPI00745363 |
| IPI00289819 | IPI00295832 | IPI00304925 | IPI00023728 | IPI00176193 | IPI00106646 |
| IPI00011252 | IPI00004440 | IPI00784519 | IPI00015315 | IPI00456736 | IPI00005292 |
| IPI00015688 | IPI00030255 | IPI00382938 | IPI00247243 | IPI00010381 | IPI00292150 |
| IPI00020091 | IPI00027350 | IPI00785200 | IPI00216318 | IPI00003919 | IPI00514676 |
| IPI00854806 | IPI00291005 | IPI00012440 | IPI00293748 | IPI00007236 | IPI00376394 |
| IPI00022429 | IPI00292791 | IPI00289204 | IPI00829640 | IPI00296176 | IPI00008087 |
| IPI00014572 | IPI00027192 | IPI00418446 | IPI00027493 | IPI00163187 | IPI00014592 |
| IPI00013303 | IPI00296141 | IPI00654755 | IPI00026199 | IPI00304962 | IPI00183487 |
| IPI00024621 | IPI00152847 | IPI00000138 | IPI00012283 | IPI00306322 | IPI00021856 |
| IPI00015902 | IPI00003269 | IPI00004946 | IPI00301961 | IPI00030887 | IPI00056357 |
| IPI00303071 | IPI00009904 | IPI00171473 | IPI00025846 | IPI00719621 | IPI00329801 |
| IPI00470607 | IPI00007752 | IPI00216882 | IPI00011732 | IPI00387168 | IPI00020987 |
| IPI00011454 | IPI00477804 | IPI00019399 | IPI00329482 | IPI00004413 | IPI00018274 |
| IPI00025110 | IPI00328488 | IPI00304865 | IPI00015049 | IPI00027078 | IPI00294615 |
| IPI00291175 | IPI00022810 | IPI00005908 | IPI00004503 | IPI00006971 | IPI00029236 |
| IPI00220292 | IPI00019906 | IPI00024825 | IPI00018206 | IPI00328257 | IPI00003176 |
| IPI00009123 | IPI00013475 | IPI00168479 | IPI00015911 | IPI00019502 | IPI00295386 |
| IPI00299571 | IPI00007702 | IPI00398715 | IPI00011140 | IPI00025204 | IPI00219131 |
| IPI00002406 | IPI00329352 | IPI00028448 | IPI00789954 | IPI00027462 | IPI00291006 |
| IPI00785079 | IPI00023598 | IPI00002236 | IPI00011605 | IPI00477611 | IPI00289058 |
| IPI00852577 | IPI00292304 | IPI00014964 | IPI00028553 | IPI00216319 | IPI00032311 |
| IPI00871326 | IPI00016870 | IPI00103471 | IPI00029061 | IPI00013897 | IPI00023824 |
| IPI00001477 | IPI00854743 | IPI00029046 | IPI00301143 | IPI00304840 | IPI00007797 |
| IPI00795055 | IPI00220739 | IPI00791134 | IPI00060310 | IPI00007750 | IPI00375364 |
| IPI00011654 | IPI00043756 | IPI00428511 | IPI00295414 | IPI00018534 | IPI00289870 |
| IPI00005707 | IPI00293303 | IPI00412988 | IPI00006154 | IPI00016645 | IPI00005126 |
| IPI00883772 | IPI00292732 | IPI00168884 | IPI00168866 | IPI00102435 | IPI00031564 |
| IPI00302592 | IPI00827650 | IPI00465436 | IPI00017704 | IPI00182438 | IPI00034319 |
| IPI00003865 | IPI00021552 | IPI00246058 | IPI00739827 | IPI00791479 | IPI00021857 |
| IPI00003366 | IPI00028614 | IPI00026270 | IPI00301255 | IPI00299652 | IPI00003102 |
| IPI00021033 | IPI00004962 | IPI00290328 | IPI00002525 | IPI00854709 | IPI00550363 |
| IPI00010295 | IPI00440580 | IPI00030075 | IPI00382500 | IPI00165975 | IPI00259102 |
| IPI00165438 | IPI00375205 | IPI00296099 | IPI00479116 | IPI00789847 | IPI00017257 |
| IPI00219465 | IPI00432592 | IPI00003648 | IPI00009030 | IPI00642861 | IPI00019533 |
| IPI00002745 | IPI00031769 | IPI00029605 | IPI00022892 | IPI00155729 | IPI00879309 |
| IPI00044369 | IPI00045841 | IPI00216983 | IPI00216602 | IPI00409640 | IPI00419595 |
| IPI00007778 | IPI00025864 | IPI00025818 | IPI00784258 | IPI00291807 | IPI00216138 |
| IPI00022296 | IPI00006644 | IPI00178767 | IPI00386630 | IPI00829590 | IPI00024048 |
| IPI00473011 | IPI00218493 | IPI00029273 | IPI00745251 | IPI00295399 | IPI00642632 |
| IPI00297224 | IPI00022391 | IPI00026103 | IPI00008207 | IPI00453473 | IPI00289924 |
| IPI00645206 | IPI00333140 | IPI00016679 | IPI00024601 | IPI00303894 | IPI00328243 |
| IPI00219757 | IPI00027466 | IPI00168626 | IPI00024034 | IPI00555693 | IPI00031821 |
| IPI00294705 | IPI00019038 | IPI00746388 | IPI00011994 | IPI00006166 | IPI00023751 |
| IPI00032292 | IPI00216298 | IPI00830047 | IPI00002925 | IPI00220827 | IPI00043215 |
| IPI00217493 | IPI00029193 | IPI00478483 | IPI00176424 | IPI00012075 | IPI00412987 |
| IPI00015346 | IPI00165972 | IPI00171410 | IPI00337351 | IPI00292300 | IPI00302840 |
| IPI00455667 | IPI00745313 | IPI00013569 | IPI00003907 | IPI00419966 | IPI00022333 |
| IPI00001895 | IPI00001734 | IPI00166892 | IPI00060715 | IPI00186903 | IPI00385007 |
| IPI00419585 | IPI00023648 | IPI00021903 | IPI00402157 | IPI00430291 | IPI00023807 |
| IPI00007798 | IPI00006128 | IPI00017569 | IPI00002142 | IPI00019954 | IPI00008215 |
| IPI00217146 | IPI00299503 | IPI00167215 | IPI00299086 | IPI00328113 | IPI00007240 |
| IPI00784119 | IPI00022792 | IPI00789234 | IPI00029275 | IPI00015351 | IPI00292218 |
| IPI00008290 | IPI00328550 | IPI00183321 | IPI00374065 | IPI00032288 | IPI00007199 |
| IPI00019190 | IPI00787265 | IPI00007102 | IPI00218474 | IPI00021833 | IPI00018396 |
| IPI00018305 | IPI00020906 | IPI00643348 | IPI00101608 | IPI00218539 | IPI00465315 |
| IPI00029606 | IPI00740545 | IPI00741710 | IPI00216457 | IPI00300838 | IPI00026259 |
| IPI00384391 | IPI00010348 | IPI00168520 | IPI00747849 | IPI00006657 | IPI00022394 |
| IPI00026050 | IPI00294834 | IPI00153049 | IPI00549330 | IPI00033560 | IPI00024035 |
| IPI00031510 | IPI00829947 | IPI00432525 | IPI00007425 | IPI00025809 | IPI00022392 |
| IPI00016862 | IPI00165044 | IPI00152850 | IPI00020984 | IPI00026197 | IPI00299150 |
| IPI00021794 | IPI00021275 | IPI00217882 | IPI00554786 | IPI00023576 | IPI00004114 |
| IPI00021447 | IPI00478892 | IPI00297188 | IPI00019359 | IPI00215767 | IPI00010896 |
| IPI00008780 | IPI00556287 | IPI00099670 | IPI00384404 | IPI00024094 | IPI00024273 |
| IPI00296197 | IPI00854841 | IPI00219930 | IPI00218834 | IPI00827510 | IPI00020977 |
| IPI00009276 | IPI00736885 | IPI00387115 | IPI00017841 | IPI00829663 | IPI00012011 |
| IPI00009145 | IPI00016467 | IPI00024129 | IPI00784154 | IPI00883855 | IPI00028931 |
| IPI00005474 | IPI00827892 | IPI00299699 | IPI00006451 | IPI00456589 | IPI00328703 |
| IPI00030205 | IPI00479997 | IPI00166339 | IPI00025476 | IPI00024036 | IPI00030739 |
| IPI00027377 | IPI00790775 | IPI00000824 | IPI00827940 | IPI00419720 | IPI00031549 |
| IPI00029997 | IPI00736860 | IPI00075248 | IPI00829752 | IPI00387118 | IPI00293849 |
| IPI00329685 | IPI00017968 | IPI00880120 | IPI00749328 | IPI00291395 | IPI00385252 |
| IPI00218570 | IPI00867665 | IPI00442911 | IPI00871227 | IPI00022367 | IPI00789477 |
| IPI00306710 | IPI00181174 | IPI00008494 | IPI00180707 | IPI00002307 | IPI00219575 |
| IPI00431738 | IPI00414467 | IPI00023858 | IPI00005652 | IPI00001399 | IPI00387120 |
| IPI00307592 | IPI00010790 | IPI00027223 | IPI00009111 | IPI00006713 | IPI00011662 |
| IPI00410122 | IPI00021983 | IPI00410585 | IPI00219301 | IPI00827482 | IPI00026991 |
| IPI00550115 | IPI00044743 | IPI00299724 | IPI00007709 | IPI00008107 | IPI00030111 |
| IPI00020672 | IPI00005981 | IPI00167710 | IPI00019209 | IPI00783024 | IPI00013162 |
| IPI00027174 | IPI00006482 | IPI00218414 | IPI00001506 | IPI00162329 | IPI00017745 |
| IPI00829701 | IPI00026174 | IPI00465255 | IPI00854667 | IPI00217345 | IPI00220361 |
| IPI00006803 | IPI00744692 | IPI00182944 | IPI00827560 | IPI00023643 | IPI00374914 |
| IPI00107886 | IPI00794450 | IPI00103871 | IPI00007249 | IPI00470484 | IPI00024105 |
| IPI00171928 | IPI00401283 | IPI00293128 | IPI00031534 | IPI00428967 | IPI00470625 |
| IPI00304331 | IPI00014439 | IPI00217236 | IPI00217466 | IPI00455739 | IPI00307276 |
| IPI00607655 | IPI00025840 | IPI00383732 | IPI00643115 | IPI00171412 | IPI00383032 |
| IPI00816799 | IPI00296058 | IPI00414717 | IPI00217759 | IPI00182138 | IPI00219425 |
| IPI00328745 | IPI00011302 | IPI00219684 | IPI00218345 | IPI00016666 | IPI00012269 |
| IPI00011730 | IPI00021855 | IPI00027341 | IPI00478890 | IPI00029131 | IPI00788824 |
| IPI00783689 | IPI00178926 | IPI00008223 | IPI00011518 | IPI00293723 | IPI00022959 |
| IPI00026237 | IPI00015525 | IPI00414909 | IPI00783184 | IPI00002334 | IPI00477714 |
| IPI00386133 | IPI00296992 | IPI00021817 | IPI00251507 | IPI00478414 | IPI00019449 |
| IPI00296558 | IPI00302944 | IPI00437751 | IPI00019501 | IPI00019176 | IPI00010182 |
| IPI00413912 | IPI00784430 | IPI00017567 | IPI00432723 | IPI00382481 | IPI00292496 |
| IPI00063048 | IPI00305380 | IPI00219025 | IPI00644472 | IPI00022977 | IPI00297655 |
| IPI00003021 | IPI00029699 | IPI00010706 | IPI00217465 | IPI00643667 | IPI00028015 |
| IPI00027721 | IPI00001893 | IPI00257508 | IPI00012058 | IPI00219219 | IPI00385253 |
| IPI00783287 | IPI00012386 | IPI00293925 | IPI00217467 | IPI00293757 | IPI00179851 |
| IPI00013096 | IPI00413451 | IPI00031789 | IPI00847670 | IPI00011094 | IPI00021347 |
| IPI00029928 | IPI00008556 | IPI00013701 | IPI00165125 | IPI00289862 | IPI00099650 |
| IPI00401264 | IPI00027463 | IPI00784368 | IPI00012048 | IPI00013945 | IPI00008756 |
| IPI00015842 | IPI00297252 | IPI00884389 | IPI00418960 | IPI00465261 | IPI00008164 |
| IPI00005222 | IPI00303476 | IPI00854644 | IPI00168847 | IPI00215997 | IPI00002511 |
| IPI00440932 | IPI00410487 | IPI00016621 | IPI00168921 | IPI00382476 | IPI00009901 |
| IPI00184019 | IPI00025447 | IPI00387116 | IPI00184851 | IPI00024307 | IPI00009477 |
| IPI00414676 | IPI00029168 | IPI00384392 | IPI00027507 | IPI00294713 | IPI00012792 |
| IPI00030871 | IPI00299145 | IPI00827876 | IPI00081836 | IPI00554752 | IPI00412492 |
| IPI00022674 | IPI00005123 | IPI00018941 | IPI00397645 | IPI00306844 | IPI00178352 |
| IPI00000044 | IPI00297181 | IPI00883765 | IPI00220748 | IPI00384016 | IPI00413587 |
| IPI00003799 | IPI00387119 | IPI00748265 | IPI00019771 | IPI00477868 | IPI00017334 |
| IPI00374732 | IPI00030882 | IPI00006556 | IPI00016014 | IPI00022640 | IPI00044600 |
| IPI00289876 | IPI00884080 | IPI00374590 | IPI00301180 | IPI00644346 | IPI00012007 |
| IPI00300725 | IPI00411656 | IPI00018860 | IPI00005038 | IPI00027847 | IPI00441498 |
| IPI00018381 | IPI00375879 | IPI00556391 | IPI00010402 | IPI00009867 | IPI00004457 |
| IPI00028082 | IPI00032050 | IPI00883711 | IPI00303161 | IPI00002966 | IPI00382493 |
| IPI00009826 | IPI00026285 | IPI00441344 | IPI00022389 | IPI00029756 | IPI00337612 |
| IPI00220301 | IPI00402293 | IPI00019862 | IPI00419442 | IPI00007853 | IPI00024466 |
| IPI00006130 | IPI00384402 | IPI00019600 | IPI00004573 | IPI00465322 | IPI00012009 |
| IPI00157454 | IPI00829740 | IPI00827829 | IPI00257882 | IPI00103755 | IPI00217963 |
| IPI00062037 | IPI00006444 | IPI00829841 | IPI00293276 | IPI00550533 | IPI00382488 |
| IPI00019755 | IPI00024929 | IPI00031506 | IPI00025812 | IPI00002283 | IPI00022445 |
| IPI00220362 | IPI00020747 | IPI00293539 | IPI00291488 | IPI00419565 | IPI00006510 |
| IPI00739099 | IPI00829812 | IPI00010470 | IPI00382478 | IPI00029623 | IPI00000914 |
| IPI00220766 | IPI00293971 | IPI00011899 | IPI00007047 | IPI00171199 | IPI00299116 |
| IPI00552852 | IPI00884092 | IPI00024587 | IPI00413641 | IPI00149097 | IPI00400935 |
| IPI00107731 | IPI00022891 | IPI00005516 | IPI00027444 | IPI00021834 | IPI00783156 |
| IPI00293464 | IPI00783393 | IPI00012510 | IPI00299083 | IPI00001610 | IPI00176581 |
| IPI00018146 | IPI00176398 | IPI00217376 | IPI00299547 | IPI00001592 | IPI00023191 |
| IPI00029817 | IPI00017562 | IPI00553138 | IPI00170814 | IPI00382682 | IPI00217005 |
| IPI00219129 | IPI00010148 | IPI00020396 | IPI00009890 | IPI00061354 | IPI00219682 |
| IPI00184094 | IPI00883879 | IPI00000832 | IPI00219468 | IPI00009054 | IPI00029700 |
| IPI00219910 | IPI00299399 | IPI00760721 | IPI00413344 | IPI00297779 | IPI00024919 |
| IPI00398918 | IPI00022649 | IPI00465377 | IPI00008586 | IPI00168920 | IPI00022039 |
| IPI00552267 | IPI00001633 | IPI00828105 | IPI00006524 | IPI00215980 | IPI00004047 |
| IPI00031708 | IPI00171438 | IPI00743194 | IPI00026800 | IPI00100154 | IPI00387106 |
| IPI00022774 | IPI00376131 | IPI00387024 | IPI00216461 | IPI00021997 | IPI00305975 |
| IPI00026240 | IPI00005517 | IPI00444605 | IPI00735451 | IPI00418169 | IPI00440493 |
| IPI00005722 | IPI00827581 | IPI00304577 | IPI00075013 | IPI00012315 | IPI00419237 |
| IPI00169285 | IPI00827637 | IPI00301364 | IPI00006009 | IPI00552943 | IPI00024067 |
| IPI00396378 | IPI00298650 | IPI00030847 | IPI00329332 | IPI00008726 | IPI00218795 |
| IPI00299758 | IPI00873863 | IPI00008533 | IPI00384400 | IPI00021727 | IPI00382482 |
| IPI00007960 | IPI00000760 | IPI00657742 | IPI00033466 | IPI00411706 | IPI00292657 |
| IPI00387117 | IPI00102575 | IPI00218667 | IPI00289926 | IPI00027972 | IPI00216694 |
| IPI00005809 | IPI00472754 | IPI00008997 | IPI00016371 | IPI00383951 | IPI00384395 |
| IPI00023754 | IPI00432766 | IPI00219067 | IPI00021091 | IPI00013933 | IPI00216914 |
| IPI00019904 | IPI00300623 | IPI00410079 | IPI00021695 | IPI00024248 | IPI00399252 |
| IPI00829827 | IPI00011865 | IPI00827929 | IPI00413781 | IPI00028714 | IPI00144243 |
| IPI00219806 | IPI00015479 | IPI00019812 | IPI00480159 | IPI00063827 | IPI00186004 |
| IPI00873344 | IPI00294619 | IPI00010343 | IPI00005690 | IPI00215899 | IPI00000070 |
| IPI00552939 | IPI00003441 | IPI00023942 | IPI00289746 | IPI00412264 | IPI00255145 |
| IPI00827939 | IPI00043810 | IPI00305833 | IPI00295767 | IPI00024887 | IPI00552874 |
| IPI00065312 | IPI00024976 | IPI00550746 | IPI00296441 | IPI00005837 | IPI00010737 |
| IPI00152524 | IPI00550949 | IPI00003470 | IPI00827906 | IPI00024138 | IPI00168806 |
| IPI00182194 | IPI00006034 | IPI00028387 | IPI00383887 | IPI00335946 | IPI00302850 |
| IPI00165936 | IPI00297180 | IPI00009396 | IPI00385985 | IPI00807609 | IPI00013681 |
| IPI00783818 | IPI00003527 | IPI00828156 | IPI00064652 | IPI00021900 | IPI00019208 |
| IPI00242905 | IPI00061520 | IPI00181743 | IPI00024572 | IPI00026358 | IPI00025019 |
| IPI00022890 | IPI00219420 | IPI00426727 | IPI00384407 | IPI00030877 | IPI00013508 |
| IPI00026546 | IPI00005774 | IPI00552937 | IPI00030431 | IPI00000265 | IPI00470766 |
| IPI00293460 | IPI00879950 | IPI00787936 | IPI00220706 | IPI00386575 | IPI00023359 |
| IPI00032227 | IPI00004494 | IPI00031907 | IPI00028193 | IPI00000190 | IPI00031696 |
| IPI00064377 | IPI00396383 | IPI00018879 | IPI00000775 | IPI00790122 | IPI00038378 |
| IPI00816775 | IPI00454695 | IPI00399089 | IPI00221117 | IPI00026125 | IPI00029819 |
| IPI00298476 | IPI00031718 | IPI00387097 | IPI00646689 | IPI00002818 | IPI00003933 |
| IPI00830035 | IPI00021119 | IPI00007812 | IPI00218319 | IPI00296259 | IPI00184997 |
| IPI00002320 | IPI00004488 | IPI00170692 | IPI00004367 | IPI00642645 | IPI00004409 |
| IPI00166613 | IPI00514517 | IPI00854707 | IPI00029591 | IPI00386131 | IPI00556643 |
| IPI00001120 | IPI00009439 | IPI00788258 | IPI00552578 | IPI00013290 | IPI00000792 |
| IPI00382442 | IPI00008504 | IPI00410675 | IPI00301288 | IPI00154858 | IPI00218130 |
| IPI00008202 | IPI00789259 | IPI00045536 | IPI00000160 | IPI00000811 | IPI00292393 |
| IPI00024580 | IPI00017557 | IPI00019180 | IPI00032405 | IPI00005719 | IPI00221332 |
| IPI00328431 | IPI00014223 | IPI00008085 | IPI00012948 | IPI00217264 | IPI00046057 |
| IPI00829834 | IPI00216699 | IPI00329538 | IPI00307612 | IPI00218628 | IPI00021364 |
| IPI00306576 | IPI00044707 | IPI00030634 | IPI00025992 | IPI00217405 | IPI00061507 |
| IPI00854624 | IPI00216348 | IPI00550677 | IPI00027875 | IPI00554521 | IPI00382499 |
| IPI00003391 | IPI00176104 | IPI00029050 | IPI00010442 | IPI00456670 | IPI00386754 |
| IPI00178727 | IPI00328826 | IPI00328520 | IPI00387113 | IPI00002535 | IPI00737969 |
| IPI00383680 | IPI00151036 | IPI00749514 | IPI00031411 | IPI00006900 | IPI00442121 |
| IPI00000871 | IPI00020407 | IPI00220281 | IPI00738499 | IPI00291922 | IPI00186826 |
| IPI00181079 | IPI00289802 | IPI00000087 | IPI00797310 | IPI00031131 | IPI00013219 |
| IPI00020884 | IPI00328587 | IPI00010810 | IPI00853073 | IPI00017163 | IPI00010207 |
| IPI00008148 | IPI00387105 | IPI00301812 | IPI00303318 | IPI00026665 | IPI00737429 |
| IPI00018246 | IPI00396961 | IPI00027239 | IPI00025318 | IPI00003406 | IPI00004480 |
| IPI00827839 | IPI00024689 | IPI00847652 | IPI00027547 | IPI00020008 | IPI00412216 |
| IPI00640292 | IPI00741608 | IPI00250724 | IPI00413778 | IPI00387110 | IPI00005969 |
| IPI00396930 | IPI00414294 | IPI00024704 | IPI00022314 | IPI00376087 | IPI00060308 |
| IPI00829810 | IPI00019907 | IPI00646291 | IPI00645194 | IPI00008282 | IPI00387101 |
| IPI00001872 | IPI00328680 | IPI00021199 | IPI00013299 | IPI00007277 | IPI00218075 |
| IPI00175989 | IPI00026031 | IPI00020692 | IPI00743963 | IPI00179589 | IPI00002790 |
| IPI00221034 | IPI00216049 | IPI00289861 | IPI00301058 | IPI00243995 | IPI00879575 |
| IPI00019372 | IPI00003111 | IPI00855918 | IPI00853516 | IPI00171196 | IPI00744825 |
| IPI00215979 | IPI00003031 | IPI00032313 | IPI00384051 | IPI00021327 | IPI00005675 |
| IPI00216963 | IPI00009532 | IPI00027626 | IPI00006146 | IPI00023505 | IPI00217989 |
| IPI00248596 | IPI00643937 | IPI00001960 | IPI00150881 | IPI00015988 | IPI00829836 |
| IPI00012391 | IPI00030876 | IPI00027009 | IPI00024346 | IPI00021812 | IPI00185661 |
| IPI00296337 | IPI00031627 | IPI00005132 | IPI00296913 | IPI00219077 | IPI00217258 |
| IPI00299147 | IPI00737920 | IPI00215746 | IPI00018342 | IPI00004084 | IPI00307729 |
| IPI00435925 | IPI00218914 | IPI00216780 | IPI00166075 | IPI00642259 | IPI00604430 |
| IPI00008274 | IPI00456578 | IPI00385918 | IPI00029107 | IPI00789398 | IPI00186621 |
| IPI00410588 | IPI00013302 | IPI00218820 | IPI00867509 | IPI00032826 | IPI00295098 |
| IPI00004534 | IPI00333197 | IPI00383603 | IPI00217871 | IPI00152326 | IPI00071824 |
| IPI00554474 | IPI00004346 | IPI00219622 | IPI00552771 | IPI00044751 | IPI00747494 |
| IPI00479669 | IPI00218131 | IPI00031086 | IPI00410488 | IPI00009943 | IPI00302962 |
| IPI00017940 | IPI00440153 | IPI00022462 | IPI00382474 | IPI00166807 | IPI00879409 |
| IPI00400986 | IPI00014516 | IPI00016112 | IPI00215610 | IPI00032187 | IPI00005153 |
| IPI00011643 | IPI00297208 | IPI00010193 | IPI00015148 | IPI00420014 | IPI00169259 |
| IPI00748955 | IPI00025084 | IPI00021048 | IPI00020431 | IPI00386314 | IPI00106506 |
| IPI00217781 | IPI00014899 | IPI00149375 | IPI00410657 | IPI00024107 | IPI00472332 |
| IPI00031008 | IPI00218398 | IPI00015913 | IPI00552591 | IPI00160369 | IPI00789245 |
| IPI00103552 | IPI00013978 | IPI00383016 | IPI00335168 | IPI00002478 | IPI00012044 |
| IPI00443909 | IPI00014398 | IPI00382486 | IPI00002491 | IPI00027438 | IPI00290744 |
| IPI00024853 | IPI00874156 | IPI00004533 | IPI00005159 | IPI00033030 | IPI00152145 |
| IPI00101927 | IPI00004373 | IPI00020019 | IPI00647217 | IPI00017964 | IPI00021770 |
| IPI00470838 | IPI00827522 | IPI00026241 | IPI00017529 | IPI00163724 | IPI00465045 |
| IPI00008580 | IPI00007010 | IPI00010863 | IPI00024664 | IPI00478640 | IPI00018803 |
| IPI00025869 | IPI00002412 | IPI00218918 | IPI00004798 | IPI00383953 | IPI00828083 |
| IPI00005531 | IPI00299778 | IPI00295577 | IPI00012877 | IPI00011051 | IPI00827580 |
| IPI00783313 | IPI00451429 | IPI00029372 | IPI00478816 | IPI00830051 | IPI00021594 |
| IPI00009335 | IPI00218407 | IPI00220271 | IPI00017256 | IPI00550720 | IPI00010360 |
| IPI00387095 | IPI00010369 | IPI00306853 | IPI00023217 | IPI00103510 | IPI00028381 |
| IPI00013860 | IPI00219005 | IPI00019530 | IPI00477616 | IPI00373872 | IPI00465044 |
| IPI00377045 | IPI00013004 | IPI00456683 | IPI00479722 | IPI00295172 | IPI00013847 |
| IPI00008433 | IPI00289334 | IPI00005614 | IPI00006470 | IPI00328522 | IPI00180386 |
| IPI00219525 | IPI00021907 | IPI00021831 | IPI00021753 | IPI00251596 | IPI00001793 |
| IPI00020501 | IPI00008303 | IPI00007682 | IPI00027984 | IPI00550906 | IPI00167638 |
| IPI00442865 | IPI00064935 | IPI00024502 | IPI00221255 | IPI00550263 | IPI00827891 |
| IPI00414320 | IPI00300407 | IPI00298738 | IPI00300207 | IPI00045219 | IPI00218292 |
| IPI00827584 | IPI00061448 | IPI00335541 | IPI00171678 | IPI00179473 | IPI00384861 |
| IPI00295618 | IPI00550364 | IPI00444331 | IPI00018909 | IPI00221235 | IPI00018980 |
| IPI00010133 | IPI00028450 | IPI00028600 | IPI00163851 | IPI00167560 | IPI00830044 |
| IPI00013991 | IPI00218465 | IPI00456635 | IPI00014340 | IPI00098902 | IPI00024012 |
| IPI00005705 | IPI00217740 | IPI00221178 | IPI00297550 | IPI00000959 | IPI00478860 |
| IPI00303882 | IPI00872550 | IPI00000977 | IPI00299263 | IPI00289271 | IPI00552735 |
| IPI00154528 | IPI00382421 | IPI00290854 | IPI00014537 | IPI00216470 | IPI00000104 |
| IPI00296219 | IPI00306959 | IPI00021274 | IPI00028932 | IPI00011400 | IPI00448465 |
| IPI00005564 | IPI00816737 | IPI00394820 | IPI00018708 | IPI00299485 | IPI00745122 |
| IPI00014850 | IPI00013466 | IPI00419253 | IPI00011578 | IPI00060146 | IPI00783471 |
| IPI00794679 | IPI00016605 | IPI00029012 | IPI00387159 | IPI00329688 | IPI00023542 |
| IPI00294519 | IPI00872861 | IPI00043201 | IPI00645089 | IPI00022989 | IPI00383832 |
| IPI00744811 | IPI00298285 | IPI00385042 | IPI00167619 | IPI00152072 | |
| IPI00031485 | IPI00166619 | IPI00410013 | IPI00374862 | IPI00060546 | IPI00027685 |
| IPI00784044 | IPI00029722 | IPI00402144 | IPI00009377 | IPI00412541 | IPI00432755 |
| IPI00514893 | IPI00005128 | IPI00478986 | IPI00300244 | IPI00043978 | IPI00796777 |
| IPI00429191 | IPI00796647 | IPI00478521 | IPI00333410 | IPI00440221 | IPI00008894 |
| IPI00514594 | IPI00852758 | IPI00420071 | IPI00018311 | IPI00011564 | IPI00847759 |
| IPI00853376 | IPI00023087 | IPI00555614 | IPI00168862 | IPI00423683 | IPI00008315 |
| IPI00400967 | IPI00297444 | IPI00026530 | IPI00025647 | IPI00026612 | IPI00031005 |
| IPI00166010 | IPI00161119 | IPI00025700 | IPI00027429 | IPI00167137 | IPI00787414 |
| IPI00793576 | IPI00016577 | IPI00016949 | IPI00013281 | IPI00155447 | IPI00019988 |
| IPI00301631 | IPI00852633 | IPI00297040 | IPI00252845 | IPI00001434 | IPI00059164 |
| IPI00396077 | IPI00163391 | IPI00454910 | IPI00171611 | IPI00854745 | IPI00175654 |
| IPI00465178 | IPI00299679 | IPI00031547 | IPI00477361 | IPI00043731 | IPI00873740 |
| IPI00291463 | IPI00797699 | IPI00384722 | IPI00397578 | IPI00645814 | IPI00032416 |
| IPI00007040 | IPI00333126 | IPI00382756 | IPI00031019 | IPI00003353 | IPI00335589 |
| IPI00220578 | IPI00794119 | IPI00604599 | IPI00017659 | IPI00007402 | IPI00843819 |
| IPI00740191 | IPI00816274 | IPI00743284 | IPI00744706 | IPI00152470 | IPI00065276 |
| IPI00044607 | IPI00032830 | IPI00021363 | IPI00025094 | IPI00006094 | IPI00644522 |
| IPI00301923 | IPI00171737 | IPI00749440 | IPI00830057 | IPI00169426 | IPI00045511 |
| IPI00059395 | IPI00385003 | IPI00216683 | IPI00023315 | IPI00816794 | IPI00604763 |
| IPI00030319 | IPI00029123 | IPI00015522 | IPI00010895 | IPI00010346 | IPI00829759 |
| IPI00018275 | IPI00218487 | IPI00877084 | IPI00739106 | IPI00013216 | IPI00235647 |
| IPI00025363 | IPI00158992 | IPI00060265 | IPI00022606 | IPI00216572 | IPI00294910 |
| IPI00015285 | IPI00394712 | IPI00024802 | IPI00550232 | IPI00644025 | IPI00217652 |
| IPI00554799 | IPI00001869 | IPI00374039 | IPI00295502 | IPI00171647 | IPI00470805 |
| IPI00478124 | IPI00027834 | IPI00302133 | IPI00216106 | IPI00020058 | IPI00167089 |
| IPI00304596 | IPI00062730 | IPI00028786 | IPI00010118 | IPI00026570 | IPI00385143 |
| IPI00012895 | IPI00298258 | IPI00167254 | IPI00300990 | IPI00027726 | IPI00783753 |
| IPI00299627 | IPI00018352 | IPI00013455 | IPI00741780 | IPI00064241 | IPI00445716 |
| IPI00023513 | IPI00744366 | IPI00026299 | IPI00454858 | IPI00295503 | IPI00030919 |
| IPI00394870 | IPI00008905 | IPI00845508 | IPI00399328 | IPI00307591 | IPI00827745 |
| IPI00166817 | IPI00377077 | IPI00745300 | IPI00479083 | IPI00290292 | IPI00005776 |
| IPI00375174 | IPI00009294 | IPI00009148 | IPI00168885 | IPI00003348 | IPI00394879 |
| IPI00296866 | IPI00186581 | IPI00787932 | IPI00010575 | IPI00299435 | IPI00401852 |
| IPI00040730 | IPI00445364 | IPI00166161 | IPI00785015 | IPI00029533 | IPI00306884 |
| IPI00829980 | IPI00021985 | IPI00180426 | IPI00106502 | IPI00815786 | IPI00748891 |
| IPI00028561 | IPI00166071 | IPI00382420 | IPI00830025 | IPI00375881 | IPI00874023 |
| IPI00743898 | IPI00217023 | IPI00166553 | IPI00187143 | IPI00008998 | IPI00020199 |
| IPI00644231 | IPI00455521 | IPI00004315 | IPI00103630 | IPI00658112 | IPI00297714 |
| IPI00007193 | IPI00034006 | IPI00796906 | IPI00216508 | IPI00479361 | IPI00005158 |
| IPI00791513 | IPI00220791 | IPI00166039 | IPI00169331 | IPI00015980 | IPI00018098 |
| IPI00013319 | IPI00015983 | IPI00009899 | IPI00783464 | IPI00830122 | IPI00243338 |
| IPI00828037 | IPI00289965 | IPI00005129 | IPI00298702 | IPI00641251 | IPI00786937 |
| IPI00020201 | IPI00374129 | IPI00217948 | IPI00185088 | IPI00103853 | IPI00027744 |
| IPI00217537 | IPI00007617 | IPI00027457 | IPI00010405 | IPI00301465 | IPI00718806 |
| IPI00002884 | IPI00297288 | IPI00002993 | IPI00873774 | IPI00102678 | IPI00024032 |
| IPI00025092 | IPI00748890 | IPI00555600 | IPI00847335 | IPI00020356 | IPI00744226 |
| IPI00003363 | IPI00376587 | IPI00419221 | IPI00877800 | IPI00008226 | IPI00792229 |
| IPI00022078 | IPI00297251 | IPI00021634 | IPI00008497 | IPI00010903 | IPI00000144 |
| IPI00290857 | IPI00438170 | IPI00384225 | IPI00010808 | IPI00735934 | IPI00786893 |
| IPI00164949 | IPI00373823 | IPI00375803 | IPI00827485 | IPI00465234 | IPI00015954 |
| IPI00029647 | IPI00550917 | IPI00023184 | IPI00514622 | IPI00010303 | IPI00099838 |
| IPI00414481 | IPI00301294 | IPI00872739 | IPI00009203 | IPI00008091 | IPI00397949 |
| IPI00640818 | IPI00021951 | IPI00241409 | IPI00790021 | IPI00329791 | IPI00418735 |
| IPI00164861 | IPI00815893 | IPI00090764 | IPI00000027 | IPI00152344 | IPI00306413 |
| IPI00411674 | IPI00002243 | IPI00141938 | IPI00033600 | IPI00465363 | IPI00018755 |
| IPI00025365 | IPI00159049 | IPI00024253 | IPI00375746 | IPI00028601 | IPI00385543 |
| IPI00020131 | IPI00297277 | IPI00387096 | IPI00180384 | IPI00011781 | IPI00299076 |
| IPI00828191 | IPI00031765 | IPI00218637 | IPI00176920 | IPI00419908 | IPI00386393 |
| IPI00249982 | IPI00016685 | IPI00216921 | IPI00045839 | IPI00479125 | IPI00878511 |
| IPI00025473 | IPI00002191 | IPI00011416 | IPI00008404 | IPI00455852 | IPI00816155 |
| IPI00328270 | IPI00007928 | IPI00260755 | IPI00658025 | IPI00174976 | IPI00442564 |
| IPI00432707 | IPI00022542 | IPI00337385 | IPI00022958 | IPI00550876 | IPI00412408 |
| IPI00442544 | IPI00028053 | IPI00304527 | IPI00006987 | IPI00795481 | IPI00008422 |
| IPI00175019 | IPI00005732 | IPI00000459 | IPI00023162 | IPI00064296 | IPI00746177 |
| IPI00027898 | IPI00293095 | IPI00030741 | IPI00019146 | IPI00002459 | IPI00550792 |
| IPI00019485 | IPI00747142 | IPI00399296 | IPI00789181 | IPI00025311 | IPI00029175 |
| IPI00023340 | IPI00040900 | IPI00233358 | IPI00742725 | IPI00216651 | IPI00045360 |
| IPI00300052 | IPI00011515 | IPI00879842 | IPI00878755 | IPI00216774 | IPI00001755 |
| IPI00383594 | IPI00164066 | IPI00021476 | IPI00152050 | IPI00792945 | IPI00749171 |
| IPI00019158 | IPI00307702 | IPI00028383 | IPI00385480 | IPI00027464 | IPI00033419 |
| IPI00005605 | IPI00307611 | IPI00001863 | IPI00465123 | IPI00293530 | IPI00170635 |
| IPI00045928 | IPI00014444 | IPI00298337 | IPI00013495 | IPI00399180 | IPI00830018 |
| IPI00306332 | IPI00479217 | IPI00044842 | IPI00398992 | IPI00007512 | IPI00026230 |
| IPI00015047 | IPI00183206 | IPI00022055 | IPI00827724 | IPI00016576 | IPI00009365 |
| IPI00001786 | IPI00220070 | IPI00328260 | IPI00012441 | IPI00290308 | IPI00445278 |
| IPI00745103 | IPI00023322 | IPI00807418 | IPI00006054 | IPI00005859 | IPI00644840 |
| IPI00827846 | IPI00784739 | IPI00183002 | IPI00008438 | IPI00025622 | IPI00301098 |
| IPI00385791 | IPI00738920 | IPI00163601 | IPI00152849 | IPI00019901 | IPI00009771 |
| IPI00145805 | IPI00056314 | IPI00428741 | IPI00029556 | IPI00102808 | IPI00444842 |
| IPI00296374 | IPI00797694 | IPI00006252 | IPI00217012 | IPI00073763 | IPI00023152 |
| IPI00021733 | IPI00294210 | IPI00007321 | IPI00218730 | IPI00018208 | IPI00296727 |
| IPI00184884 | IPI00787020 | IPI00217617 | IPI00215914 | IPI00166865 | IPI00005347 |
| IPI00103874 | IPI00167941 | IPI00306850 | IPI00290826 | IPI00477468 | IPI00470913 |
| IPI00243221 | IPI00016701 | IPI00024662 | IPI00553092 | IPI00005668 | IPI00023407 |
| IPI00306046 | IPI00884353 | IPI00217831 | IPI00657936 | IPI00434711 | |
| IPI00445315 | IPI00852725 | IPI00030037 | IPI00329593 | IPI00009881 | |
| IPI00065931 | IPI00005600 | IPI00013749 | IPI00296461 | IPI00020329 | |
| IPI00149044 | IPI00791593 | IPI00645078 | IPI00376383 | IPI00243451 | |
| IPI00007834 | IPI00293396 | IPI00395866 | IPI00015973 | IPI00184650 | |
| IPI00003971 | IPI00018914 | IPI00240793 | IPI00304789 | IPI00328298 | |
| IPI00746681 | IPI00185146 | IPI00376237 | IPI00792759 | IPI00386284 | |
| IPI00383808 | IPI00143753 | IPI00847723 | IPI00446588 | IPI00289329 | |
| IPI00871556 | IPI00217791 | IPI00169115 | IPI00419836 | IPI00018429 | |
| IPI00746666 | IPI00167006 | IPI00020966 | IPI00217435 | IPI00032904 | |
| IPI00003814 | IPI00028481 | IPI00456827 | IPI00021766 | IPI00424119 | |
| IPI00245940 | IPI00294215 | IPI00747657 | IPI00221006 | IPI00387004 | |
| IPI00657699 | IPI00027806 | IPI00215777 | IPI00290358 | IPI00218946 | |
| IPI00290094 | IPI00071185 | IPI00028912 | IPI00013272 | IPI00024766 | |
| IPI00413826 | IPI00166776 | IPI00003807 | IPI00009070 | IPI00009471 | |
| IPI00030706 | IPI00853400 | IPI00830107 | IPI00386576 | IPI00455967 | |
| IPI00410093 | IPI00473033 | IPI00019242 | IPI00022295 | IPI00186966 | |
| IPI00168404 | IPI00749245 | IPI00786946 | IPI00747420 | IPI00045939 | |
| IPI00644766 | IPI00152182 | IPI00166257 | IPI00432226 | IPI00165009 | |
| IPI00167154 | IPI00783604 | IPI00011592 | IPI00171874 | IPI00374670 | |
| IPI00871533 | IPI00456969 | IPI00442745 | IPI00165229 | IPI00289837 | |
| IPI00877615 | IPI00001796 | IPI00374301 | IPI00006746 | | |
| IPI00030352 | IPI00549972 | IPI00024920 | IPI00178894 | | |
| IPI00221080 | IPI00604551 | IPI00026262 | IPI00878436 | | |
| IPI00032338 | IPI00470468 | IPI00018843 | IPI00640810 | | |
| IPI00293679 | IPI00382515 | IPI00027782 | IPI00845229 | | |
| IPI00152769 | IPI00853312 | IPI00030009 | IPI00306549 | | |
| IPI00748682 | IPI00644191 | IPI00177878 | IPI00003392 | | |
| IPI00019888 | IPI00470490 | IPI00104907 | IPI00293361 | | |
| IPI00291987 | IPI00001433 | IPI00827978 | IPI00827788 | | |
| IPI00170766 | IPI00220156 | IPI00005107 | IPI00253281 | | |
| IPI00032597 | IPI00024818 | IPI00152216 | IPI00005607 | | |
| IPI00783855 | IPI00335437 | IPI00028520 | IPI00014371 | | |
| IPI00746987 | IPI00000076 | IPI00027264 | IPI00329605 | | |
| IPI00295469 | IPI00290078 | IPI00878962 | IPI00032425 | | |
| IPI00044326 | IPI00100980 | IPI00103891 | IPI00300020 | | |
| IPI00884004 | IPI00328361 | IPI00328709 | IPI00001568 | | |
| IPI00478997 | IPI00022277 | IPI00398154 | IPI00020470 | | |
| IPI00456599 | IPI00216288 | IPI00291939 | IPI00718821 | | |
| IPI00607831 | IPI00025499 | IPI00444272 | IPI00018387 | | |
| IPI00399254 | IPI00030757 | IPI00641181 | IPI00398229 | | |
| IPI00171230 | IPI00787083 | IPI00418125 | IPI00027248 | | |
| IPI00719505 | IPI00856012 | IPI00151990 | IPI00438286 | | |
| IPI00784880 | IPI00001712 | IPI00024272 | IPI00023780 | | |
| IPI00741005 | IPI00216592 | IPI00003384 | IPI00018027 | | |

1. A method for diagnosing multiple sclerosis in a patient, the method comprising:
a) isolating a biological sample from the patient; and
b) analyzing the biological sample for expression of a panel of biomarkers, wherein the panel of biomarkers is set forth in any one of Tables 1m, 3m, 4H-m or 2m, to determine if at least two of the biomarkers listed in any one of Tables 1m, 3m, 4H-m or Table 2m are in the biological sample, wherein identification of at least two of the biomarkers in the biological sample indicates a positive diagnosis of multiple sclerosis in the patient.

2. The method of claim 1, wherein the at least two biomarkers identified in the biological sample are present in the biological sample and set forth in Table 1m, 3m, or 4H-m, and the presence of the at least two biomarkers indicates a positive diagnosis of multiple sclerosis.

3. The method of claim 1, wherein the at least two biomarkers identified in the biological sample are absent from the biological sample and set forth in Table 2m and the absence of the at least two biomarkers indicates a positive diagnosis of multiple sclerosis.

4. A method for differentially diagnosing a neuropsychiatric disease in a patient, wherein the patient exhibits clinical symptoms common to both Neurologic Lyme disease and Chronic Fatigue Syndrome, the method comprising:
a) isolating a biological sample from the patient; and
b) analyzing the biological sample for expression of a first panel of biomarkers, wherein the first panel of biomarkers is set forth in any one of Tables 4H-1, 7L, or 8L, to determine if at least two of the biomarkers listed in any one of Tables 4H-1, 7L, or 8L are identified in the biological sample, wherein identification of at least two of the biomarkers in the biological sample indicates a positive diagnosis of Neurologic Lyme disease.

5. The method of claim 4, wherein the at least two biomarkers identified in the biological sample are present in the biological sample and set forth in Table 4H-1 or 7L and the presence of the at least two biomarkers indicates a positive diagnosis of Neurologic Lyme disease.

6. The method of claim 4, wherein the at least two biomarkers identified in the biological sample are absent from the biological sample and set forth in Table 8L and the absence of the at least two biomarkers indicates a positive diagnosis of Neurologic Lyme disease.

7. The method of claim 4, further comprising analyzing the biological sample for expression of a second panel of biomarkers, wherein the second panel of biomarkers is set forth in any one of Tables 4H-c, 5c, or 6c, to determine if at least two of the biomarkers listed in any one of Tables 4H-c, 5c, or 6c are identified in the biological sample, wherein identification of at least two of the biomarkers in the biological sample indicates a positive diagnosis of Chronic Fatigue Syndrome.

8. The method of claim 7, wherein the at least two biomarkers identified in the biological sample are present in the biological sample and set forth in Table 4H-c or 5c and the presence of the at least two biomarkers indicates a positive diagnosis of Chronic Fatigue Syndrome.

9. The method of claim 7, wherein the at least two biomarkers identified in the biological sample are absent from the biological sample and set forth in Table 6c and the absence of the at least two biomarkers indicates a positive diagnosis of Chronic Fatigue Syndrome.

10. The method of claim 1, wherein the biological sample is cerebrospinal fluid, blood or a component thereof, tissue or tissue-related fluids, urine, or saliva.

11. A method for diagnosing a neuropsychiatric disease in a patient, wherein the patient exhibits clinical symptoms common to both Chronic Fatigue Syndrome and Neurologic Lyme disease, the method comprising:
a) isolating a biological sample from the patient;
b) analyzing the biological sample to evaluate expression levels of the biomarkers listed in Table 9 to determine a first level of at least two of the biomarkers indicative of the presence of the neuropsychiatric disease in the biological sample; and
c) comparing the first level of the at least two of the biomarkers to a second level of the at least two of the biomarkers determined for a subject, wherein the subject does not have the neuropsychiatric disease, and wherein at least a two-fold difference in the ratio of the first level to the second level indicates a positive diagnosis of the neuropsychiatric disease.

12. The method of claim 11, wherein the neuropsychiatric disease is Chronic Fatigue Syndrome or Neurologic Lyme disease.

13. The method of claim 11, wherein the at least a two-fold difference is at least a two-fold increase or at least a two-fold decrease.

14. The method of claim 11, wherein the subject is a healthy subject.

15-28. (canceled)

29. The method of claim 4, wherein the biological sample is cerebrospinal fluid, blood or a component thereof, tissue or tissue-related fluids, urine, or saliva.

30. The method of claim 11, wherein the biological sample is cerebrospinal fluid, blood or a component thereof, tissue or tissue-related fluids, urine, or saliva.
